(19) United States
(12) Patent Application Publication
(10) Pub. No.: US 2024/0184200 A1
(43) Pub. Date: Jun. 6, 2024

(54) AMINE COMPOUND, CHEMICALLY AMPLIFIED RESIST COMPOSITION AND PATTERNING PROCESS

(71) Applicant: Shin-Etsu Chemical Co., Ltd., Tokyo (JP)

(72) Inventors: Tomomi Watanabe, Joetsu-shi (JP); Masahiro Fukushima, Joetsu-shi (JP)

(73) Assignee: Shin-Etsu Chemical Co., Ltd., Tokyo (JP)

(21) Appl. No.: 18/383,101

(22) Filed: Oct. 24, 2023

(30) Foreign Application Priority Data
Nov. 8, 2022 (JP) 2022-178631

(51) Int. Cl. G03F 7/004; C07C 219/14; C07D 295/088; C08F 212/14; C08F 220/18; C08F 220/28; G03F 7/038; G03F 7/039

(52) U.S. Cl. CPC G03F 7/0045; C07C 219/14; C07D 295/088; C08F 212/24; C08F 220/1806; C08F 220/1808; C08F 220/1809; C08F 220/1818; C08F 220/283; G03F 7/0382; G03F 7/0392; C07C 2601/08; C07C 2601/14

(57) ABSTRACT

A chemically amplified resist composition comprising a quencher in the form of an amine compound of specific structure is provided. The resist composition has a high sensitivity and forms a pattern with improved LWR or CDU, independent of whether it is of positive or negative tone.

### CROSS-REFERENCE TO RELATED APPLICATIONS

This non-provisional application claims priority under 35 U.S.C. § 119(a) on Patent Application No. 2022-178631 filed in Japan on Nov. 8, 2022, the entire contents of which are hereby incorporated by reference.

### TECHNICAL FIELD

This invention relates to an amine compound, a chemically amplified resist composition, and a pattern forming process.

### BACKGROUND ART

To meet the demand for higher integration density and operating speed of LSIs, the effort to reduce the pattern rule is in rapid progress. In particular, the enlargement of the logic memory market to comply with the wide-spread use of smart phones drives forward the miniaturization technology. As the advanced miniaturization technology, manufacturing of microelectronic devices at the 10-nm node by double patterning of the ArF immersion lithography has been implemented in a mass scale. Manufacturing of 7-nm node devices as the next generation by the double patterning technology is approaching to the verge of high-volume application. The candidate for 5-nm node devices as the next generation but one is EUV lithography.

With the progress of miniaturization in logic devices, the flash memory now takes the form of devices having stacked layers of gate, known as 3D-NAND. The capacity is increased by increasing the number of stacked layers. As the number of stacked layers increases, the hard mask used in processing of layers becomes thicker and the photoresist film also becomes thicker. While the resist film for logic devices becomes thinner, the resist film for 3D-NAND becomes thicker.

As the pattern feature size is reduced, approaching to the diffraction limit of light, light contrast lowers. In the case of positive resist film, a lowering of light contrast leads to reductions of resolution and focus margin of hole and trench patterns. The thickening of resist film means return to the thickness of resist film for previous devices of the old generation. While a further improvement in dimensional uniformity (CDU) is demanded, previous resist compositions cannot accommodate the demand. For the purpose of preventing the resolution of resist pattern from lowering by a lowering of light contrast associated with size reduction, or improving CDU despite the thickening of resist film, an attempt is made to enhance the dissolution contrast of resist film.

Chemically amplified resist compositions comprising an acid generator capable of generating an acid upon exposure to light or EB include chemically amplified positive resist compositions wherein deprotection reaction takes place under the action of acid and chemically amplified negative resist compositions wherein polarity switch or crosslinking reaction takes place under the action of acid. Quenchers (or acid diffusion controlling agents) are often added to these resist compositions for the purpose of controlling the diffusion of the acid to unexposed region to improve the contrast. The addition of quenchers is fully effective to this purpose. A number of amine quenchers were proposed as disclosed in Patent Documents 1 and 2.

With respect to the acid labile group used in (meth)acrylate polymers for the ArF lithography resist material, deprotection reaction takes place when a photoacid generator capable of generating a sulfonic acid having fluorine substituted at α-position (referred to “α-fluorinated sulfonic acid”) is used, but not when an acid generator capable of generating a sulfonic acid not having fluorine substituted at α-position (referred to “α-non-fluorinated sulfonic acid”) or carboxylic acid is used. If a sulfonium or iodonium salt capable of generating an α-fluorinated sulfonic acid is combined with a sulfonium or iodonium salt capable of generating an α-non-fluorinated sulfonic acid, the sulfonium or iodonium salt capable of generating an α-non-fluorinated sulfonic acid undergoes ion exchange with the α-fluorinated sulfonic acid. Through the ion exchange, the α-fluorinated sulfonic acid thus generated by fight exposure is converted back to the sulfonium or iodonium salt while the sulfonium or iodonium salt of an α-non-fluorinated sulfonic acid or carboxylic acid functions as a quencher. Patent Document 3 discloses a resist composition comprising a sulfonium or iodonium salt capable of generating a carboxylic acid as a quencher.

Like photoacid generators, the quenchers of sulfonium or iodonium salt type are photo-decomposable. This means that the amount of quencher in the exposed region is reduced. Since acid is generated in the exposed region, the concentration of acid becomes relatively high as the amount of quencher is reduced. This leads to a contrast enhancement. However, the acid diffusion in the exposed region cannot be suppressed, indicating a difficulty of acid diffusion control. It is also pointed out that the quencher tends to agglomerate to reduce the CDU of resist patterns.

Since a sulfonium or iodonium salt type quencher absorbs ArF radiation of wavelength 193 nm, a resist film in which the quencher is combined with a sulfonium or iodonium salt type acid generator has a reduced transmittance to that radiation. As a result, in the case of a resist film having a thickness of at least 100 nm, the cross-sectional profile of a pattern as developed becomes tapered. For resist films having a thickness of at least 100 nm, especially at least 150 nm, a highly transparent quencher is necessary.

Lowering the PEB temperature is effective for suppressing acid diffusion. However, the dissolution contrast is accordingly reduced, inviting degradations of resolution and edge roughness (LWR). There is the need for a resist composition of new concept featuring controlled acid diffusion and a high contrast. It is also necessary to improve the dimensional uniformity (CDU) of a resist pattern as developed, by preventing the quencher in a resist film from agglomerating for thereby achieving a uniform distribution of the quencher.

There are known amine quenchers for inviting a polarity switch under the action of acid catalyst. Patent Documents 4 and 5 disclose an amine quencher having an acid labile group. This amine compound generates a carboxylic acid via the acid-aided deprotection reaction of a tertiary ester having a carbonyl group positioned on the nitrogen atom side whereby alkaline solubility increases. In this case, however, since the molecular weight on the nitrogen atom side is not increased, the acid diffusion controlling ability is low, and the contrast improving effect is weak. Patent Document 6 describes a quencher having a tert-butoxycarbonyl group which undergoes deprotection reaction with the aid of acid, to generate an amino group. This mechanism is adapted to generate a quencher upon light exposure, achieving a reverse effect to contrast enhancement. The contrast is enhanced by the mechanism that the quencher disappears or loses its quenching ability upon light exposure or under the action of acid. Patent Document 7 discloses a quencher in the form of an amine compound which cyclizes under the action of acid to form a lactam structure. The conversion of the strong base amine compound to the weak base lactam compound causes the acid to change its activity whereby the contrast is improved. Although it is confirmed that certain performance improvements are achieved by applying these amine quenchers, such improvements are still insufficient for the control of acid diffusion. It is desired to have a quencher having a further improved acid diffusion control ability.

### CITATION LIST

Patent Document 1: JP 3751518
Patent Document 2: JP 4320520
Patent Document 3: WO 2008/066011
Patent Document 4: JP 4044741
Patent Document 5: JP-A 2012-008550
Patent Document 6: JP 3790649
Patent Document 7: JP 5617799

### SUMMARY OF THE INVENTION

For the acid-catalyzed chemically amplified resist material, it is desired to develop a quencher capable of reducing the LWR of line patterns or improving the CDU of hole patterns and increasing sensitivity. To this end, it is necessary to reduce the distance of acid diffusion significantly and to increase the contrast at the same time, that is, to improve ambivalent properties at the same time.

An object of the invention is to provide a chemically amplified resist composition which exhibits a high sensitivity and a reduced LWR or improved CDU, independent of whether it is of positive tone or negative tone, a quencher for use in the resist composition, and a pattern forming process using the resist composition.

It is desired to have an amine quencher affording a high sensitivity, fully controlled acid diffusion, solvent solubility, and pattern collapse resistance.

The inventors have found that using an amine compound of specific structure as the quencher, a chemically amplified resist composition having improved properties including reduced LWR, improved CDU, high contrast, high resolution, wide process margin, prevention of swell during development, and precise micropatterning is obtainable.

In one aspect, the invention provides an amine compound having the formula (1).

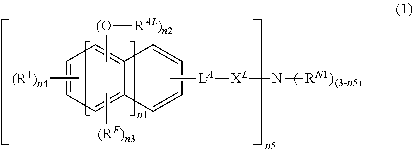

Herein n1 is an integer of 0 or 1, n2 is an integer of 1 to 3, n3 is an integer of 1 to 4, n4 is an integer of 0 to 4, with the proviso that n2+n3+n4≤5 in case of n1=0, and n2+n3+n4≤7 in case of n1=1, n5 is an integer of 1 to 3.
R^{AL} forms an acid labile group with the neighboring oxygen atom,
R^{F} is fluorine, a C₁-C₆ fluorinated saturated hydrocarbyl group, C₁-C₆ fluorinated saturated hydrocarbyloxy group, or C₁-C₆ fluorinated saturated hydrocarbylthio group, a plurality of R^{F} may be the same or different in case of n3≥2,
R^{F} and —O—R^{AL} are attached to adjacent carbon atoms,
R¹ is a C₁-C₂₀ hydrocarbyl group which may contain a heteroatom,
L^{A} is a single bond, ether bond, ester bond, sulfonic ester bond, carbonate bond or carbamate bond,
X^{L} is a single bond or a C₁-C₄₀ hydrocarbylene group which may contain a heteroatom,
R^{N1} is hydrogen or a C₁-C₂₀ hydrocarbyl group in which some or all of the hydrogen atoms may be substituted by halogen and some constituent —CH₂— may be replaced by —O— or —C(═O)—, and in case of n5=1, two R^{N1} may bond together to form a ring with the nitrogen atom to which they are attached, the ring optionally containing —O— or —S—, excluding that two R^{N1} are hydrogen at the same time.

In a preferred embodiment, R^{AL} is a group having the formula (AL-1) or (AL-2).

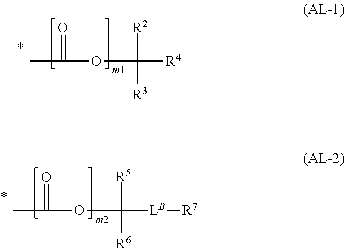

Herein R², R³ and R⁴ are each independently a C₁-C₁₂ hydrocarbyl group in which some constituent —CH₂— may be replaced by —O— or —S—, when the hydrocarbyl group contains an aromatic ring, some or all of the hydrogen atoms on the aromatic ring may be substituted by halogen, cyano, nitro, optionally halogenated C₁-C₄ alkyl moiety, or optionally halogenated C₁-C₄ alkoxy moiety, R² and R³ may bond together to form a ring with the carbon atom to which they are attached, some constituent —CH₂— in the ring may be replaced by —O— or —S—,
R⁵ and R⁶ are each independently hydrogen or a C₁-C₁₀ hydrocarbyl group, R⁷ is a C₁-C₂₀ hydrocarbyl group in which some constituent —CH₂— may be replaced by —O— or —S—, R⁶ and R⁷ may bond together to form a C₃-C₂₀ heterocyclic group with the carbon atom and L^{B} to which they are attached, some constituent —CH₂— in the heterocyclic group may be replaced by —O— or —S—,
L^{B} is —O— or —S—,
m1 is 0 or 1, m2 is 0 or 1,
* designates a point of attachment to the neighboring —O—.

The preferred amine compound has the formula (1A).

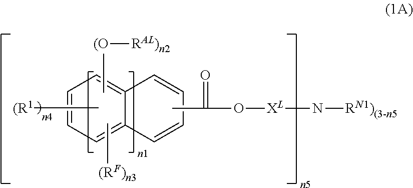

Herein R^{AL}, R^{F}, R¹, R^{N1}, X^{L} and n1 to n5 are as defined above.

The more preferred amine compound has the formula (1B).

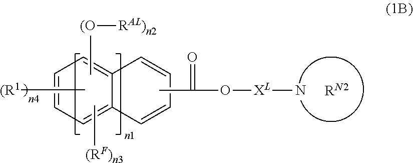

Herein R^{AL}, R^{F}, R¹, X^{L} and n1 to n4 are as defined above,
the ring R^{N2} forms a C₃-C₂₀ alicyclic hydrocarbon group with the nitrogen atom in the formula, some constituent —CH₂— in the ring R^{N2} may be replaced by —O— or —S—.

In another aspect, the invention provides a chemically amplified resist composition comprising a quencher in the form of the amine compound defined herein.

In a preferred embodiment, the resist composition further comprises a base polymer comprising repeat units having the formula (a1) or (a2).

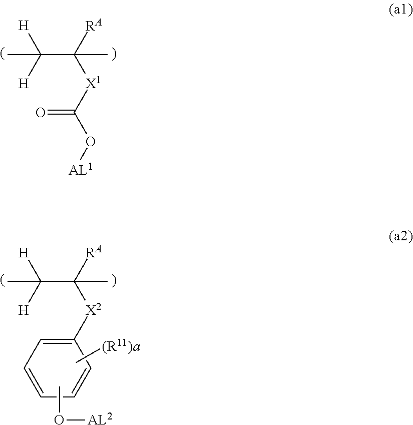

Herein R^{A} is each independently hydrogen, fluorine, methyl or trifluoromethyl,
X¹ is a single bond, phenylene, naphthylene, or *—C(═O)—O—X¹¹—, the phenylene or naphthylene group may be substituted with an optionally fluorinated C₁-C₁₀ alkoxy moiety or halogen, X¹¹ is a C₁-C₁₀ saturated hydrocarbylene group which may contain a hydroxy moiety, ether bond, ester bond or lactone ring, or phenylene group or naphthylene group,
X² is a single bond or *—C(═O)—O—,
the asterisk (*) designates a point of attachment to the carbon atom in the backbone,
R¹¹ is a C₁-C₂₀ hydrocarbyl group which may contain a heteroatom,
AL¹ and AL² are each independently an acid labile group, and
a is an integer of 0 to 4.

In a preferred embodiment, the base polymer further comprises repeat units having the formula (b1) or (b2).

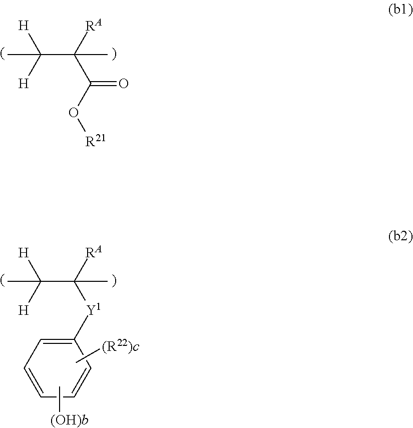

Herein R^{A} is each independently hydrogen, fluorine, methyl or trifluoromethyl,
Y¹ is a single bond or *—C(═O)—O—, the asterisk (*) designates a point of attachment to the carbon atom in the backbone,
R²¹ is hydrogen or a C₁-C₂₀ group containing at least one structure selected from a hydroxy moiety other than phenolic hydroxy, cyano moiety, carbonyl moiety, carboxy moiety, ether bond, ester bond, sulfonic ester bond, carbonate bond, lactone ring, sultone ring, and carboxylic anhydride (—C(═O)—O—C(═O)—),
R²² is a C₁-C₂₀ hydrocarbyl group which may contain a heteroatom,
b is an integer of 1 to 4, c is an integer of 0 to 4, and 1≤b+c≤5.

In a more preferred embodiment, the base polymer further comprises repeat units of at least one type selected from repeat units having the formulae (c1) to (c4).

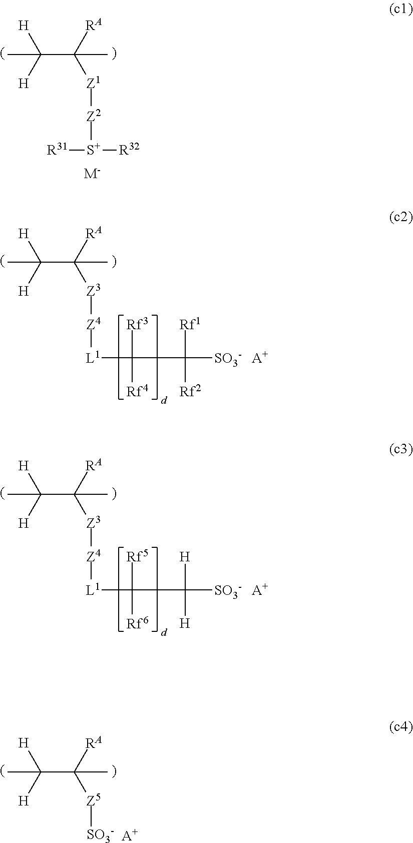

Herein R^{A} is each independently hydrogen, fluorine, methyl or trifluoromethyl,
Z¹ is a single bond or phenylene group,
Z² is *—C(═O)—O—Z²¹—, —C(═O)—NH—Z²¹— or *—O—Z²¹—, Z²¹ is a C₁-C₆ aliphatic hydrocarbylene group, phenylene group or a divalent group obtained by combining the foregoing, which may contain a carbonyl moiety, ester bond, ether bond or hydroxy moiety,
Z³ is a single bond, phenylene group, naphthylene group or *—C(═O)—O—Z³¹—, Z³¹ is a C₁-C₁₀ aliphatic hydrocarbylene group which may contain a hydroxy moiety, ether bond, ester bond or lactone ring, phenylene group or naphthylene group,
Z⁴ is a single bond or **—Z⁴¹—C(═O)—O—, Z⁴¹ is a C₁-C₂₀ hydrocarbylene group which may contain a heteroatom,
Z⁵ is a single bond, methylene group, ethylene group, phenylene group, fluorinated phenylene group, trifluoromethyl-substituted phenylene group, *—C(═O)—O—, Z⁵¹—, *—C(═O)—N(H)—Z⁵¹—, or *—O—Z⁵¹—, Z⁵¹ is a C₁-C₆ aliphatic hydrocarbylene group, phenylene group, fluorinated phenylene group, trifluoromethyl-substituted phenylene group, which may contain a carbonyl moiety, ester bond, ether bond or hydroxy moiety,
* designates a point of attachment to the carbon atom in the backbone, ** designates a point of attachment to Z³,
R³¹ and R³² are each independently a C₁-C₂₀ hydrocarbyl group which may contain a heteroatom, R³¹ and R³² may bond together to form a ring with the sulfur atom to which they are attached,
L¹ is a single bond, ether bond, ester bond, carbonyl group, sulfonic ester bond, carbonate bond or carbamate bond,
Rf¹ and Rf² are each independently fluorine or a C₁-C₆ fluorinated saturated hydrocarbyl group,
Rf³ and Rf⁴ are each independently hydrogen, fluorine or a C₁-C₆ fluorinated saturated hydrocarbyl group,
Rf⁵ and Rf⁶ are each independently hydrogen, fluorine or a C₁-C₆ fluorinated saturated hydrocarbyl group, excluding that all Rf⁵ and Rf⁶ are hydrogen at the same tune,
M⁻ is a non-nucleophilic counter ion,
A⁺ is an onium cation, and
d is an integer of 0 to 3.

The resist composition may further comprise an organic solvent, a photoacid generator, a quencher other than the amine compound having formula (1), and/or a surfactant.

In a further aspect, the invention provides a pattern forming process comprising the steps of applying the chemically amplified resist composition defined herein onto a substrate to form a resist film thereon, exposing the resist film to high-energy radiation, and developing the exposed resist film in a developer.

Typically, the high-energy radiation is KrF excimer laser radiation, ArF excimer laser radiation, EB or EUV of wavelength 3 to 15 nm.

### Advantageous Effects of Invention

The amine compound of the invention effectively functions as a quencher in a resist composition and offers a high sensitivity. Since the compound has an acid labile group, the compound in the exposed region is decomposed with the acid to bring a polarity switch whereby the dissolution contrast is enhanced. As a result, a pattern profile having unproved LWR or CDU, rectangularity, and high resolution is constructed. Additionally, the swell of a resist pattern during alkaline development is restrained, and a pattern which is resistant to collapse is formed. The amine compound is best suited for precise micropatterning.

### BRIEF DESCRIPTION OF THE DRAWINGS

FIG. 1 is a diagram showing the ¹H-NMR (DMSO-d₆) spectrum of Intermediate In-1 in Example 1-1.

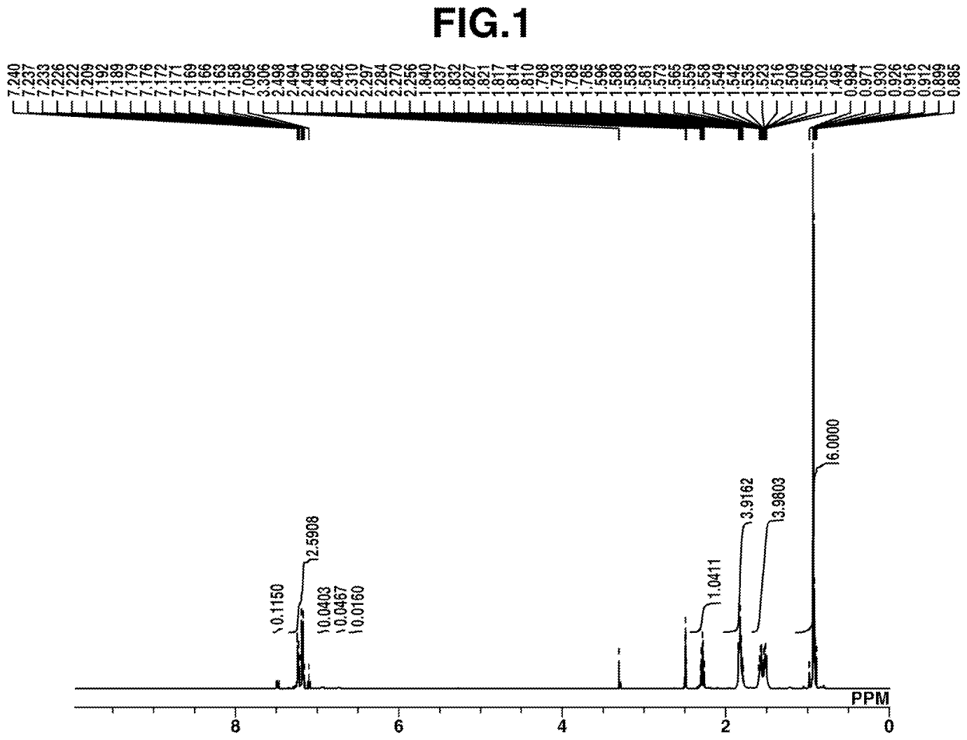

FIG. 2 is a diagram showing the ¹⁹F-NMR (DMSO-d₆) spectrum of Intermediate In-1 in Example 1-1.

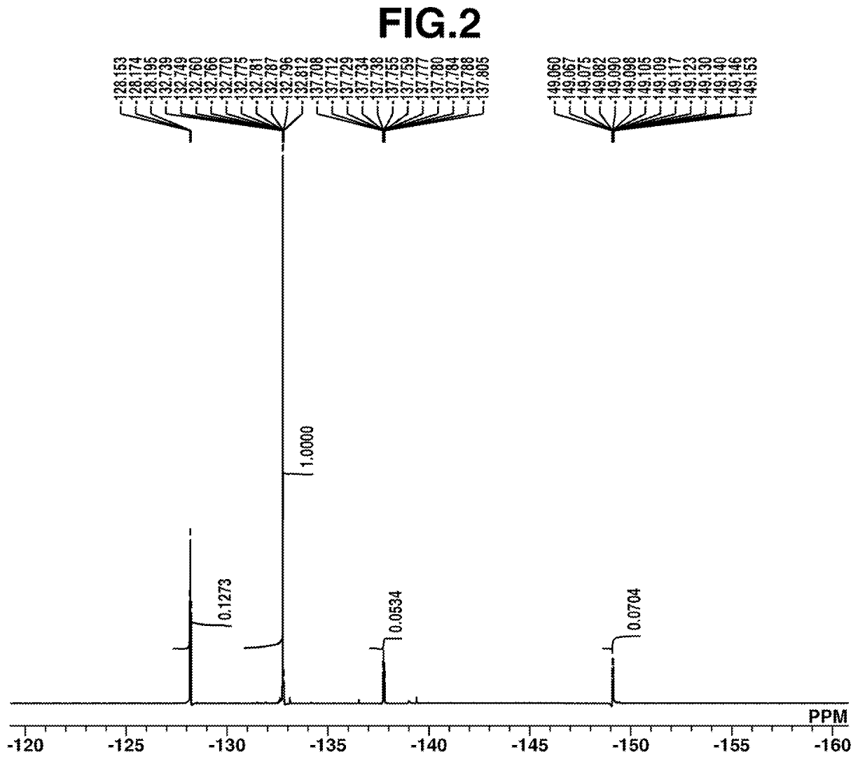

FIG. 3 is a diagram showing the ¹H-NMR (DMSO-d₆) spectrum of Intermediate In-2 in Example 1-1.

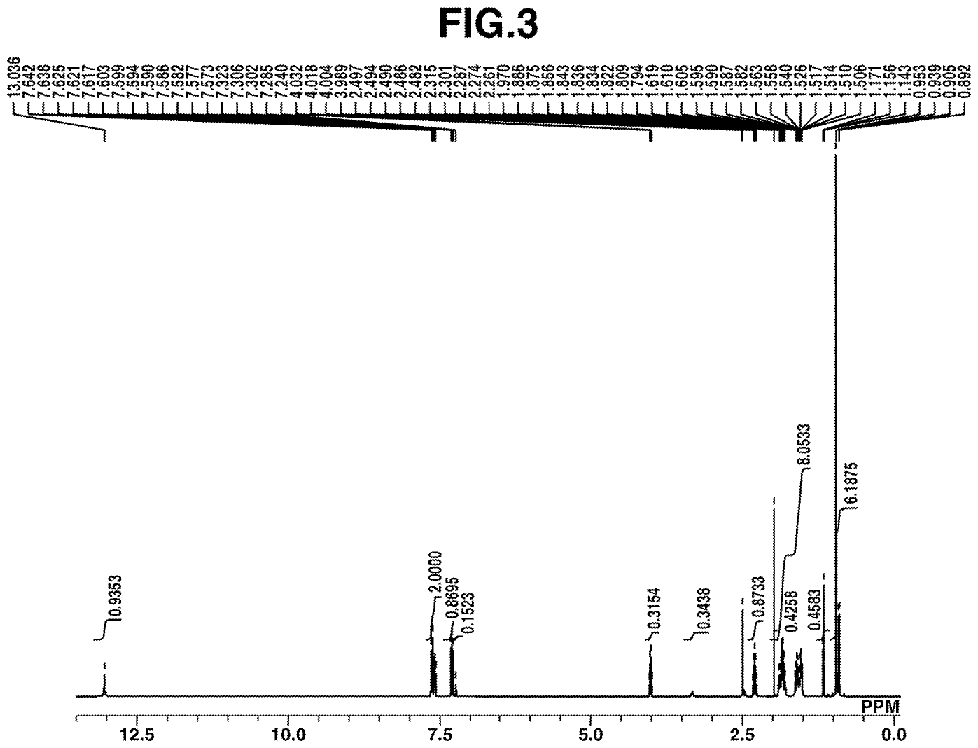

FIG. 4 is a diagram showing the ¹⁹F NMR (DMSO-d₆) spectrum of Intermediate In-2 in Example 1-1.

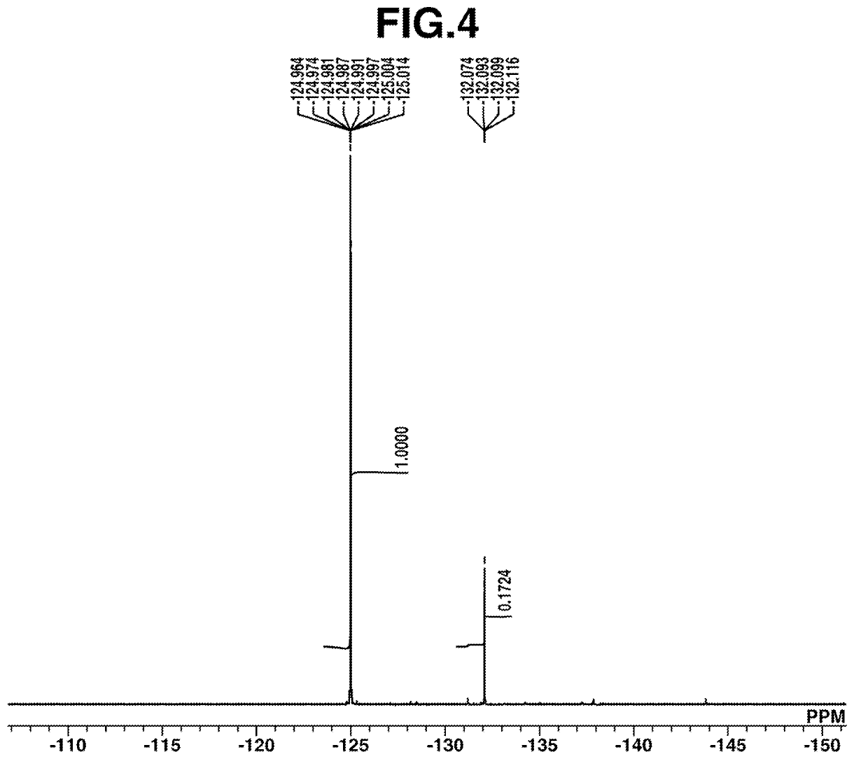

FIG. 5 is a diagram showing the ¹H-NMR (DMSO-d₆) spectrum of amine compound AQ-1 in Example 1-1.

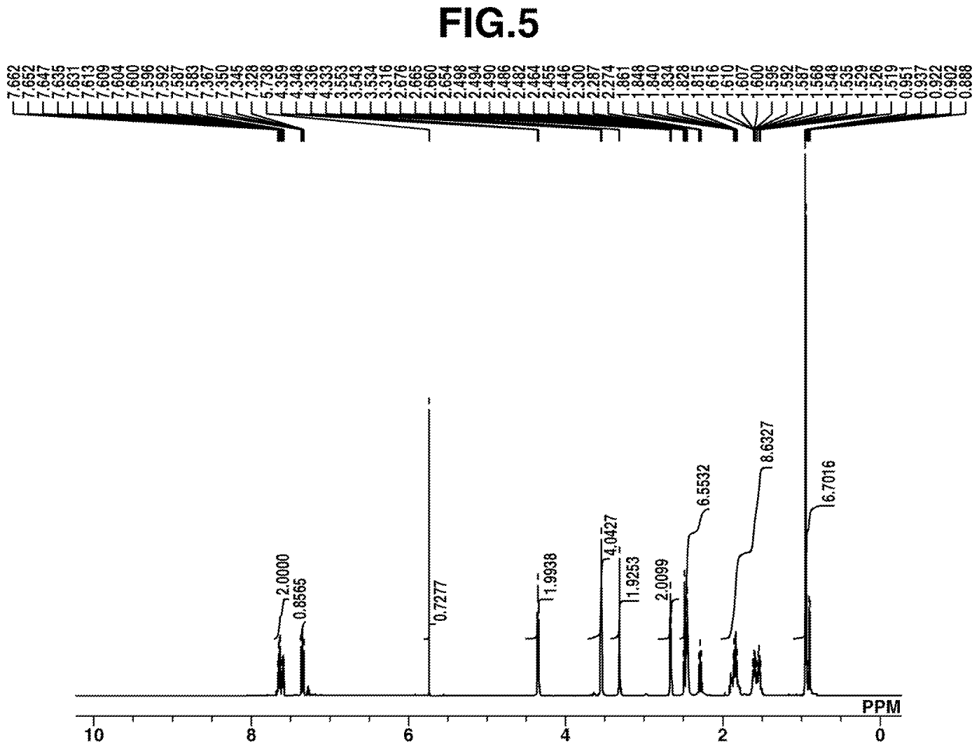

FIG. 6 is a diagram showing the ¹⁹F-NMR (DMSO-d₆) spectrum of amine compound AQ-1 in Example 1-1.

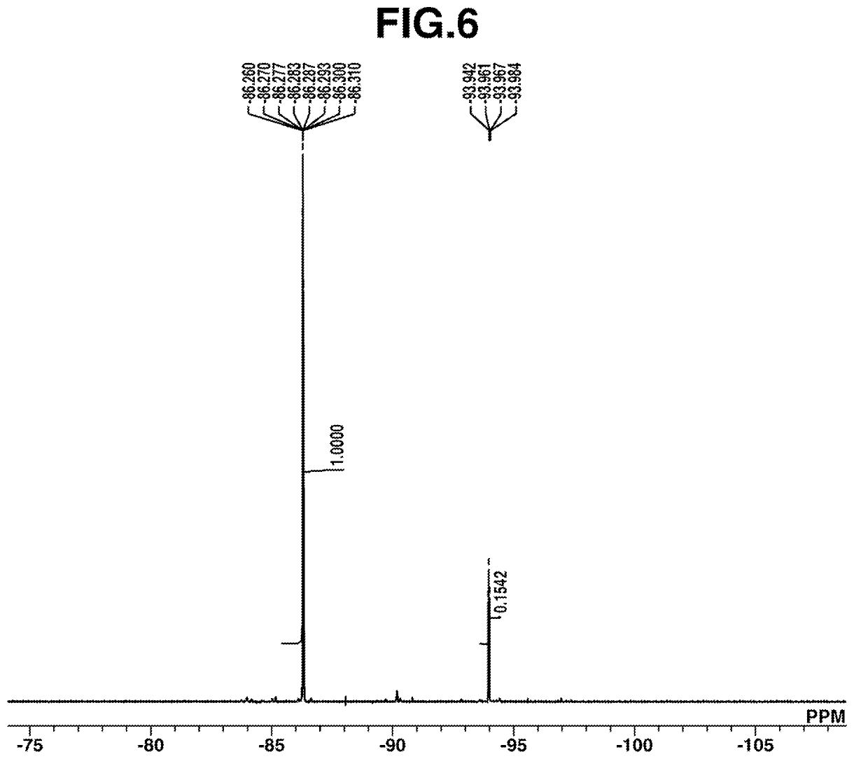

### DETAILED DESCRIPTION OF THE INVENTION

As used herein, the singular forms “a,” “an” and “the” include plural referents unless the context clearly dictates otherwise. “Optional” or “optionally” means that the subsequently described event or circumstances may or may not occur, and that description includes instances where the event or circumstance occurs and instances where it does not. The notation (Cₙ-Cₘ) means a group containing from n to m carbon atoms per group. The term “group” and “moiety” are interchangeable. In chemical formulae, the broken line (---) and asterisk (*) each designate a point of attachment, namely valence bond. Me stands for methyl and Ac for acetyl.

The abbreviations and acronyms have the following meaning.
EB: electron beam
EUV: extreme ultraviolet
Mw: weight average molecular weight
Mn: number average molecular weight
Mw/Mn: molecular weight dispersity
GPC: gel permeation chromatography
PEB: post-exposure bake
PAG: photoacid generator
LWR: line width roughness
CDU: critical dimension uniformity

Amine Compound

One embodiment of the invention is an amine compound having the formula (1).

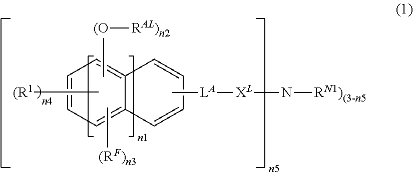

In formula (1), n1 is an integer of 0 or 1. The relevant structure represents a benzene ring in case of n1=0 and a naphthalene ring in case of n1=1. From the aspect of solvent solubility, a benzene ring corresponding to n1=0 is preferred. The subscript n2 is an integer of 1 to 3. In view of availability of starting reactants, n2=1 is preferred. The subscript n3 is an integer of 1 to 4. In case of n3≥2, a plurality of R^{F} may be the same or different. The subscript n4 is an integer of 0 to 4. It is provided that n2+n3+n4≤5 in case of n1=0, and n2+n3+n4≤7 in case of n1=1. The subscript n5 is an integer of 1 to 3, preferably 1 or 2.

In formula (1), R^{AL} forms an acid labile group with the neighboring oxygen atom. The acid labile group has the formula (AL-1) or (AL-2).

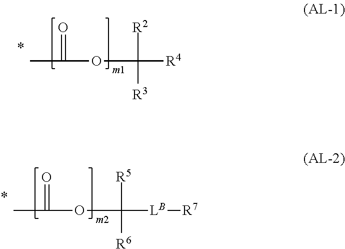

In formula (AL-1), R², R³ and R⁴ are each independently a C₁-C₁₂, preferably C₁-C₁₀ hydrocarbyl group in which some constituent —CH₂— may be replaced by —O— or —S—. When the hydrocarbyl group contains an aromatic ring, some or all of the hydrogen atoms on the aromatic ring may be substituted by halogen, cyano, nitro, optionally halogenated C₁-C₄ alkyl moiety, or optionally halogenated C₁-C₄ alkoxy moiety.

The C₁-C₁₂ hydrocarbyl group represented by R², R³ and R⁴ may be saturated or unsaturated and straight, branched or cyclic. Examples thereof include C₁-C₁₂ alkyl groups such as methyl, ethyl, n-propyl, isopropyl, n-butyl, isobutyl, sec butyl, tert-butyl, n-pentyl, tert-pentyl, n-hexyl, n-octyl, 2-ethylhexyl, n-nonyl, n-decyl, n-undecyl and n-dodecyl; C₃-C₁₂ cyclic saturated hydrocarbyl groups such as cyclopentyl, cyclohexyl, cyclopentylmethyl, cyclopentylethyl, cyclopentylbutyl, cyclohexylmethyl, cyclohexylethyl, cyclohexylbutyl, norbornyl, norbornylmethyl, adamantyl, adamantylmethyl, tricyclo[5.2.1.0^{2,6}]decyl, and tetracyclo[6.2.1.1^{3,6}.0^{2,7}]dodecyl; C₂-C₁₂ alkenyl groups such as vinyl, alkyl, propenyl, butenyl, pentenyl, and hexenyl; C₂-C₁₂ alkynyl groups such as ethynyl, propynyl, butynyl, pentynyl and hexynyl; C₃-C₁₂ cyclic unsaturated aliphatic hydrocarbyl groups such as cyclopentenyl and cyclohexenyl; C₆-C₁₂ aryl groups such as phenyl, naphthyl and indanyl; C₇-C₁₂ aralkyl groups such as benzyl, 1-phenylethyl and 2-phenylethyl; and combinations thereof.

Also, R² and R³ may bond together to form a ring with the carbon atom to which they are attached. Examples of the ring thus formed include cyclopropane, cyclobutane, cyclopentane, cyclohexane, cycloheptane, cyclooctane, norbornane, adamantine, tricyclo[5.2.1.0^{2,6}]decane, and tetracyclo[6.2.1.1^{3,6}.0^{2,7}]dodecane rings. In the ring, some constituent —CH₂— may be replaced by —O— or —S—. It is noted that when R² and R³ do not bond together or form a ring, at least one of R² and R³ has a cyclic structure, preferably C₃-C₃₀ alicyclic structure or C₆-C₃₀ aromatic ring structure.

In formula (AL-1), m1 is 0 or 1. The asterisk (*) designates a point of attachment to the neighboring —O—.

In formula (AL-2). R⁵ and R⁶ are each independently hydrogen or a C₁-C₁₀ hydrocarbyl group. The C₁-C₁₀ hydrocarbyl group represented by R⁵ and R⁶ may be saturated or unsaturated and straight, branched or cyclic. Examples thereof are as exemplified above for the C₁-C₁₂ hydrocarbyl group represented by R², R³ and R⁴, but of 1 to 10 carbon atoms.

In formula (AL-2), R⁷ is a C₁-C₂₀ hydrocarbyl group in which some constituent —CH₂— may be replaced by —O— or —S—. The hydrocarbyl group may be saturated or unsaturated and straight, branched or cyclic. Examples thereof include C₁-C₂₀ alkyl groups such as methyl, ethyl, n-propyl, isopropyl, n-butyl, isobutyl, sec-butyl, tert-butyl, n-pentyl, n-hexyl, n-octyl, n-nonyl, n-decyl, undecyl, dodecyl, tridecyl, tetradecyl, pentadecyl, heptadecyl, octadecyl, nonadecyl, and icosyl; C₃-C₂₀ cyclic saturated hydrocarbyl groups such as cyclopropyl, cyclopentyl, cyclohexyl, cyclopropylmethyl, 4-methylcyclohexyl, cyclohexylmethyl, norbornyl, norbornylmethyl, adamantyl, adamantylmethyl, tricyclo[5.2.1.0^{2,6}]decyl, and tetracyclo[6.2.1.1^{3,6}.0^{2,7}]dodecyl; C₂-C₂₀ alkenyl groups such as vinyl, propenyl, butenyl, pentenyl, and hexenyl; C₂-C₂₀ alkynyl groups such as ethynyl, propynyl, butynyl, pentynyl and hexynyl; C₃-C₂₀ cyclic unsaturated aliphatic hydrocarbyl groups such as cyclopentenyl, cyclohexenyl and norbornenyl; C₆-C₂₀ aryl groups such as phenyl, methylphenyl, ethylphenyl, n-propylphenyl, isopropylphenyl, n-butylphenyl, isobutylphenyl, sec-butylphenyl, tert-butylphenyl, naphthyl, methylnaphthyl, ethylnaphthyl, n-propylnaphthyl, isopropylnaphthyl, n-butylnaphthyl, isobutylnaphthyl, sec-butylnaphthyl, and tert-butylnaphthyl; C₇-C₂₀ aralkyl groups such as benzyl and phenethyl; and combinations thereof. R⁶ and R⁷ may bond together to form a C₃-C₂₀ heterocyclic group with the carbon atom and L^{B} to which they are attached. Some constituent —CH₂— in the heterocyclic group may be replaced by —O— or —S—.

In formula (AL-2), L^{B} is —O— or —S—.

In formula (AL-2), m2 is 0 or 1. The asterisk (*) designates a point of attachment to the neighboring —O—.

Examples of the acid labile group having formula (AL-1) are shown below, but not limited thereto. Notably, * designates a point of attachment to the neighboring —O—.

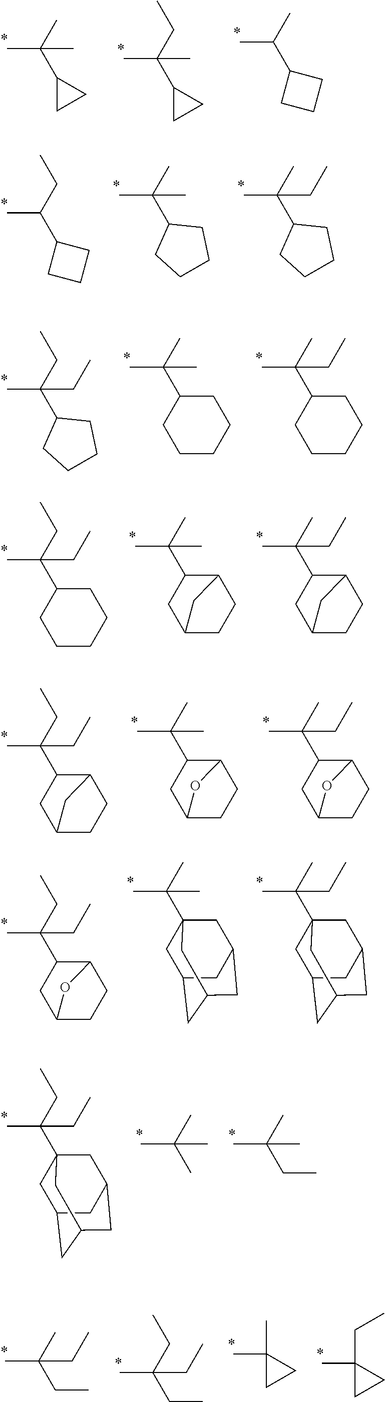

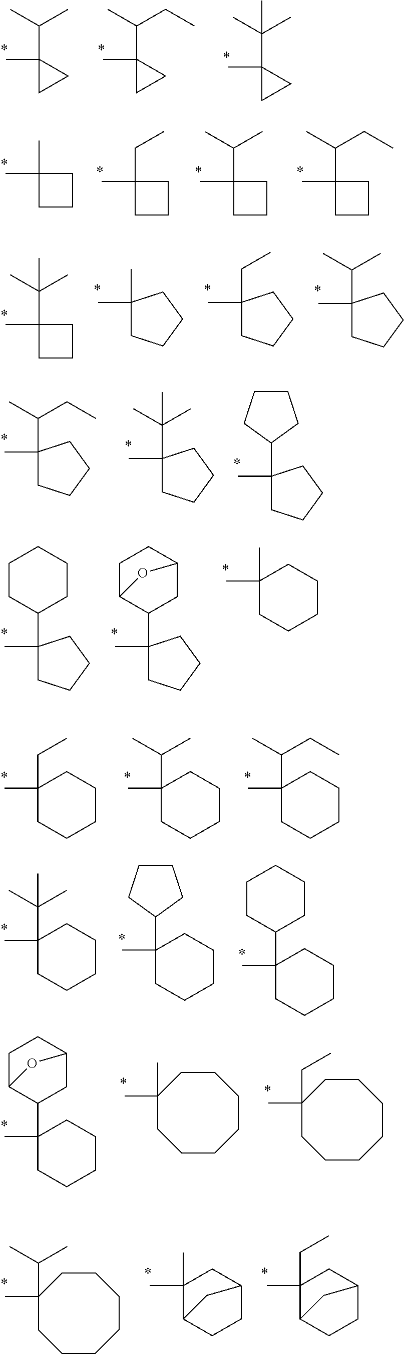

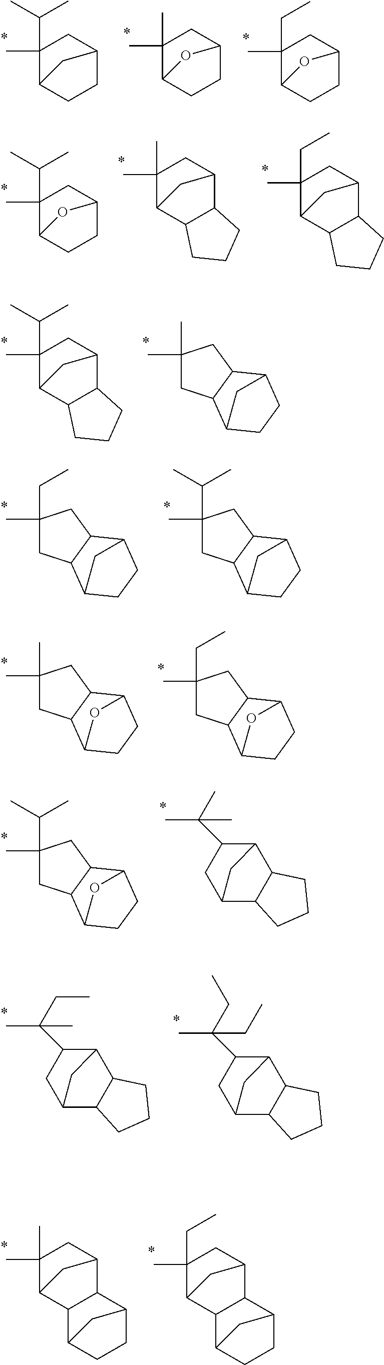

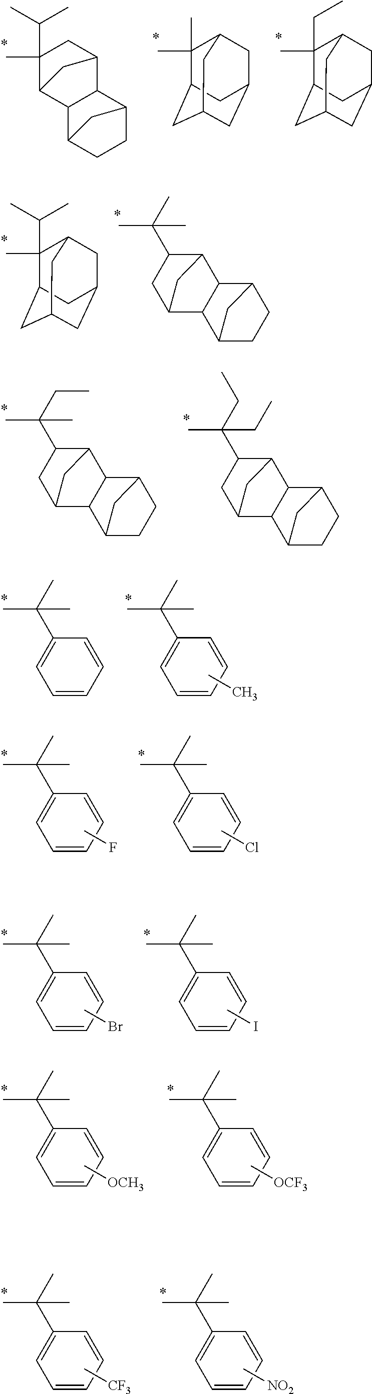

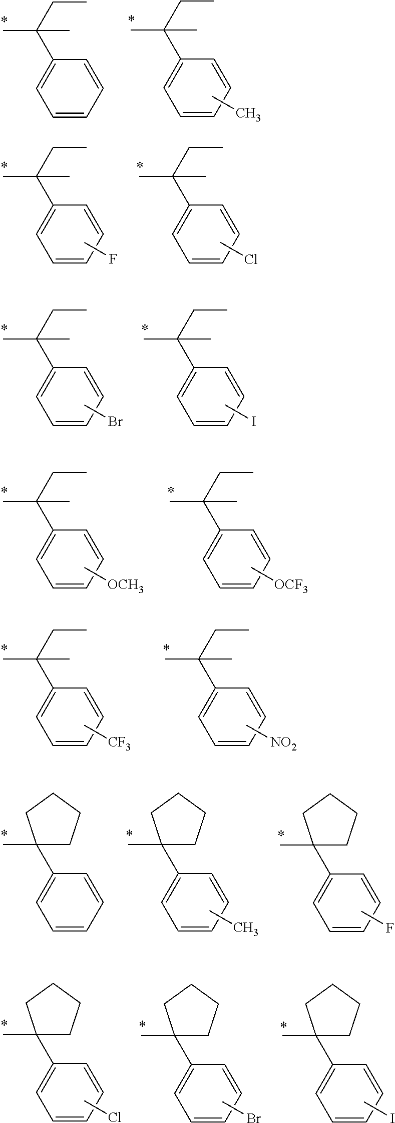

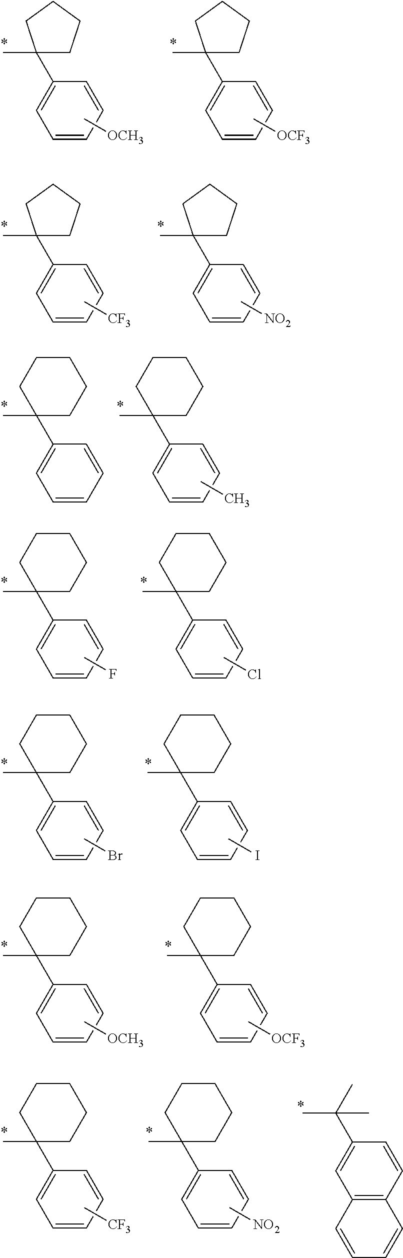

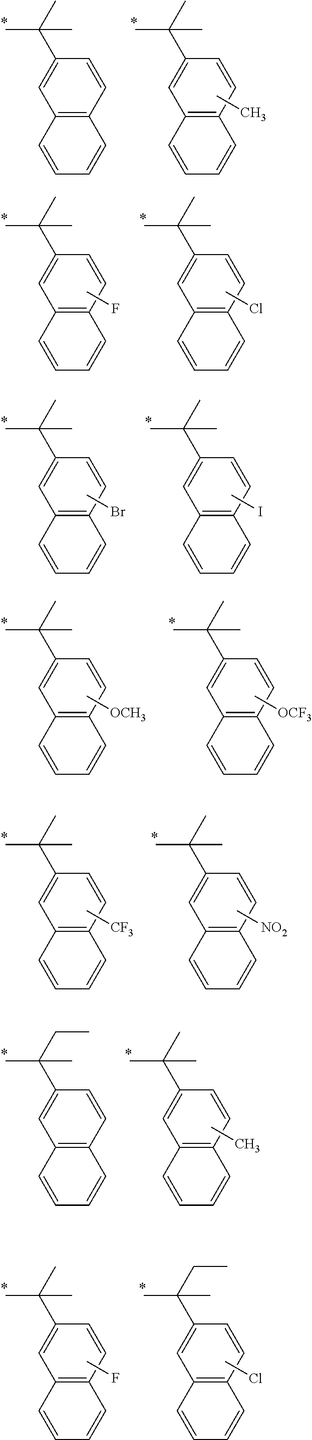

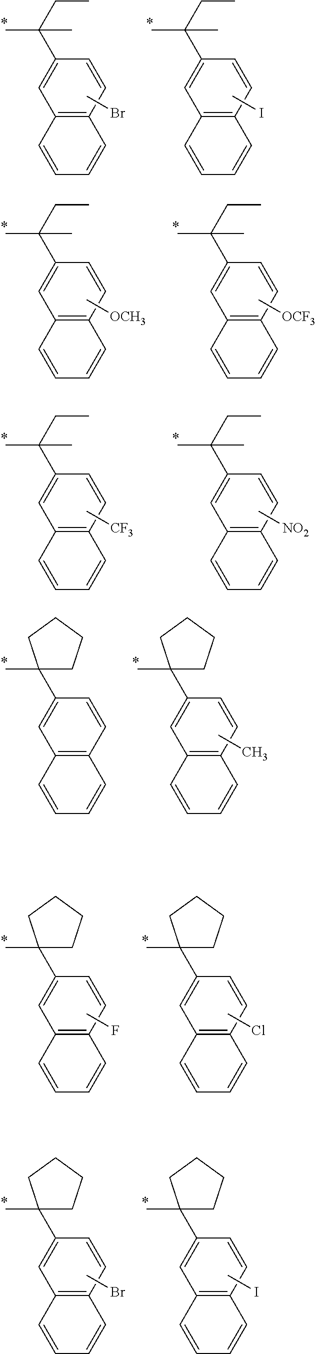

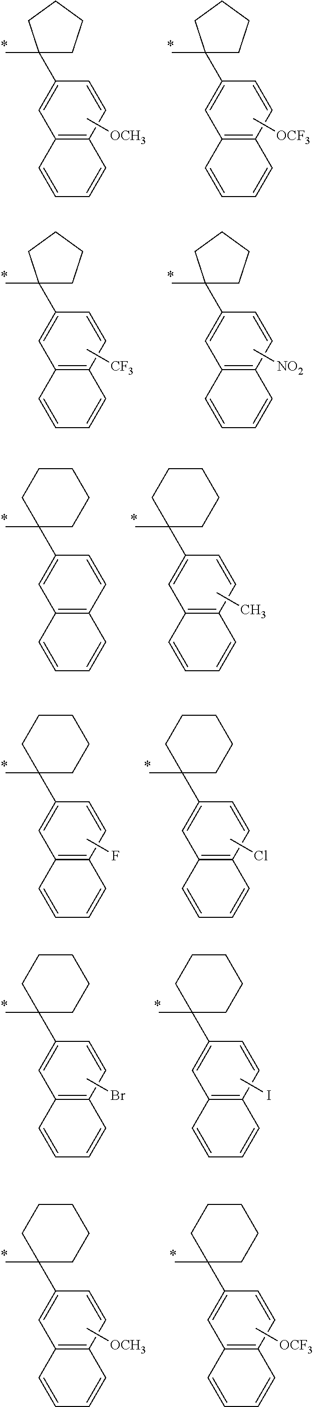

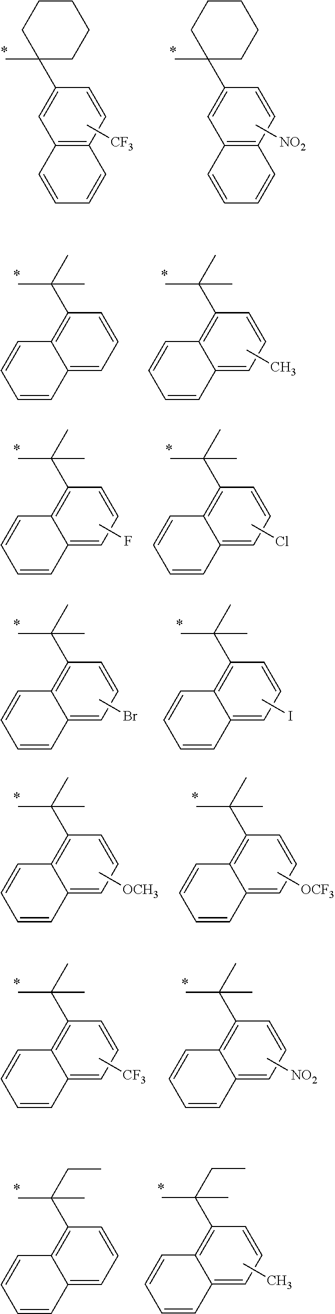

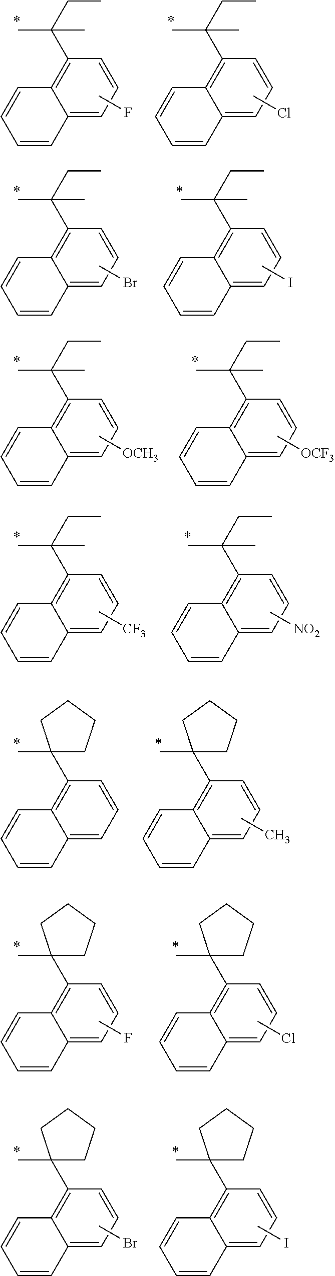

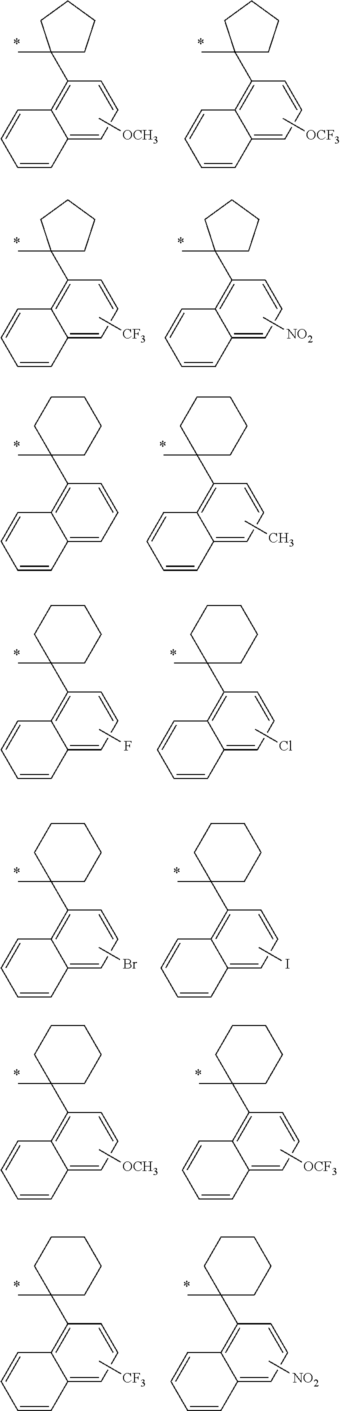

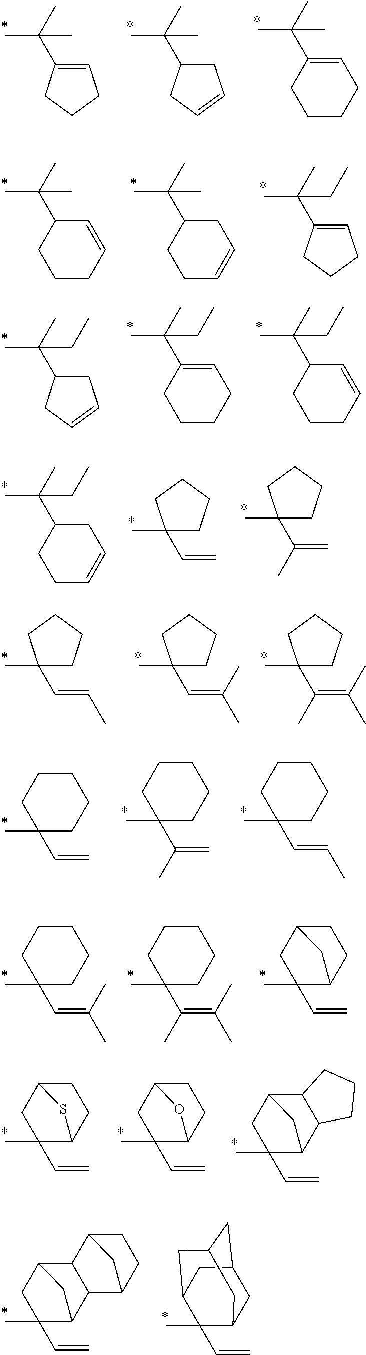

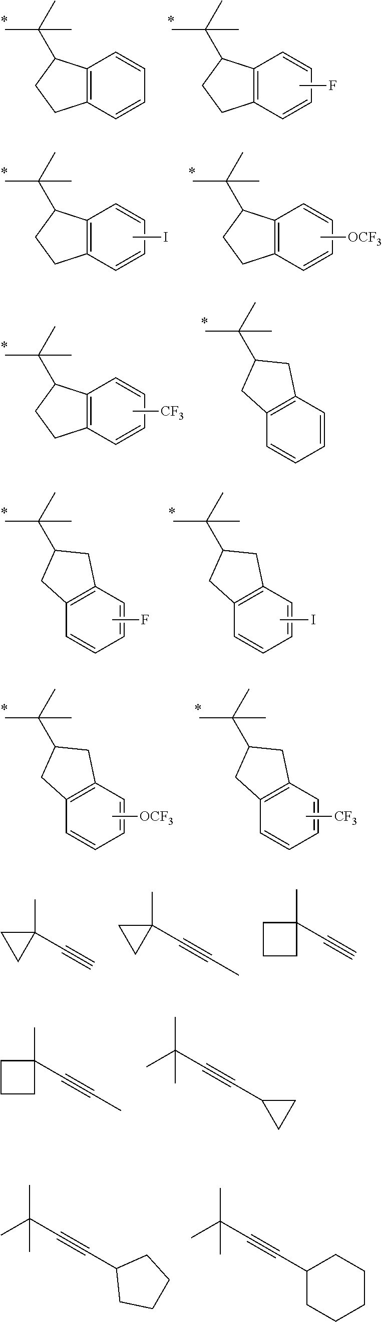

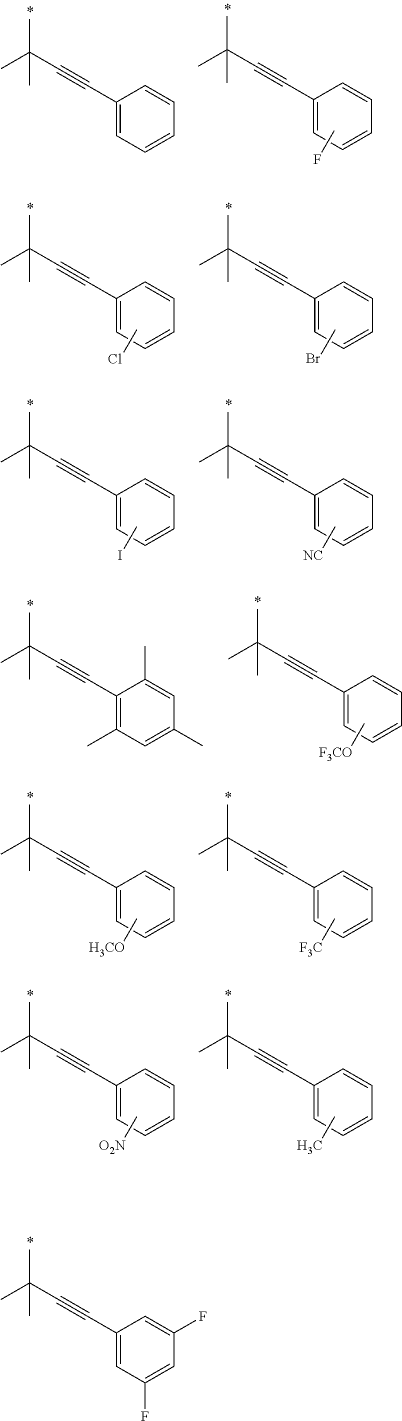

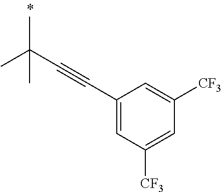

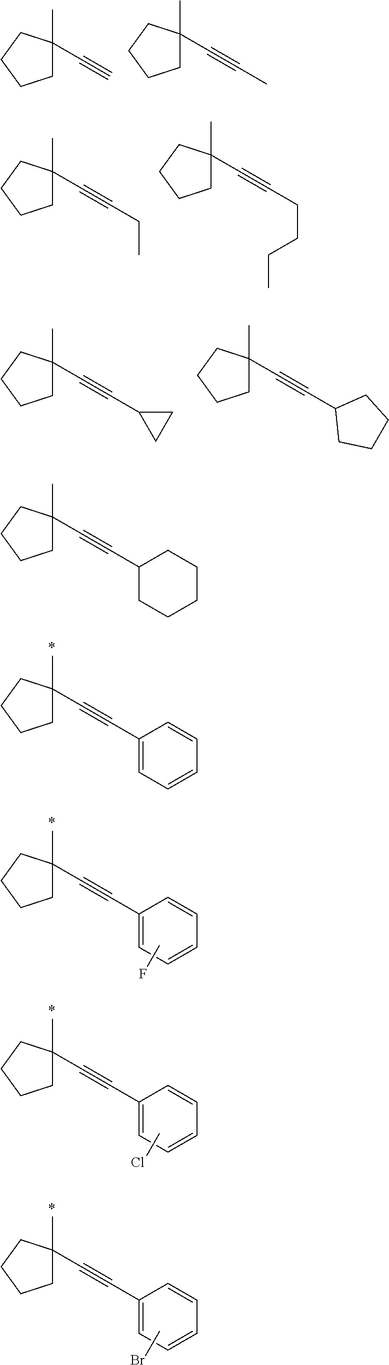

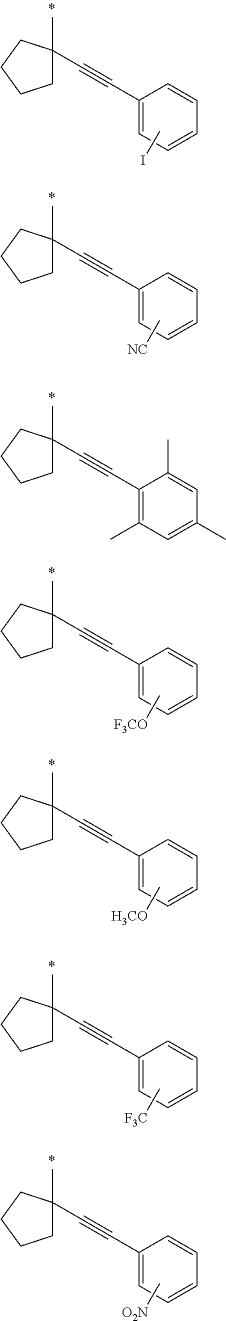

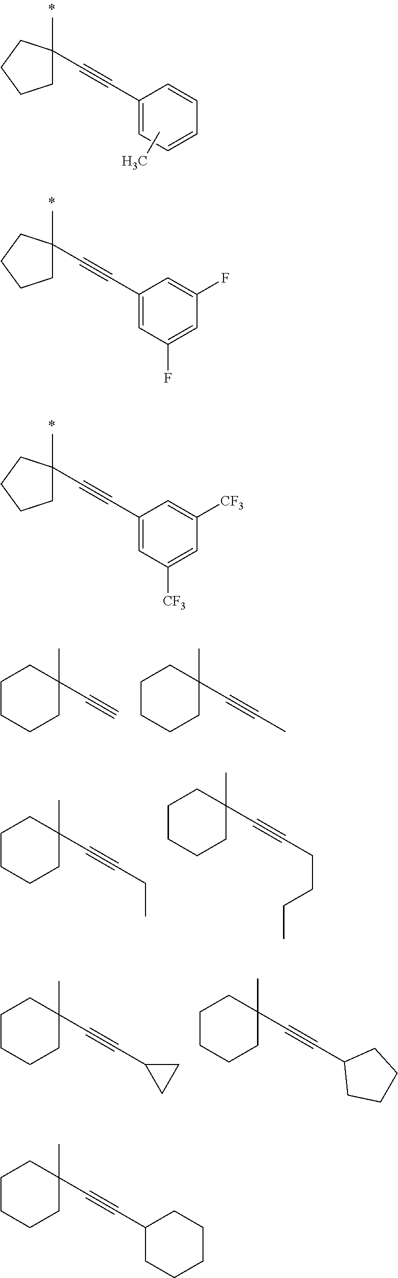

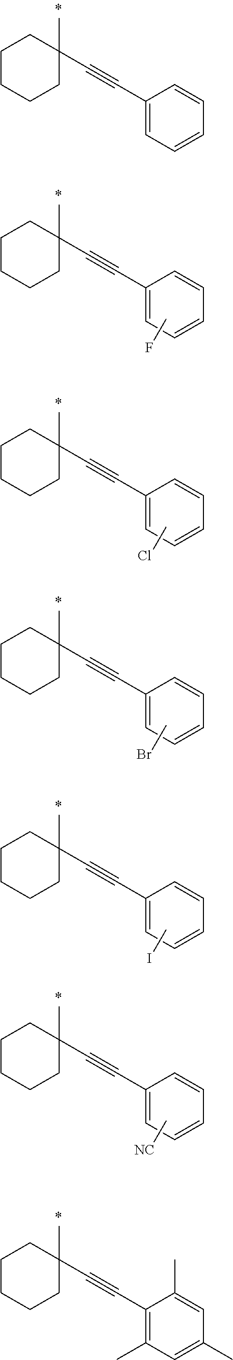

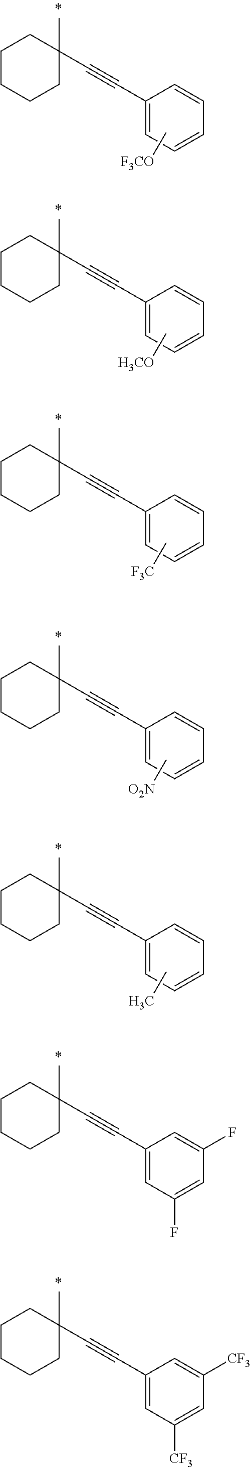

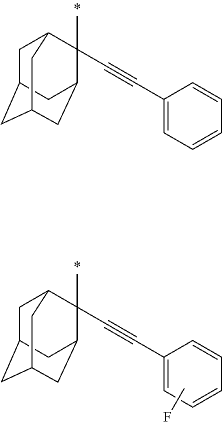

Examples of the acid labile group having formula (AL-2) are shown below, but not limited thereto. Notably, * designates a point of attachment to the neighboring —O—.

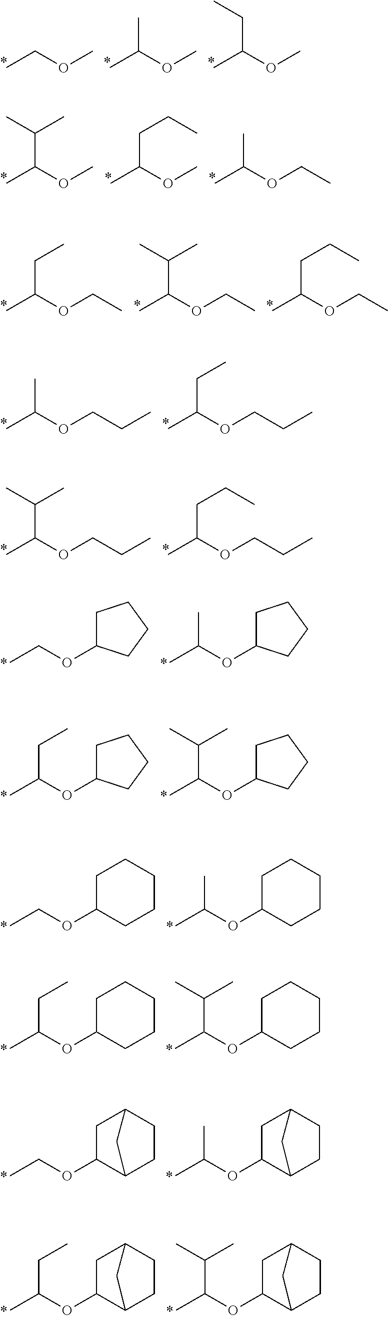

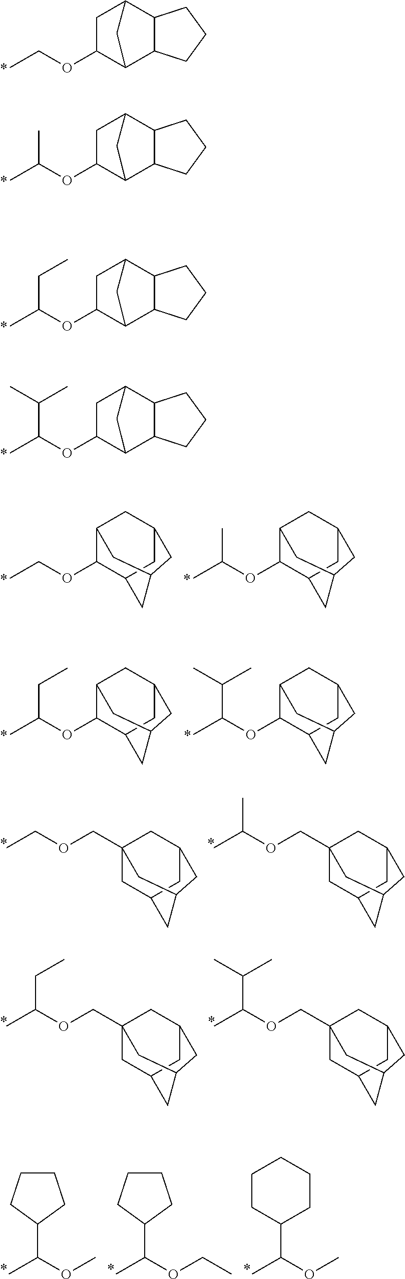

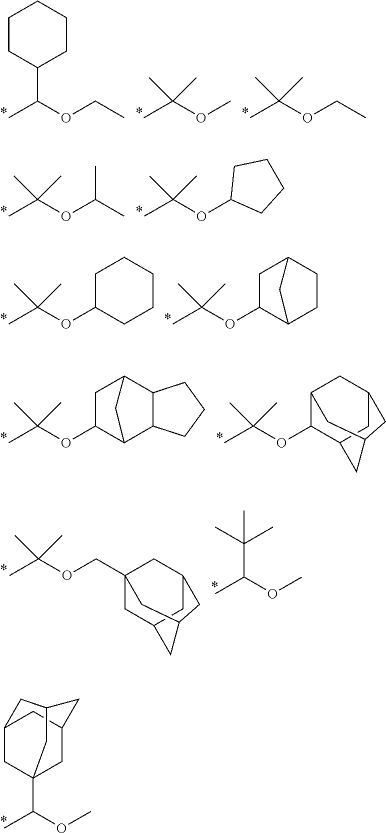

In formula (1), R^{F} is fluorine, a C₁-C₆ fluorinated saturated hydrocarbyl group, C₁-C₆ fluorinated saturated hydrocarbyloxy group, or C₁-C₆ fluorinated saturated hydrocarbylthio group. Inter alia, fluorine and C₁-C₆ fluorinated saturated hydrocarbyl groups are preferred. Of the C₁-C₆ fluorinated saturated hydrocarbyl groups, trifluoromethyl is most preferred. In case of n3≥2, a plurality of R^{F} may be the same or different.

In formula (1), R^{F} and —O—R^{AL} are attached to adjacent carbon atoms. Specifically, in case of n2=n3=1, R^{F} and —O—R^{AL} are attached to adjacent carbon atoms. When at least one of n2 and n3 is 2, at least one R^{F} and at least one —O—R^{AL} are attached to adjacent carbon atoms. The adjacent positioning of R^{F} and —O—R^{AL} ensures that a phenol created as a result of the acid labile group being eliminated from —O—R¹ has a high acidity, which leads to a high solubility in alkaline developer and a significant swell-restraining effect.

In formula (1). R¹ is a C₁-C₂₀ hydrocarbyl group which may contain a heteroatom. The hydrocarbyl group may be saturated or unsaturated and straight, branched or cyclic. Examples thereof include C₁-C₂₀ alkyl groups such as methyl, ethyl, n-propyl, isopropyl, n-butyl, isobutyl, sec-butyl, tert-butyl, n-pentyl, n-hexyl, n-octyl, n-nonyl, n-decyl, undecyl, dodecyl, tridecyl, tetradecyl, pentadecyl, heptadecyl, octadecyl, nonadecyl, and icosyl; C₃-C₂₀ cyclic saturated hydrocarbyl groups such as cyclopropyl, cyclopentyl, cyclohexyl, cyclopropylmethyl, 4-methylcyclohexyl, cyclohexylmethyl, norbornyl, and adamantyl; C₂-C₂₀ alkenyl groups such as vinyl, allyl, propenyl, butenyl, and hexenyl; C₃-C₂₀ cyclic unsaturated hydrocarbyl groups such as cyclohexenyl; C₆-C₂₀ aryl groups such as phenyl and naphthyl; C₇-C₂₀ aralkyl groups such as benzyl, 1-phenylethyl and 2-phenylethyl; and combinations thereof. Inter alia, aryl groups are preferred. In the hydrocarbyl group, some or all of the hydrogen atoms may be substituted by a moiety containing a heteroatom such as oxygen, sulfur, nitrogen or halogen, and some constituent —CH₂— may be replaced by a moiety containing a heteroatom such as oxygen, sulfur or nitrogen, so that the group may contain a hydroxy moiety, fluorine, chlorine, bromine, iodine, cyano moiety, carbonyl moiety, ether bond, ester bond, sulfonic ester bond, carbonate bond, lactone ring, sultone ring, carboxylic anhydride (—C(═O)—O—C(═O)—) or haloalkyl moiety.

In case of n4≥2, a plurality of R¹ may bond together to form a ring with the carbon atom on the aromatic ring to which they are attached. The ring is preferably a 5- or 6-membered ring.

In formula (1), R^{N1} is hydrogen or a C₁-C₂₀ hydrocarbyl group in which some or all of the hydrogen atoms may be substituted by halogen and some constituent —CH₂— may be replaced by —O— or —C(═O)—. In case of n5=1, two R^{N1} may bond together to form a ring with the nitrogen atom to which they are attached. The ring may contain —O— or —S— therein. It is excluded that two R^{N1} are hydrogen at the same time.

The hydrocarbyl group R^{N1} may be saturated or unsaturated and straight, branched or cyclic. Examples thereof include C₁-C₂₀ alkyl groups such as methyl, ethyl, n-propyl, isopropyl, n-butyl, isobutyl, and tert-butyl; C₃-C₂₀ cyclic saturated hydrocarbyl groups such as cyclopropyl, cyclopentyl, cyclohexyl, cyclopropylmethyl, 4-methylcyclohexyl, cyclohexylmethyl, norbornyl, and adamantyl; C₂-C₂₀ alkenyl groups such as vinyl, allyl, propenyl, butenyl, and hexenyl; C₃-C₂₀ cyclic unsaturated hydrocarbyl groups such as cyclohexenyl; C₆-C₂₀ aryl groups such as phenyl and naphthyl: C₇-C₂₀ aralkyl groups such as benzyl, 1-phenylethyl and 2-phenylethyl; and combinations thereof.

The ring that two R^{N1}, taken together, form with the nitrogen atom to which they are attached is preferably alicyclic. Examples of the ring include aziridine, azetidine, pyrrolidine, and piperidine rings, but are not limited thereto. In the nitrogen-containing heterocycle, some —CH₂— may be replaced by —O— or —S—.

In formula (1), L^{A} is a single bond, ether bond, ester bond, sulfonic ester bond, carbonate bond or carbamate bond, preferably a single bond, ether bond or ester bond, more preferably an ether bond or ester bond.

In formula (1), X^{L} is a single bond or a C₁-C₄₀ hydrocarbylene group which may contain a heteroatom. A C₁-C₁₀ hydrocarbylene group is preferred. Examples of X^{L} are shown below, but not limited thereto. Each asterisk (*) designates a point of attachment to L^{A} or the nitrogen atom.

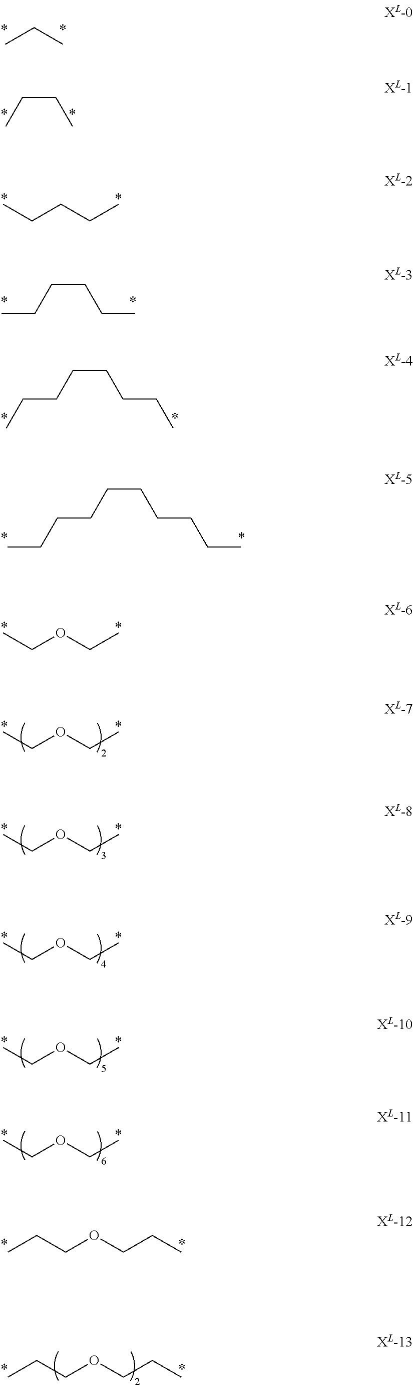

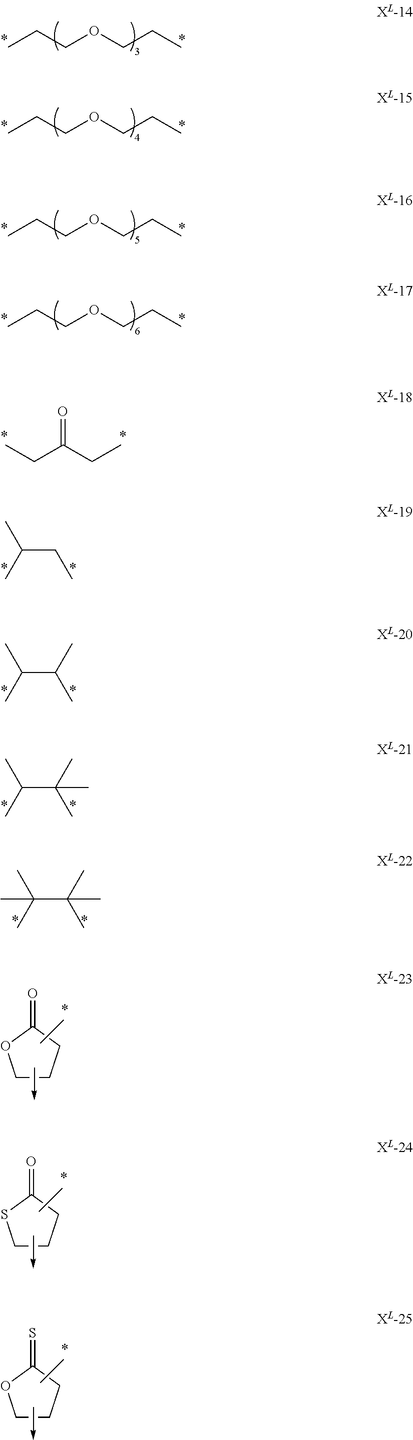

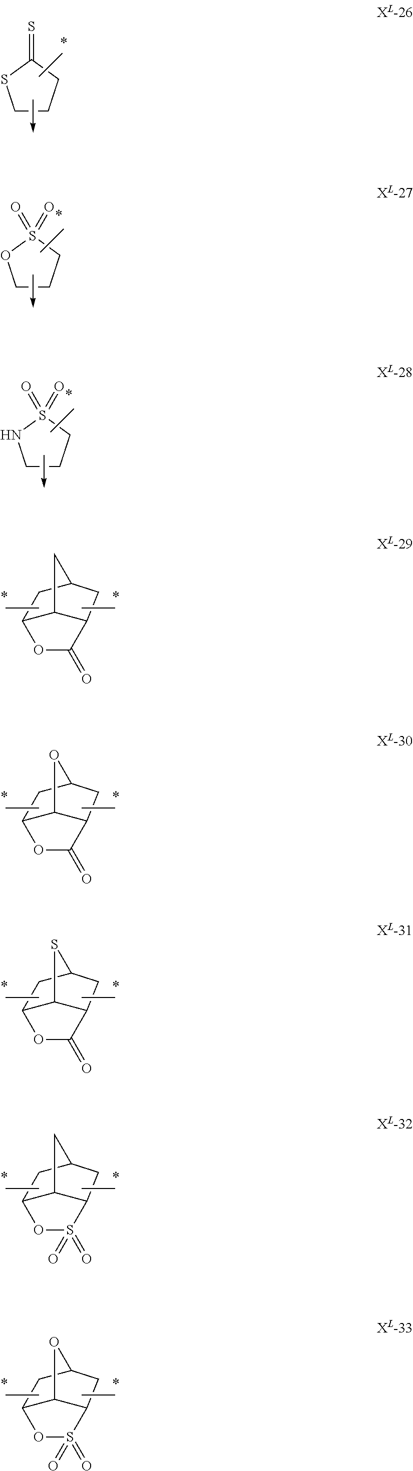

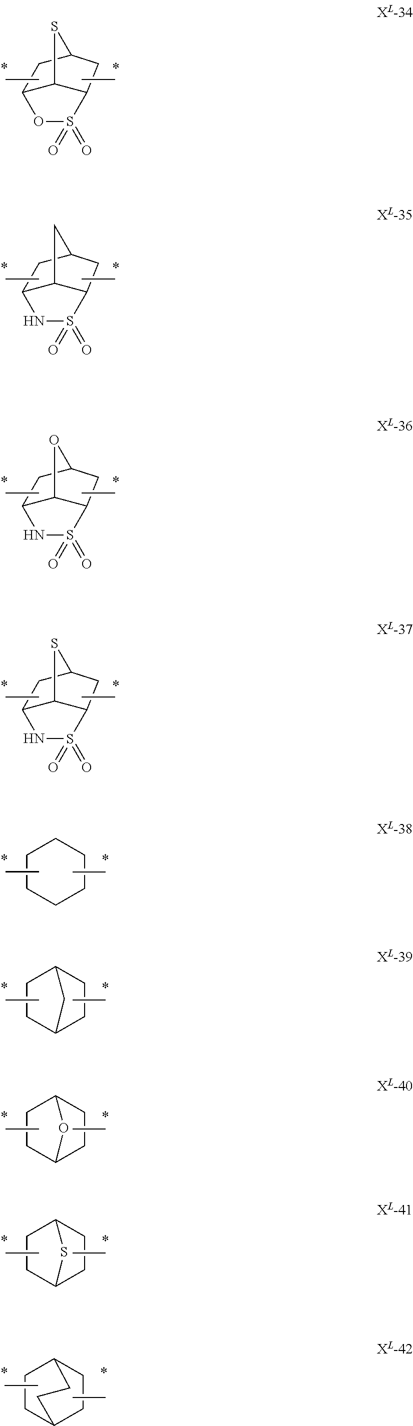

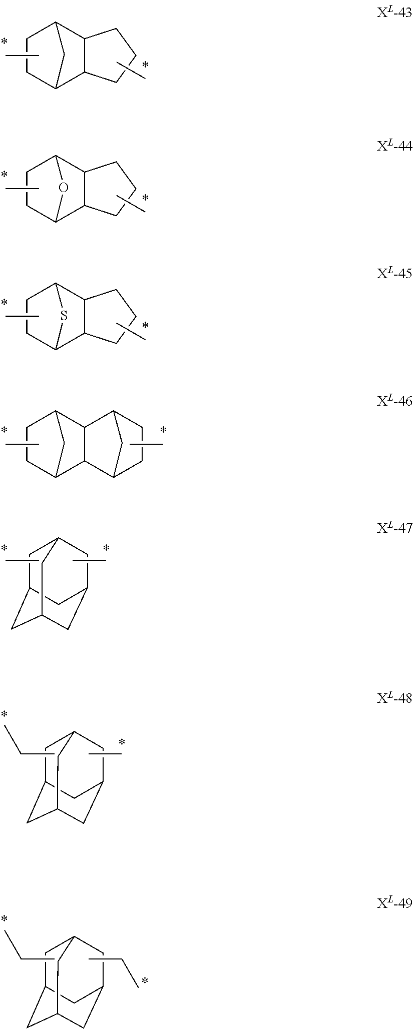

Of the foregoing examples, X^{L}-0 to X^{L}-22 and X^{L}-47 to X^{L}-49 are preferred, with X^{L}-0 to X^{L}-17 being more preferred.

Examples of the amine compound having formula (1) are shown below, but not limited thereto. The substitution positions of substituents on the aromatic ring are not limited to the illustrated ones as long as —O—R^{AL} and R^{F} are positioned adjacent to each other.

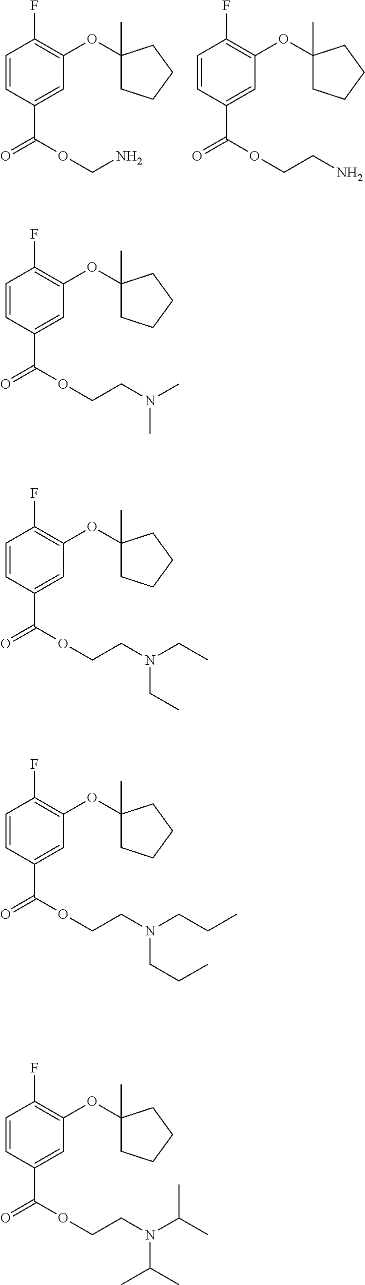

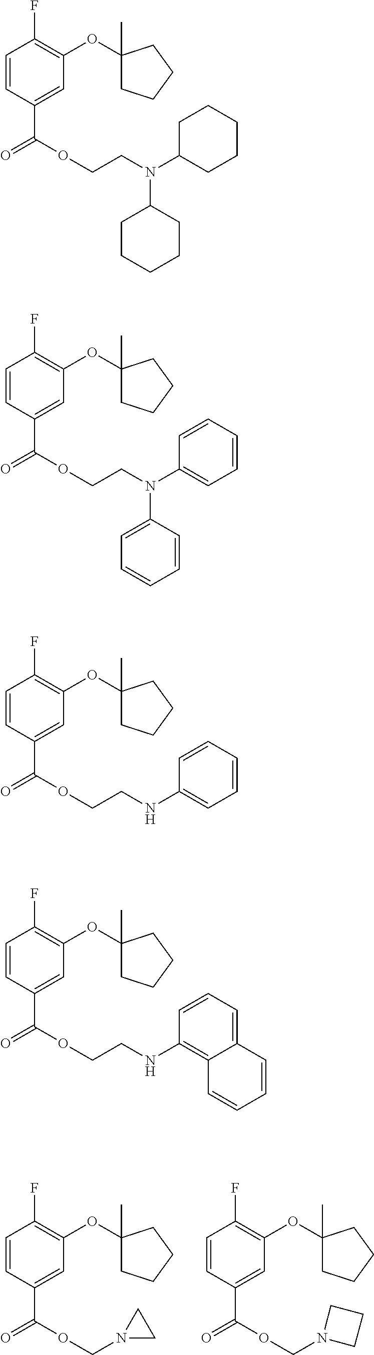

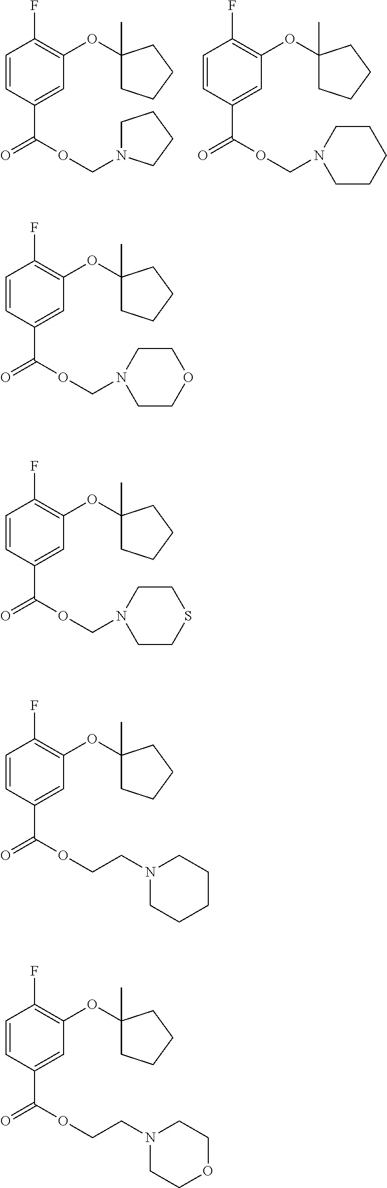

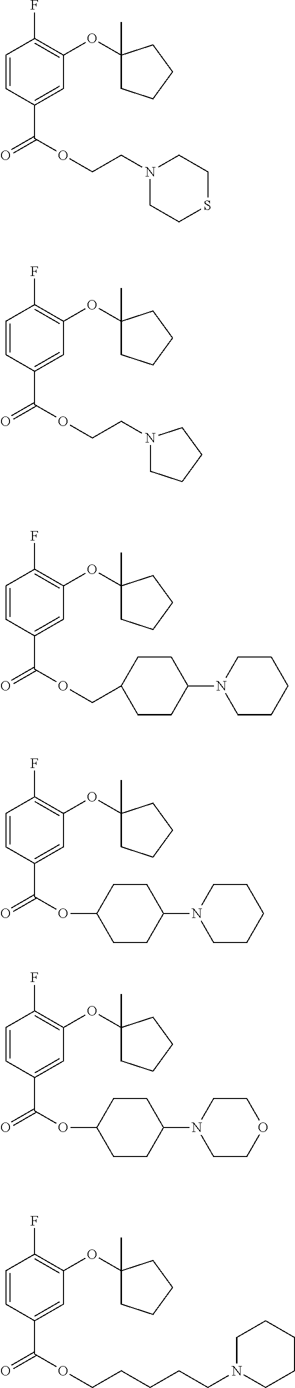 [SMILES: CC1(Oc2cc(C(=O)OCCN3CCSCC3)ccc2F)CCCC1 ; CC1(Oc2cc(C(=O)OCCN3CCCC3)ccc2F)CCCC1 ; CC1(Oc2cc(C(=O)OCC3CCC(N4CCCCC4)CC3)ccc2F)CCCC1 ; CC1(Oc2cc(C(=O)OC3CCC(N4CCCCC4)CC3)ccc2F)CCCC1 ; CC1(Oc2cc(C(=O)OC3CCC(N4CCOCC4)CC3)ccc2F)CCCC1 ; CC1(Oc2cc(C(=O)OCCCCCN3CCCCC3)ccc2F)CCCC1]

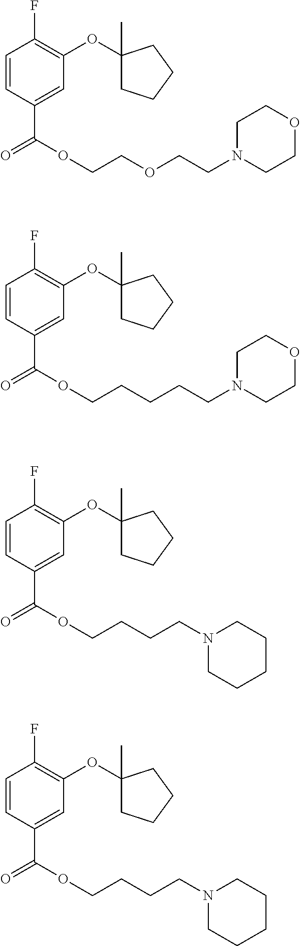 [SMILES: CC1(Oc2cc(C(=O)OCCOCCN3CCOCC3)ccc2F)CCCC1 ; CC1(Oc2cc(C(=O)OCCCCCN3CCOCC3)ccc2F)CCCC1 ; CC1(Oc2cc(C(=O)OCCCCN3CCCCC3)ccc2F)CCCC1 ; CC1(Oc2cc(C(=O)OCCCCN3CCCCC3)ccc2F)CCCC1]

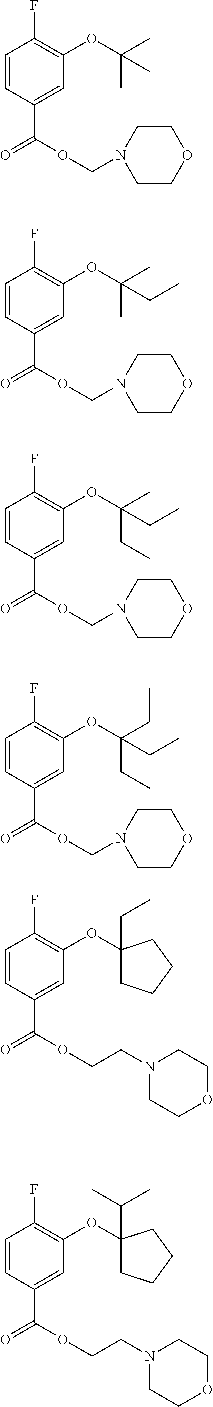

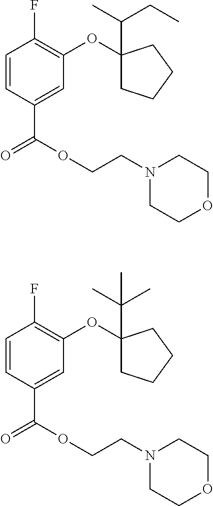 [SMILES: CCC(C)C1(Oc2cc(C(=O)OCCN3CCOCC3)ccc2F)CCCC1 ; CC(C)(C)C1(Oc2cc(C(=O)OCCN3CCOCC3)ccc2F)CCCC1]

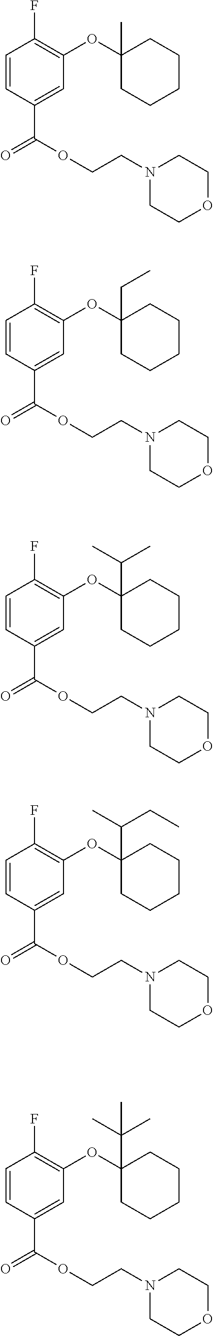 [SMILES: CC1(Oc2cc(C(=O)OCCN3CCOCC3)ccc2F)CCCCC1 ; CCC1(Oc2cc(C(=O)OCCN3CCOCC3)ccc2F)CCCCC1 ; CC(C)C1(Oc2cc(C(=O)OCCN3CCOCC3)ccc2F)CCCCC1 ; CCC(C)C1(Oc2cc(C(=O)OCCN3CCOCC3)ccc2F)CCCCC1 ; CC(C)(C)C1(Oc2cc(C(=O)OCCN3CCOCC3)ccc2F)CCCCC1]

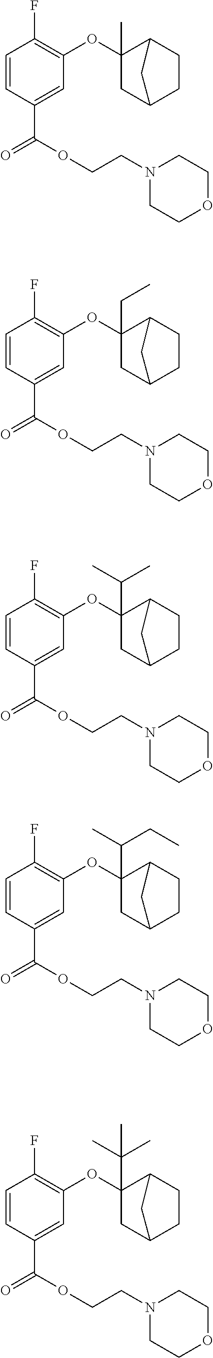 [SMILES: CC1(Oc2cc(C(=O)OCCN3CCOCC3)ccc2F)CC2CCC1C2 ; CCC1(Oc2cc(C(=O)OCCN3CCOCC3)ccc2F)CC2CCC1C2 ; CC(C)C1(Oc2cc(C(=O)OCCN3CCOCC3)ccc2F)CC2CCC1C2 ; CCC(C)C1(Oc2cc(C(=O)OCCN3CCOCC3)ccc2F)CC2CCC1C2 ; CC(C)(C)C1(Oc2cc(C(=O)OCCN3CCOCC3)ccc2F)CC2CCC1C2]

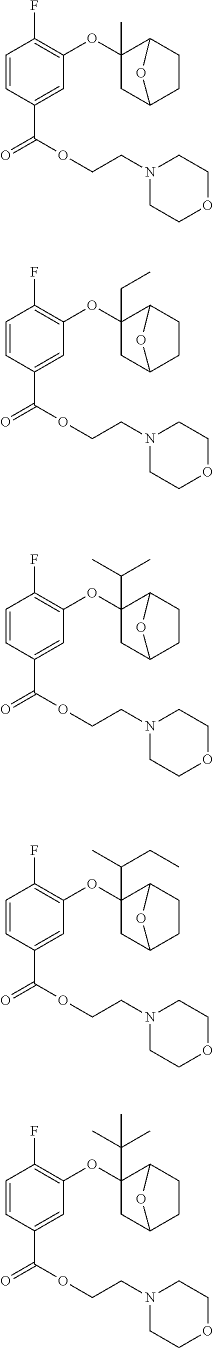 [SMILES: CC1(Oc2cc(C(=O)OCCN3CCOCC3)ccc2F)CC2CCC1O2 ; CCC1(Oc2cc(C(=O)OCCN3CCOCC3)ccc2F)CC2CCC1O2 ; CC(C)C1(Oc2cc(C(=O)OCCN3CCOCC3)ccc2F)CC2CCC1O2 ; CCC(C)C1(Oc2cc(C(=O)OCCN3CCOCC3)ccc2F)CC2CCC1O2 ; CC(C)(C)C1(Oc2cc(C(=O)OCCN3CCOCC3)ccc2F)CC2CCC1O2]

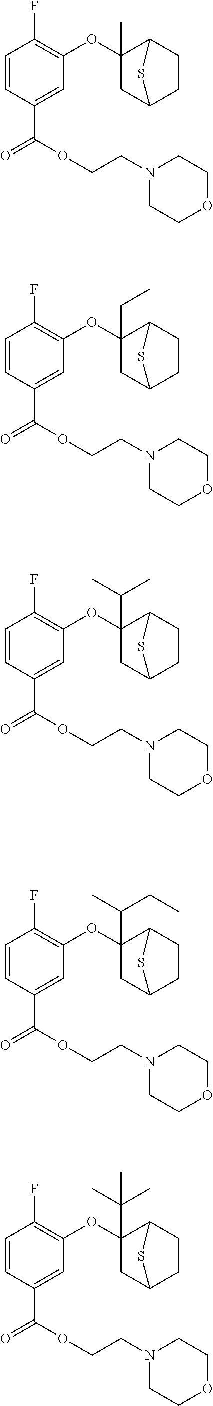 [SMILES: CC1(Oc2cc(C(=O)OCCN3CCOCC3)ccc2F)CC2CCC1S2 ; CCC1(Oc2cc(C(=O)OCCN3CCOCC3)ccc2F)CC2CCC1S2 ; CC(C)C1(Oc2cc(C(=O)OCCN3CCOCC3)ccc2F)CC2CCC1S2 ; CCC(C)C1(Oc2cc(C(=O)OCCN3CCOCC3)ccc2F)CC2CCC1S2 ; CC(C)(C)C1(Oc2cc(C(=O)OCCN3CCOCC3)ccc2F)CC2CCC1S2]

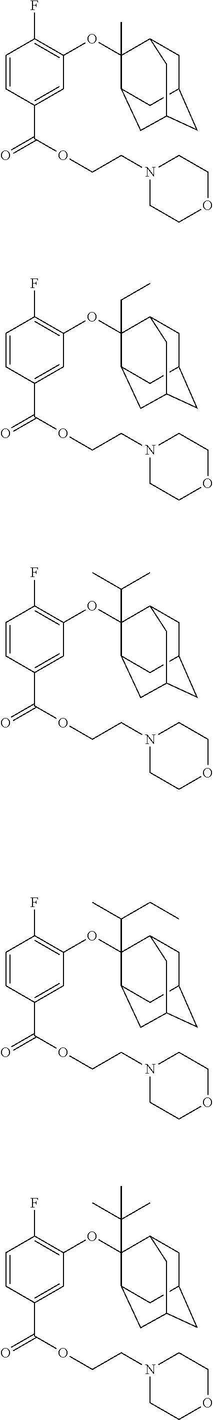 [SMILES: CC1(Oc2cc(C(=O)OCCN3CCOCC3)ccc2F)C2CC3CC(C2)CC1C3 ; CCC1(Oc2cc(C(=O)OCCN3CCOCC3)ccc2F)C2CC3CC(C2)CC1C3 ; CC(C)C1(Oc2cc(C(=O)OCCN3CCOCC3)ccc2F)C2CC3CC(C2)CC1C3 ; CCC(C)C1(Oc2cc(C(=O)OCCN3CCOCC3)ccc2F)C2CC3CC(C2)CC1C3 ; CC(C)(C)C1(Oc2cc(C(=O)OCCN3CCOCC3)ccc2F)C2CC3CC(C2)CC1C3]

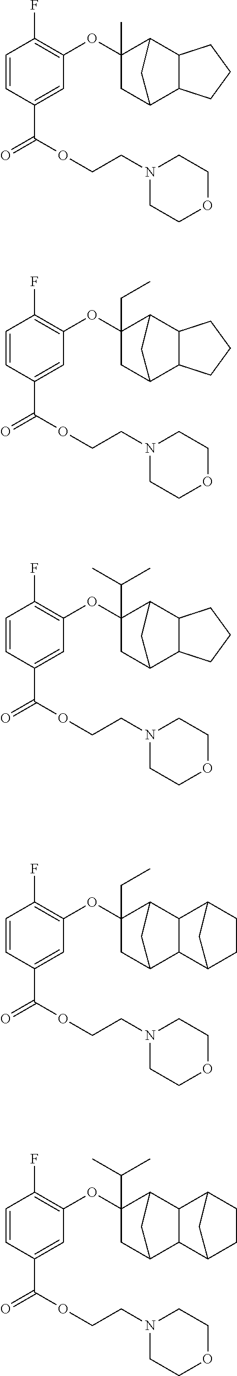 [SMILES: CC1(Oc2cc(C(=O)OCCN3CCOCC3)ccc2F)CC2CC1C1CCCC21 ; CCC1(Oc2cc(C(=O)OCCN3CCOCC3)ccc2F)CC2CC1C1CCCC21 ; CC(C)C1(Oc2cc(C(=O)OCCN3CCOCC3)ccc2F)CC2CC1C1CCCC21 ; CCC1(Oc2cc(C(=O)OCCN3CCOCC3)ccc2F)CC2CC1C1C3CCC(C3)C21 ; CC(C)C1(Oc2cc(C(=O)OCCN3CCOCC3)ccc2F)CC2CC1C1C3CCC(C3)C21]

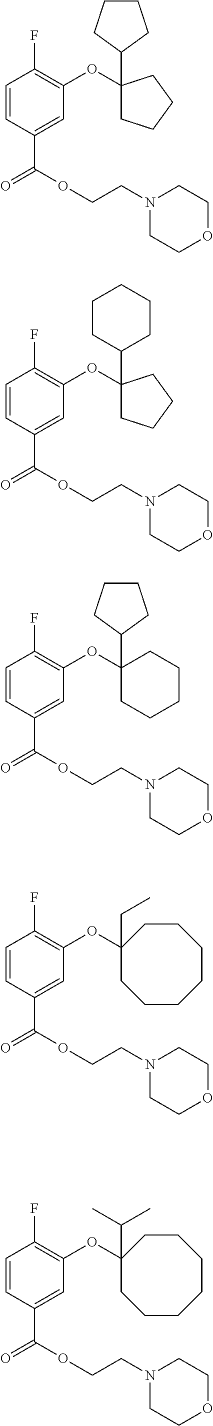 [SMILES: O=C(OCCN1CCOCC1)c1ccc(F)c(OC2(C3CCCC3)CCCC2)c1 ; O=C(OCCN1CCOCC1)c1ccc(F)c(OC2(C3CCCCC3)CCCC2)c1 ; O=C(OCCN1CCOCC1)c1ccc(F)c(OC2(C3CCCC3)CCCCC2)c1 ; CCC1(Oc2cc(C(=O)OCCN3CCOCC3)ccc2F)CCCCCCC1 ; CC(C)C1(Oc2cc(C(=O)OCCN3CCOCC3)ccc2F)CCCCCCC1]

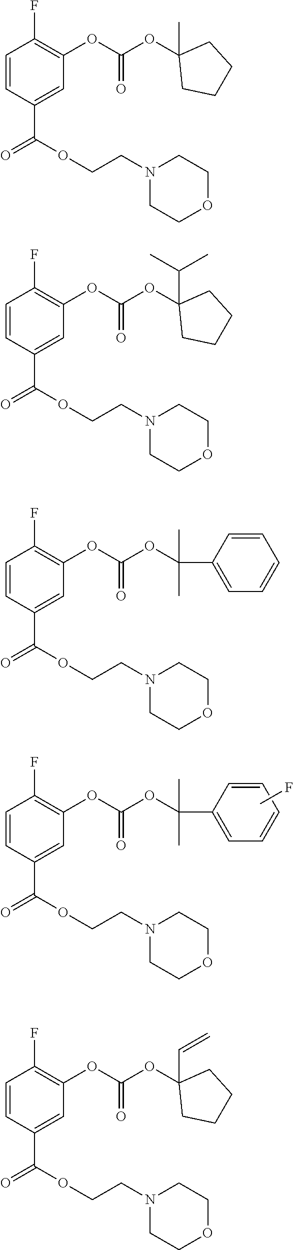

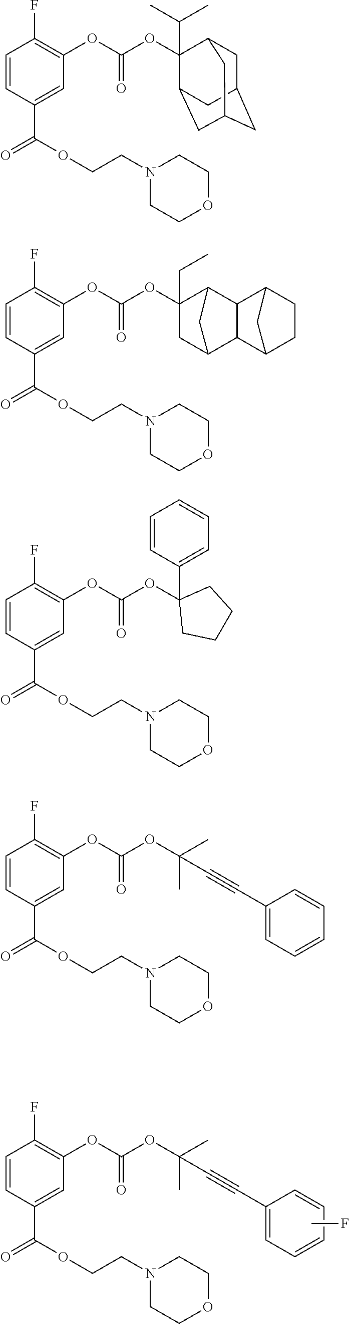

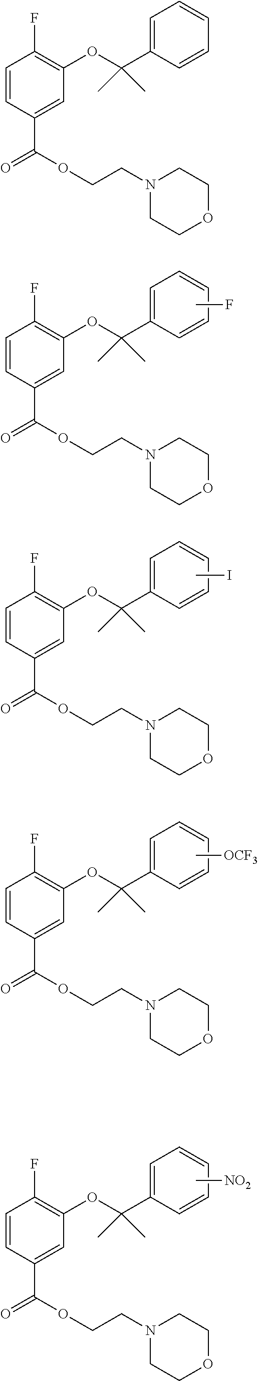

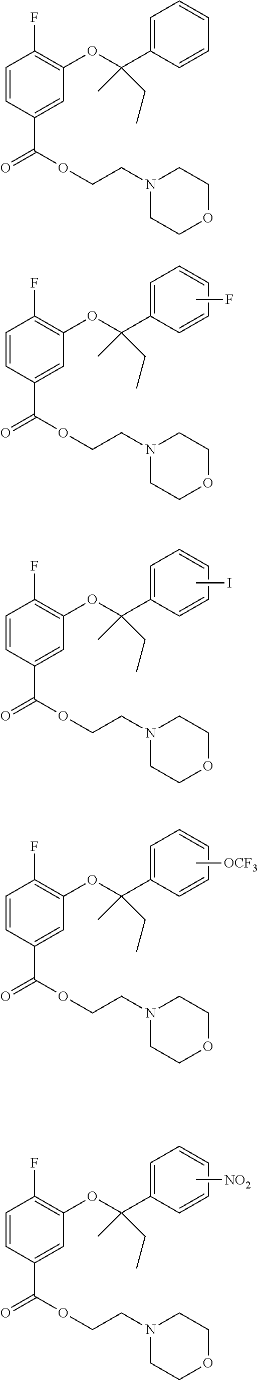

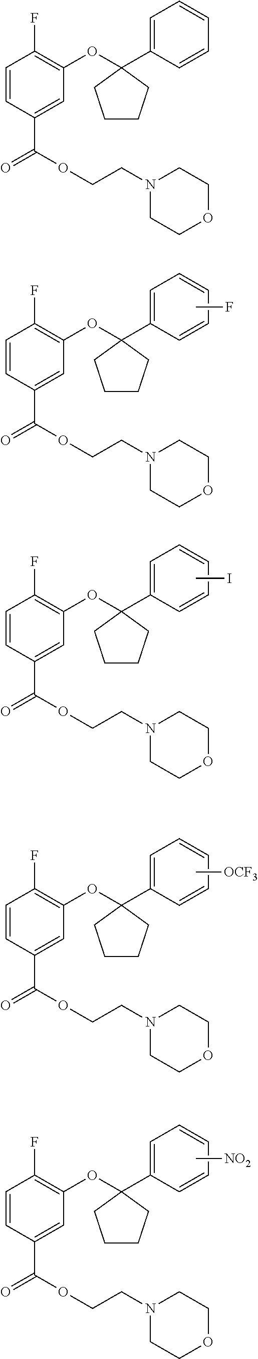

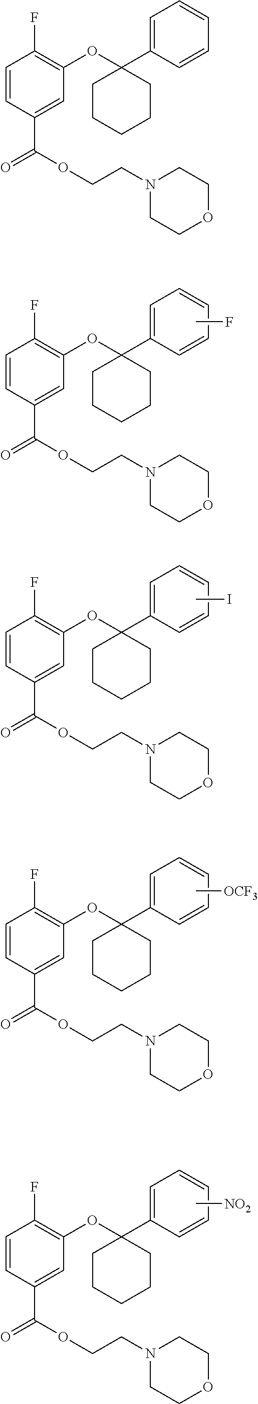

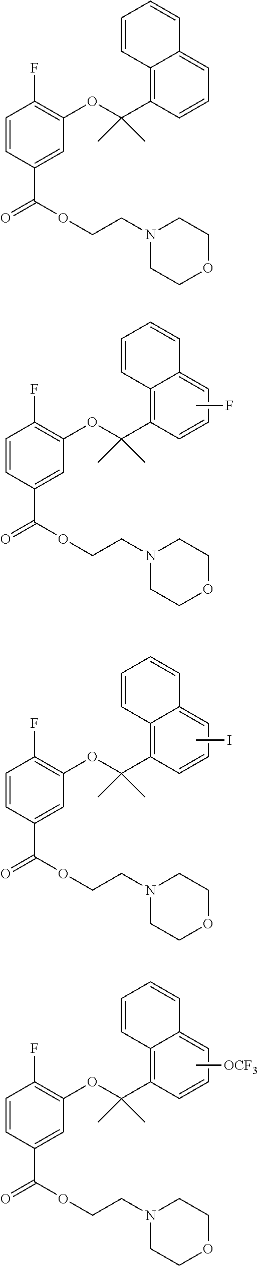

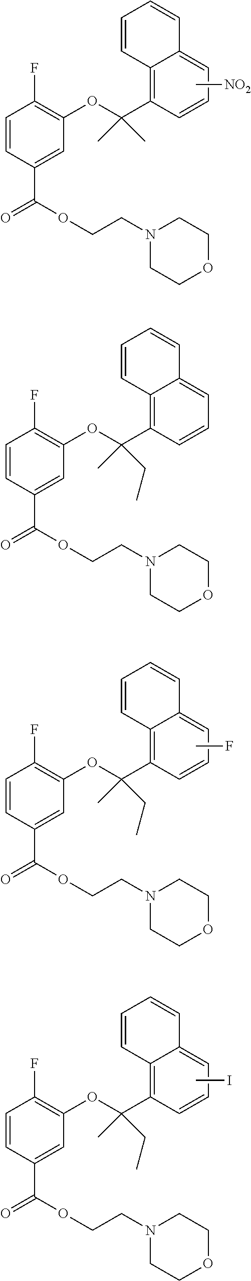

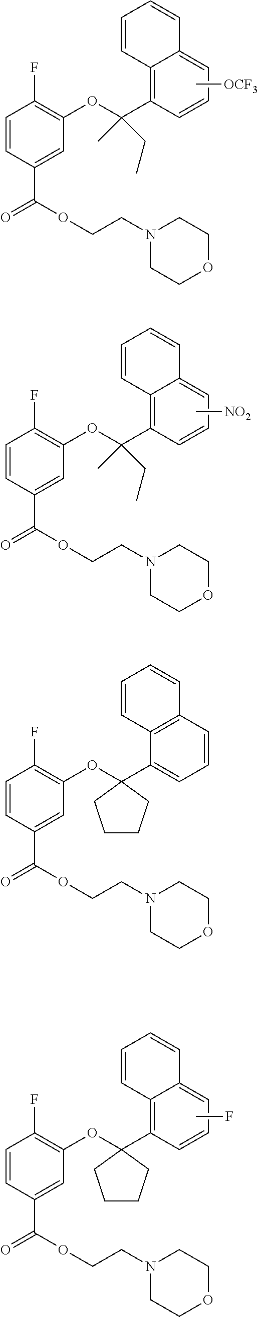

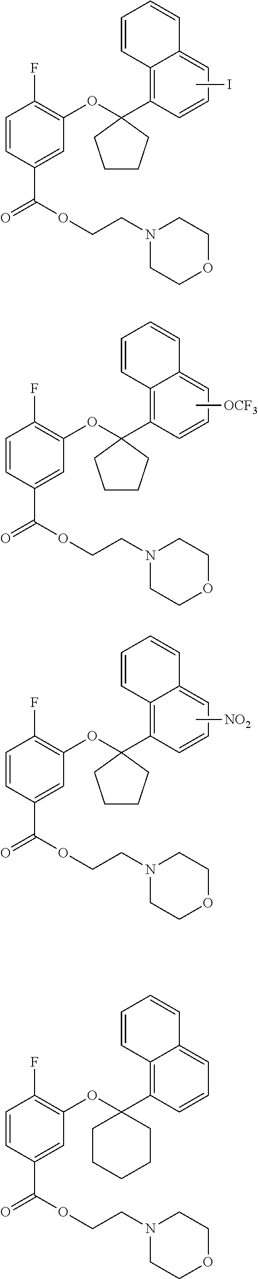

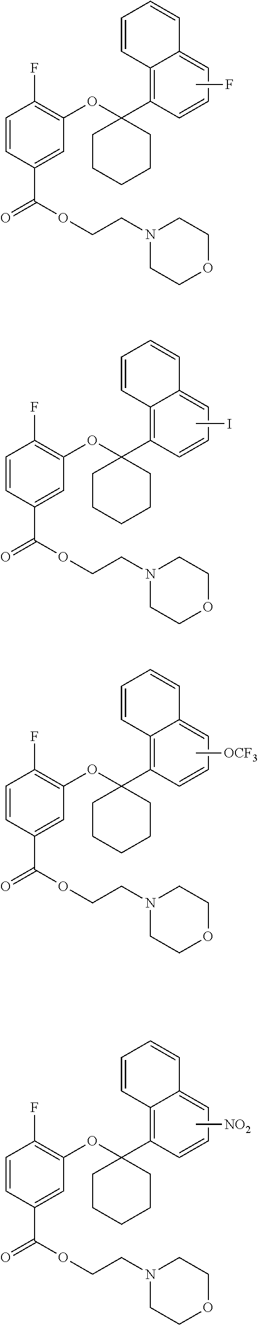

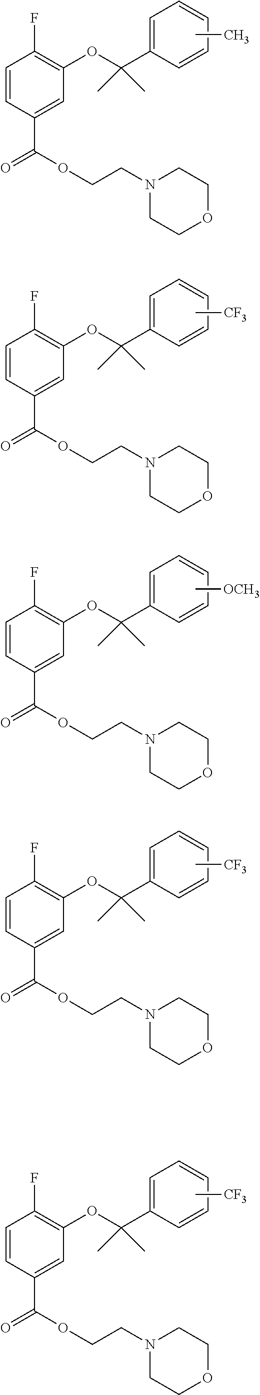

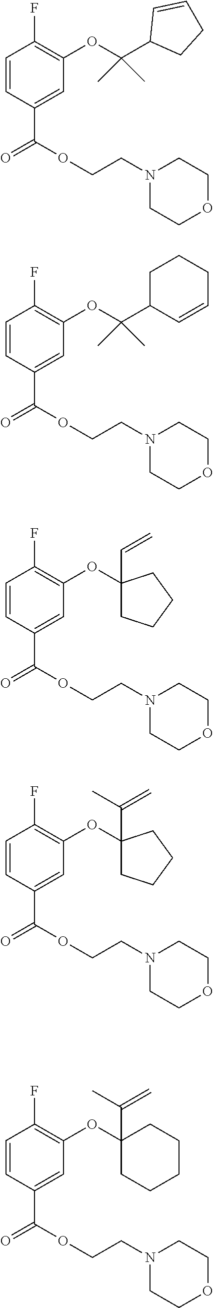

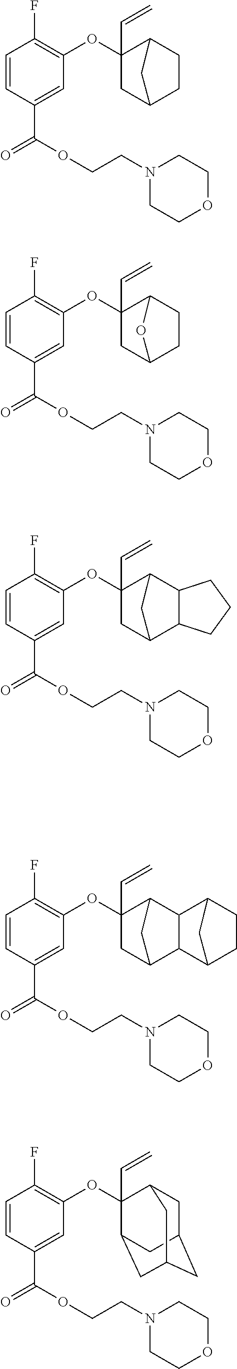 [SMILES: C=CC1(Oc2cc(C(=O)OCCN3CCOCC3)ccc2F)CC2CCC1C2 ; C=CC1(Oc2cc(C(=O)OCCN3CCOCC3)ccc2F)CC2CCC1O2 ; C=CC1(Oc2cc(C(=O)OCCN3CCOCC3)ccc2F)CC2CC1C1CCCC21 ; C=CC1(Oc2cc(C(=O)OCCN3CCOCC3)ccc2F)CC2CC1C1C3CCC(C3)C21 ; C=CC1(Oc2cc(C(=O)OCCN3CCOCC3)ccc2F)C2CC3CC(C2)CC1C3]

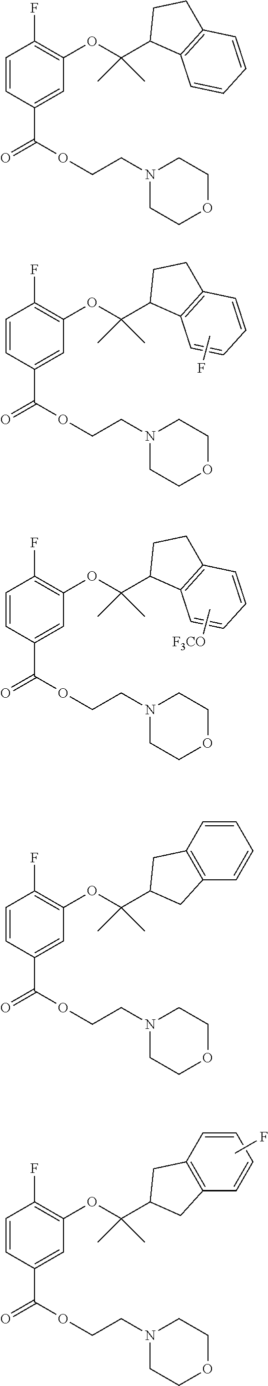

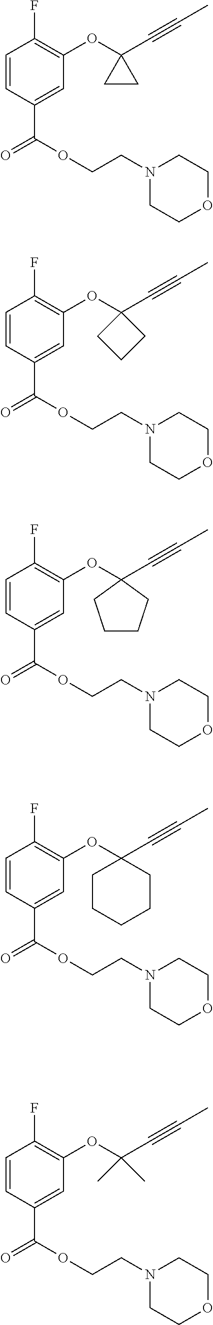 [SMILES: CC#CC1(Oc2cc(C(=O)OCCN3CCOCC3)ccc2F)CC1 ; CC#CC1(Oc2cc(C(=O)OCCN3CCOCC3)ccc2F)CCC1 ; CC#CC1(Oc2cc(C(=O)OCCN3CCOCC3)ccc2F)CCCC1 ; CC#CC1(Oc2cc(C(=O)OCCN3CCOCC3)ccc2F)CCCCC1 ; CC#CC(C)(C)Oc1cc(C(=O)OCCN2CCOCC2)ccc1F]

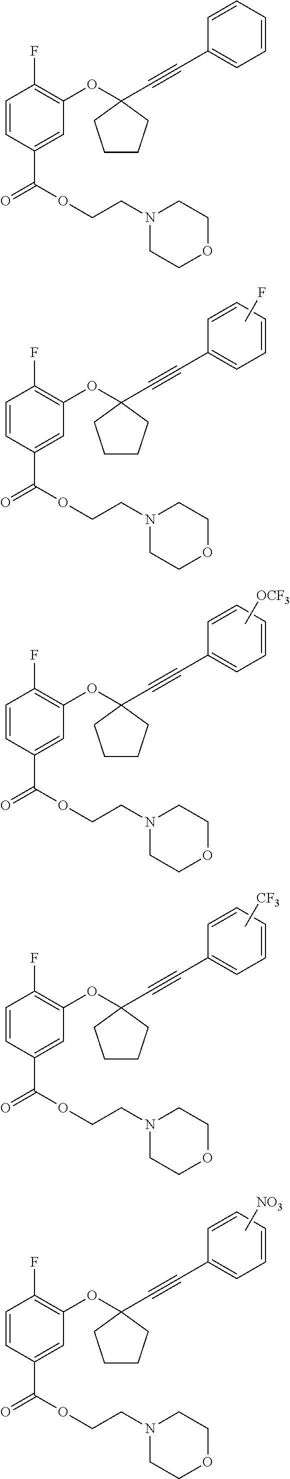

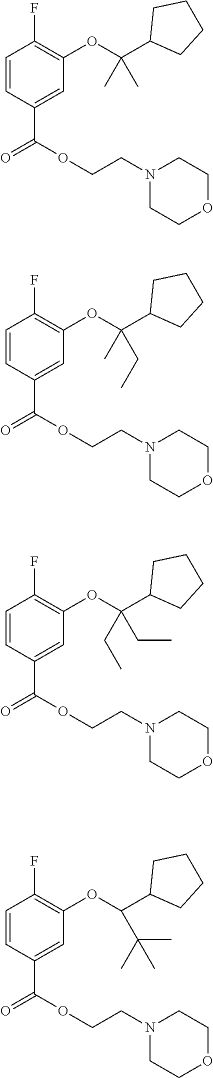 [SMILES: CC(C)(Oc1cc(C(=O)OCCN2CCOCC2)ccc1F)C1CCCC1 ; CCC(C)(Oc1cc(C(=O)OCCN2CCOCC2)ccc1F)C1CCCC1 ; CCC(CC)(Oc1cc(C(=O)OCCN2CCOCC2)ccc1F)C1CCCC1 ; CC(C)(C)C(Oc1cc(C(=O)OCCN2CCOCC2)ccc1F)C1CCCC1]

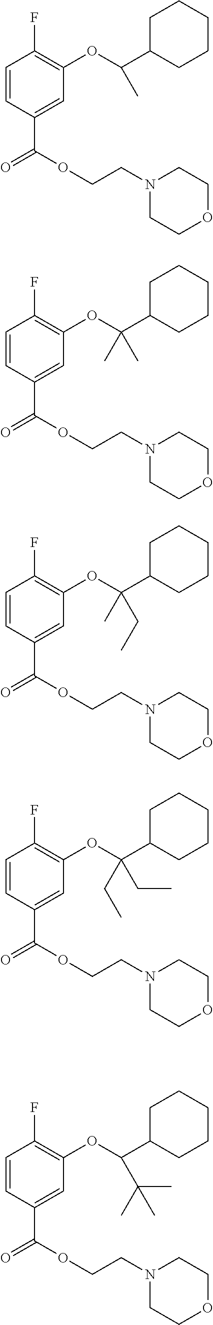

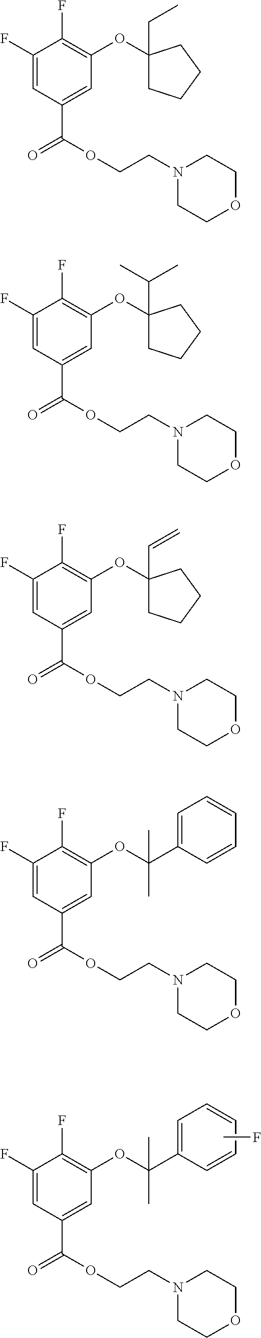

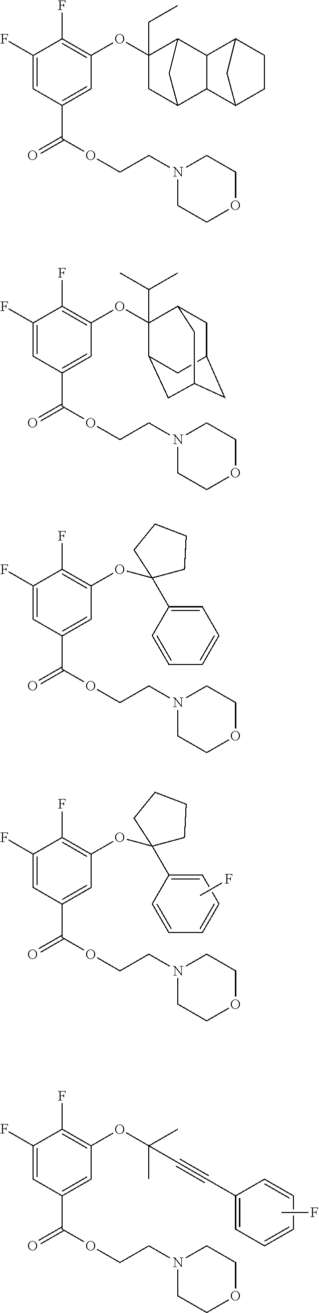

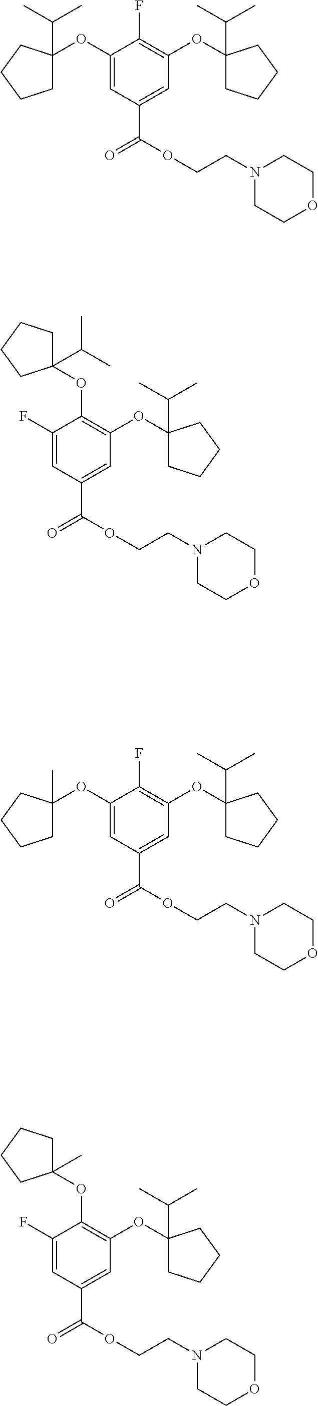 [SMILES: CC(C)C1(Oc2cc(C(=O)OCCN3CCOCC3)cc(OC3(C(C)C)CCCC3)c2F)CCCC1 ; CC(C)C1(Oc2cc(C(=O)OCCN3CCOCC3)cc(F)c2OC2(C(C)C)CCCC2)CCCC1 ; CC(C)C1(Oc2cc(C(=O)OCCN3CCOCC3)cc(OC3(C)CCCC3)c2F)CCCC1 ; CC(C)C1(Oc2cc(C(=O)OCCN3CCOCC3)cc(F)c2OC2(C)CCCC2)CCCC1]

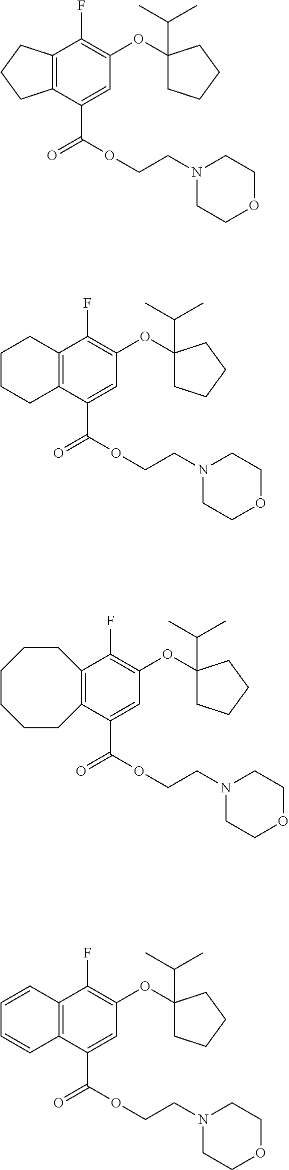 [SMILES: CC(C)C1(Oc2cc(C(=O)OCCN3CCOCC3)c3c(c2F)CCC3)CCCC1 ; CC(C)C1(Oc2cc(C(=O)OCCN3CCOCC3)c3c(c2F)CCCC3)CCCC1 ; CC(C)C1(Oc2cc(C(=O)OCCN3CCOCC3)c3c(c2F)CCCCCC3)CCCC1 ; CC(C)C1(Oc2cc(C(=O)OCCN3CCOCC3)c3ccccc3c2F)CCCC1]

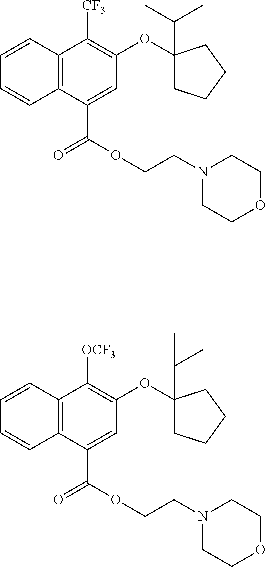 [SMILES: CC(C)C1(Oc2cc(C(=O)OCCN3CCOCC3)c3ccccc3c2C(F)(F)F)CCCC1 ; CC(C)C1(Oc2cc(C(=O)OCCN3CCOCC3)c3ccccc3c2OC(F)(F)F)CCCC1]

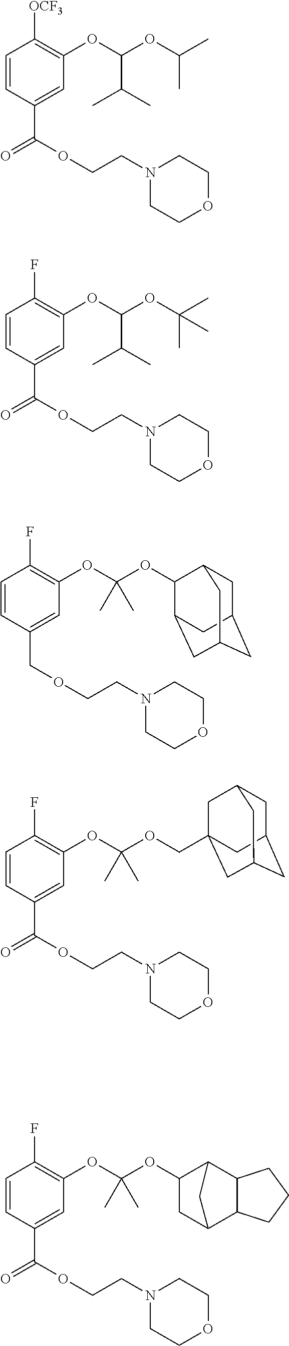 [SMILES: CC(C)OC(Oc1cc(C(=O)OCCN2CCOCC2)ccc1OC(F)(F)F)C(C)C ; CC(C)C(Oc1cc(C(=O)OCCN2CCOCC2)ccc1F)OC(C)(C)C ; CC(C)(Oc1cc(COCCN2CCOCC2)ccc1F)OC1C2CC3CC(C2)CC1C3 ; CC(C)(OCC12CC3CC(CC(C3)C1)C2)Oc1cc(C(=O)OCCN2CCOCC2)ccc1F ; CC(C)(Oc1cc(C(=O)OCCN2CCOCC2)ccc1F)OC1CC2CC1C1CCCC21]

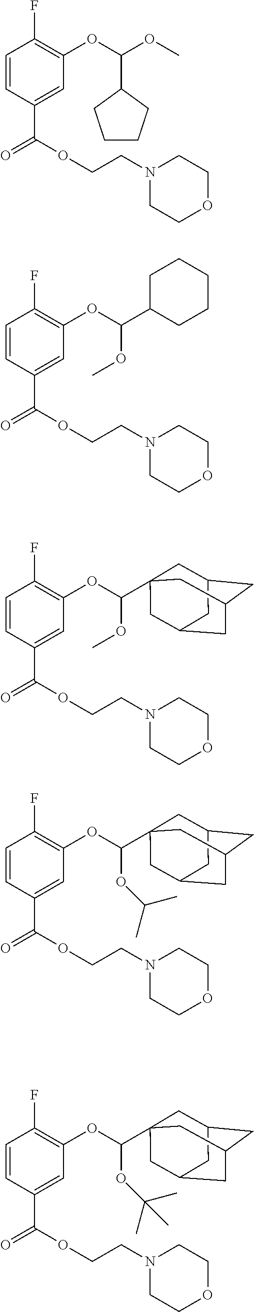

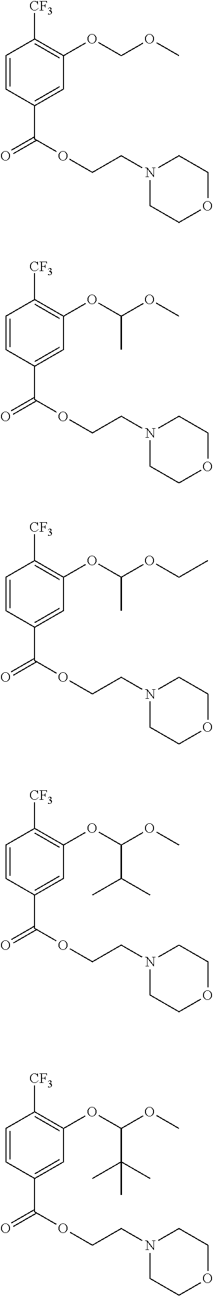 [SMILES: COCOc1cc(C(=O)OCCN2CCOCC2)ccc1C(F)(F)F ; COC(C)Oc1cc(C(=O)OCCN2CCOCC2)ccc1C(F)(F)F ; CCOC(C)Oc1cc(C(=O)OCCN2CCOCC2)ccc1C(F)(F)F ; COC(Oc1cc(C(=O)OCCN2CCOCC2)ccc1C(F)(F)F)C(C)C ; COC(Oc1cc(C(=O)OCCN2CCOCC2)ccc1C(F)(F)F)C(C)(C)C]

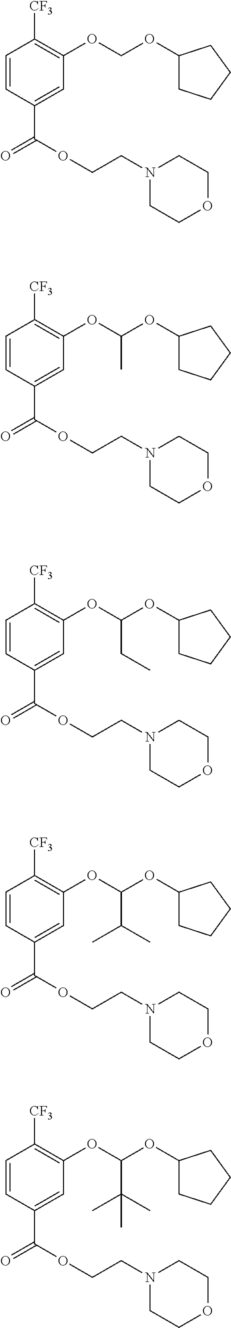 [SMILES: O=C(OCCN1CCOCC1)c1ccc(C(F)(F)F)c(OCOC2CCCC2)c1 ; CC(Oc1cc(C(=O)OCCN2CCOCC2)ccc1C(F)(F)F)OC1CCCC1 ; CCC(Oc1cc(C(=O)OCCN2CCOCC2)ccc1C(F)(F)F)OC1CCCC1 ; CC(C)C(Oc1cc(C(=O)OCCN2CCOCC2)ccc1C(F)(F)F)OC1CCCC1 ; CC(C)(C)C(Oc1cc(C(=O)OCCN2CCOCC2)ccc1C(F)(F)F)OC1CCCC1]

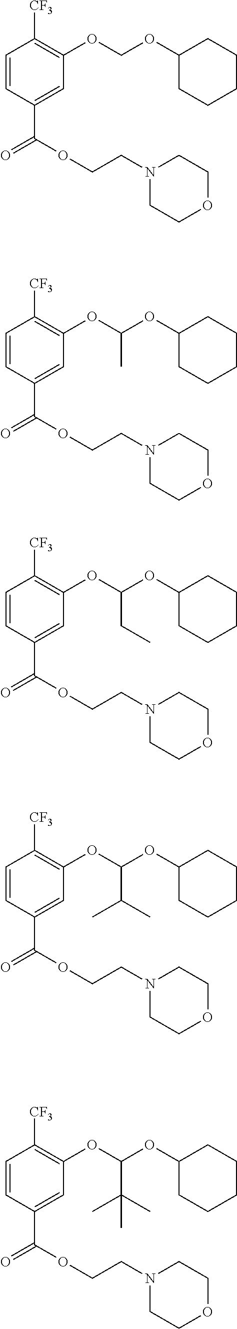 [SMILES: O=C(OCCN1CCOCC1)c1ccc(C(F)(F)F)c(OCOC2CCCCC2)c1 ; CC(Oc1cc(C(=O)OCCN2CCOCC2)ccc1C(F)(F)F)OC1CCCCC1 ; CCC(Oc1cc(C(=O)OCCN2CCOCC2)ccc1C(F)(F)F)OC1CCCCC1 ; CC(C)C(Oc1cc(C(=O)OCCN2CCOCC2)ccc1C(F)(F)F)OC1CCCCC1 ; CC(C)(C)C(Oc1cc(C(=O)OCCN2CCOCC2)ccc1C(F)(F)F)OC1CCCCC1]

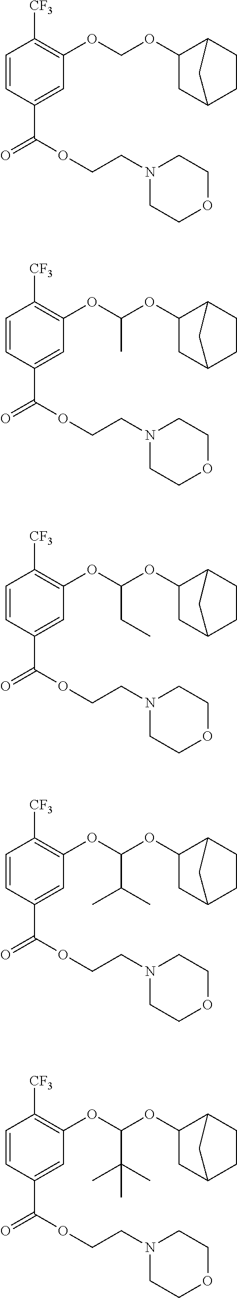 [SMILES: O=C(OCCN1CCOCC1)c1ccc(C(F)(F)F)c(OCOC2CC3CCC2C3)c1 ; CC(Oc1cc(C(=O)OCCN2CCOCC2)ccc1C(F)(F)F)OC1CC2CCC1C2 ; CCC(Oc1cc(C(=O)OCCN2CCOCC2)ccc1C(F)(F)F)OC1CC2CCC1C2 ; CC(C)C(Oc1cc(C(=O)OCCN2CCOCC2)ccc1C(F)(F)F)OC1CC2CCC1C2 ; CC(C)(C)C(Oc1cc(C(=O)OCCN2CCOCC2)ccc1C(F)(F)F)OC1CC2CCC1C2]

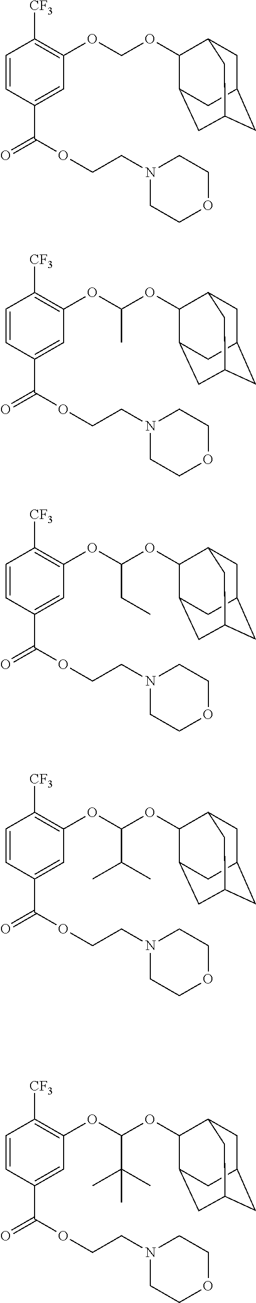 [SMILES: O=C(OCCN1CCOCC1)c1ccc(C(F)(F)F)c(OCOC2C3CC4CC(C3)CC2C4)c1 ; CC(Oc1cc(C(=O)OCCN2CCOCC2)ccc1C(F)(F)F)OC1C2CC3CC(C2)CC1C3 ; CCC(Oc1cc(C(=O)OCCN2CCOCC2)ccc1C(F)(F)F)OC1C2CC3CC(C2)CC1C3 ; CC(C)C(Oc1cc(C(=O)OCCN2CCOCC2)ccc1C(F)(F)F)OC1C2CC3CC(C2)CC1C3 ; CC(C)(C)C(Oc1cc(C(=O)OCCN2CCOCC2)ccc1C(F)(F)F)OC1C2CC3CC(C2)CC1C3]

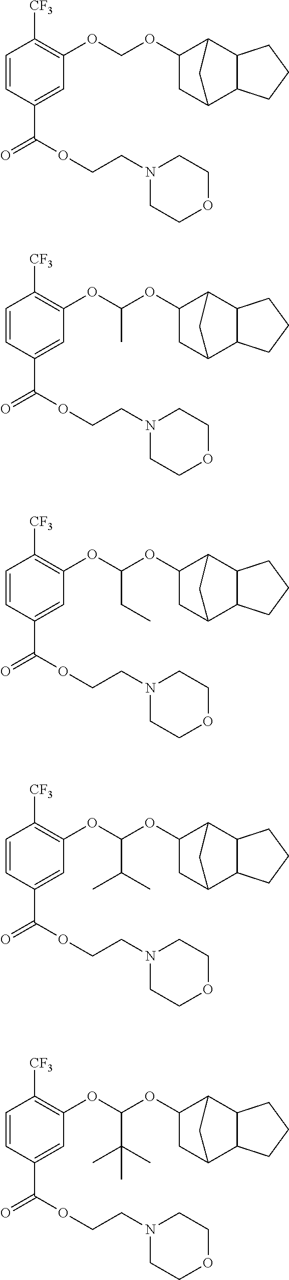 [SMILES: O=C(OCCN1CCOCC1)c1ccc(C(F)(F)F)c(OCOC2CC3CC2C2CCCC32)c1 ; CC(Oc1cc(C(=O)OCCN2CCOCC2)ccc1C(F)(F)F)OC1CC2CC1C1CCCC21 ; CCC(Oc1cc(C(=O)OCCN2CCOCC2)ccc1C(F)(F)F)OC1CC2CC1C1CCCC21 ; CC(C)C(Oc1cc(C(=O)OCCN2CCOCC2)ccc1C(F)(F)F)OC1CC2CC1C1CCCC21 ; CC(C)(C)C(Oc1cc(C(=O)OCCN2CCOCC2)ccc1C(F)(F)F)OC1CC2CC1C1CCCC21]

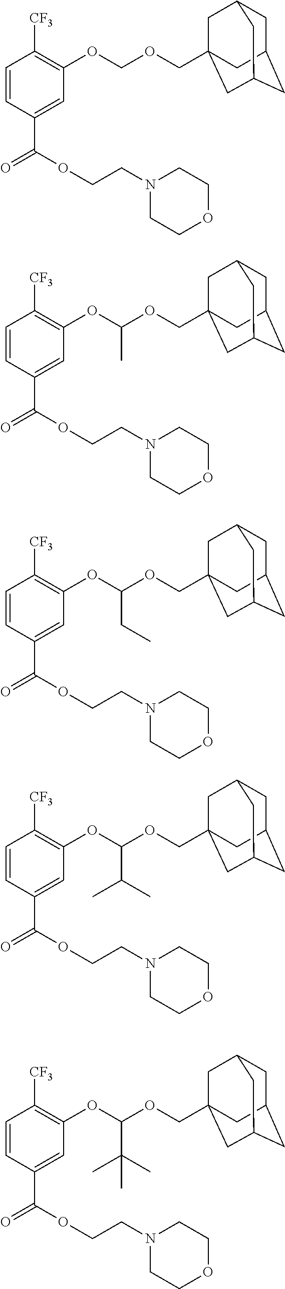 [SMILES: O=C(OCCN1CCOCC1)c1ccc(C(F)(F)F)c(OCOCC23CC4CC(CC(C4)C2)C3)c1 ; CC(OCC12CC3CC(CC(C3)C1)C2)Oc1cc(C(=O)OCCN2CCOCC2)ccc1C(F)(F)F ; CCC(OCC12CC3CC(CC(C3)C1)C2)Oc1cc(C(=O)OCCN2CCOCC2)ccc1C(F)(F)F ; CC(C)C(OCC12CC3CC(CC(C3)C1)C2)Oc1cc(C(=O)OCCN2CCOCC2)ccc1C(F)(F)F ; CC(C)(C)C(OCC12CC3CC(CC(C3)C1)C2)Oc1cc(C(=O)OCCN2CCOCC2)ccc1C(F)(F)F]

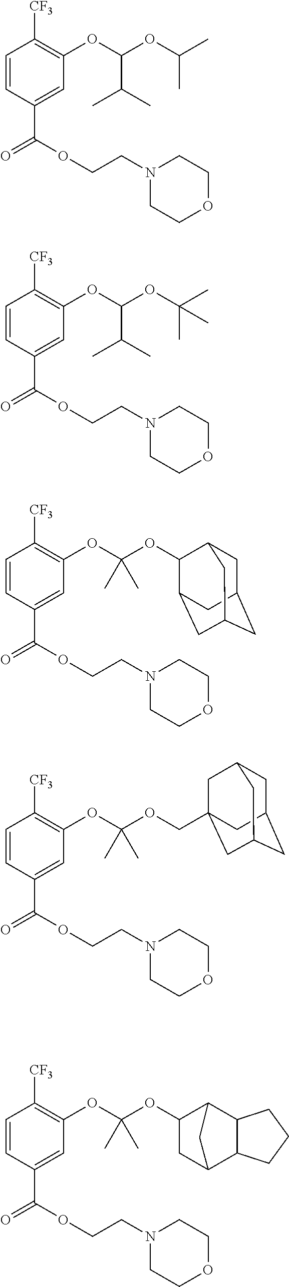 [SMILES: CC(C)OC(Oc1cc(C(=O)OCCN2CCOCC2)ccc1C(F)(F)F)C(C)C ; CC(C)C(Oc1cc(C(=O)OCCN2CCOCC2)ccc1C(F)(F)F)OC(C)(C)C ; CC(C)(Oc1cc(C(=O)OCCN2CCOCC2)ccc1C(F)(F)F)OC1C2CC3CC(C2)CC1C3 ; CC(C)(OCC12CC3CC(CC(C3)C1)C2)Oc1cc(C(=O)OCCN2CCOCC2)ccc1C(F)(F)F ; CC(C)(Oc1cc(C(=O)OCCN2CCOCC2)ccc1C(F)(F)F)OC1CC2CC1C1CCCC21]

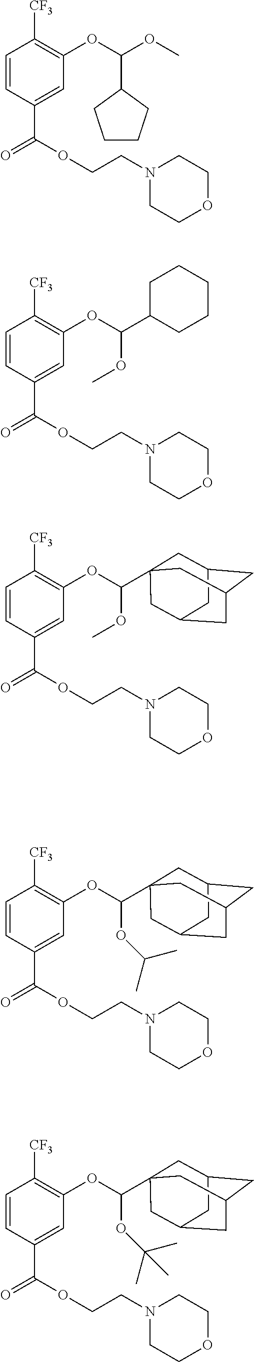

The amine compound having formula (1) can be prepared according to the following scheme.

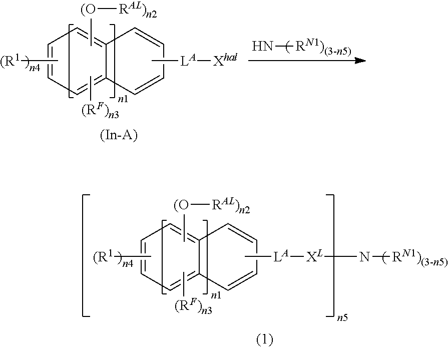

Herein, n1 to n5, R^{AL}, R^{F}, R¹, L^{A}, X^{L}, and R^{N1} are as defined above, and X^{hal} is chlorine, bromine or iodine.

Namely, the amine compound having formula (1) can be prepared by substitution reaction of intermediate In-A, which can be synthesized by a well-known method, with a primary or secondary amine.

The synthesis can be carried out by any well-known organic synthesis method. Specifically, reaction is carried out by dissolving intermediate In-A in a polar aprotic solvent such as acetone, acetonitrile, dimethylformamide or dimethyl sulfoxide, and adding a primary or secondary amine to the solution. In the case of intermediate In-A wherein X^{hal} is chlorine or bromine, the reaction may be accelerated by adding a catalytic amount of an alkali metal or quaternary ammonium iodide. Exemplary alkali metal iodides include sodium iodide and potassium iodide. Exemplary quaternary ammonium iodides include tetraethylammonium iodide and benzyltrimethylammonium iodide. The reaction temperature preferably ranges from room temperature to nearly the boiling point of the solvent used. While it is desirable from the aspect of yield to monitor the reaction by gas chromatography (GC) or silica gel thin layer chromatography (TLC) until the reaction is complete, the reaction time is typically about 30 minutes to about 20 hours. The amine compound having formula (1) may be collected from the reaction mixture by standard aqueous work-up. If necessary, the amine compound is purified by a standard technique such as chromatography or recrystallization.

The above preparation method is merely exemplary and the method of preparing the amine compound is not limited thereto.

The amine compound of the invention is structurally characterized in that it has an acid labile group attached to a hydroxy group on aromatic ring and a fluorinated substituent group on aromatic ring, which are attached to the neighboring carbon atoms. In the exposed region, the acid labile group undergoes deprotection reaction with the aid of the generated acid, whereby an aromatic hydroxy group is generated. This leads to an improvement in contrast between exposed and unexposed regions. Also, the neighboring fluorinated substituent group serves to improve the solubility in resist solvent and its electron attraction serves to increase the acidity of aromatic hydroxy group generated in the exposed region. When the resist film after exposure is developed in alkaline developer, the increased affinity of the generated aromatic hydroxy group to the alkaline developer ensures that the exposed region is effectively removed by the developer. The aromatic hydroxy group neighboring the fluorinated substituent group ensures that the alkaline developer is not taken more into the unexposed region than the carboxy group due to the water repellent effect of fluorine. The effect of suppressing swell in the alkaline developer is thus exerted. This restrains the resist pattern from collapsing in the unexposed region. By the synergy of these effects, the inventive amine compound enables to form a resist pattern having a high dissolution contrast, reduced LWR of line patterns, improved CDU of hole patterns, and high collapse resistance. The amine compound is thus suited for formulating a positive resist composition.

Chemically Amplified Resist Composition

Another embodiment of the invention is a chemically amplified resist composition essentially comprising (A) a quencher in the form of the amine compound having formula (1). As used herein, the “quencher” refers to a compound capable of trapping an acid generated from a photoacid generator in the resist composition to prevent the acid from diffusing to the unexposed region for thereby forming the desired pattern.

In the chemically amplified resist composition, the amount of the quencher (A) in the form of the amine compound having formula (1) blended is preferably 0.1 to 20 parts by weight, more preferably 0.5 to 15 parts by weight per 80 parts by weight of the base polymer to be described below. With the amount of quencher (A) in the range, sensitivity and resolution are good, and there is no risk of raising the problem of foreign particles after development or stripping of the resist film. The quencher (A) may be used alone or in admixture of two or more.

(B) Base Polymer

The chemically amplified resist composition may comprise (B) a base polymer. Preferably the base polymer comprises repeat units having the formula (a1) or repeat units having the formula (a2). These units are also referred to as repeat units (a1) and (a2).

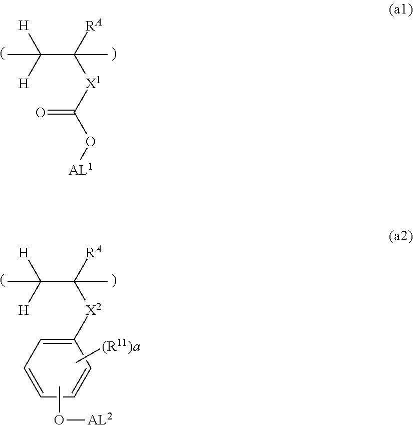

In formulae (a1) and (a2), R^{A} is each independently hydrogen, fluorine, methyl or trifluoromethyl. X¹ is a single bond, phenylene, naphthylene, or *—C(═O)—O—X¹¹—. The phenylene or naphthylene group may be substituted with an optionally fluorinated C₁-C₁₀ alkoxy moiety or halogen. X¹¹ is a C₁-C₁₀ saturated hydrocarbylene group, which may contain a hydroxy moiety, ether bond, ester bond or lactose ring, or a phenylene group or naphthylene group. X² is a single bond or *—C(═O)—O—. The asterisk (*) designates a point of attachment to the carbon atom in the backbone. AL¹ and AL² are each independently an acid labile group.

In formula (a2), R¹¹ is a C₁-C₂₀ hydrocarbyl group which may contain a heteroatom. The hydrocarbyl group may be saturated or unsaturated and straight, branched or cyclic. Examples thereof are as exemplified above for the C₁-C₂₀ hydrocarbyl group R¹ in formula (1).

In formula (a2), “a” is an integer of 0 to 4, preferably 0 or 1.

Examples of the structure of formula (a1) wherein X¹ is a variant are illustrated below, but not limited thereto. Herein R^{A} and AL¹ are as defined above.

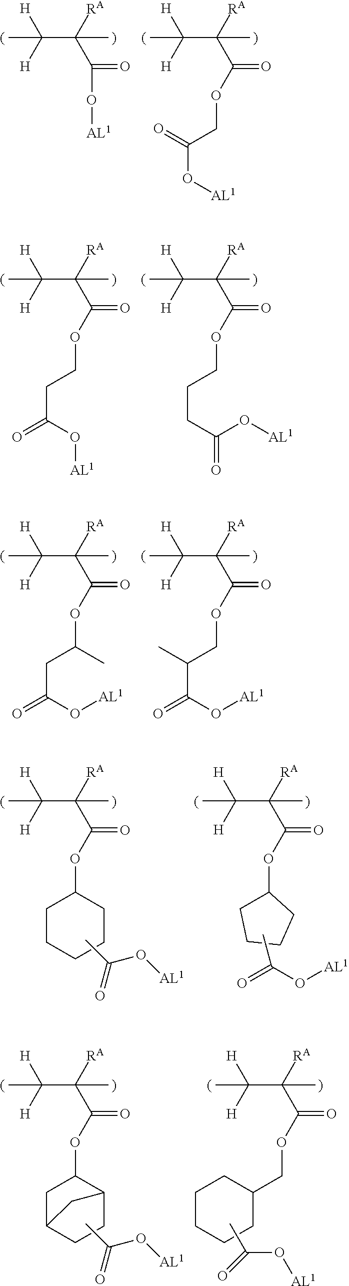

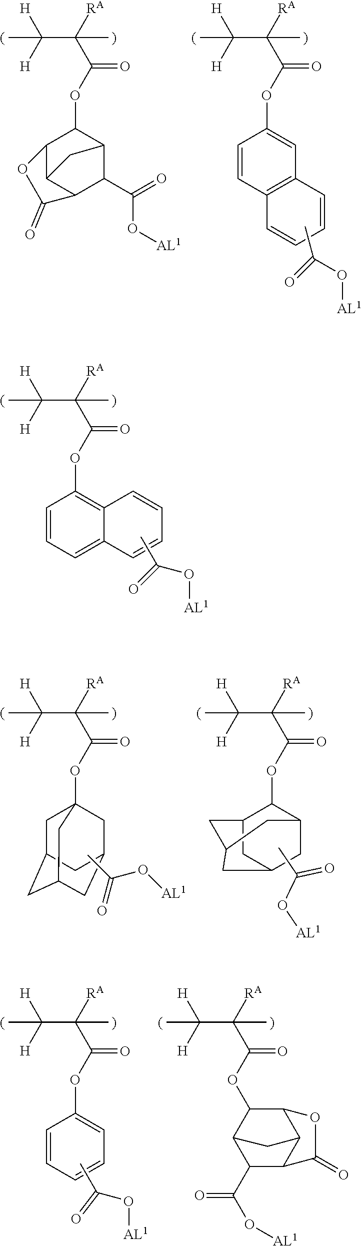

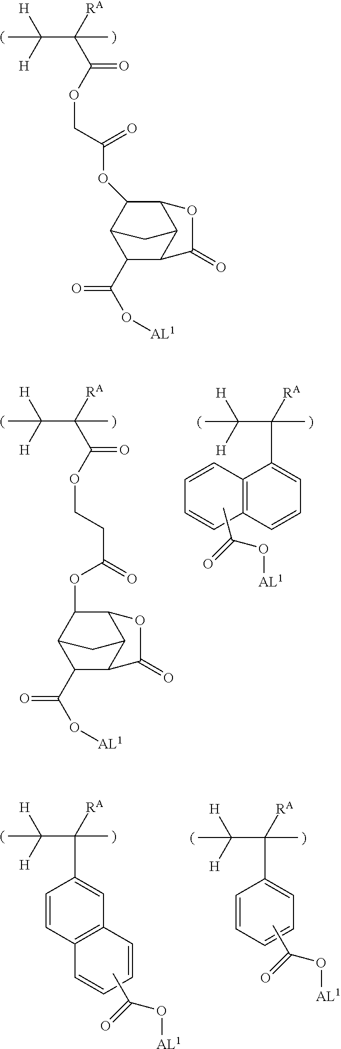

A polymer comprising repeat units (a1) turns alkali soluble through the mechanism that it is decomposed to generate a carboxy group under the action of acid.

The acid labile groups represented by AL¹ and AL² may be selected from a variety of such groups. Preferred examples of the acid labile group are groups of the following formulae (L1) to (L4), C₄-C₂₀, preferably C₄-C₁₅ tertiary hydrocarbyl groups, trialkylsilyl groups in which each alkyl moiety has 1 to 6 carbon atoms, and C₄-C₂₀ saturated hydrocarbyl groups containing a carbonyl moiety, ether bond or ester bond.

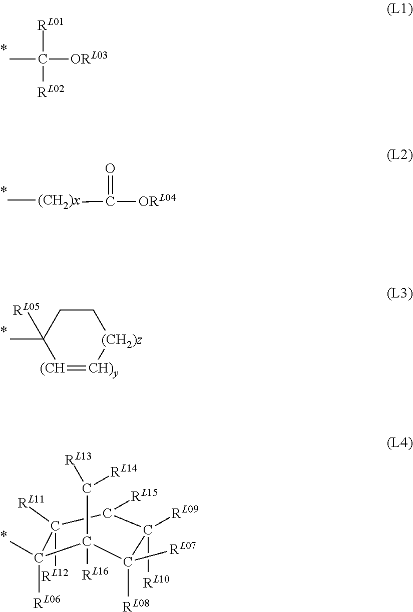

In formula (L1), R^{L01} and R^{L02} are each independently hydrogen or a C₁-C₁₈ saturated hydrocarbyl group. The saturated hydrocarbyl group may be straight, branched or cyclic and examples thereof include alkyl groups such as methyl, ethyl, n-propyl, isopropyl, n-butyl, sec-butyl, tert-butyl, n-octyl, and 2-ethylhexyl, and cyclic saturated hydrocarbyl groups such as cyclopentyl, cyclohexyl, norbornyl, tricyclodecanyl, tetracyclododecanyl, and adamantyl. Of the saturated hydrocarbyl groups, those of 1 to 10 carbon atoms are preferred.

R^{L03} is a C₁-C₁₈, preferably C₁-C₁₀ hydrocarbyl group which may contain a moiety containing a heteroatom. The hydrocarbyl group may be saturated or unsaturated and straight, branched or cyclic. Saturated hydrocarbyl groups are preferred. In the saturated hydrocarbyl group, some or all of the hydrogen atoms may be substituted by hydroxy, saturated hydrocarbyloxy, oxo, amino, saturated hydrocarbylamino or the like, or some constituent —CH₂— may be replaced by a moiety containing a heteroatom, typically oxygen. Suitable saturated hydrocarbyl groups are as exemplified above for the saturated hydrocarbyl groups R^{L01} and R^{L02}. Examples of the substituted saturated hydrocarbyl group are shown below.

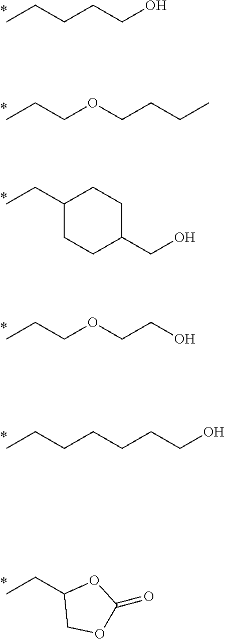

Any two of R^{L01}, R^{L02}, and R^{L03} may bond together to form a ring with the carbon atom or the carbon and oxygen atoms to which they are attached. When any two of R^{L01}, R^{L02} and R^{L03} form a ring, each is independently a C₁-C₁₈, preferably C₁-C₁₀ alkanediyl group.

In formula (L2), R^{L04} is a C₄-C₂₀, preferably C₄-C₁₅ tertiary hydrocarbyl group, a trialkylsilyl group in which each alkyl moiety has 1 to 6 carbon atoms, a C₄-C₂₀ saturated hydrocarbyl group containing a carbonyl moiety, ether bond or ester bond, or a group of formula (L1). The subscript x is an integer of 0 to 6.

Of the groups R^{L04}, the tertiary hydrocarbyl group may be branched or cyclic, and examples thereof include tert-butyl, tert-pentyl, 1,1-diethylpropyl, 2-cyclopentylpropan-2-yl, 2-cyclohexylpropan-2-yl, 2-(bicyclo[2.2.1]heptan-2-yl)propan-2-yl, 2-(adamantan-1-yl)propan-2-yl, 1-ethylcyclopentyl, 1-butylcyclopentyl, 1-ethylcyclohexyl, 1-butylcyclohexyl, 1-ethyl-2-cyclopentenyl, 1-ethyl-2-cyclohexenyl, 2-methyl-2-adamantyl, and 2-ethyl-2-adamantyl. Exemplary trialkylsilyl groups include trimethylsilyl, triethylsilyl, and dimethyl-tert-butylsilyl. Exemplary saturated hydrocarbyl groups containing a carbonyl, ether bond or ester bond include 3-oxocyclohexyl, 4-methyl-2-oxooxan-4-yl, and 5-methyl-2-oxooxolan-5-yl.

In formula (L3), R^{L05} is an optionally substituted C₁-C₈ saturated hydrocarbyl group or an optionally substituted C₆-C₂₀ aryl group. The optionally substituted saturated hydrocarbyl group may be straight, branched or cyclic and examples thereof include alkyl groups such as methyl, ethyl, n-propyl, isopropyl, n-butyl, sec-butyl, tert-butyl, n-pentyl, tert-pentyl, and n-hexyl, cyclic saturated hydrocarbyl groups such as cyclopentyl and cyclohexyl, and substituted forms of the foregoing in which some or all of the hydrogen atoms are substituted by hydroxy, C₁-C₆ saturated hydrocarbyloxy, carboxy, C₁-C₆ saturated hydrocarbylcarbonyl, oxo, amino, C₁-C₆ saturated hydrocarbylamino, cyano, mercapto, C₁-C₆ saturated hydrocarbylthio, sulfo or the like. Examples of the optionally substituted aryl group include phenyl, methylphenyl, naphthyl, anthryl, phenanthryl, and pyrenyl, and substituted forms of the foregoing in which some or all of the hydrogen atoms are substituted by hydroxy, C₁-C₁₀ saturated hydrocarbyloxy, carboxy. C₁-C₁₀ saturated hydrocarbylcarbonyl, oxo, amino, C₁-C₁₀ saturated hydrocarbylamino, cyano, mercapto, C₁-C₁₀ saturated hydrocarbylthio, sulfo or the like.

In formula (L3), y is equal to 0 or 1, z is an integer of 0 to 3, and 2y+z is equal to 2 or 3.

In formula (L4), R^{L06} is an optionally substituted C₁-C₈ saturated hydrocarbyl group or an optionally substituted C₆-C₂₀ aryl group. Examples of the optionally substituted saturated hydrocarbyl and optionally substituted aryl groups are the same as exemplified above for R^{L05}.

R^{L07} to R^{L16} are each independently hydrogen or an optionally substituted C₁-C₁₅ hydrocarbyl group. The hydrocarbyl group may be saturated or unsaturated and straight, branched or cyclic, with saturated hydrocarbyl groups being preferred. Examples of the hydrocarbyl group include alkyl groups such as methyl, ethyl, n-propyl, isopropyl, n-butyl, sec-butyl, tert-butyl, n-pentyl, tert-pentyl, n-hexyl, n-octyl, n-pony 1, and n-decyl; cyclic saturated hydrocarbyl groups such as cyclopentyl, cyclohexyl, cyclopentylmethyl, cyclopentylethyl, cyclopentylbutyl, cyclohexylmethyl, cyclohexylethyl and cyclohexylbutyl; and substituted forms of the foregoing in which some or all of the hydrogen atoms are substituted by hydroxy, C₁-C₁₀ saturated hydrocarbyloxy, carboxy. C₁-C₁₀ saturated hydrocarbyloxycarbonyl, oxo, amino. C₁-C₁₀ saturated hydrocarbylamino, cyano, mercapto, C₁-C₁₀ saturated hydrocarbylthio, sulfo or the like. Alternatively, two of R^{L07} to R^{L16} may bond together to form a ring with the carbon atom to which they are attached (for example, a pair of R^{L07} and R^{L08}, R^{L07} and R^{L09}, R^{L07} and R^{L10}, R^{L08} and R^{L10}, R^{L09} and R^{L10}, R^{L11} and R^{L12}, R^{L13} and R^{L14}, or a similar pair form a ring). Each of ring-forming R^{L07} to R^{L16} represents a C₁-C¹⁵ hydrocarbylene group, examples of which are the ones exemplified above for the hydrocarbyl groups, with one hydrogen atom being eliminated. Two of R^{L07} to R^{L16} which are attached to vicinal carbon atoms may bond together directly to form a double bond (for example, a pair of R^{L07} and R^{L09}, R^{L09} and R^{L15}, R^{L13} and R^{L15}, R^{L14} and R^{L15}, or a similar pair).

Of the acid labile groups having formula (L1), the straight and branched ones are exemplified by the following groups, but not limited thereto.

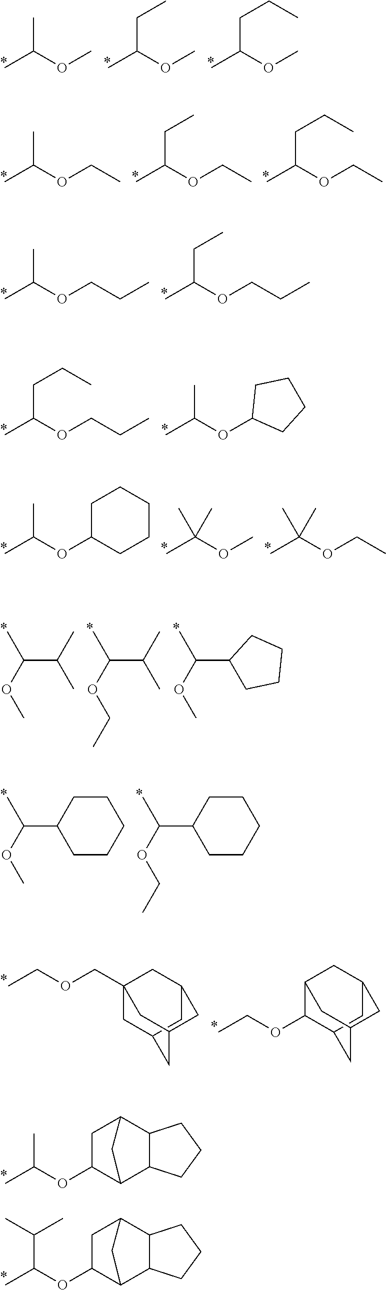

Of the acid labile groups having formula (L1), the cyclic ones are, for example, tetrahydrofuran-2-yl, 2-methyltetrahydrofuran-2-yl, tetrahydropyran-2-yl, and 2-methyltetrahydropyran-2-yl.

Examples of the acid labile group having formula (L2) include tert-butoxycarbonyl, tert-butoxycarbonylmethyl, tert-pentyloxycarbonyl, tert-pentyloxycarbonylmethyl, 1,1-diethylpropyloxycarbonyl, 1,1-diethylpropyloxycarbonylmethyl, 1-ethylcyclopentyloxycarbonyl, 1-ethylcyclopentyloxycarbonylmethyl, 1-ethyl-2-cyclopentenyloxycarbonyl, 1-ethyl-2-cyclopentenyloxycarbonylmethyl, 1-ethoxyethoxycarbonylmethyl, 2-tetrahydropyranyloxycarbonylmethyl, and 2-tetrahydrofuranyloxycarbonylmethyl groups.

Examples of the acid labile group having formula (L3) include 1-methylcyclopentyl, 1-ethylcyclopentyl, 1-n-propylcyclopentyl, 1-isopropylcyclopentyl, 1-n-butylcyclopentyl, 1-sec-butylcyclopentyl, 1-cyclohexylcyclopentyl, 1-(4-methoxy-n-butyl)cyclopentyl, 1-methylcyclohexyl, 1-ethylcyclohexyl, 3-methyl-1-cyclopenten-3-yl, 3-ethyl-1-cyclopenten-3-yl, 3-methyl-1-cyclohexen-3-yl, and 3-ethyl-1-cyclohexen-3-yl groups.

Of the acid labile groups having formula (L4), groups having the following formulae (L4-1) to (L4-4) are preferred.

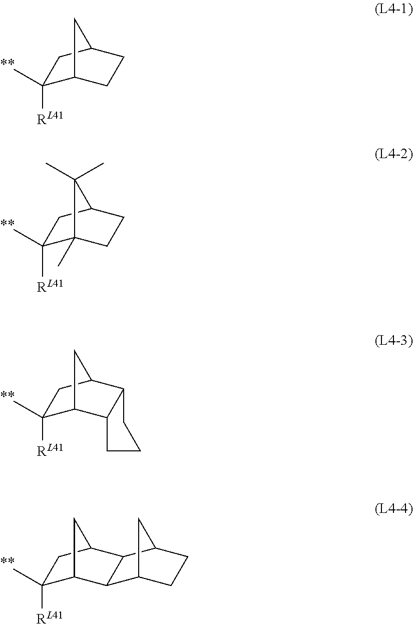

In formulae (L4-1) to (L4-4), the double asterisks (**) denotes a bonding site and direction. R^{L41} is each independently a C₁-C₁₀ hydrocarbyl group. The hydrocarbyl group may be saturated or unsaturated and straight, branched or cyclic, with saturated hydrocarbyl groups being preferred. Suitable hydrocarbyl groups include alkyl groups such as methyl, ethyl, n-propyl, isopropyl, n-butyl, isobutyl, sec-butyl, tert-butyl, n-pentyl, tert-pentyl, and n-hexyl, and cyclic saturated hydrocarbyl groups such as cyclopentyl and cyclohexyl.

For formulae (L4-1) to (L4-4), there can exist stereoisomers (enantiomers or diastereomers). Each of formulae (L4-1) to (L4-4) collectively represents all such stereoisomers. When the acid labile group is of formula (L4), there may be contained a plurality of stereoisomers.

For example, the formula (L4-3) represents one or a mixture of two selected from groups having the following formulae (L4-3-1) and (L4-3-2).

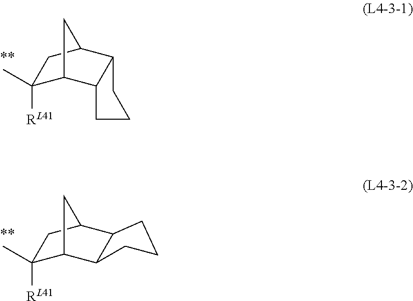

Herein R^{L41} and double asterisks (**) are as defined above.

Similarly, the formula (L4-4) represents one or a mixture of two or more selected from groups having the following formulae (L4-4-1) to (L4-4-4).

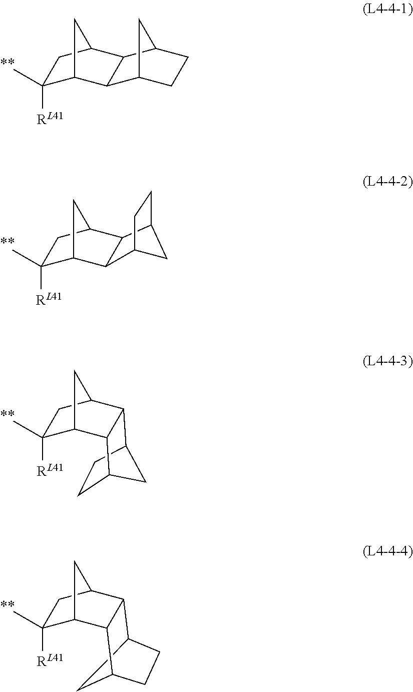

Herein R^{L41} and double asterisks (**) are as defined above.

Each of formulae (L4-1) to (L4-4), (L4-3-1), (L4-3-2), and (L4-4-1) to (L4-4-4) collectively represents an enantiomer thereof and a mixture of enantiomers.

It is noted that in the above formulae (L4-1) to (L4-4), (L4-3-1), (L4-3-2), and (L4-4-1) to (L4-4-4), the bond direction is on the exo side relative to the bicyclo[2.2.1]heptane ring, which ensures high reactivity for acid catalyzed elimination reaction (see JP-A 2000-336121). In preparing these monomers having a tertiary exo-saturated hydrocarbyl group of bicyclo[2.2.1]heptane skeleton as a substituent group, there may be contained monomers substituted with an endo-alkyl group as represented by the following formulae (L4-1-endo) to (L4-4-endo). For good reactivity, an exo proportion of at least 50 mol % is preferred, with an exo proportion of at least 80 mol % being more preferred.

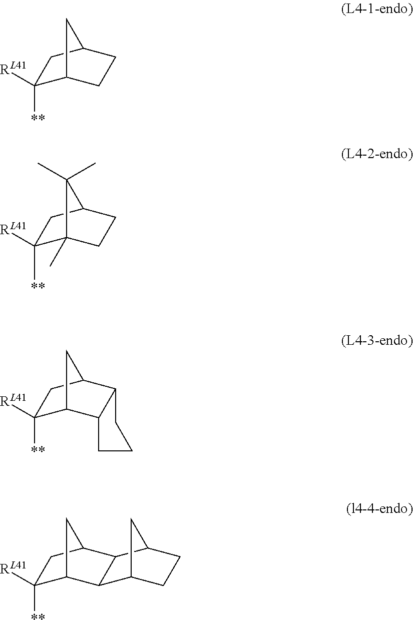

Herein R^{L41} and double asterisks (**) are as defined above.

Illustrative examples of the acid labile group having formula (L4) are given below, but not limited thereto.

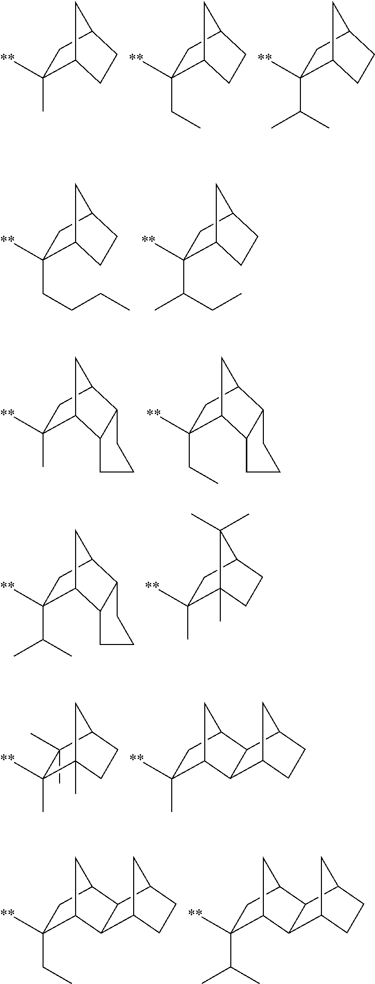

Herein double asterisks (**) is as defined above.

Of the acid labile groups represented by AL¹ and AL², examples of the C₄-C₂₀ tertiary hydrocarbyl groups, trialkylsilyl groups in which each alkyl moiety has 1 to 6 carbon atoms, and C₄-C₂₀ saturated hydrocarbyl groups containing carbonyl, ether bond or ester bond are as exemplified above for R^{L04}.

Illustrative examples of the repeat units (a1) are given below, but not limited thereto. Herein R^{A} is as defined above.

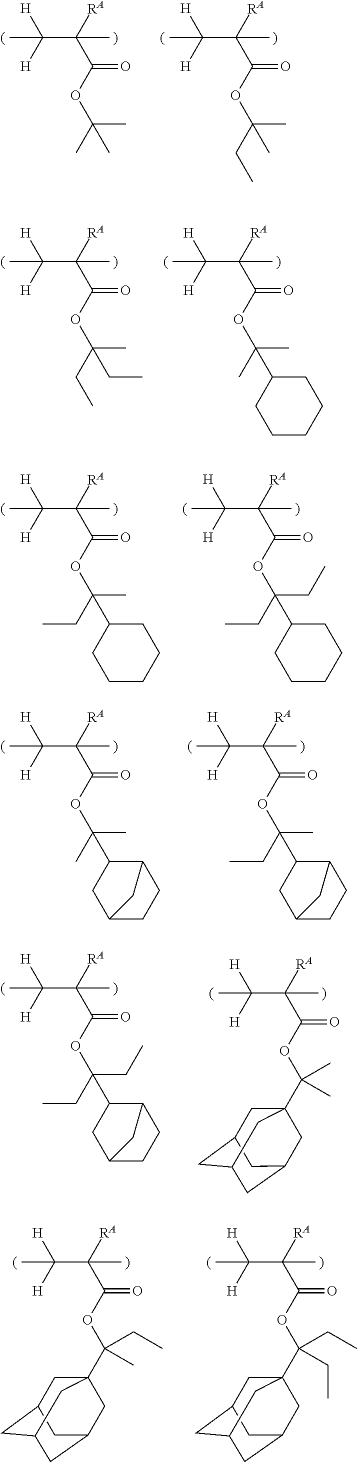

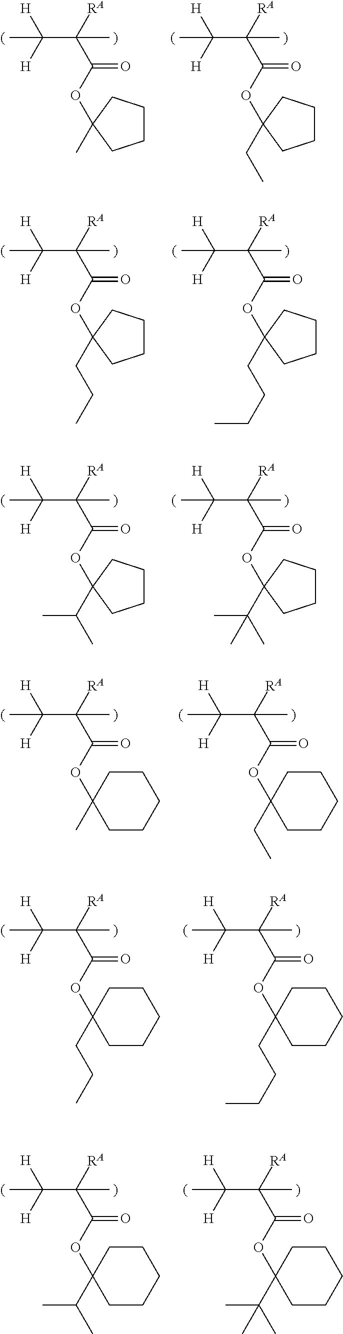

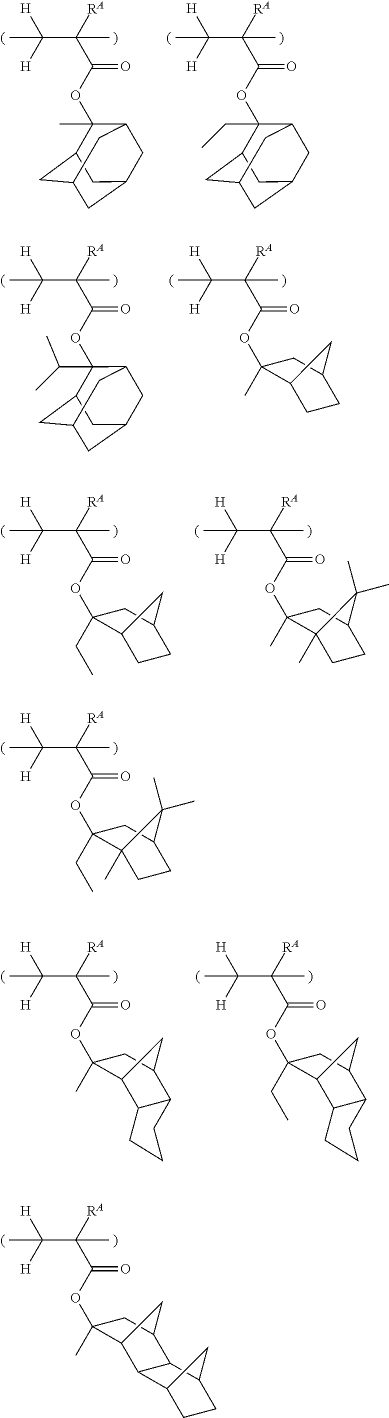

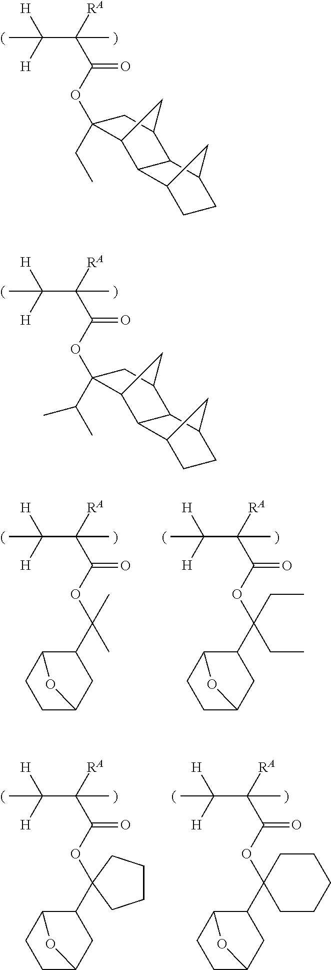

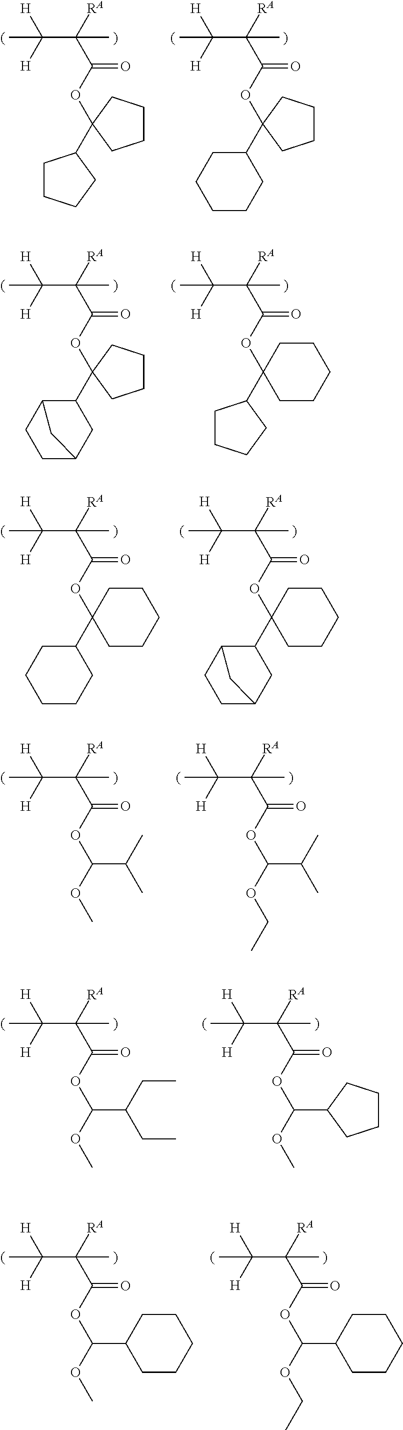

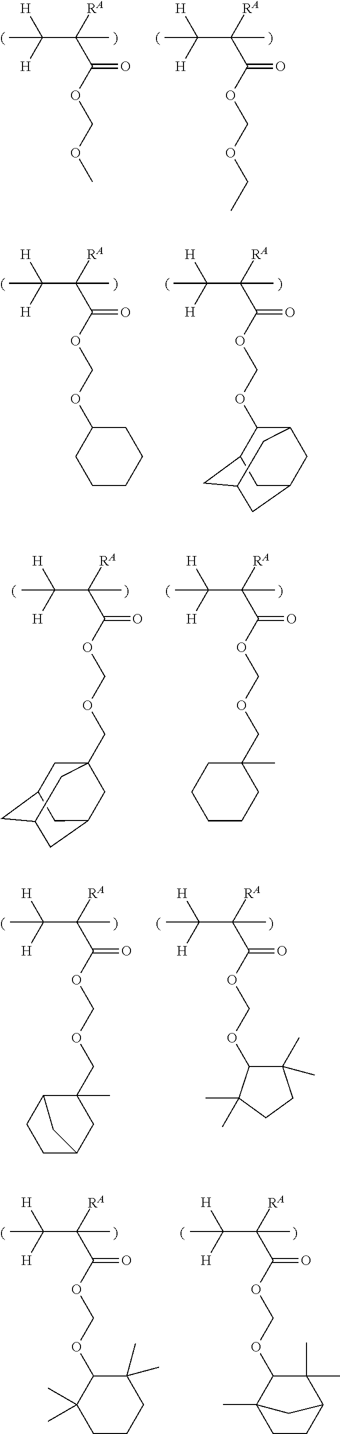

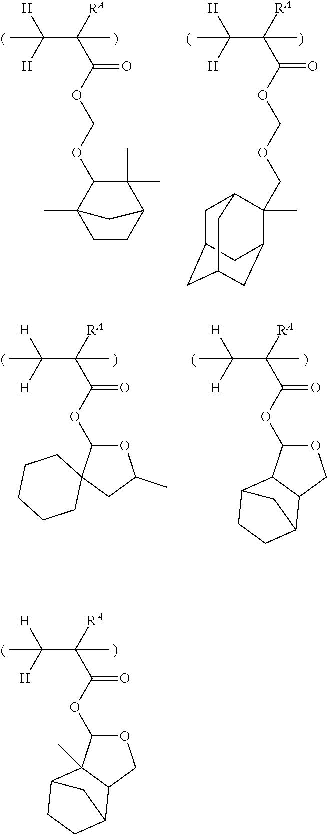

While the foregoing examples correspond to those units wherein X¹ is a single bond, X¹ which is other than a single bond may be combined with similar acid labile groups. Examples of units wherein X¹ is other than a single bond are substantially the same as illustrated above.

Like the repeat units (a1), a polymer comprising repeat units (a2) turns alkali soluble through the mechanism that it is decomposed to generate a hydroxy group under the action of acid. Illustrative examples of the repeat units (a2) are given below, but not limited thereto. Herein R^{A} is as defined above.

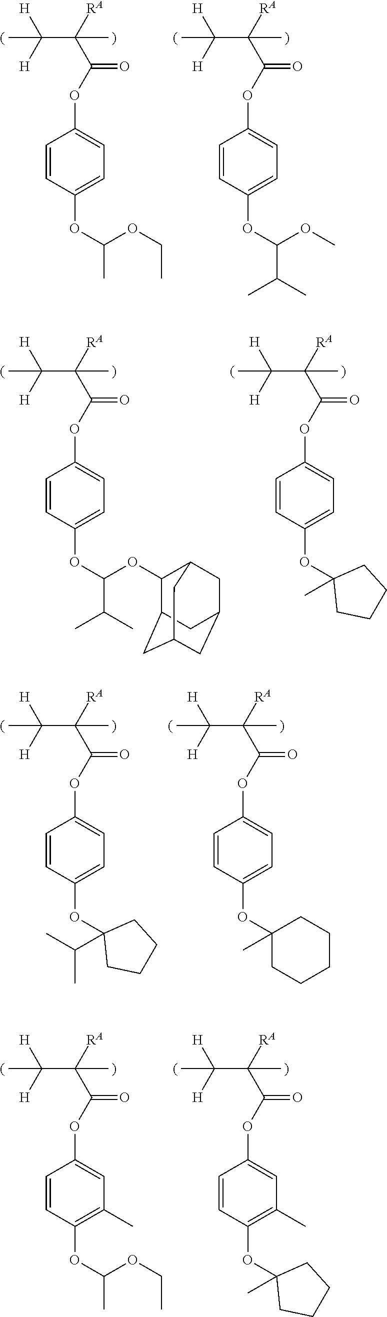

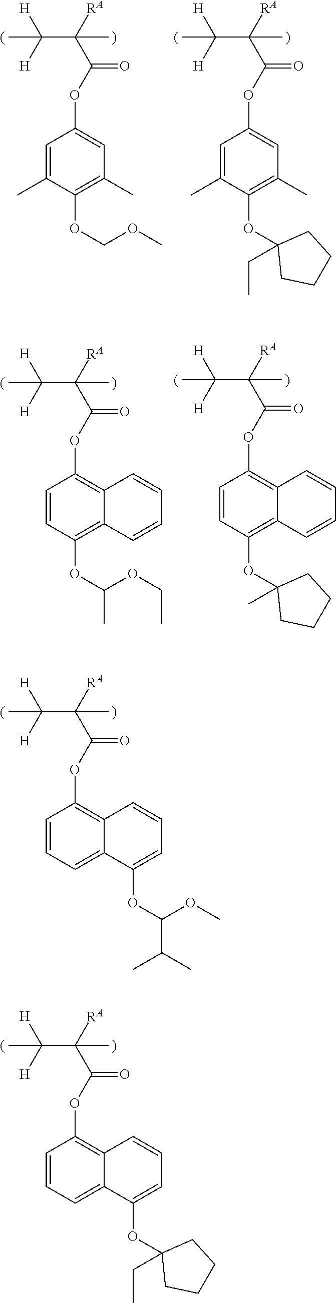

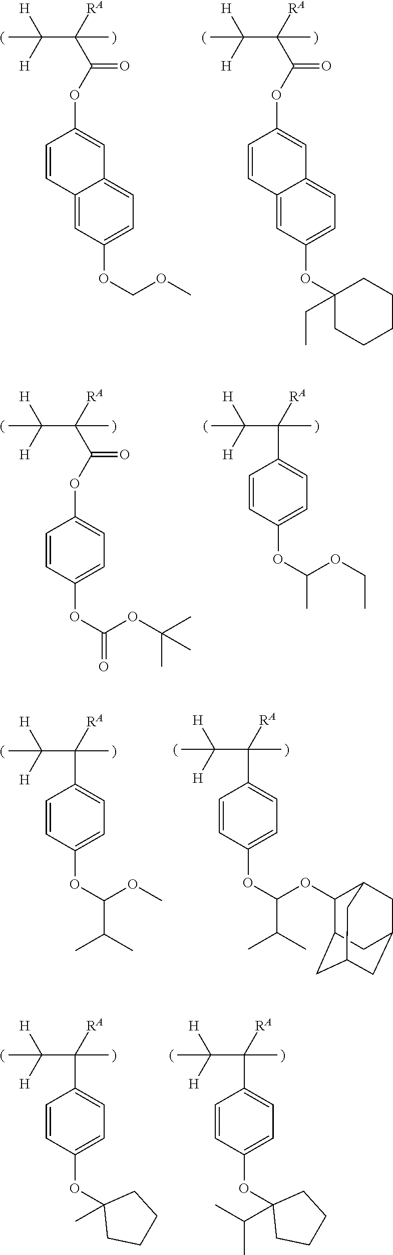

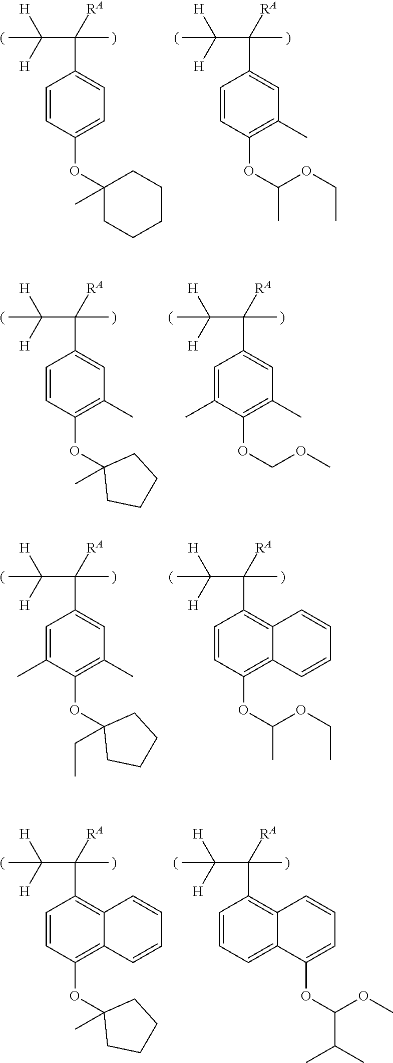

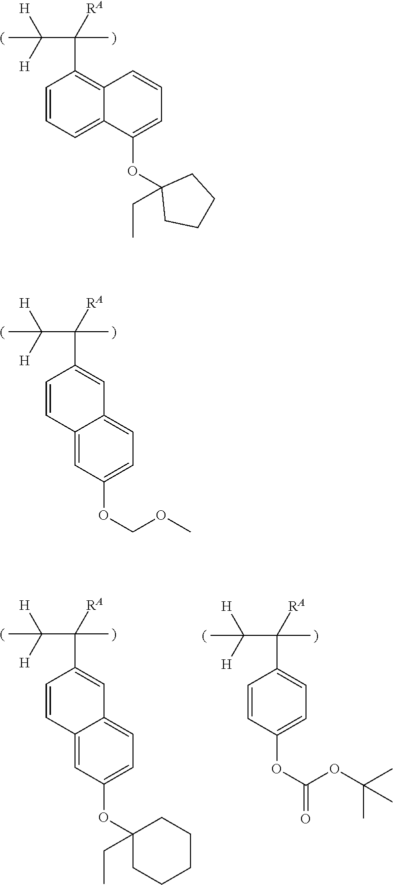

In a preferred embodiment, the base polymer further comprises repeat units having the formula (b1) or repeat units having the formula (b2), which are simply referred to as repeat units (b1) or (b2).

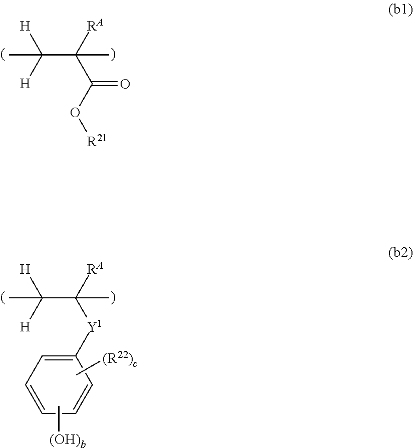

In formulae (b1) and (b2), R^{A} is each independently hydrogen, fluorine, methyl or trifluoromethyl. Y¹ is a single bond or *—C(═O)—O—. The asterisk (*) designates a point of attachment to the carbon atom in the backbone. R²¹ is hydrogen or a C₁-C₂₀ group containing at least one structure selected from among hydroxy other than phenolic hydroxy, cyano, carbonyl, carboxy, ether bond, ester bond, sulfonic ester bond, carbonate bond, lactone ring, sultone ring, and carboxylic anhydride (—C(═O)—O—C(═O)—). R²² is a C₁-C₂₀ hydrocarbyl group which may contain a heteroatom. The subscript b is an integer of 1 to 4, c is an integer of 0 to 4, and b+c is from 1 to 5.

Examples of the repeat unit (b1) are shown below, but not limited thereto. Herein, R^{A} is as defined above.

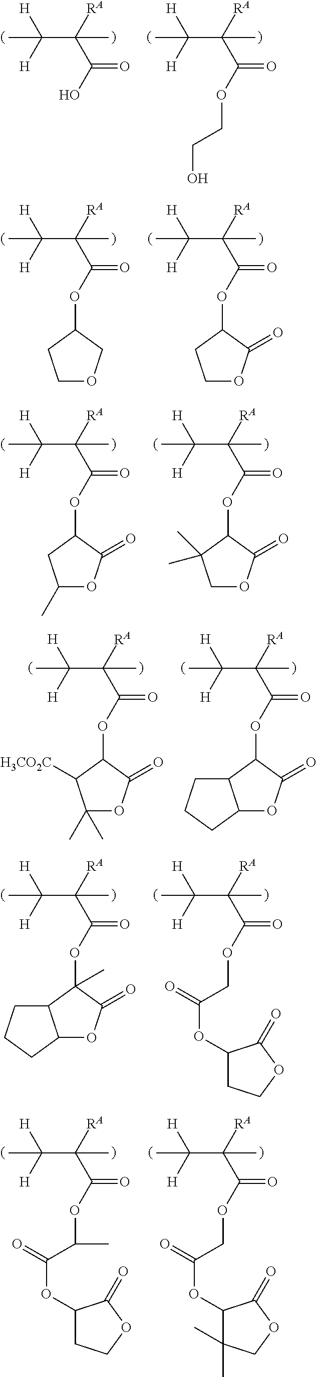

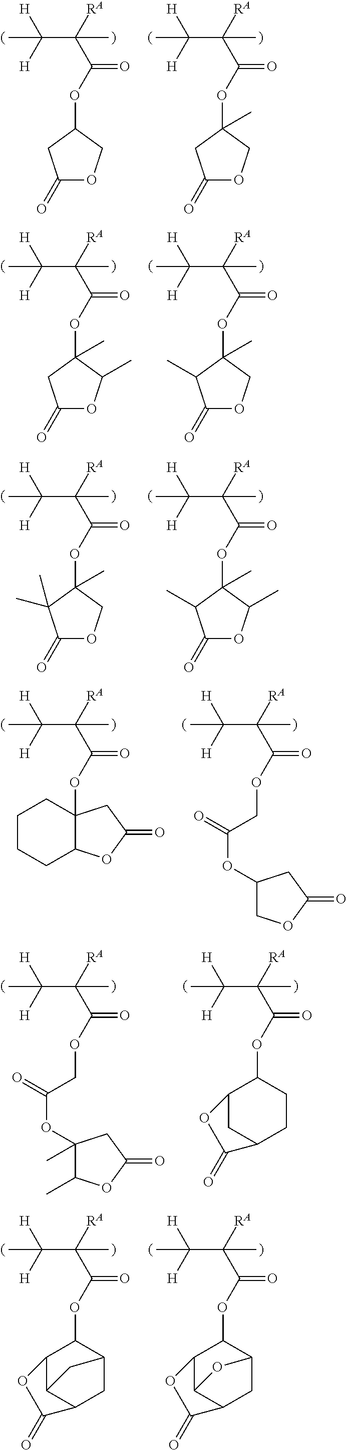

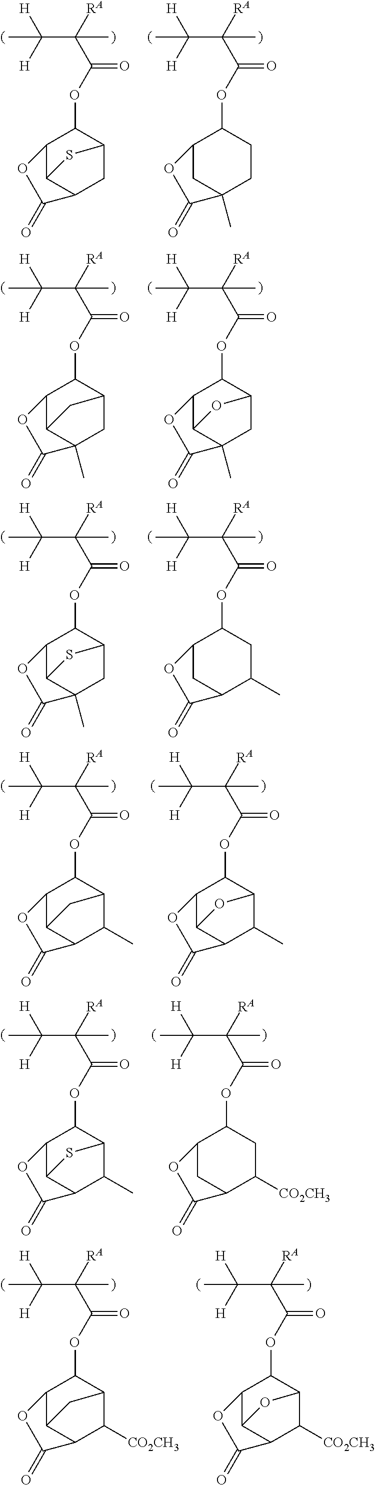

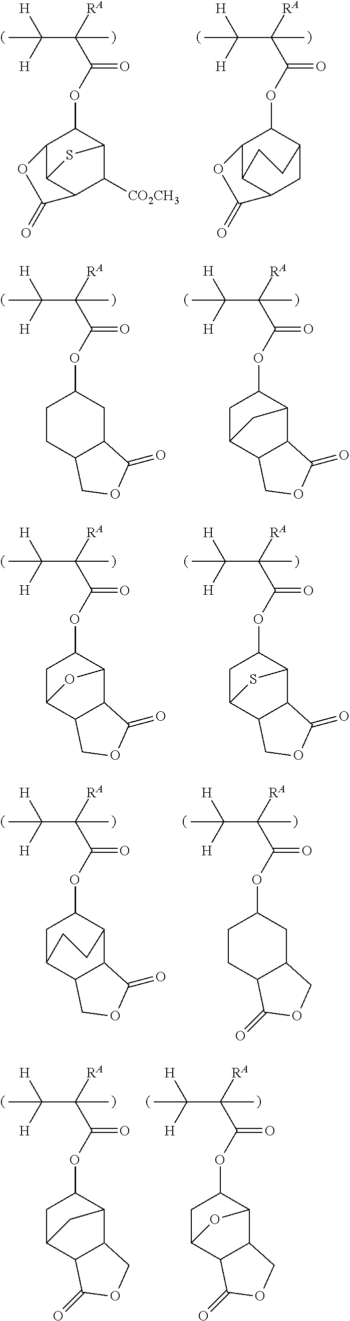

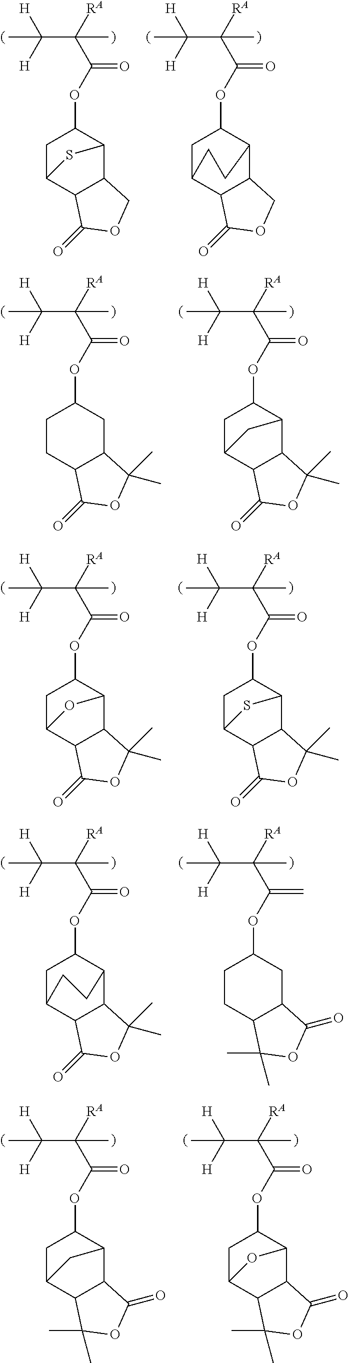

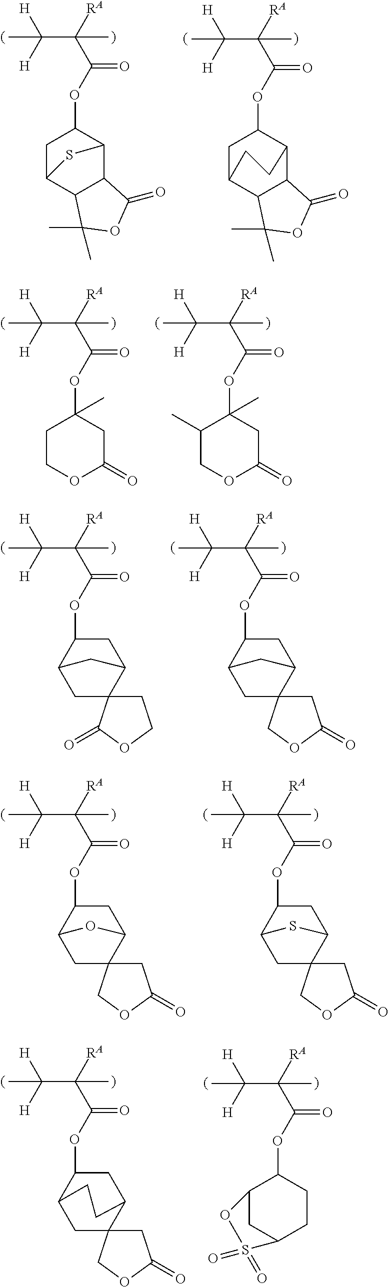

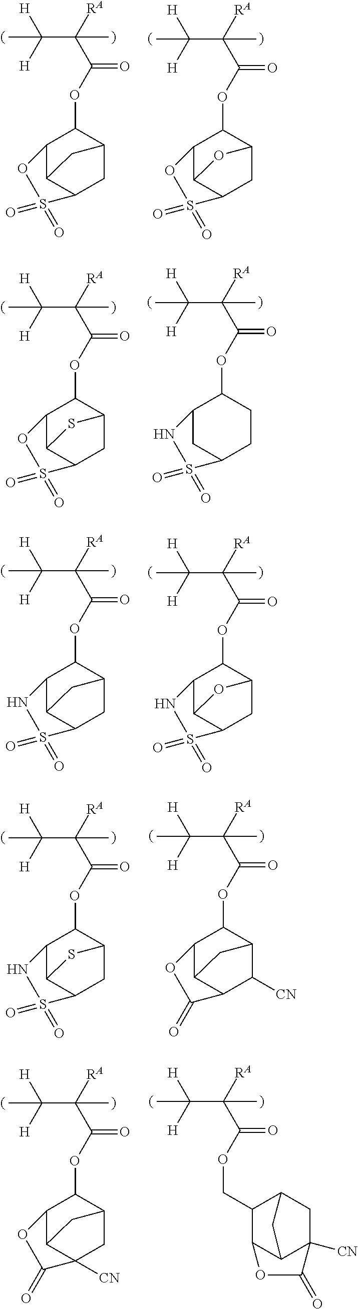

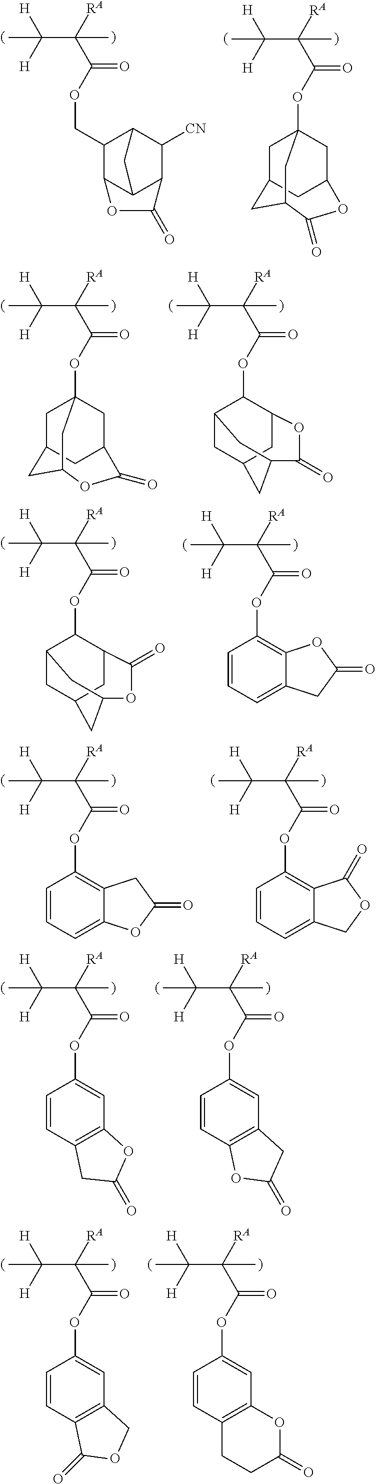

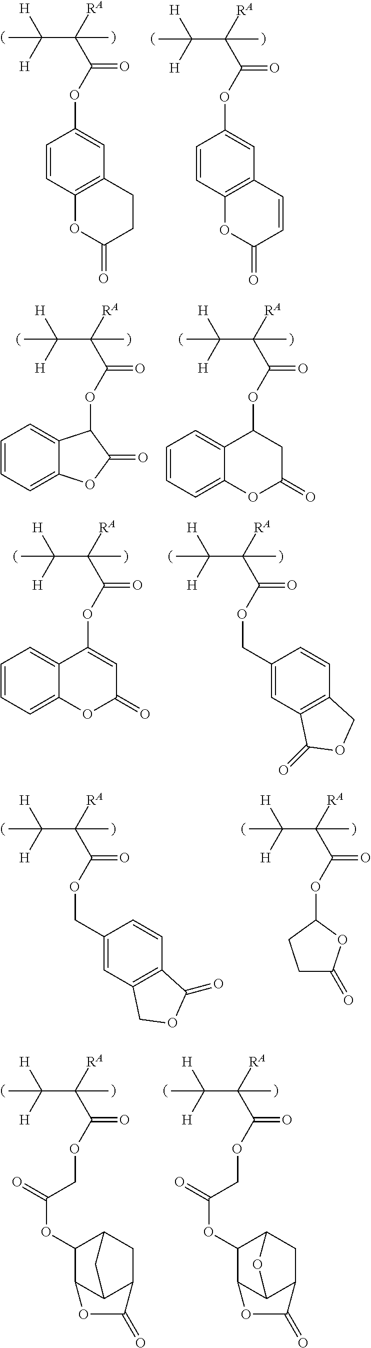

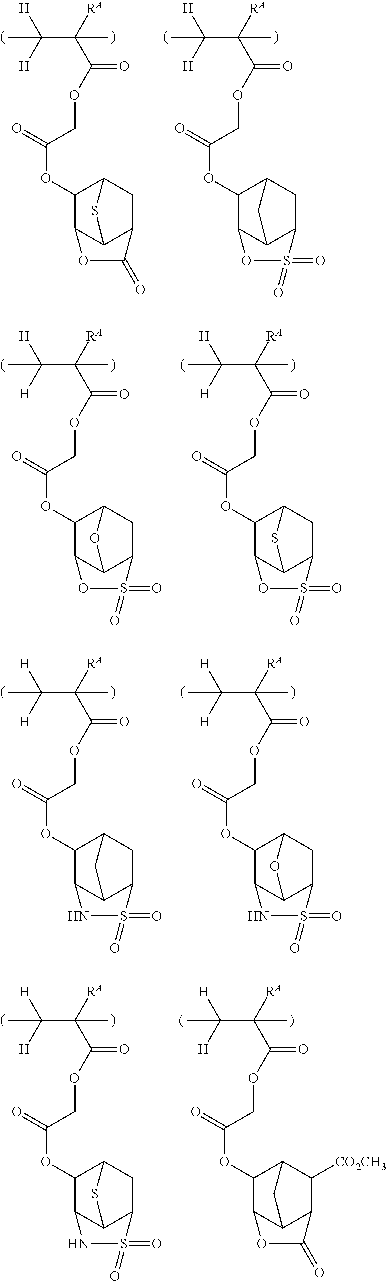

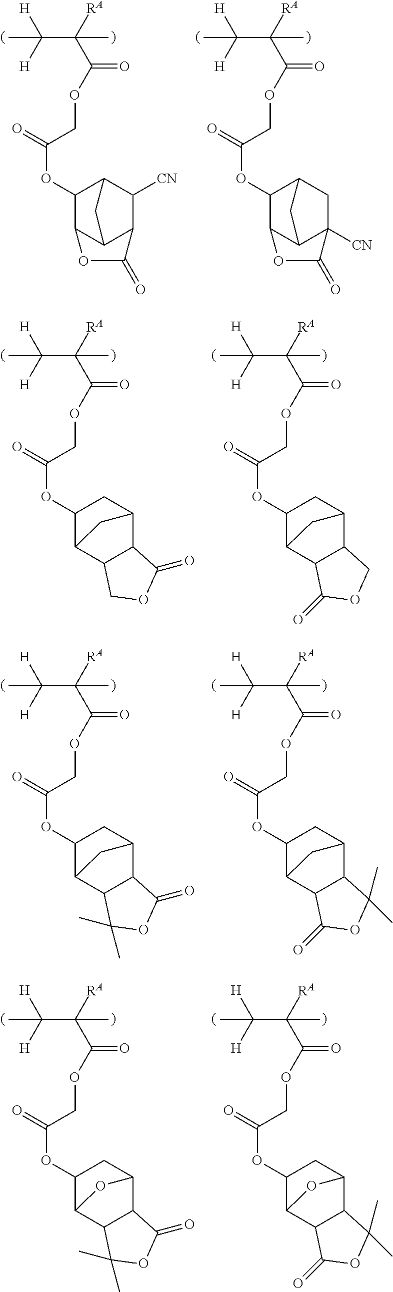

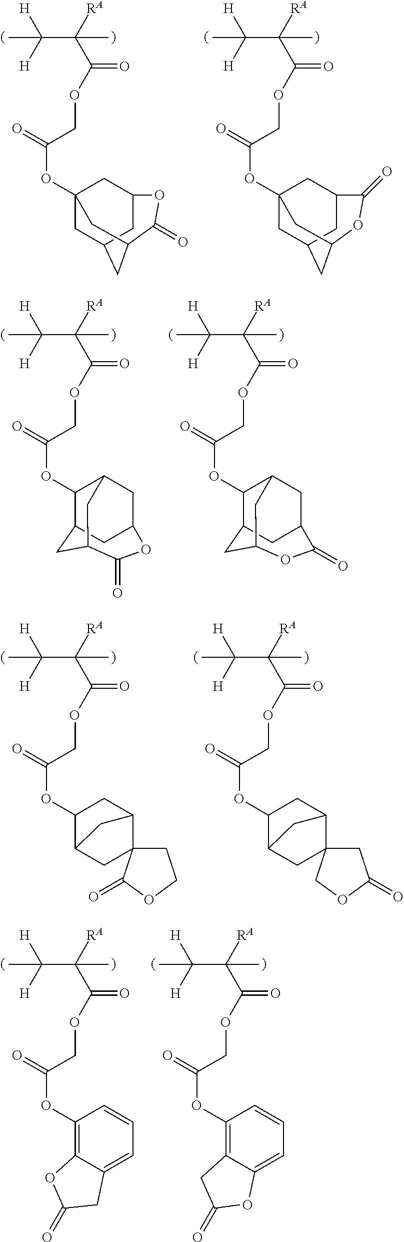

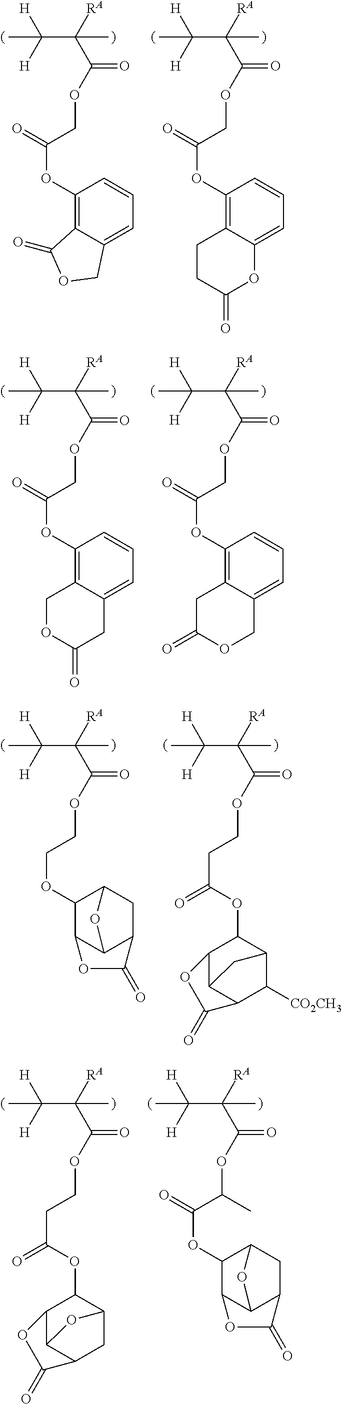

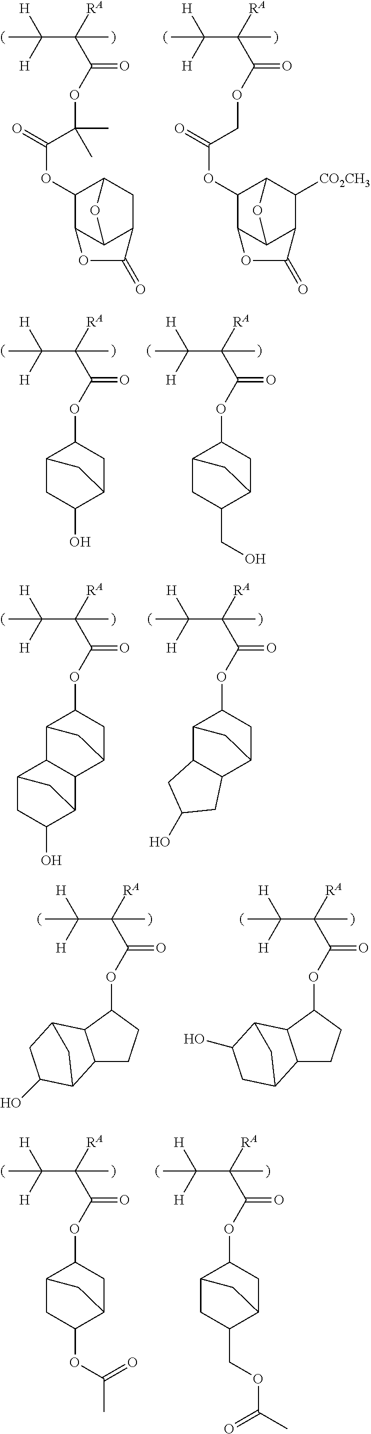

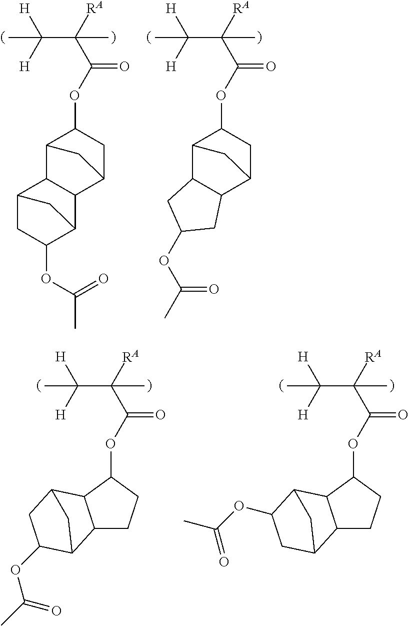

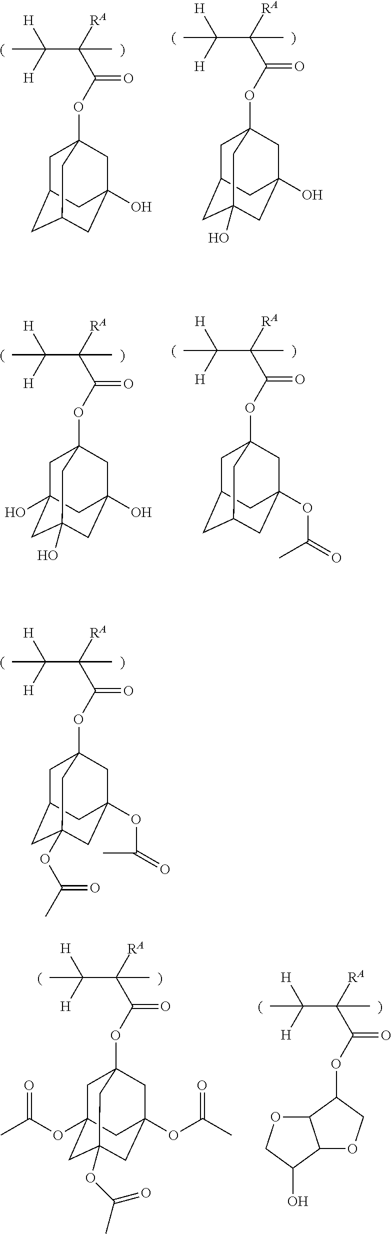

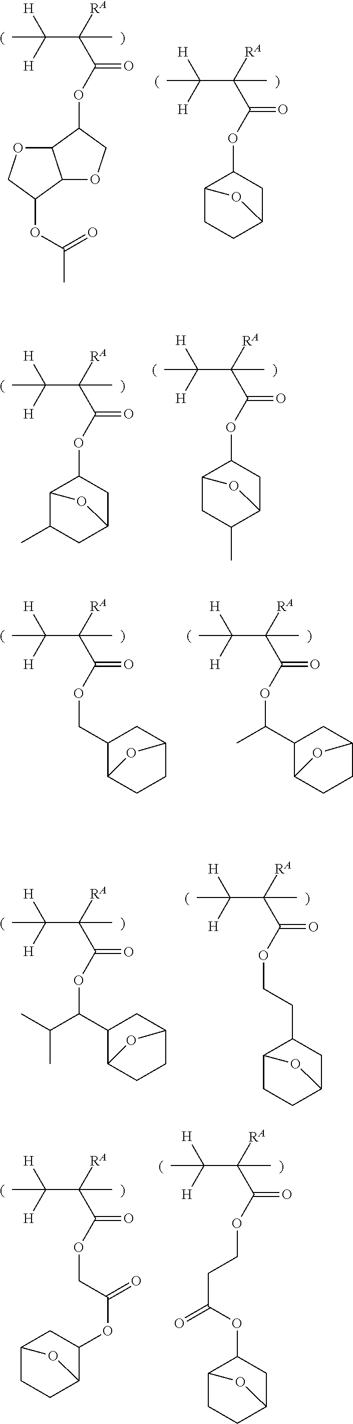

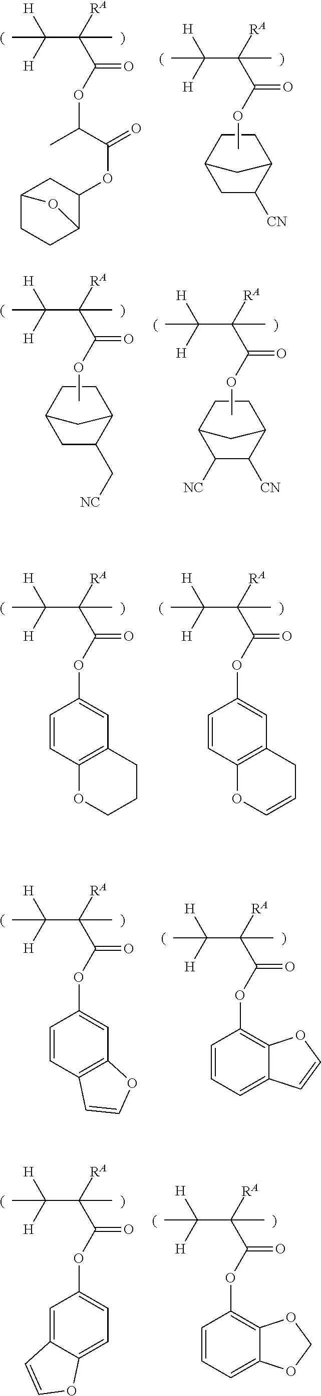

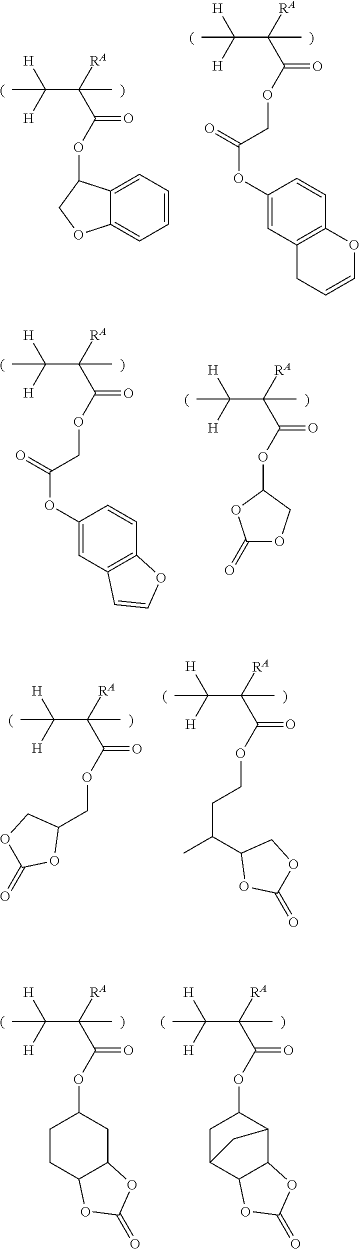

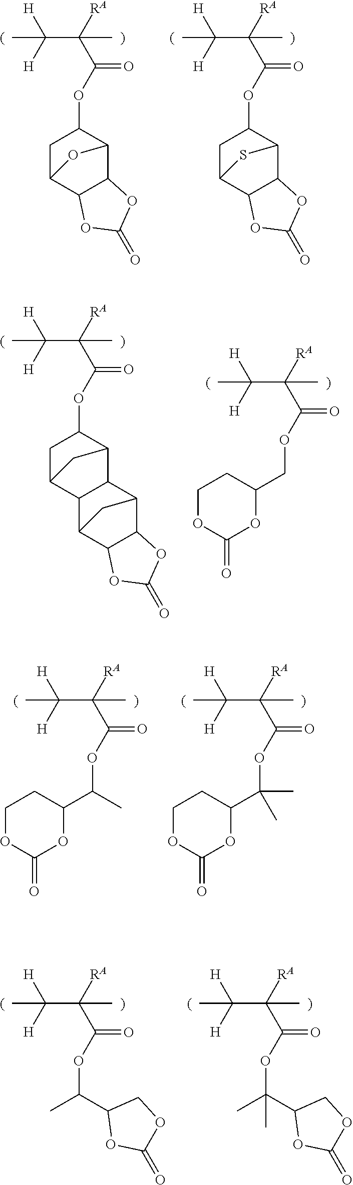

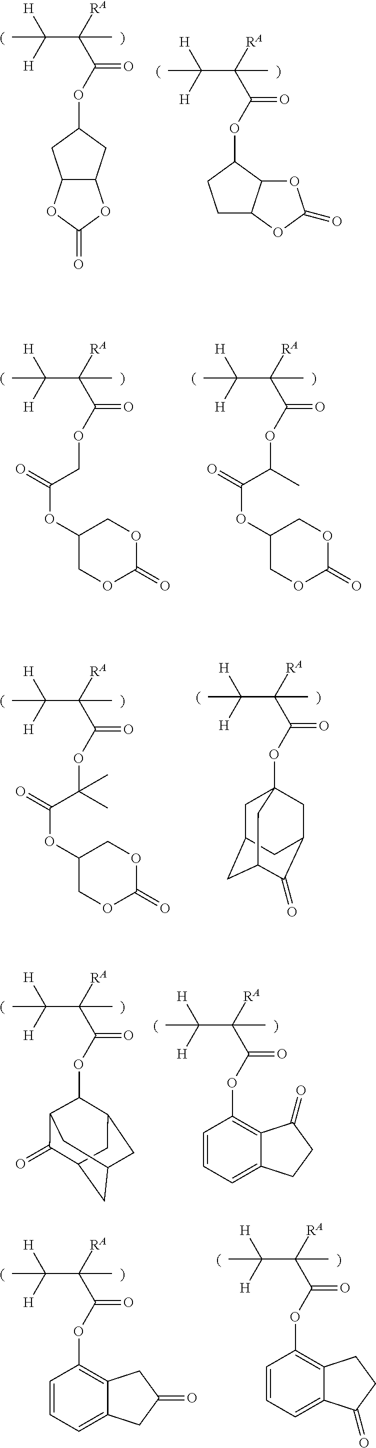

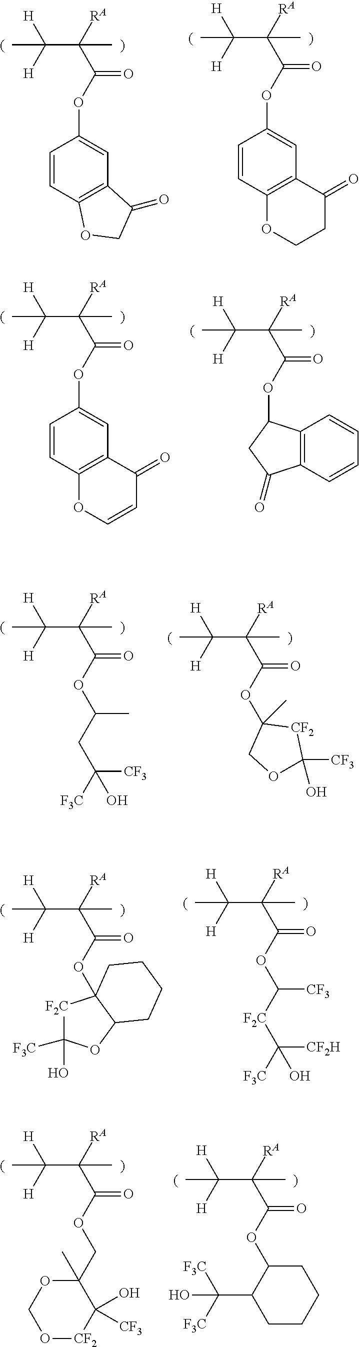

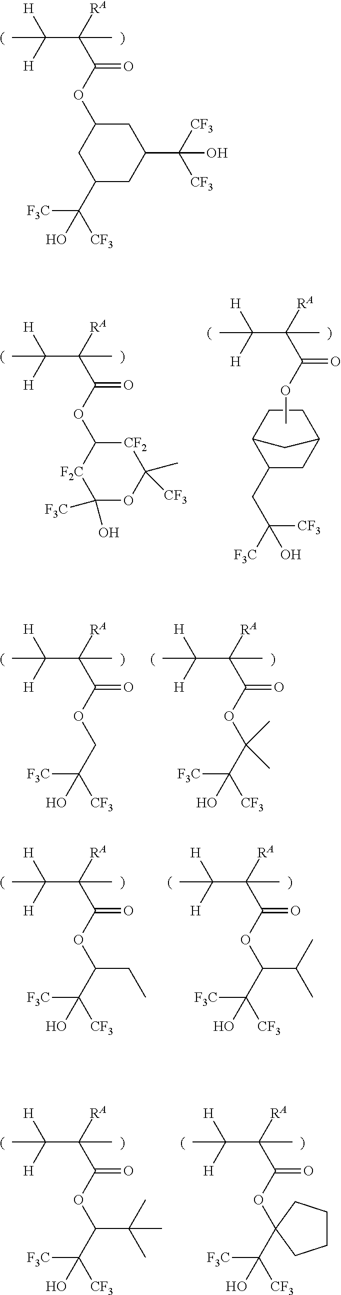

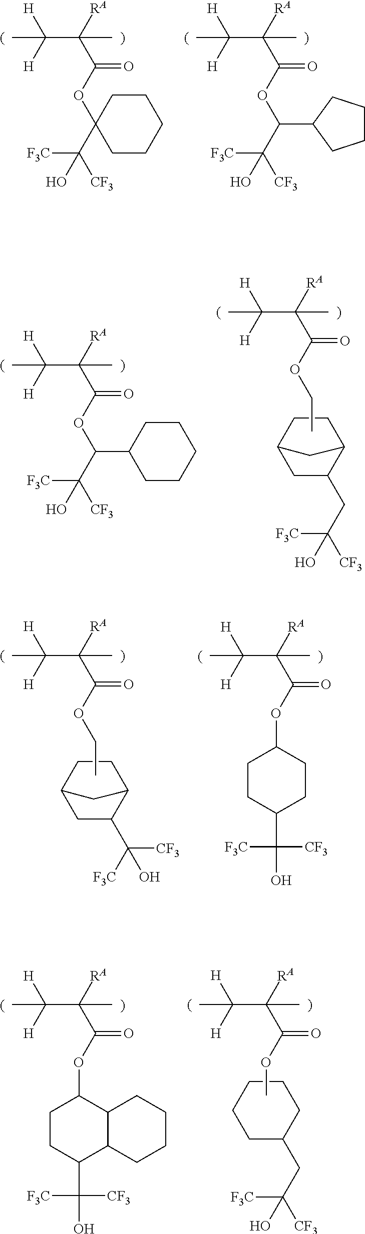

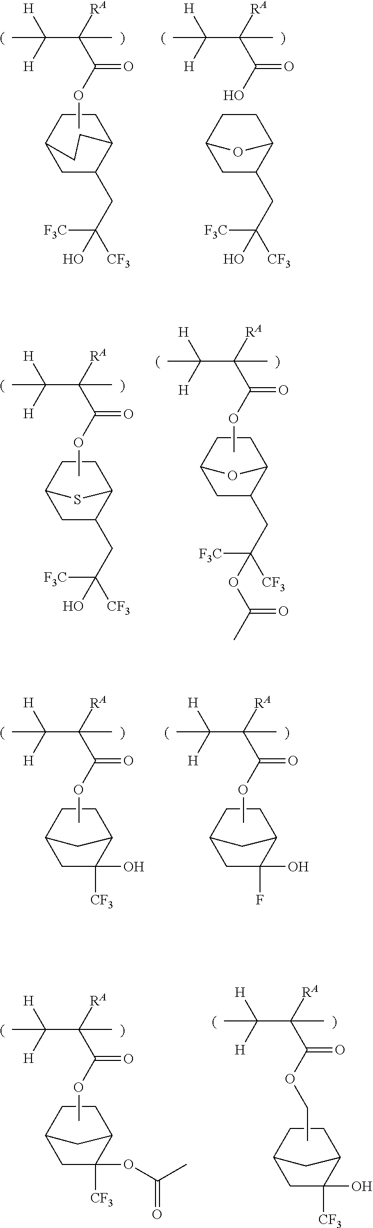

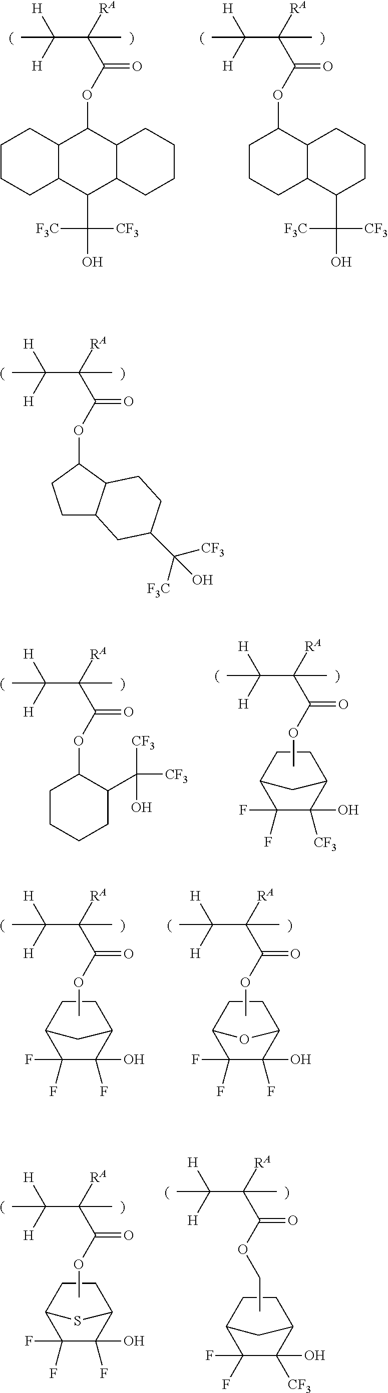

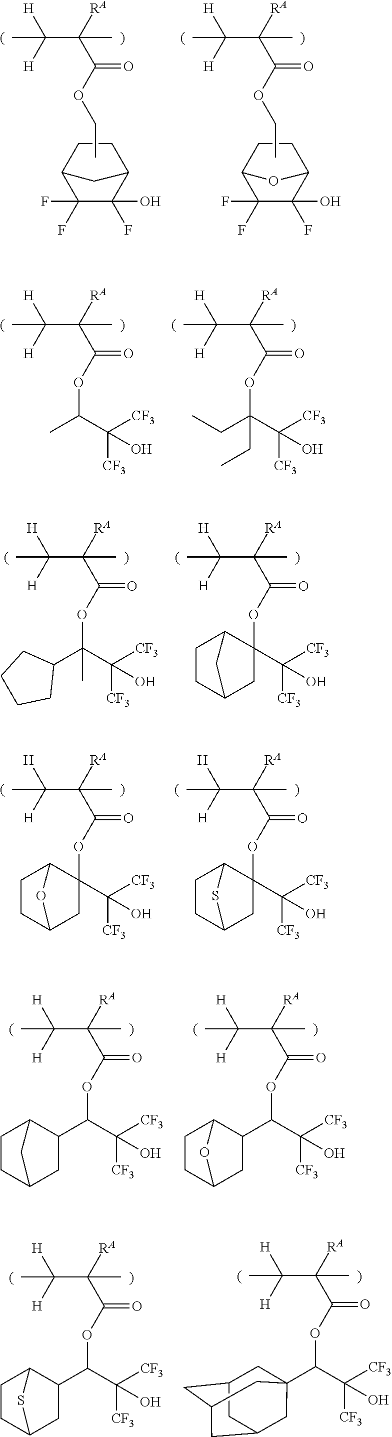

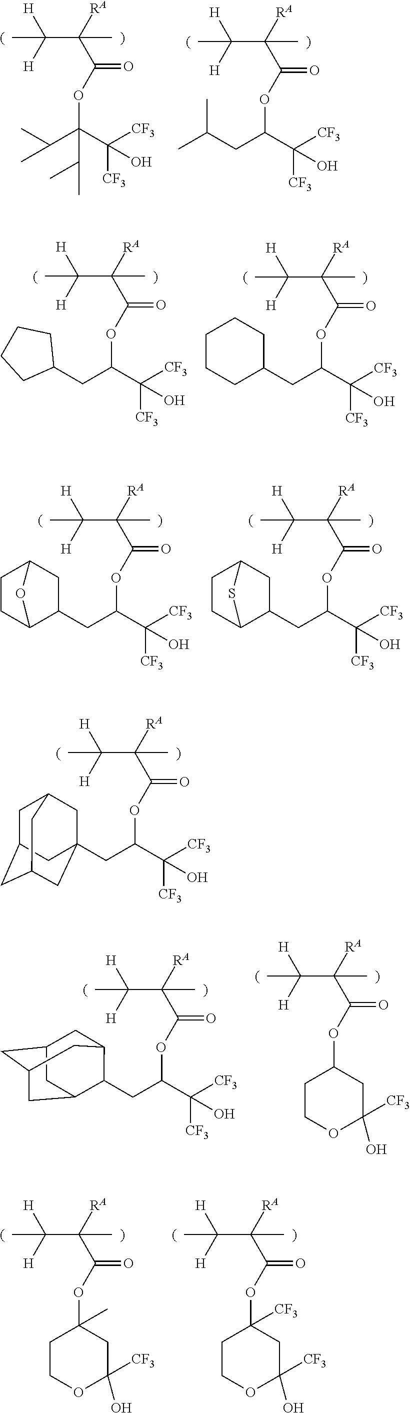

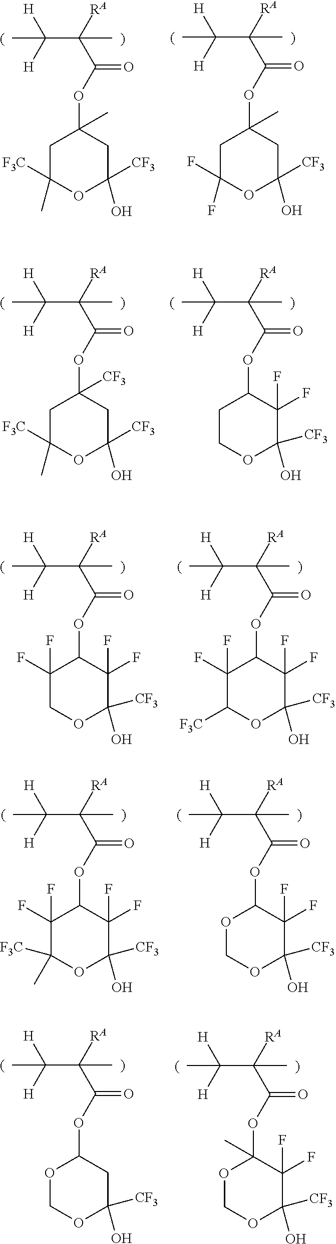

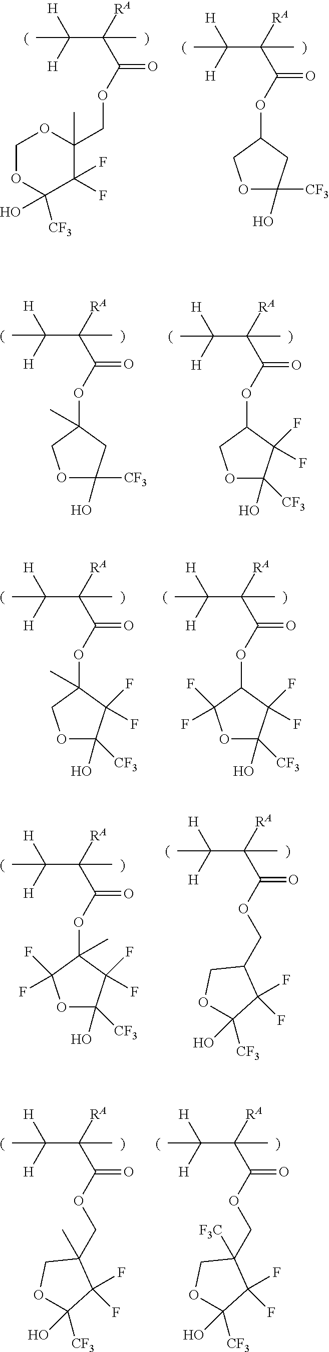

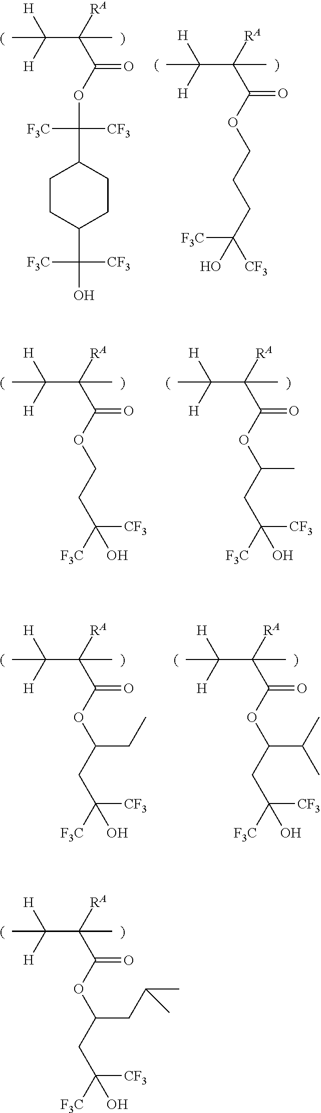

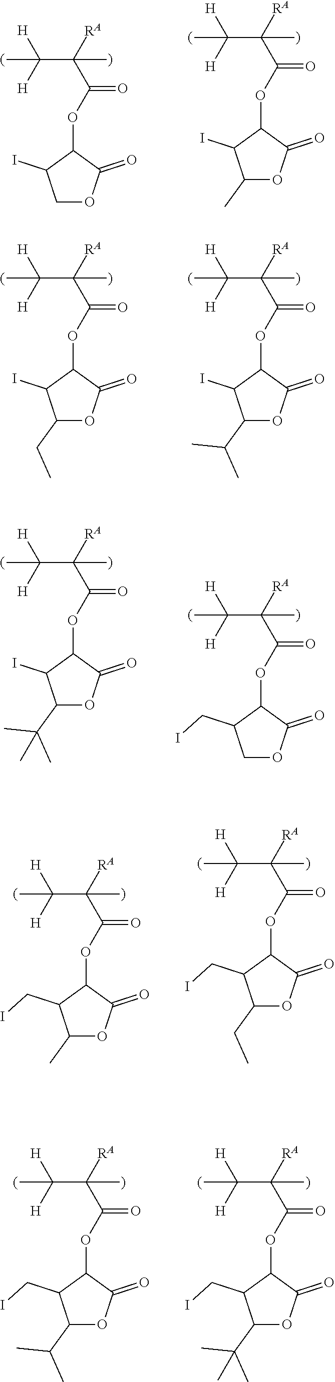

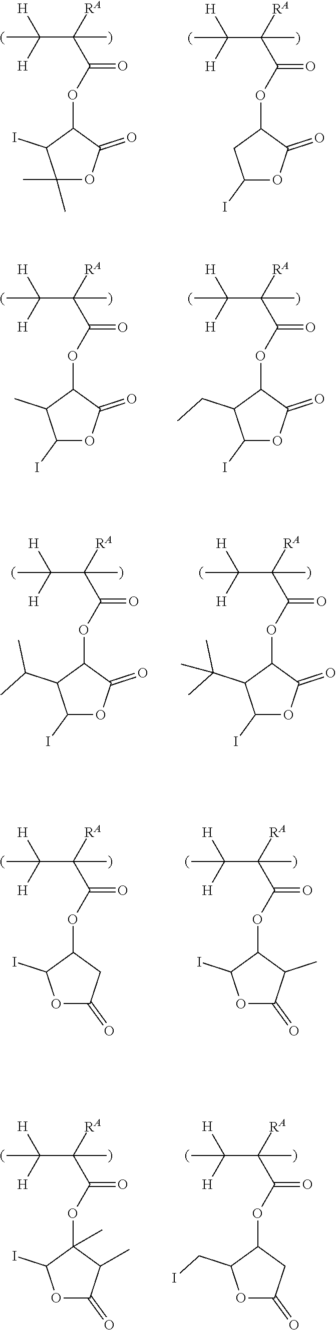

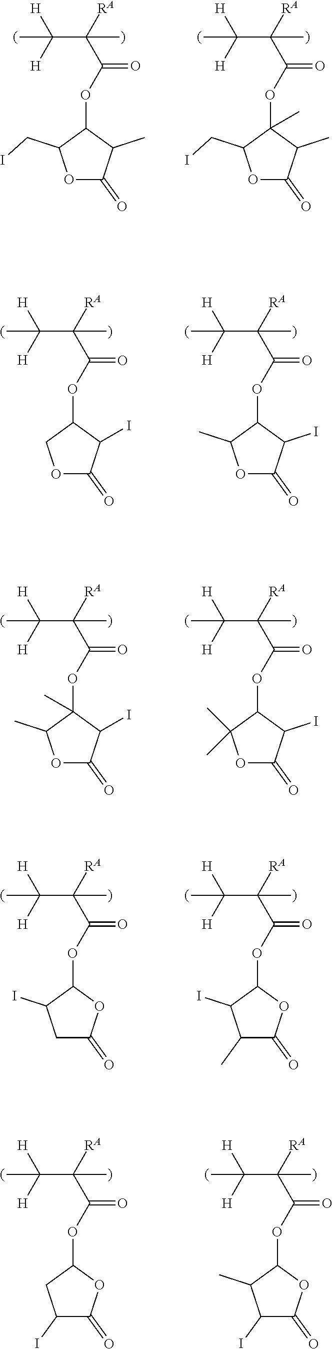

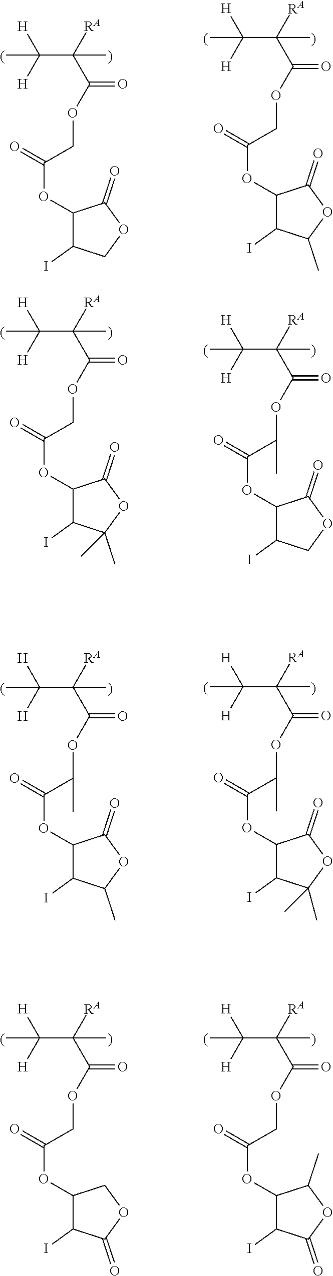

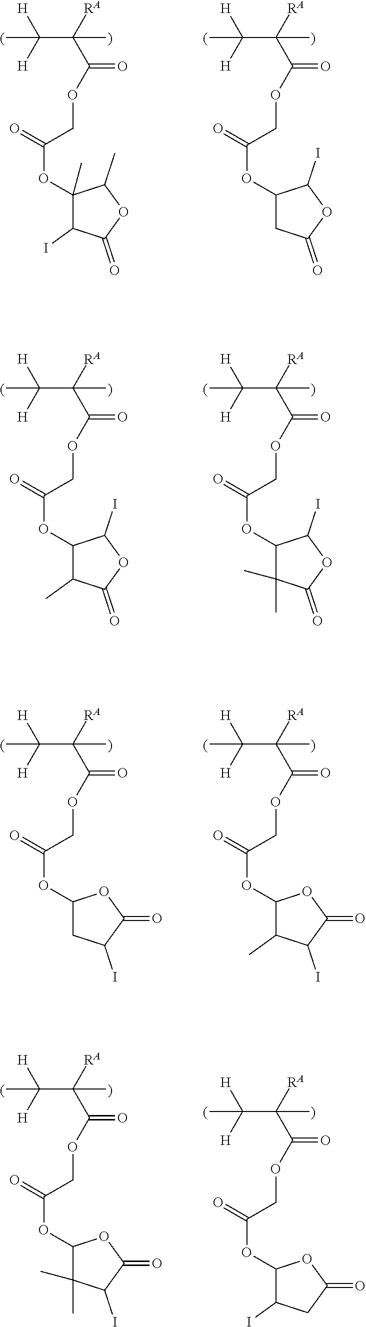

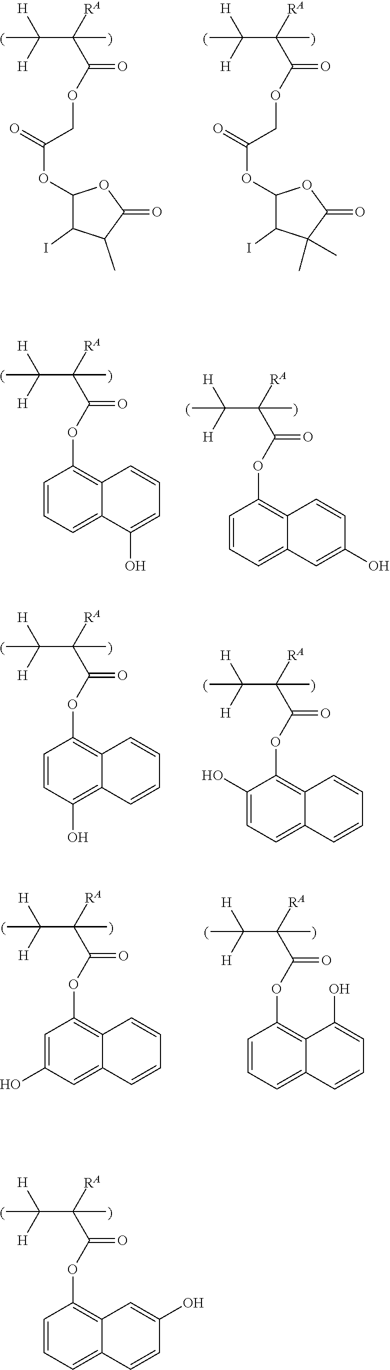

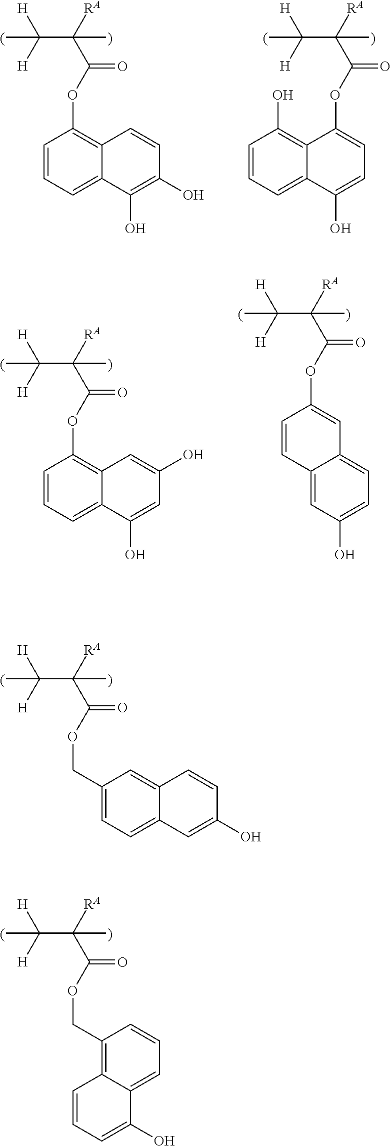

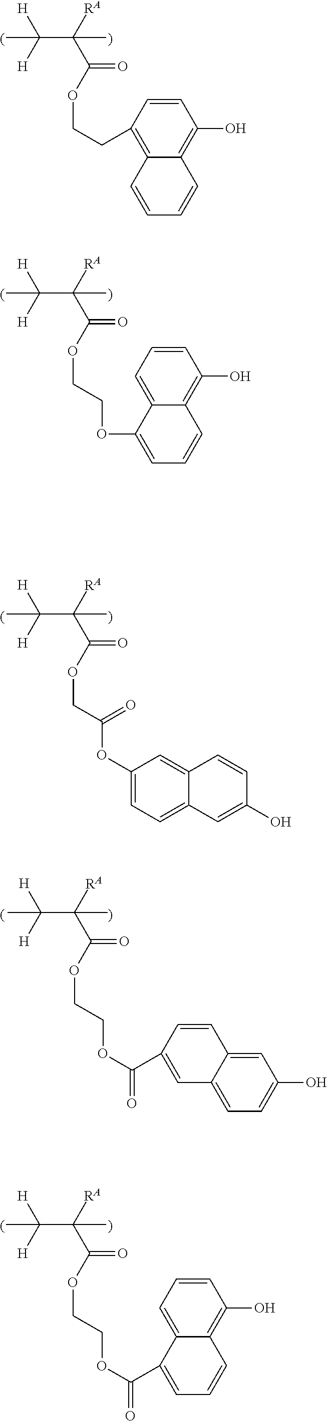

Examples of the repeat unit (b2) are shown below, but not limited thereto. Herein, R^{A} is as defined above.

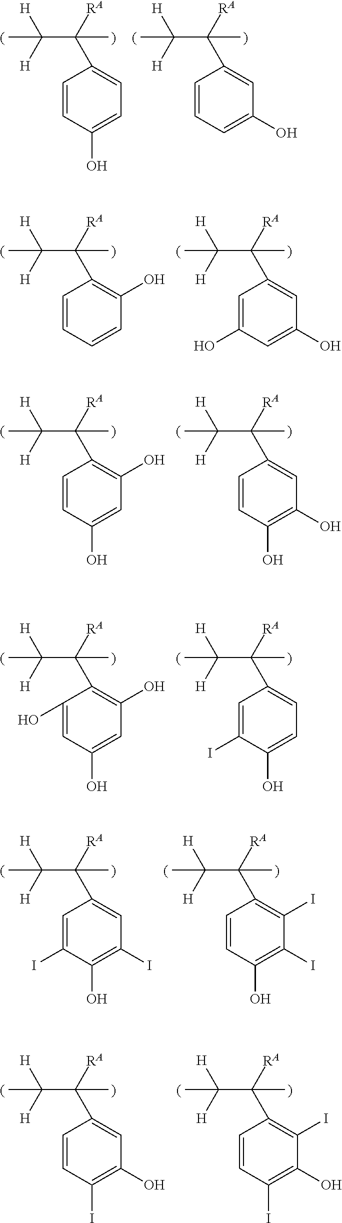

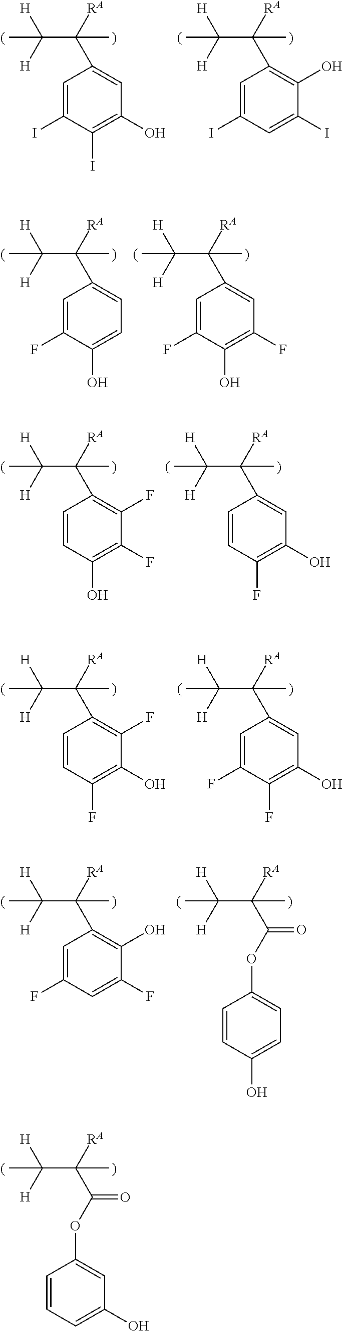

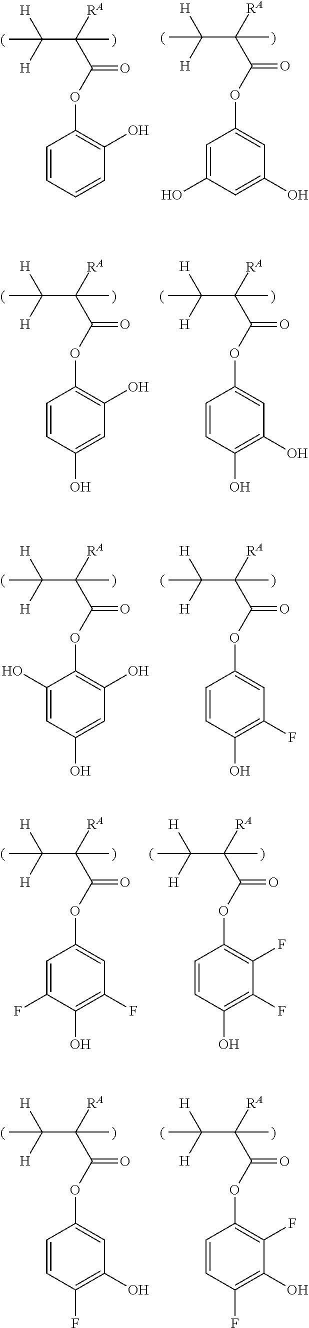

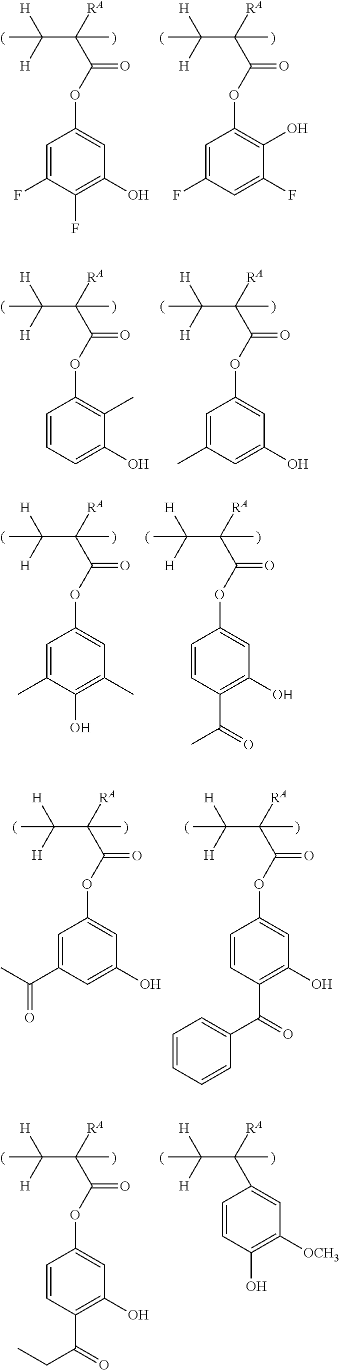

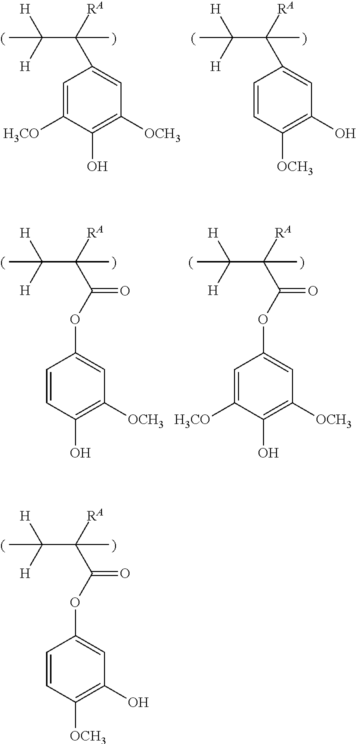

Of the repeat units (b1) and (b2), those units having a lactone ring as the polar group are preferred in the ArF lithography and those units having a phenolic site are preferred in the KrF, EB and EUV lithography.

The base polymer may further comprise repeat units of at least one type selected from repeat units having the formulae (c1) to (c4), which are simply referred to as repeat units (c1) to (c4). Since these units function as a photoacid generator, a photoacid generator to be described later as component (D) may be omitted when a base polymer containing these units is used.

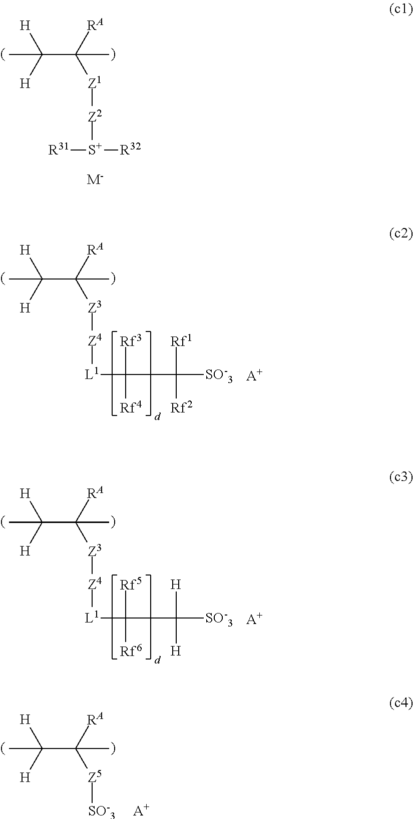

In formulae (c1) to (c4), R^{A} is each independently hydrogen, fluorine, methyl or trifluoromethyl. Z is a single bond or phenylene group. Z² is *—C(═O)—O—Z²¹—, *—C(═O)—NH—Z²¹— or *—O—Z²¹—. Z²¹ is a C₁-C₆ aliphatic hydrocarbylene group, a phenylene group or a divalent group obtained by combining the foregoing, which may contain a carbonyl moiety, ester bond, ether bond or hydroxy moiety. Z³ is a single bond, phenylene group, naphthylene group or *—C(═O)—O—Z³¹—. Z³¹ is a C₁-C₁₀ aliphatic hydrocarbylene group which may contain a hydroxy moiety, ether bond, ester bond or lactone ring, or phenylene or naphthylene group. Z⁴ is a single bond or **—Z⁴¹—C(═O)—O—. Z⁴¹ is a C₁-C₂₀ hydrocarbylene group which may contain a heteroatom. Z⁵ is a single bond, methylene, ethylene, phenylene, fluorinated phenylene, trifluoromethyl-substituted phenylene group, *—C(═O)—O—Z⁵¹—, *—C(═O)—NH—Z⁵¹—, or *—O—Z⁵¹—. Z⁵¹ is a C₁-C₆ aliphatic hydrocarbylene group, phenylene group, fluorinated phenylene group or trifluoromethyl-substituted phenylene group, which may contain a carbonyl moiety, ester bond, ether bond or hydroxy moiety. The asterisk (*) designates a point of attachment to the carbon atom in the backbone, and the double asterisks (**) designates a point of attachment to Z³.

In formula (c1), R³¹ and R³² are each independently a C₁-C₂₀ hydrocarbyl group which may contain a heteroatom. R³¹ and R³² may bond together to form a ring with the sulfur atom to which they are attached.

The hydrocarbyl groups R³¹ and R³² may be saturated or unsaturated and straight, branched or cyclic. Examples thereof include C₁-C₂₀ alkyl groups such as methyl, ethyl, n-propyl, isopropyl, n-butyl, tert-butyl; C₃-C₂₀ cyclic saturated hydrocarbyl groups such as cyclopropyl, cyclopentyl, cyclohexyl, cyclopropylmethyl, 4-methylcyclohexyl, cyclohexylmethyl, norbornyl, and adamantyl; C₂-C₂₀ alkenyl groups such as vinyl, allyl, propenyl, butenyl, hexenyl; C₃-C₂₀ cyclic unsaturated hydrocarbyl groups such as cyclohexenyl: C₆-C₂₀ aryl groups such as phenyl and naphthyl. C₇-C₂₀ aralkyl groups such as benzyl, 1-phenylethyl and 2-phenylethyl, and combinations thereof. Inter alia, aryl groups are preferred. In these hydrocarbyl groups, some or all of the hydrogen atoms may be substituted by a moiety containing a heteroatom such as oxygen, sulfur, nitrogen or halogen, and some constituent —CH₂— may be replaced by a moiety containing a heteroatom such as oxygen, sulfur or nitrogen, so that the group may contain a hydroxy moiety, fluorine, chlorine, bromine, iodine, cyano moiety, carbonyl moiety, ether bond, ester bond, sulfonic ester bond, carbonate bond, lactone ring, sultone ring, carboxylic anhydride (—C(═O)—O—C(═O)—) or haloalkyl moiety.

Examples of the cation in the repeat units having formula (c1) are shown below, but not limited thereto. Herein. R^{A} is as defined above.

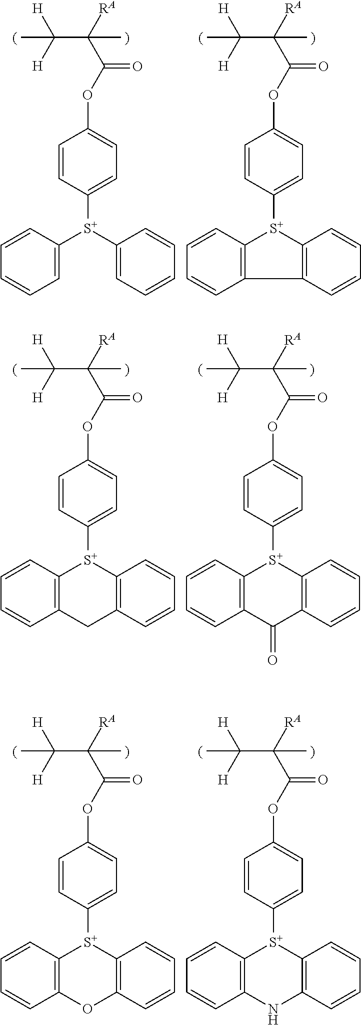

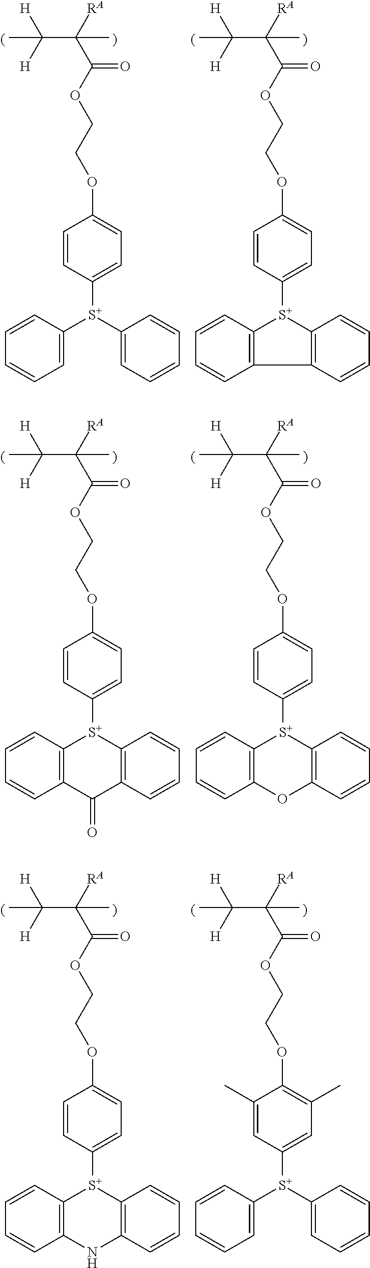

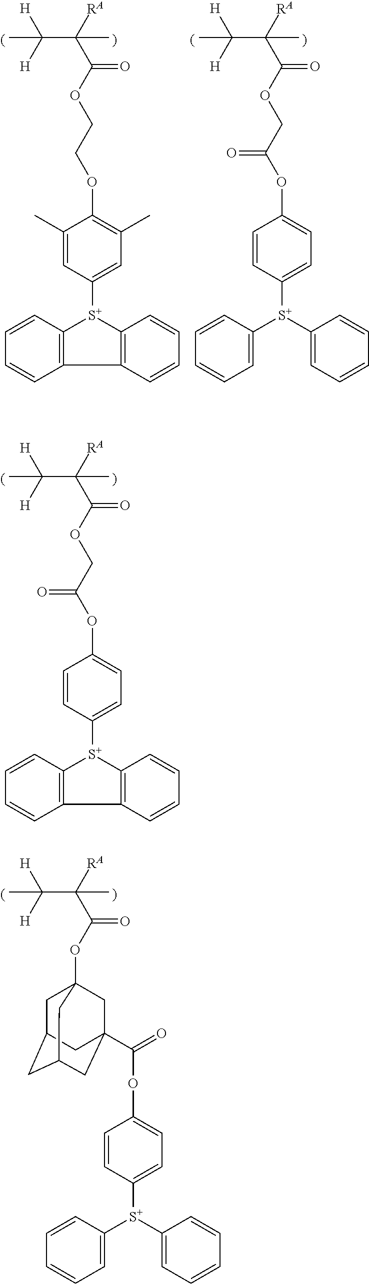

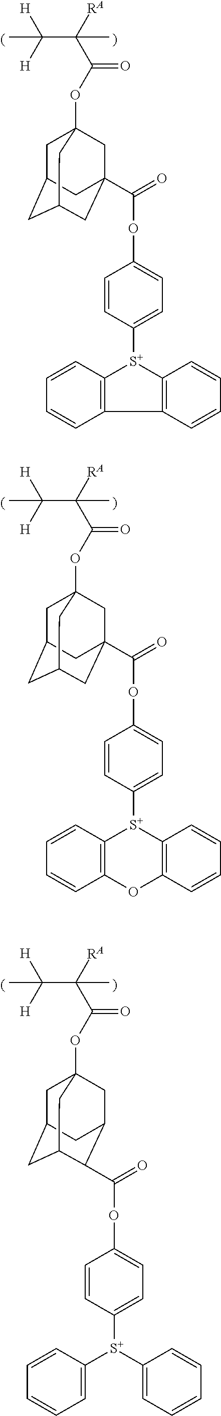

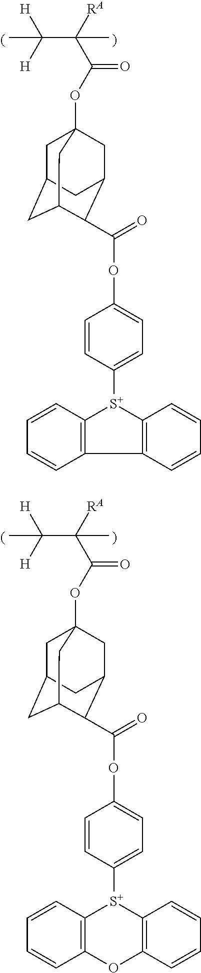

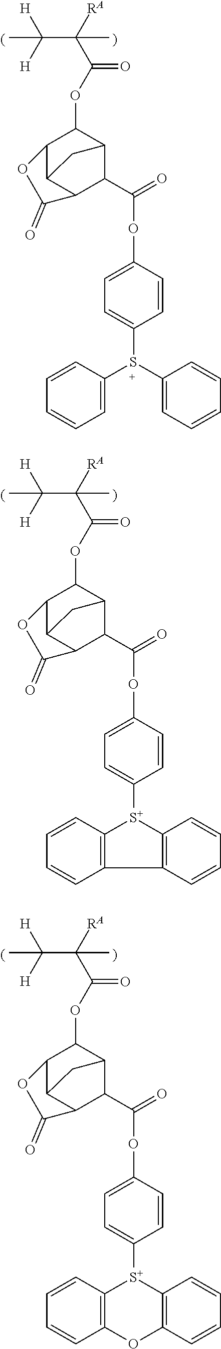

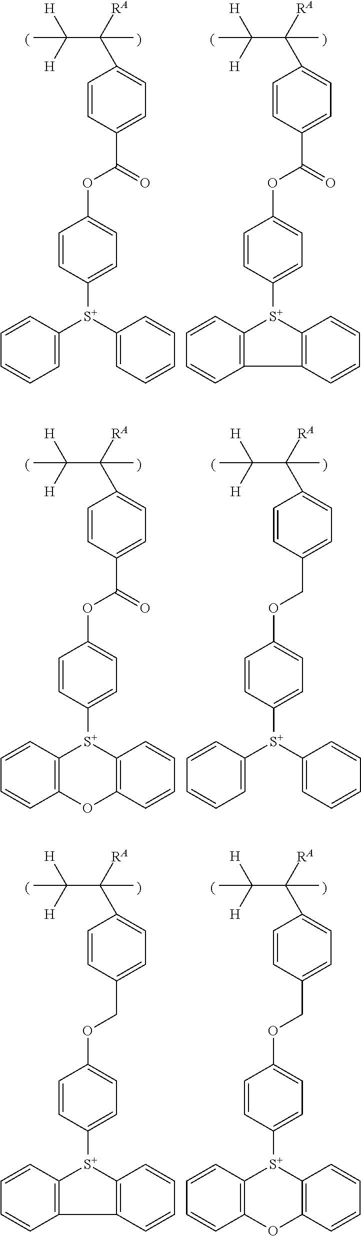

In formula (c1), M⁻ is a non-nucleophilic counter ion. Examples of the non-nucleophilic counter ion include halide ions such as chloride and bromide ions; fluoroalkylsulfonate ions such as triflate, 1,1,1-trifluoroethanesulfonate, and nonafluorobutanesulfonate, arylsulfonate ions such as tosylate, benzenesulfonate, 4-fluorobenzenesulfonate, and 1,2,3,4,5-pentafluorobenzenesulfonate; alkylsulfonate ions such as mesylate and butanesulfonate, imide ions such as bis(trifluoromethylsulfonyl)imide, bis(perfluoroethylsulfonyl)imide and bis(perfluorobutylsulfonyl)imide: and methide ions such as tris(trifluoromethylsulfonyl)methide and tris(perfluoroethylsulfonyl)methide.

Also included are a sulfonate anion which is fluorinated at α-position as represented by the formula (c1-1) and a sulfonate anion which is substituted with fluorine at α-position and trifluoromethyl at β-position as represented by the formula (c1-2).

R³³—CF₂—SO₃⁻ (c1-1)

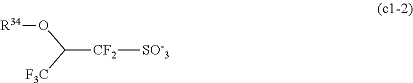

In formula (c1-1), R³³ is hydrogen or a hydrocarbyl group which may contain an ether bond ester bond, carbonyl moiety, lactone ring or fluorine atom. The hydrocarbyl group may be saturated or unsaturated and straight, branched or cyclic, and examples thereof are as will be exemplified later for R¹¹¹ in formula (2A′).

In formula (c1-2), R³⁴ is hydrogen, a C₁-C₃₀ hydrocarbyl group, or C₆-C₂₀ hydrocarbylcarbonyl group. The hydrocarbyl group and hydrocarbylcarbonyl group may contain an ether bond, ester bond, carbonyl moiety or lactone ring. The hydrocarbyl group and hydrocarbyl moiety in the hydrocarbylcarbonyl group may be saturated or unsaturated and straight, branched or cyclic, and examples thereof are as will be exemplified later for R¹¹¹ in formula (2A′).

Examples of the sulfonate anions which are exemplary of the non-nucleophilic counter ion are shown below, but not limited thereto. Herein R³⁵ is hydrogen, fluorine or C₁-C₆ fluoroalkyl.

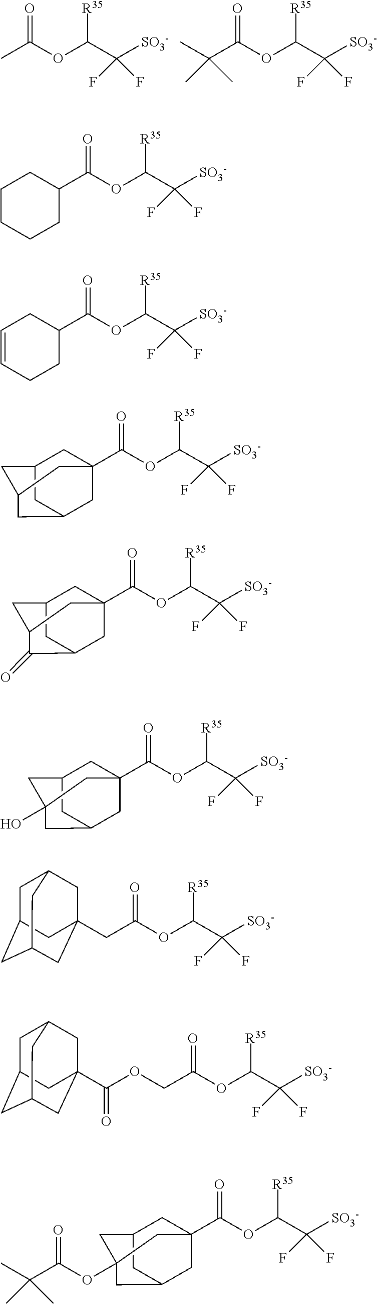

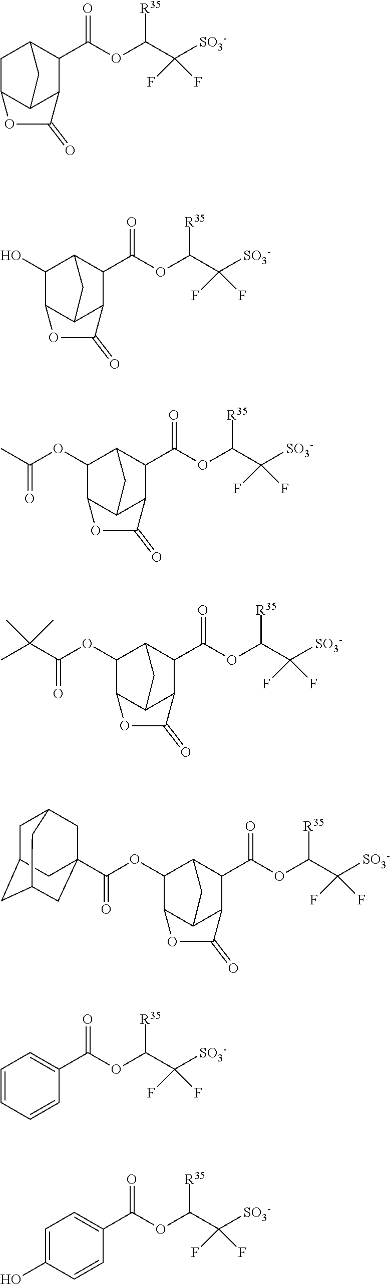

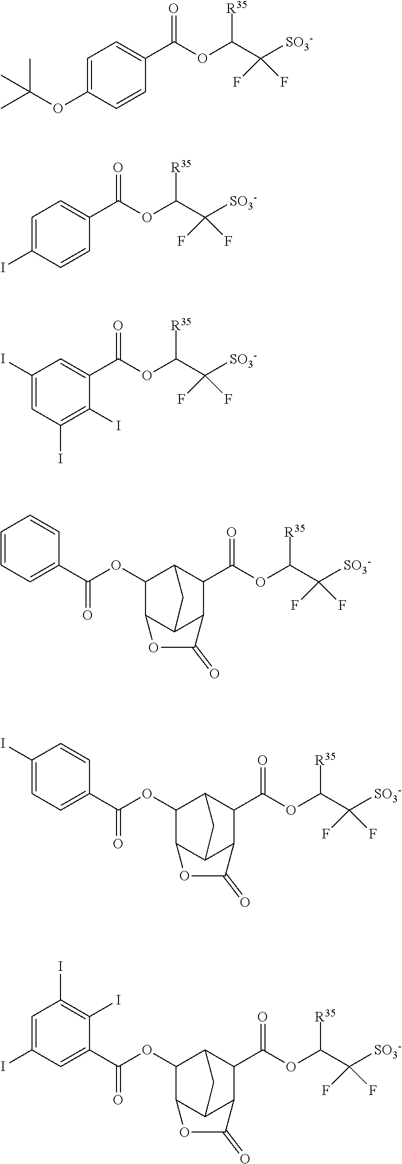

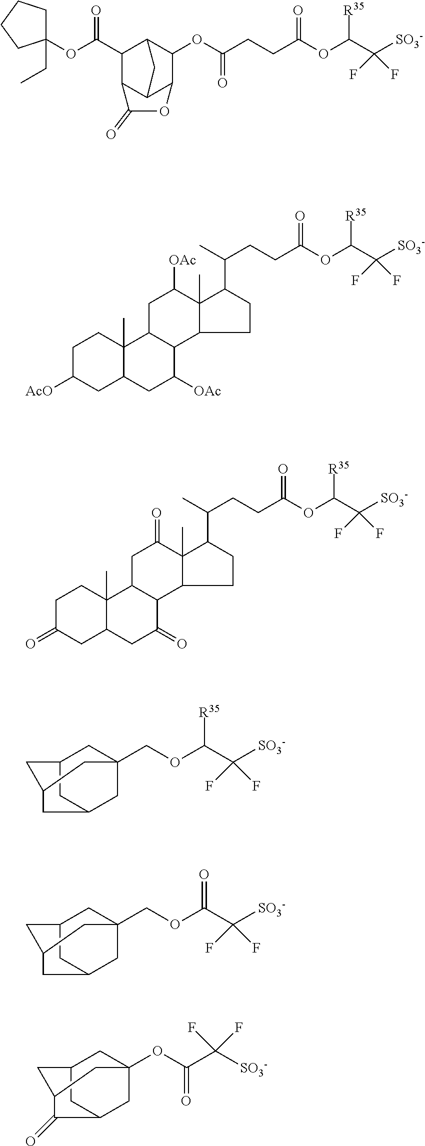

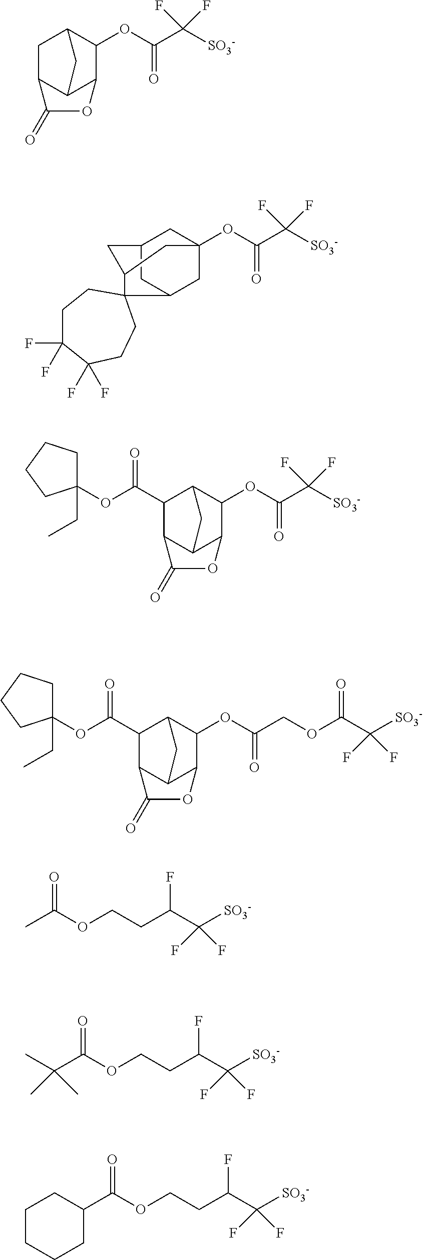

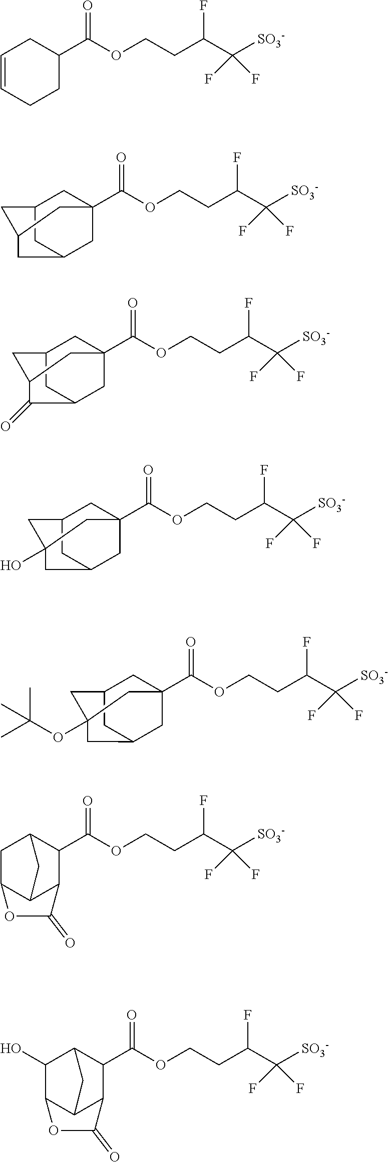

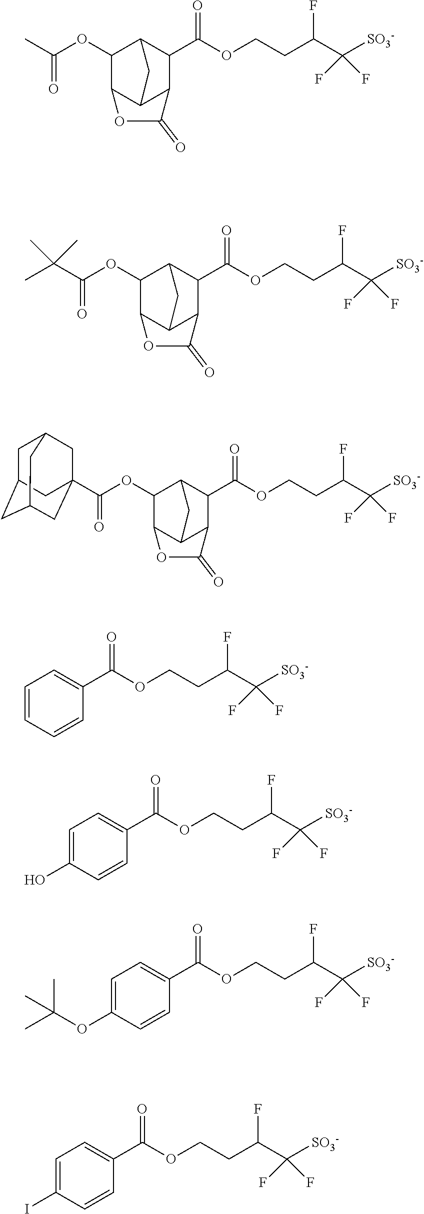

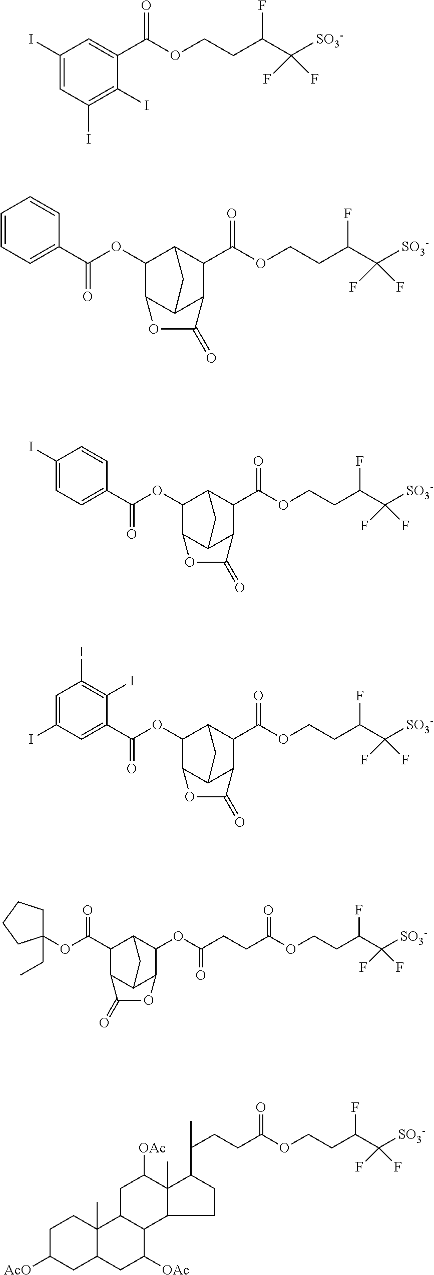

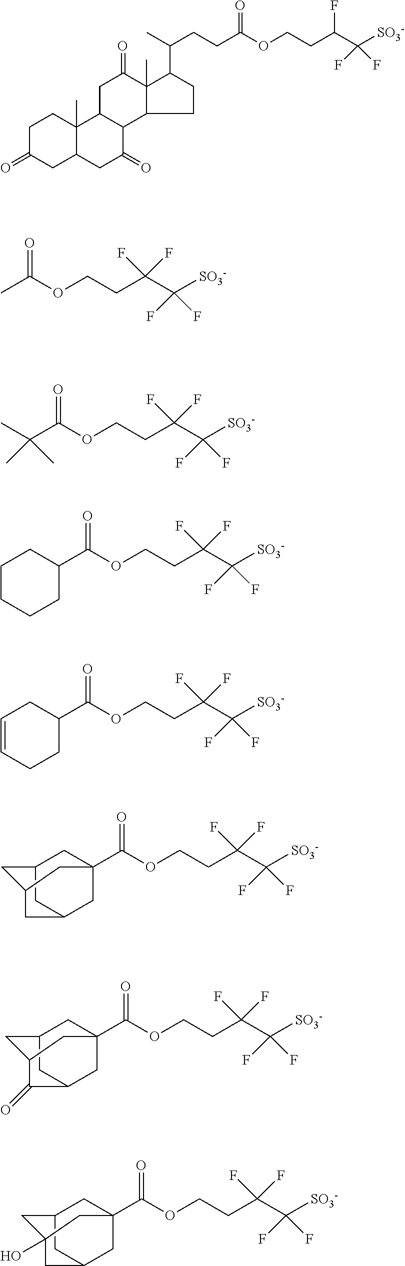

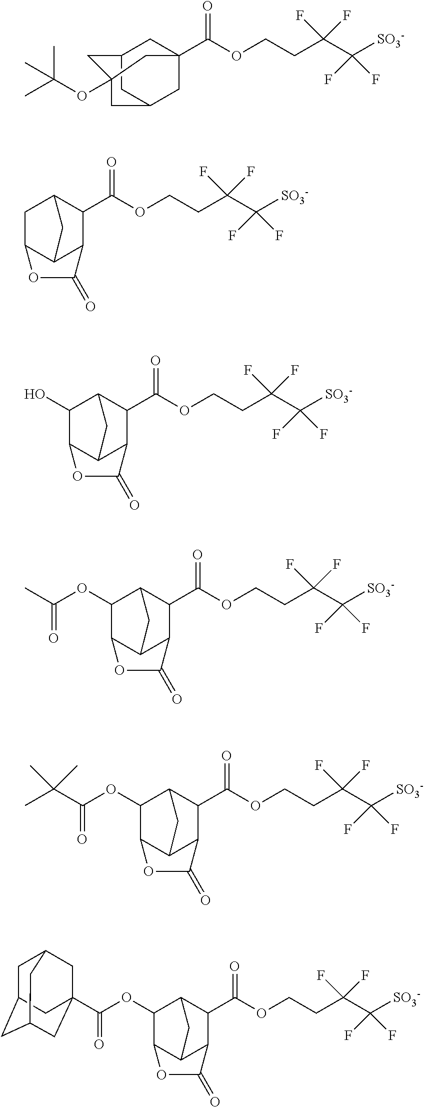

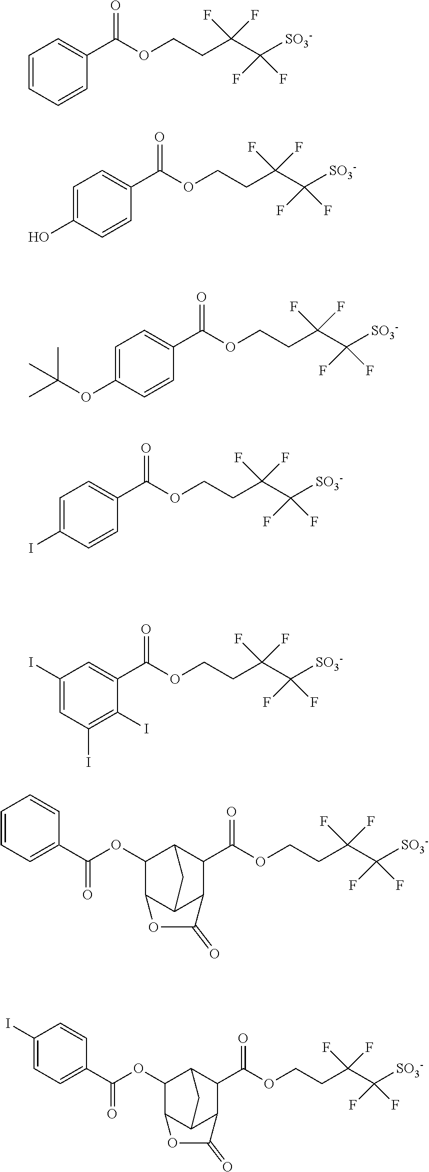

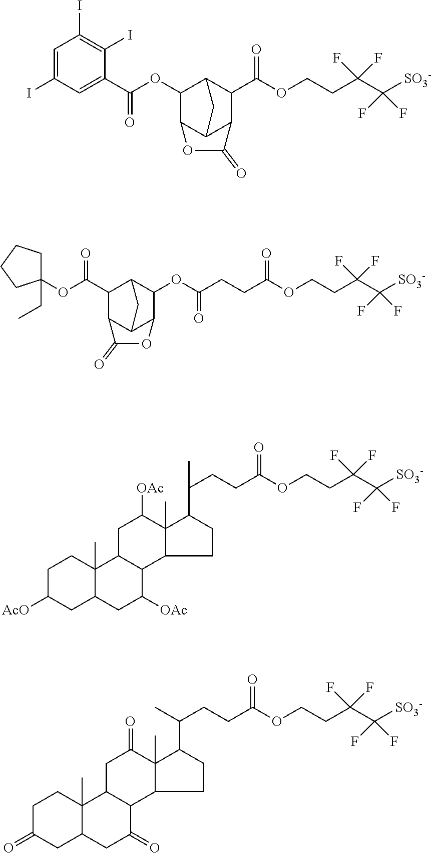

In formula (c2), examples of the optionally heteroatom-containing C₁-C₂₀ hydrocarbylene group Z⁴¹ are shown below, but not limited thereto.

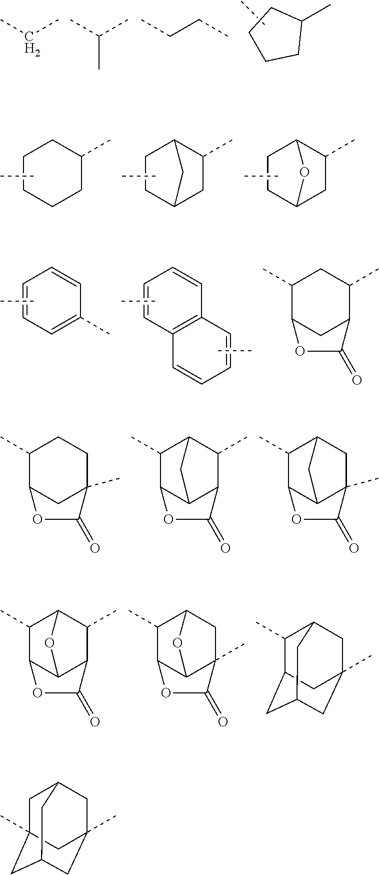

In formulae (c2) and (c3), L¹ is a single bond, ether bond, ester bond, carbonyl group, sulfonic ester bond, carbonate bond or carbamate bond.

In formulae (c2) and (c3), Rf¹ and Rf² are each independently fluorine or a C₁-C₆ fluorinated saturated hydrocarbyl group. It is preferred for enhancing the acid strength of the generated acid that both Rf¹ and Rf² be fluorine. Rf³ and Rf⁴ are each independently hydrogen, fluorine or a C₁-C₆ fluorinated saturated hydrocarbyl group. It is preferred for enhancing solvent solubility that at least one of Rf³ and Rf⁴ be trifluoromethyl. Rf⁵ and Rf⁶ are each independently hydrogen, fluorine or a C₁-C₆ fluorinated saturated hydrocarbyl group. It is excluded that all Rf⁵ and Rf⁶ are hydrogen at the same time. The subscript d is an integer of 0 to 3, preferably 1.

Examples of the anion in repeat unit having formula (c2) are shown below, but not limited thereto. Herein R^{A} is as defined above.

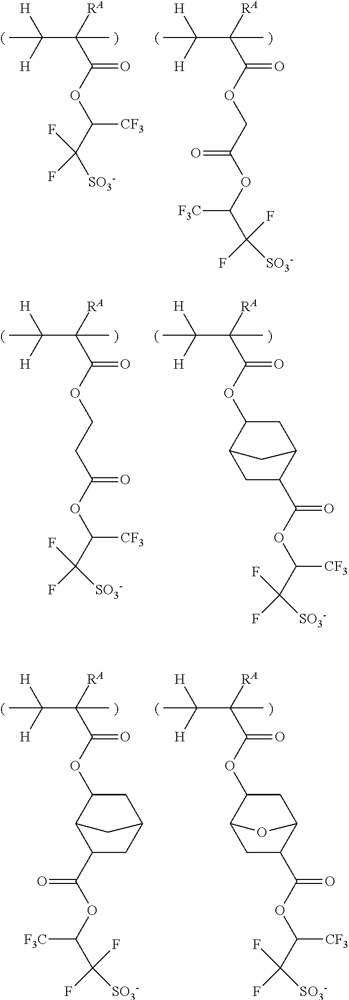

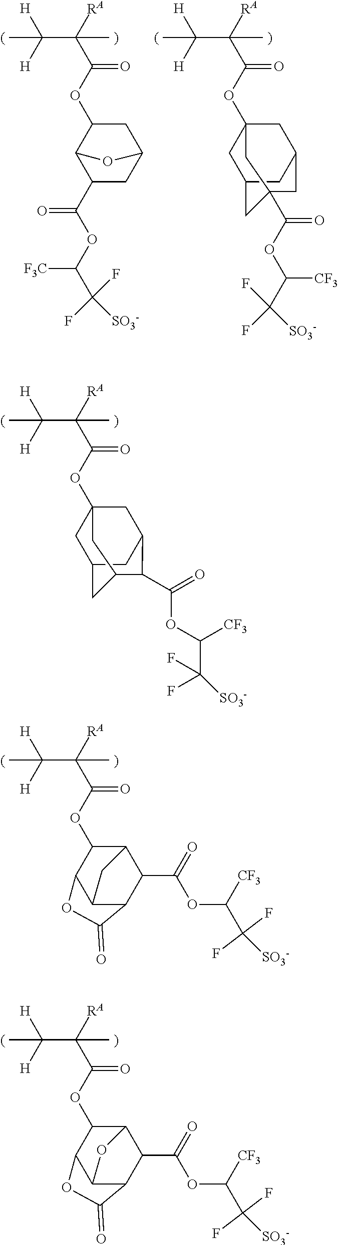

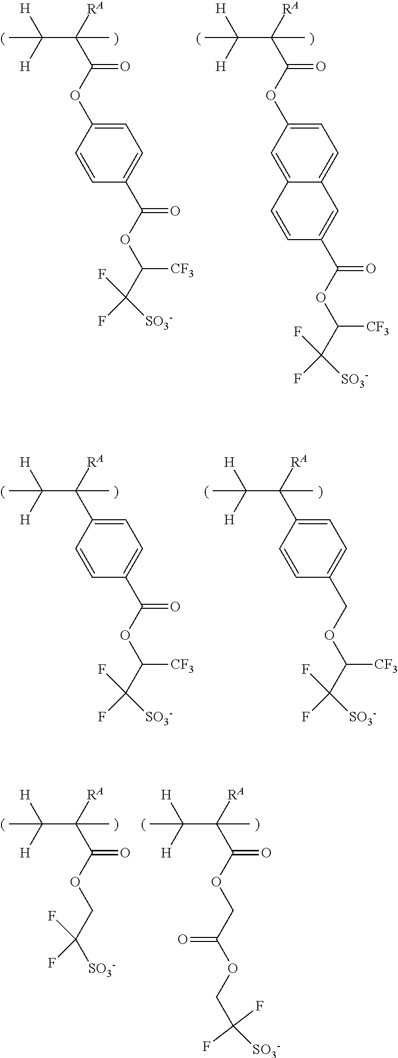

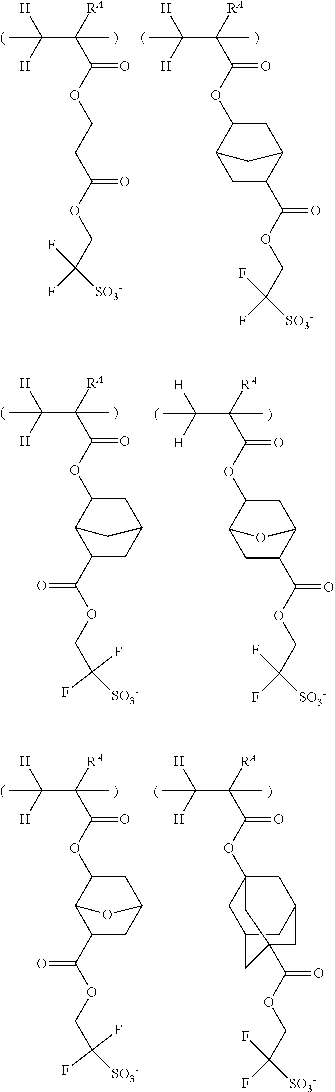

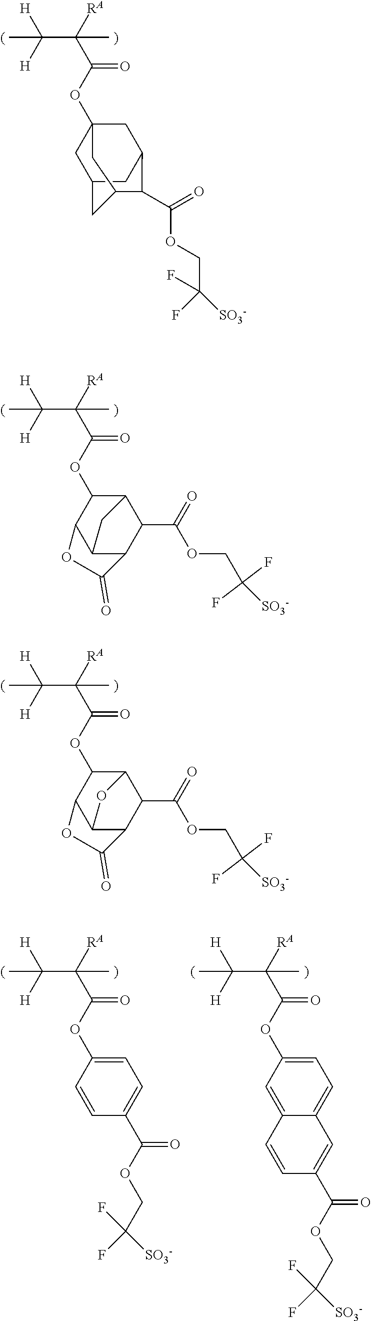

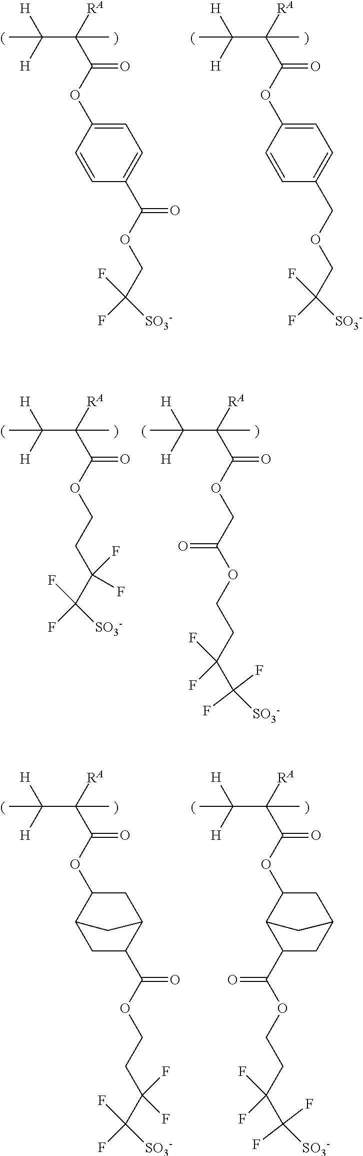

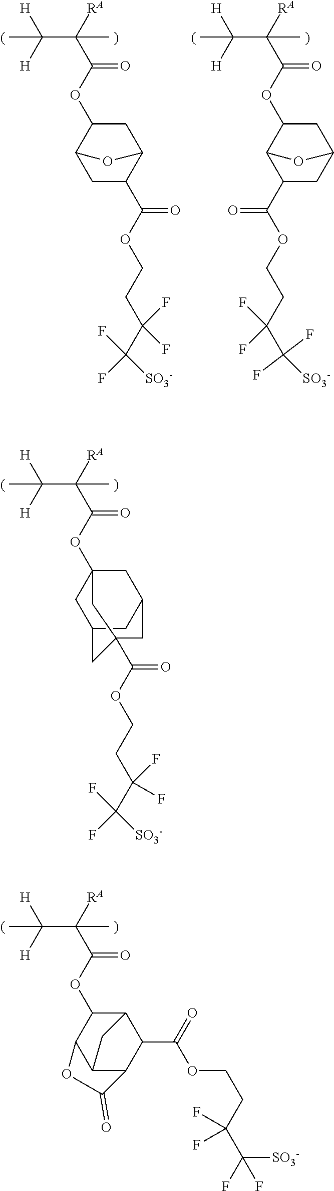

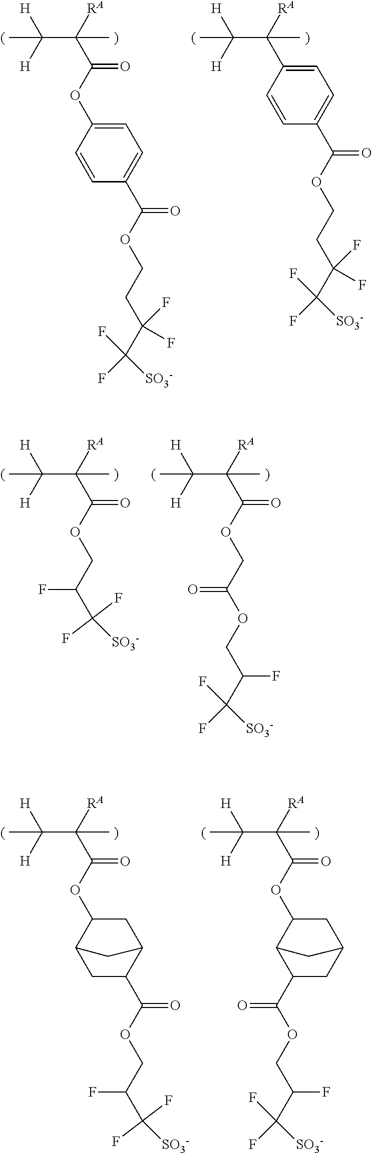

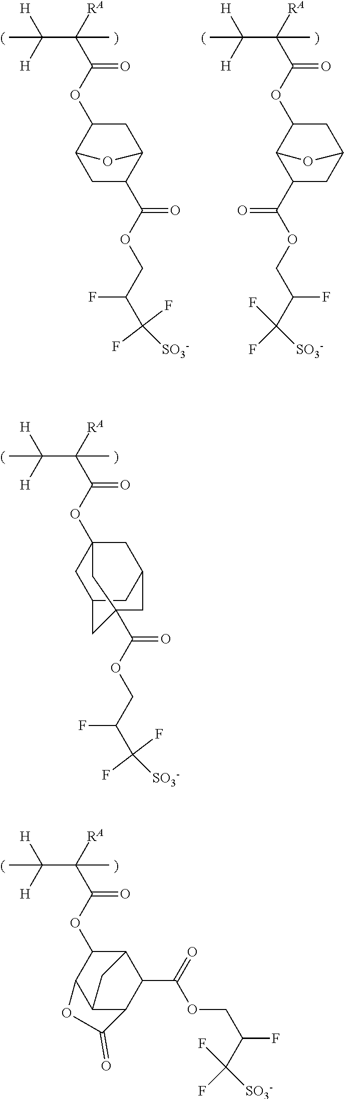

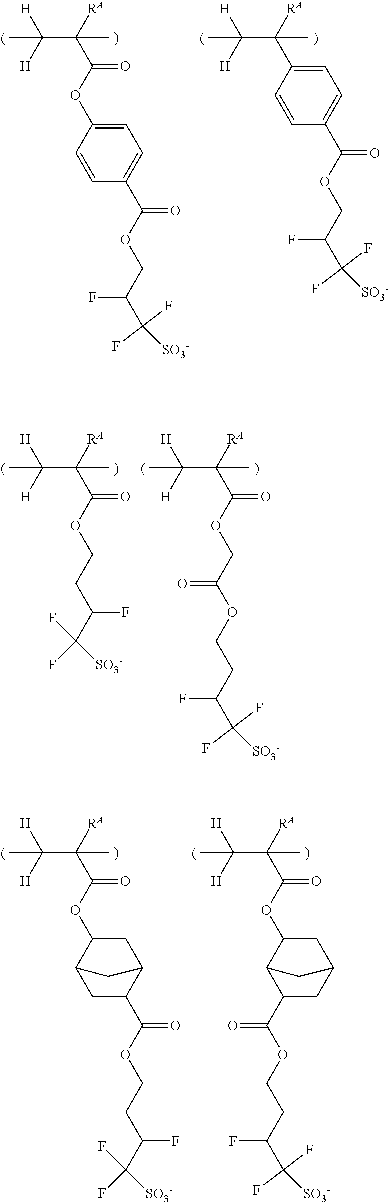

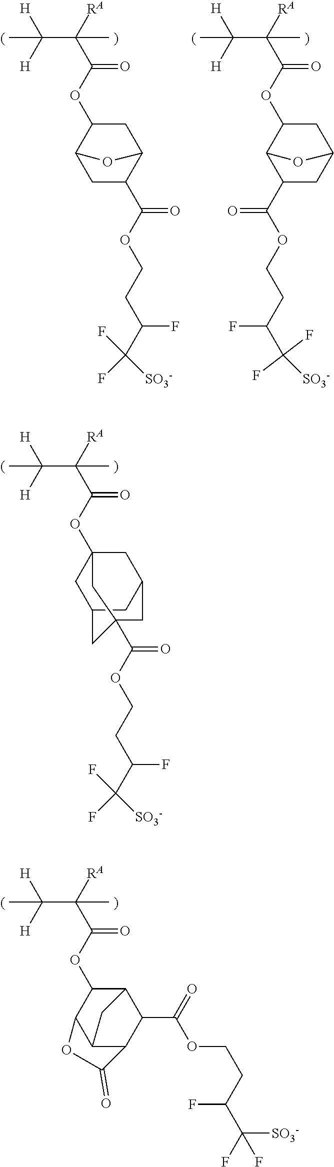

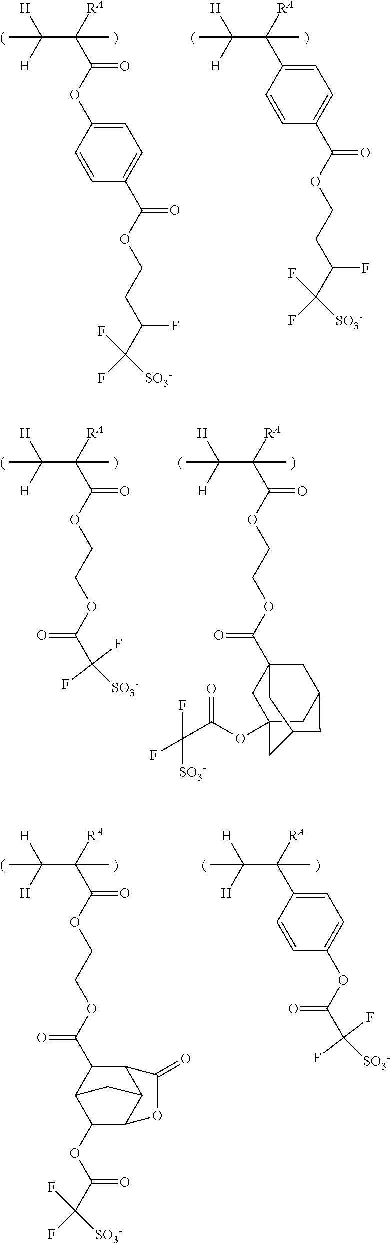

Examples of the anion in repeat rout having formula (c3) are shown below, but not limited thereto. Herein R^{A} is as defined above.

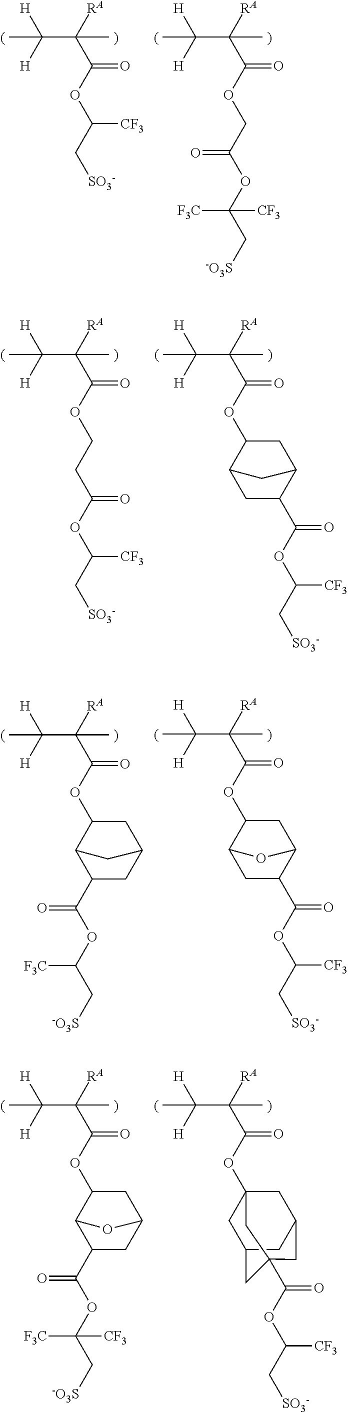

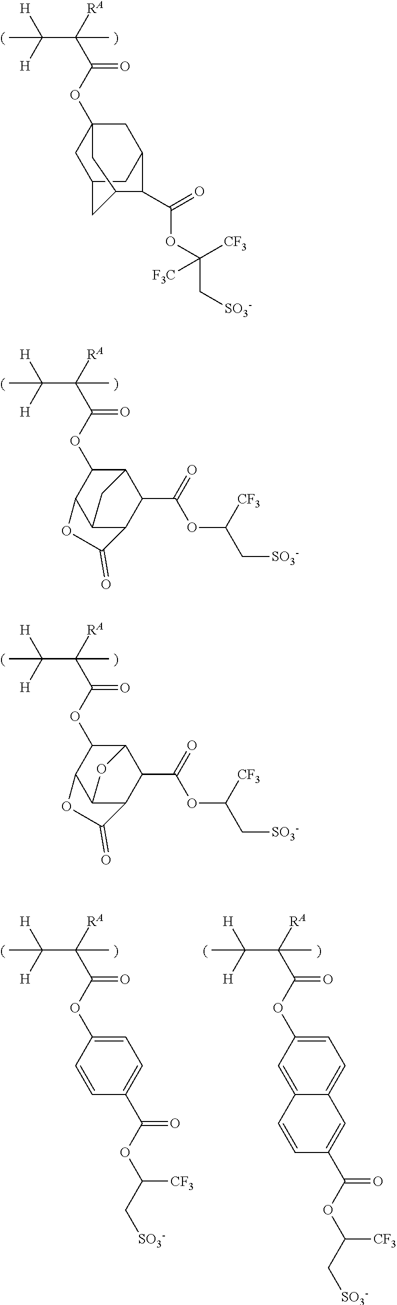

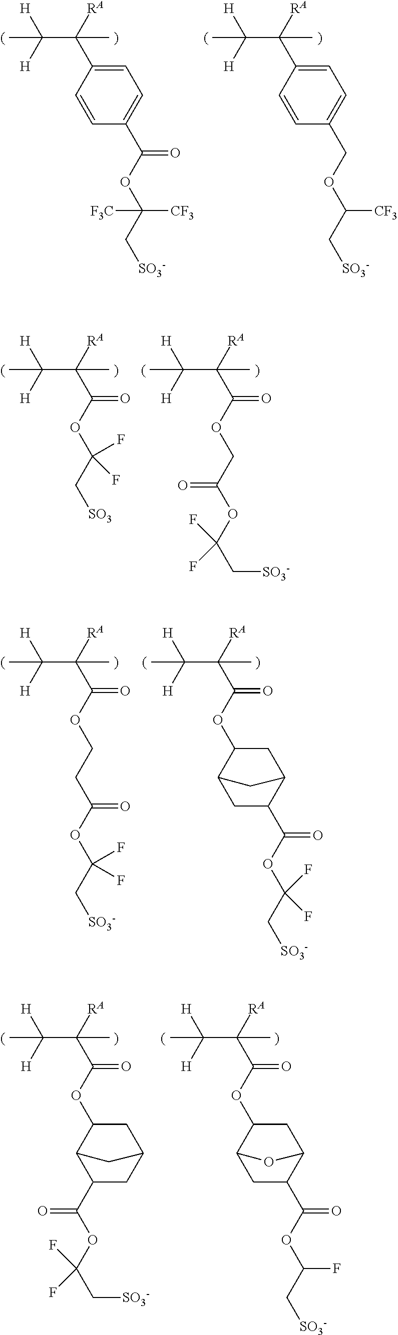

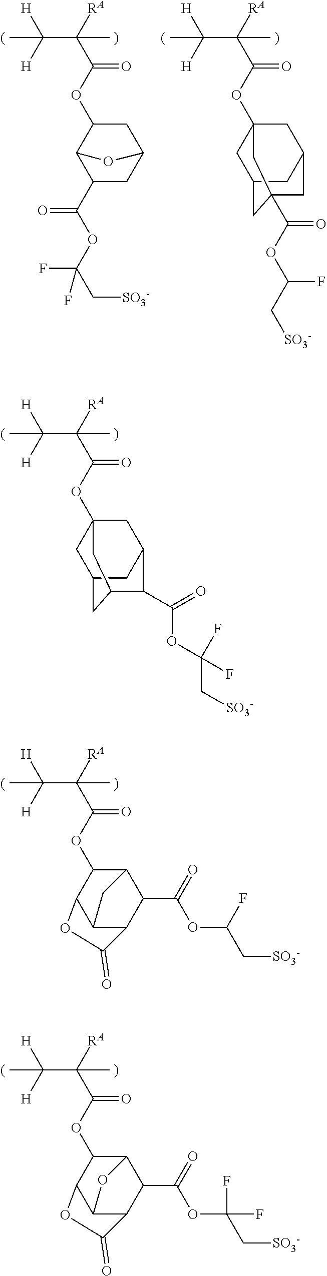

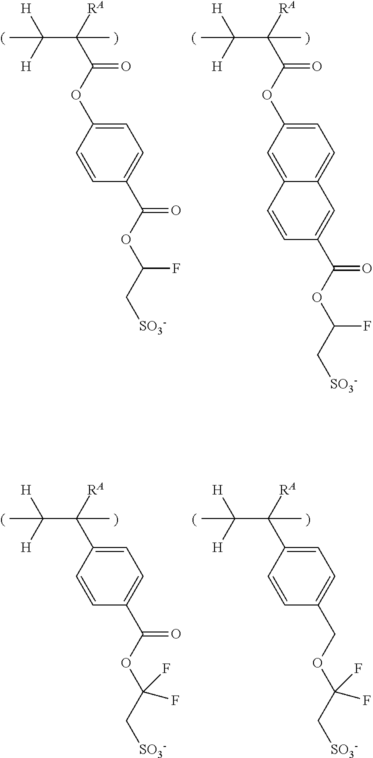

Examples of the anion in repeat unit having formula (c4) are shown below, but not limited thereto. Herein R^{A} is as defined above.

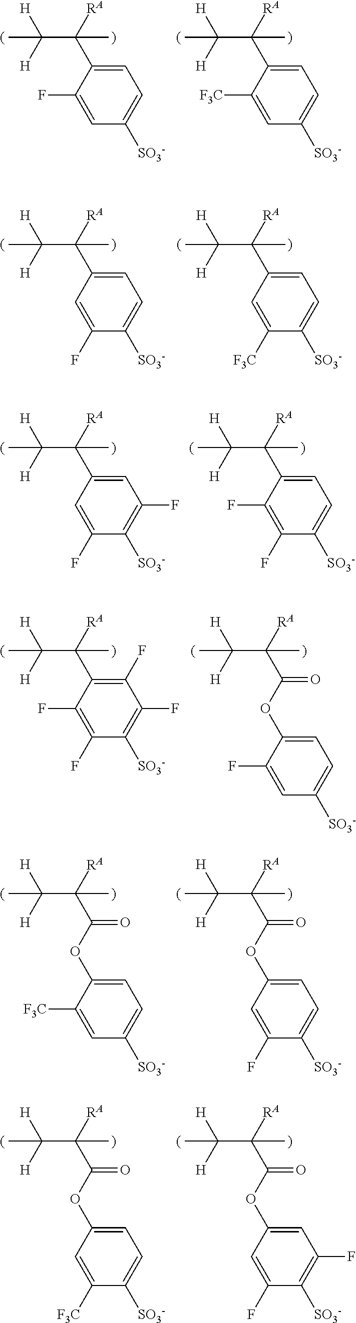

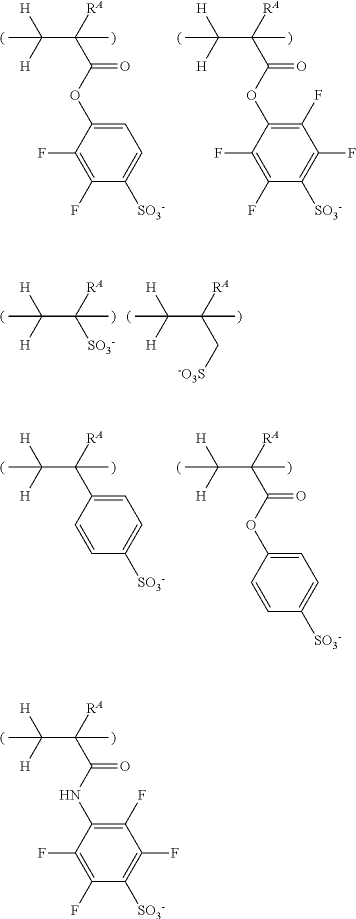

In formulae (c2), (c3) and (c4). A⁺ is an onium cation. Suitable onium cations include sulfonium, iodonium and ammonium cations, with the sulfonium and iodonium cations being preferred. More preferred are sulfonium cations having the formula (c5) and iodonium cations having the formula (c6).

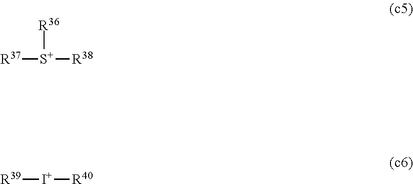

In formulae (c5) and (c6). R³⁶ to R⁴⁰ are each independently a C₁-C₂₀ hydrocarbyl group which may contain a heteroatom. The hydrocarbyl group may be saturated or unsaturated and straight, branched or cyclic. Examples thereof include C₁-C₂₀ alkyl groups such as methyl, ethyl, n-propyl, isopropyl, n-butyl, isobutyl, sec-butyl, and tert-butyl; C₃-C₂₀ cyclic saturated hydrocarbyl groups such as cyclopropyl, cyclopentyl, cyclohexyl, cyclopropylmethyl, 4-methylcyclohexyl, cyclohexylmethyl, norbornyl, and adamantyl; C₂-C₂₀ alkenyl groups such as vinyl, allyl, propenyl, butenyl, and hexenyl; C₃-C₂₀ cyclic unsaturated hydrocarbyl groups such as cyclohexenyl; C₆-C₂₀ aryl groups such as phenyl and naphthyl; and C₇-C₂₀ aralkyl groups such as benzyl, 1-phenylethyl, and 2-phenylethyl, and combinations thereof. Of these, aryl groups are preferred. In the hydrocarbyl group, some or all of the hydrogen atoms may be substituted by a moiety containing a heteroatom such as oxygen, sulfur, nitrogen or halogen, and some constituent —CH₂— may be replaced by a moiety containing a heteroatom such as oxygen, sulfur or nitrogen, so that the group may contain a hydroxy, fluorine, chlorine, bromine, iodine, cyano, carbonyl, ether bond, ester bond, sulfonic ester bond, carbonate bond, lactone ring, sultone ring, carboxylic anhydride (—C(═O)—O—C(═O)—) or haloalkyl moiety.

R³⁶ and R³⁷ may bond together to form a ring with the sulfur atom to which they are attached. Examples of the sulfonium cation having formula (c5) wherein R³⁶ and R³⁷ form a ring are shown below.

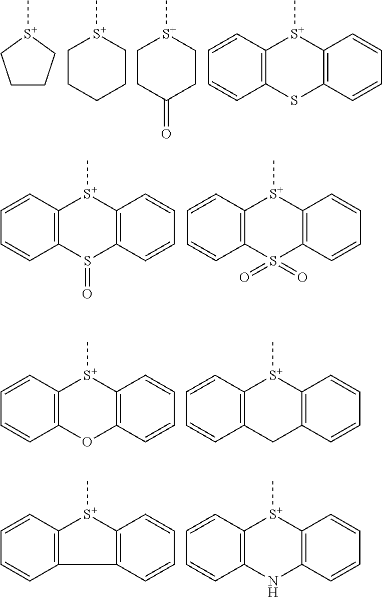

Herein, the broken line designates a point of attachment to R³⁸.

Examples of the sulfonium cation having formula (c5) are given below, but not limited thereto.

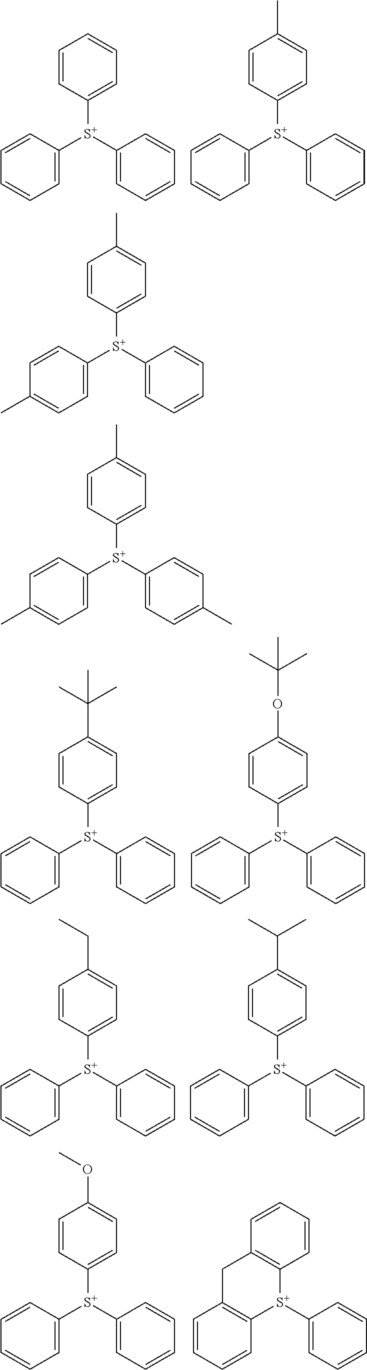

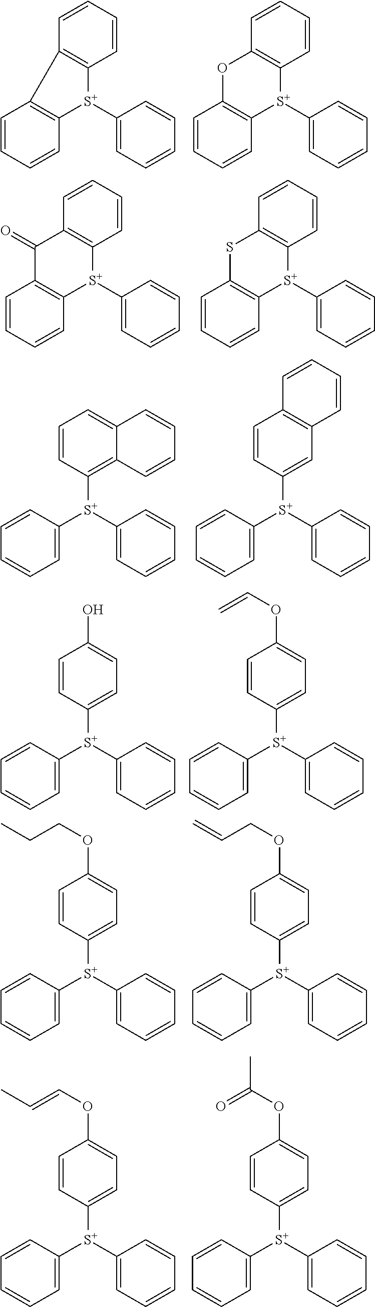

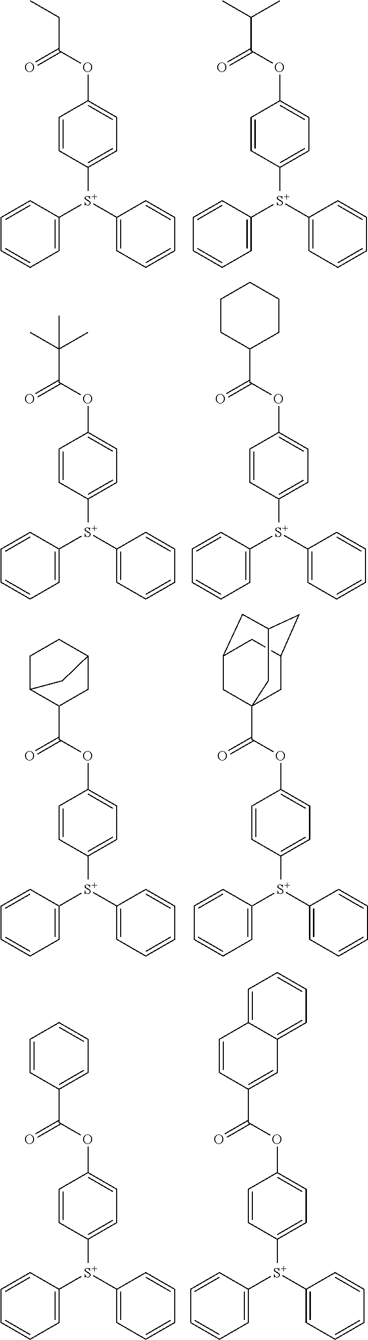

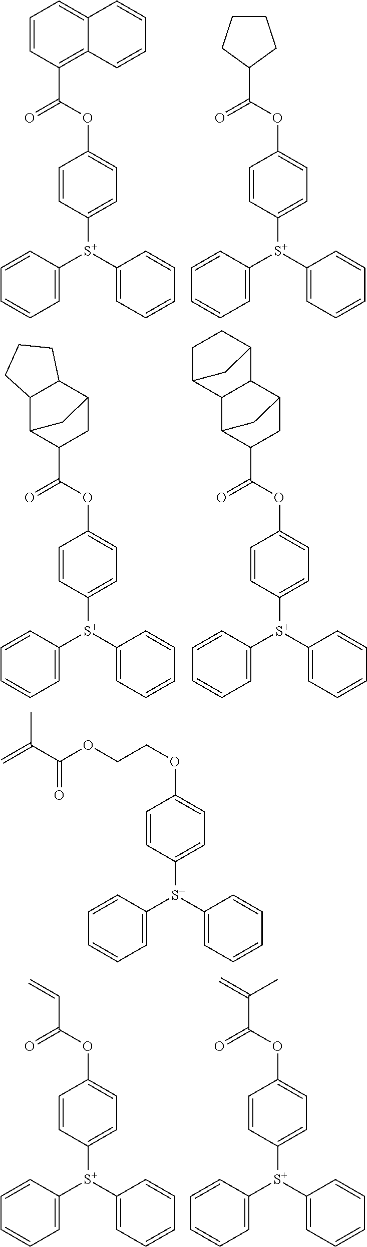

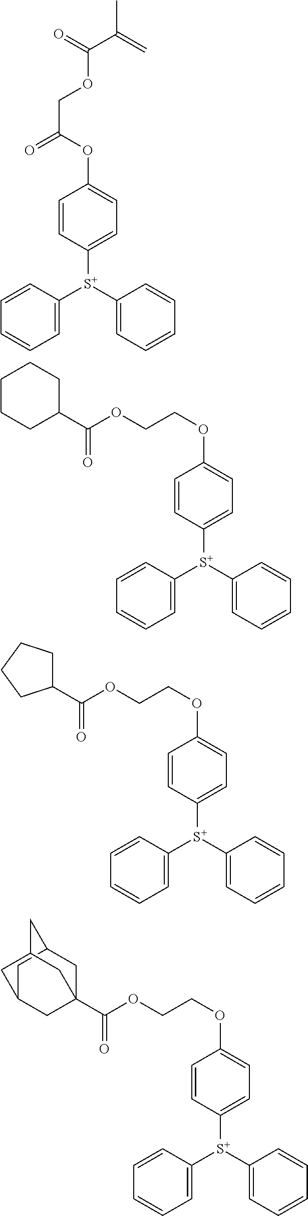 [SMILES: C=C(C)C(=O)OCC(=O)Oc1ccc([S+](c2ccccc2)c2ccccc2)cc1 ; O=C(OCCOc1ccc([S+](c2ccccc2)c2ccccc2)cc1)C1CCCCC1 ; O=C(OCCOc1ccc([S+](c2ccccc2)c2ccccc2)cc1)C1CCCC1 ; O=C(OCCOc1ccc([S+](c2ccccc2)c2ccccc2)cc1)C12CC3CC(CC(C3)C1)C2]

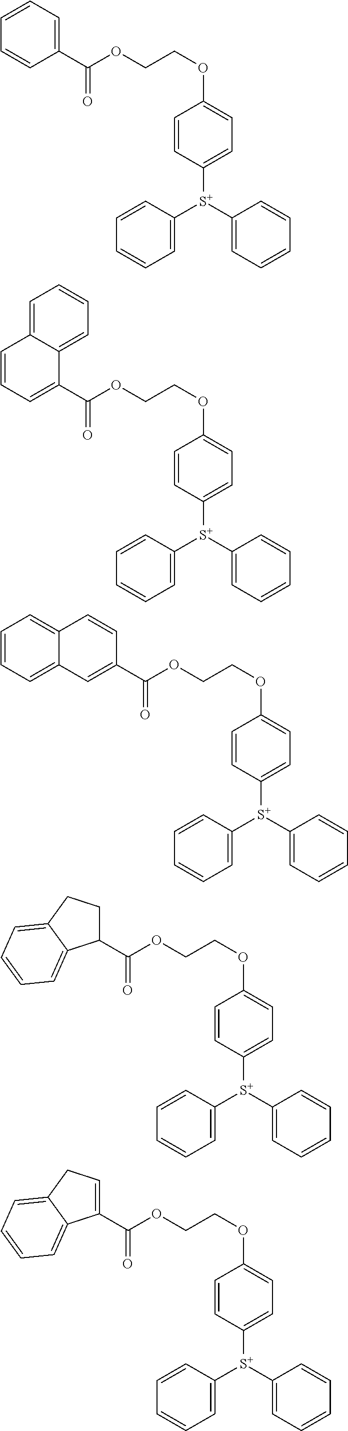 [SMILES: O=C(OCCOc1ccc([S+](c2ccccc2)c2ccccc2)cc1)c1ccccc1 ; O=C(OCCOc1ccc([S+](c2ccccc2)c2ccccc2)cc1)c1cccc2ccccc12 ; O=C(OCCOc1ccc([S+](c2ccccc2)c2ccccc2)cc1)c1ccc2ccccc2c1 ; O=C(OCCOc1ccc([S+](c2ccccc2)c2ccccc2)cc1)C1CCc2ccccc21 ; O=C(OCCOc1ccc([S+](c2ccccc2)c2ccccc2)cc1)C1=CCc2ccccc21]

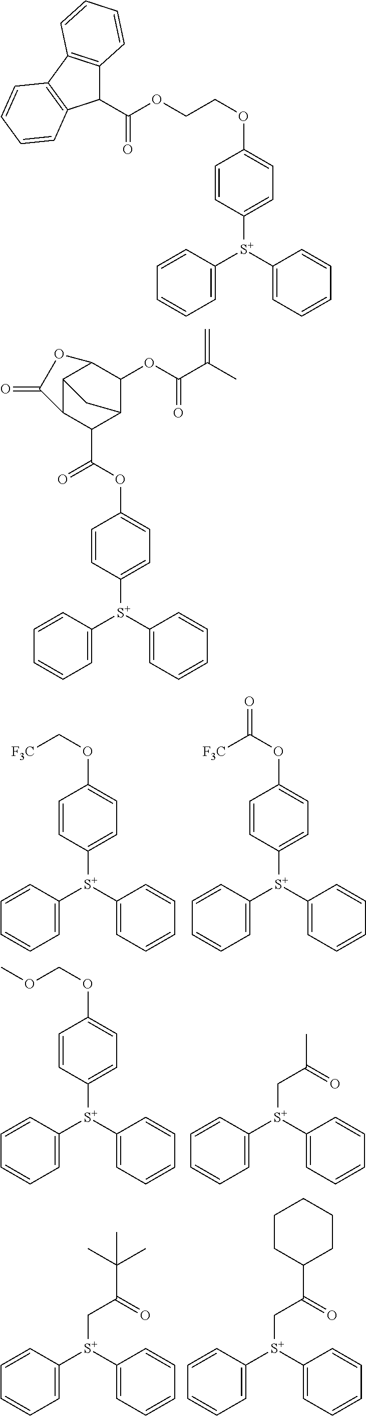

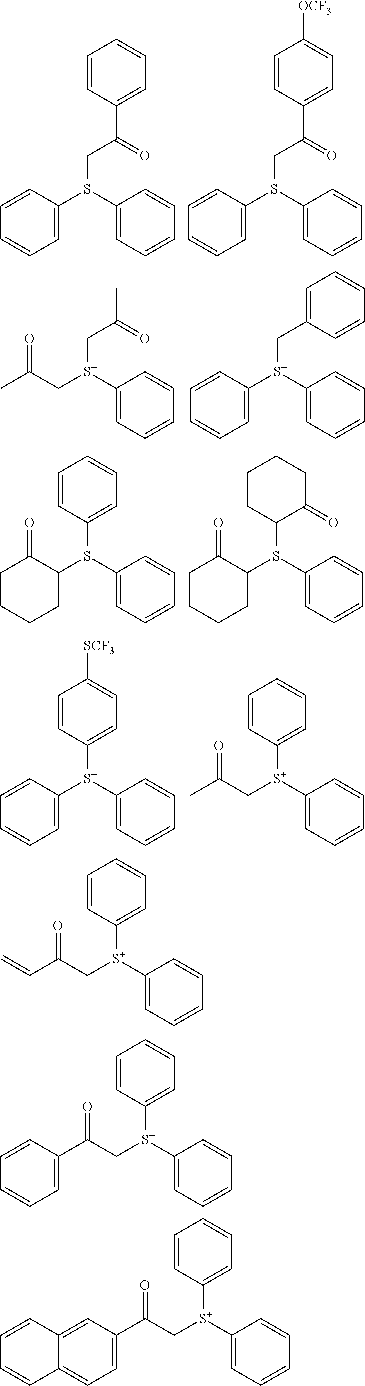

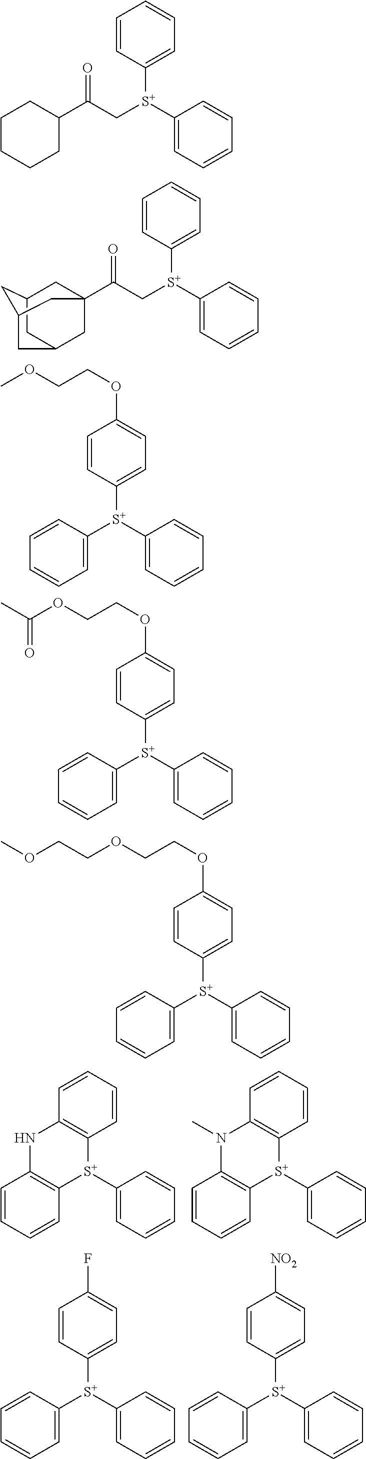

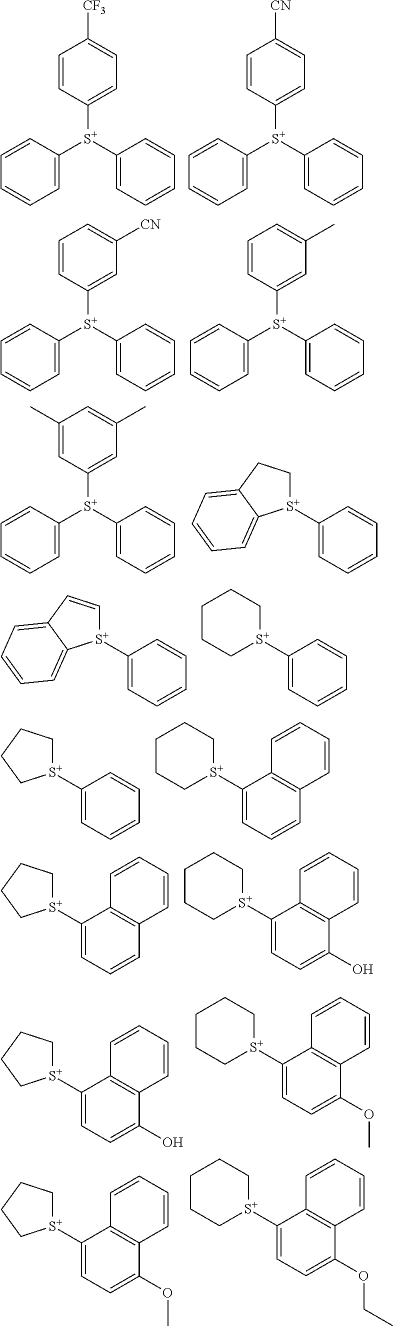

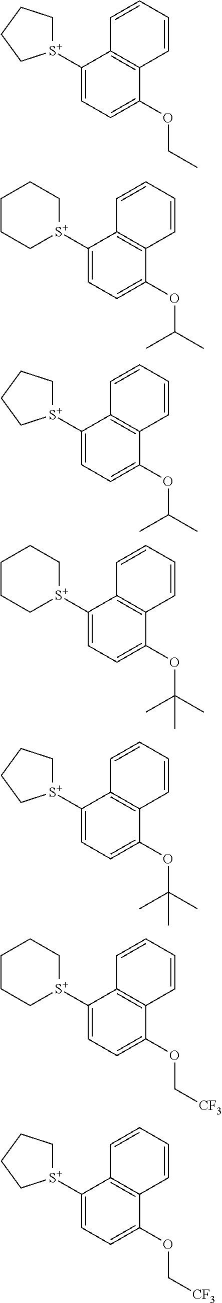

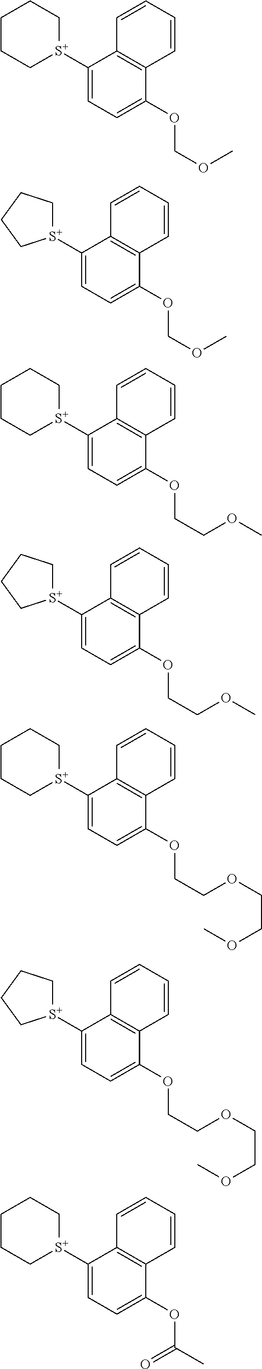

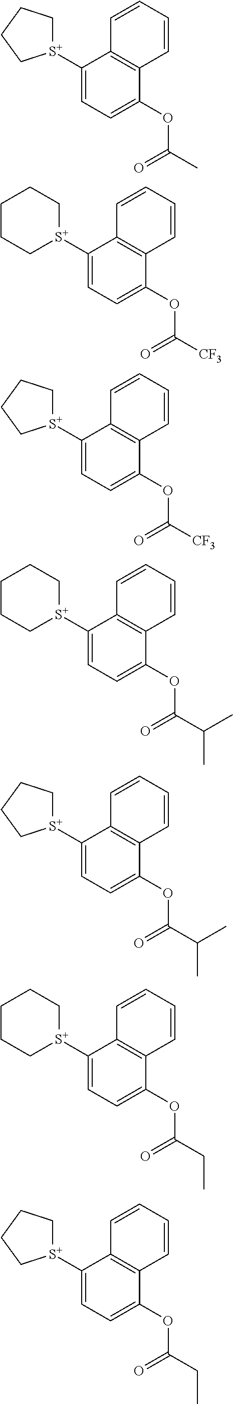

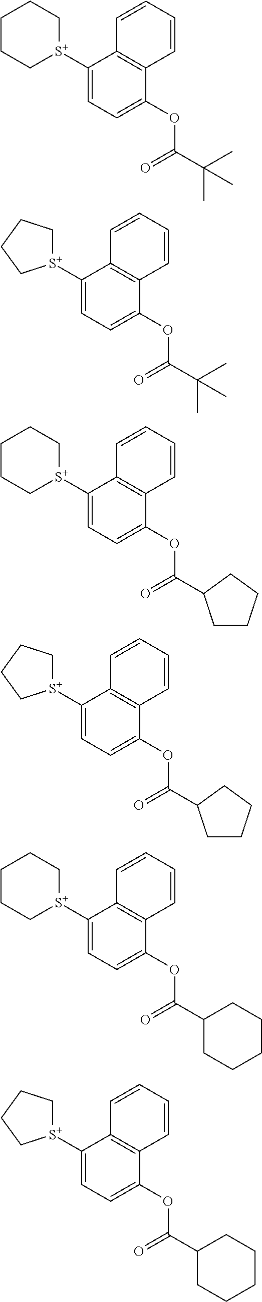 [SMILES: CC(C)(C)C(=O)Oc1ccc([S+]2CCCCC2)c2ccccc12 ; CC(C)(C)C(=O)Oc1ccc([S+]2CCCC2)c2ccccc12 ; O=C(Oc1ccc([S+]2CCCCC2)c2ccccc12)C1CCCC1 ; O=C(Oc1ccc([S+]2CCCC2)c2ccccc12)C1CCCC1 ; O=C(Oc1ccc([S+]2CCCCC2)c2ccccc12)C1CCCCC1 ; O=C(Oc1ccc([S+]2CCCC2)c2ccccc12)C1CCCCC1]

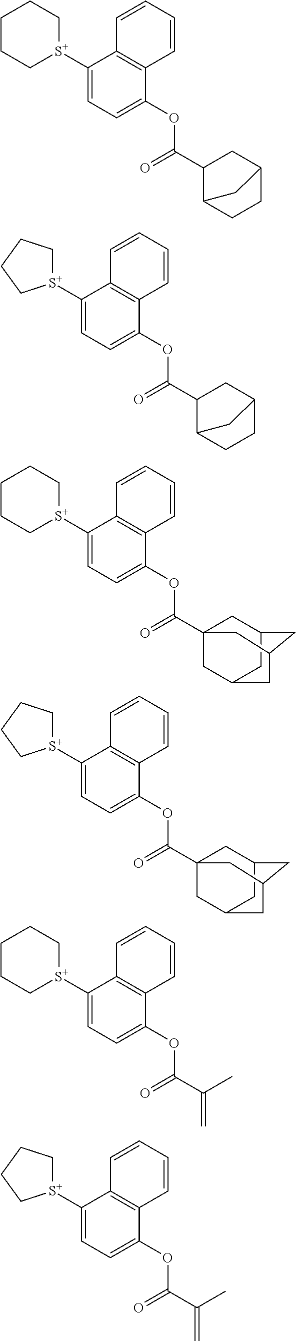 [SMILES: O=C(Oc1ccc([S+]2CCCCC2)c2ccccc12)C1CC2CCC1C2 ; O=C(Oc1ccc([S+]2CCCC2)c2ccccc12)C1CC2CCC1C2 ; O=C(Oc1ccc([S+]2CCCCC2)c2ccccc12)C12CC3CC(CC(C3)C1)C2 ; O=C(Oc1ccc([S+]2CCCC2)c2ccccc12)C12CC3CC(CC(C3)C1)C2 ; C=C(C)C(=O)Oc1ccc([S+]2CCCCC2)c2ccccc12 ; C=C(C)C(=O)Oc1ccc([S+]2CCCC2)c2ccccc12]

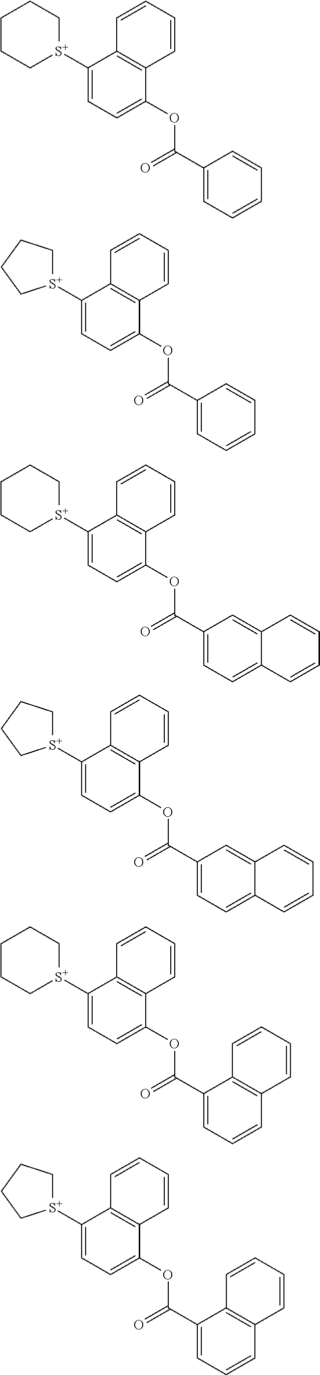 [SMILES: O=C(Oc1ccc([S+]2CCCCC2)c2ccccc12)c1ccccc1 ; O=C(Oc1ccc([S+]2CCCC2)c2ccccc12)c1ccccc1 ; O=C(Oc1ccc([S+]2CCCCC2)c2ccccc12)c1ccc2ccccc2c1 ; O=C(Oc1ccc([S+]2CCCC2)c2ccccc12)c1ccc2ccccc2c1 ; O=C(Oc1ccc([S+]2CCCCC2)c2ccccc12)c1cccc2ccccc12 ; O=C(Oc1ccc([S+]2CCCC2)c2ccccc12)c1cccc2ccccc12]

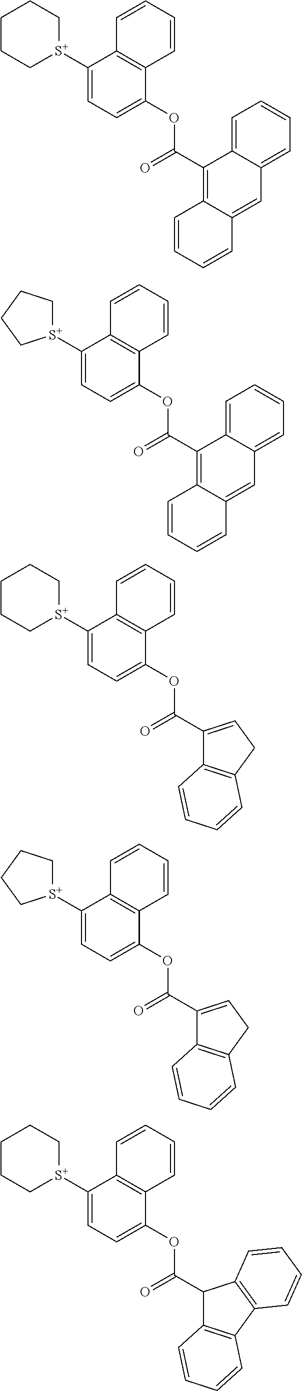 [SMILES: O=C(Oc1ccc([S+]2CCCCC2)c2ccccc12)c1c2ccccc2cc2ccccc12 ; O=C(Oc1ccc([S+]2CCCC2)c2ccccc12)c1c2ccccc2cc2ccccc12 ; O=C(Oc1ccc([S+]2CCCCC2)c2ccccc12)C1=CCc2ccccc21 ; O=C(Oc1ccc([S+]2CCCC2)c2ccccc12)C1=CCc2ccccc21 ; O=C(Oc1ccc([S+]2CCCCC2)c2ccccc12)C1c2ccccc2-c2ccccc21]

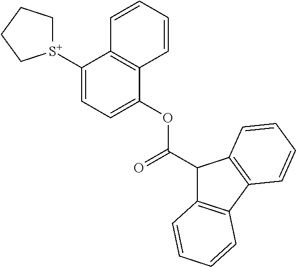 [SMILES: O=C(Oc1ccc([S+]2CCCC2)c2ccccc12)C1c2ccccc2-c2ccccc21]

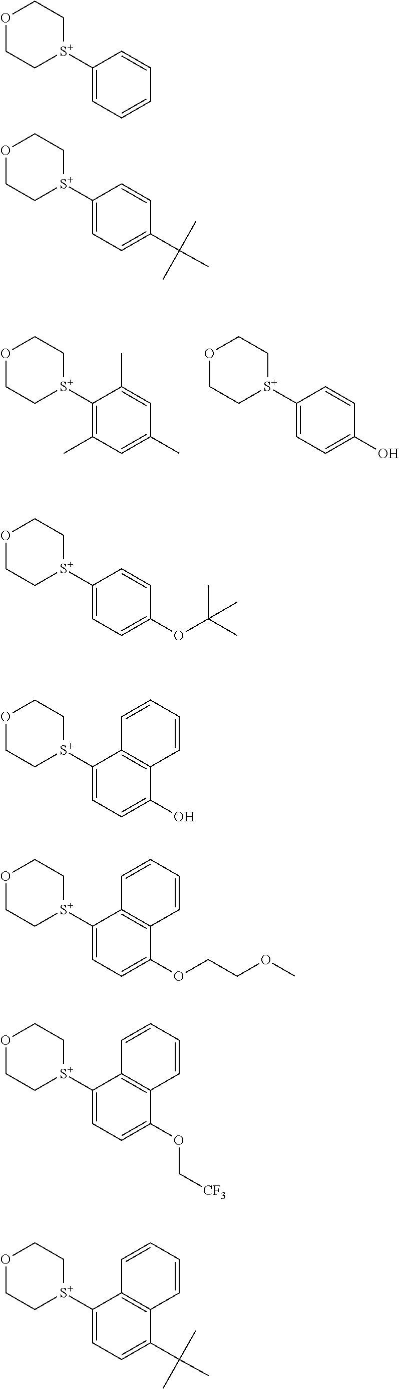

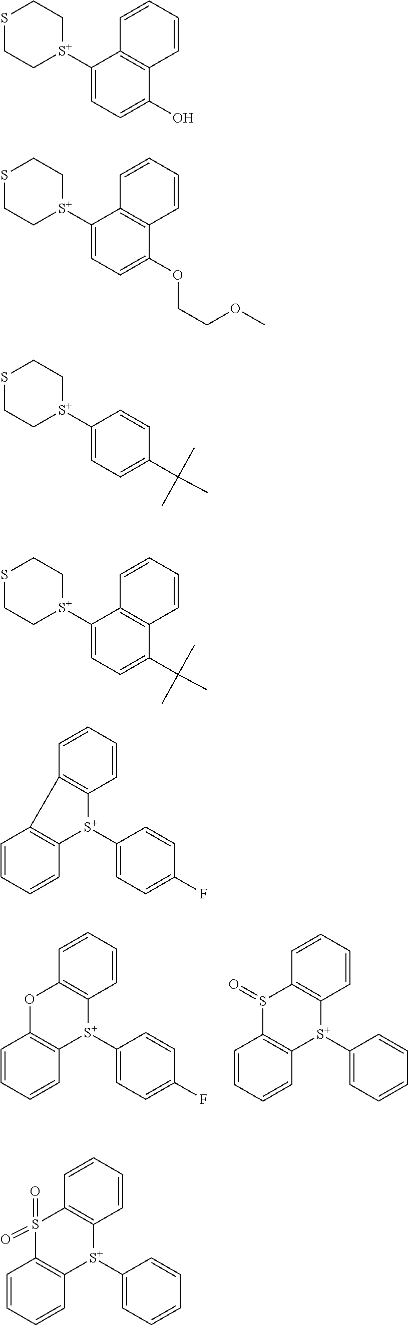

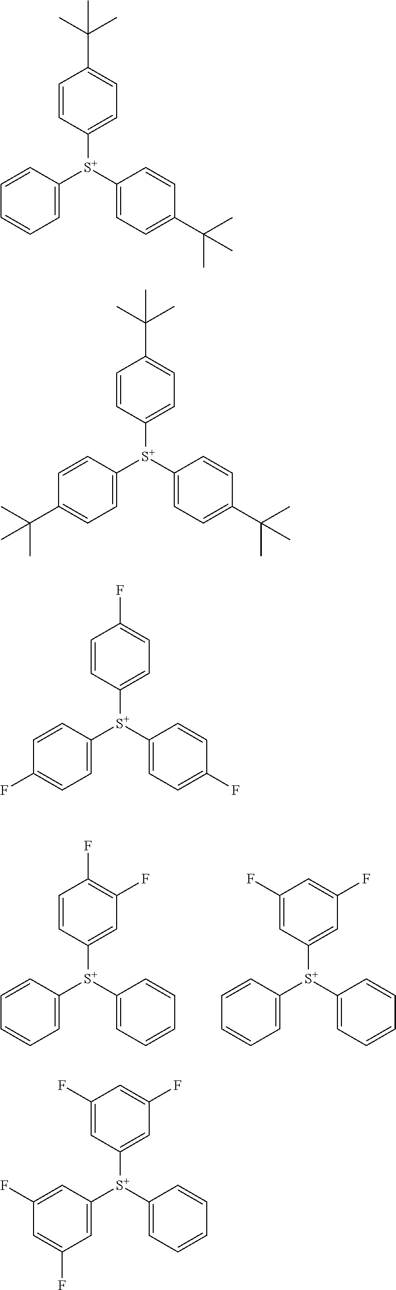 [SMILES: CC(C)(C)c1ccc([S+](c2ccccc2)c2ccc(C(C)(C)C)cc2)cc1 ; CC(C)(C)c1ccc([S+](c2ccc(C(C)(C)C)cc2)c2ccc(C(C)(C)C)cc2)cc1 ; Fc1ccc([S+](c2ccc(F)cc2)c2ccc(F)cc2)cc1 ; Fc1ccc([S+](c2ccccc2)c2ccccc2)cc1F ; Fc1cc(F)cc([S+](c2ccccc2)c2ccccc2)c1 ; Fc1cc(F)cc([S+](c2ccccc2)c2cc(F)cc(F)c2)c1]

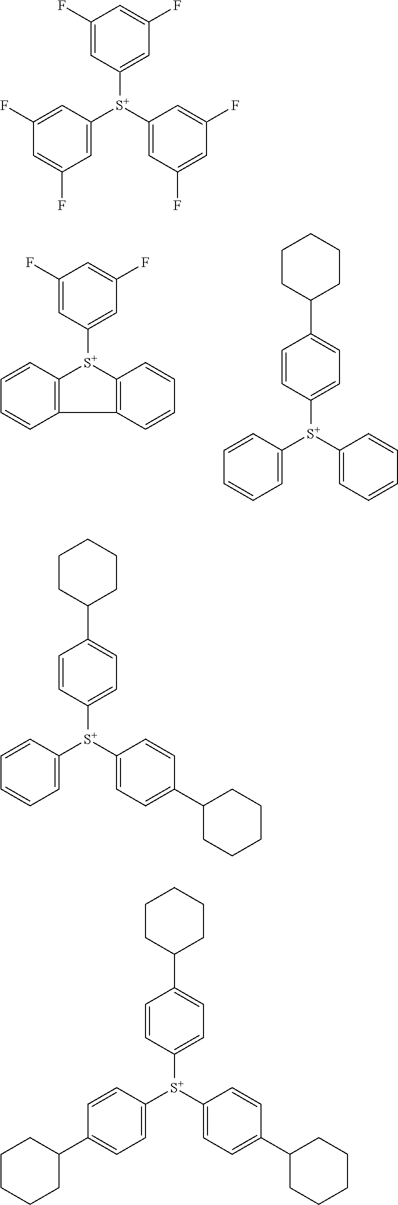 [SMILES: Fc1cc(F)cc([S+](c2cc(F)cc(F)c2)c2cc(F)cc(F)c2)c1 ; Fc1cc(F)cc(-[s+]2c3ccccc3c3ccccc32)c1 ; c1ccc([S+](c2ccccc2)c2ccc(C3CCCCC3)cc2)cc1 ; c1ccc([S+](c2ccc(C3CCCCC3)cc2)c2ccc(C3CCCCC3)cc2)cc1 ; c1cc([S+](c2ccc(C3CCCCC3)cc2)c2ccc(C3CCCCC3)cc2)ccc1C1CCCCC1]

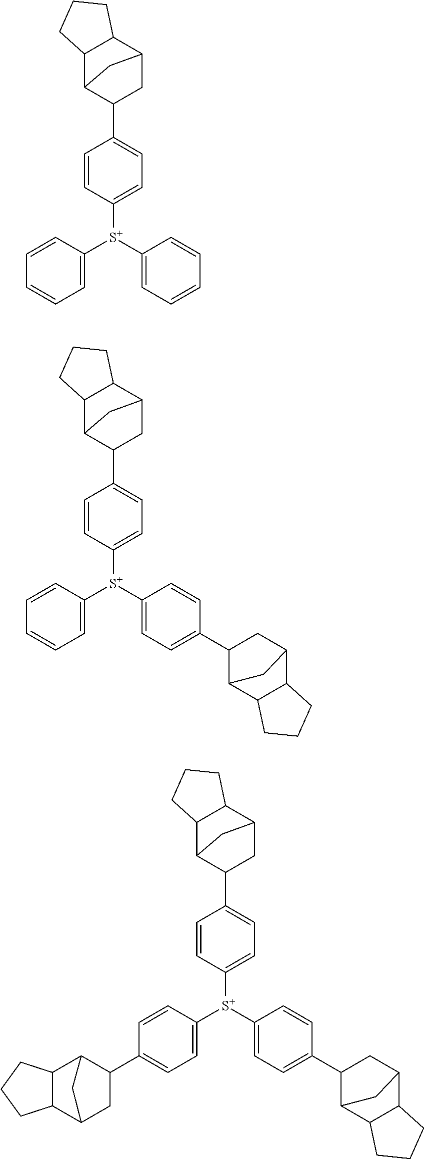 [SMILES: c1ccc([S+](c2ccccc2)c2ccc(C3CC4CC3C3CCCC43)cc2)cc1 ; c1ccc([S+](c2ccc(C3CC4CC3C3CCCC43)cc2)c2ccc(C3CC4CC3C3CCCC43)cc2)cc1 ; c1cc([S+](c2ccc(C3CC4CC3C3CCCC43)cc2)c2ccc(C3CC4CC3C3CCCC43)cc2)ccc1C1CC2CC1C1CCCC21]

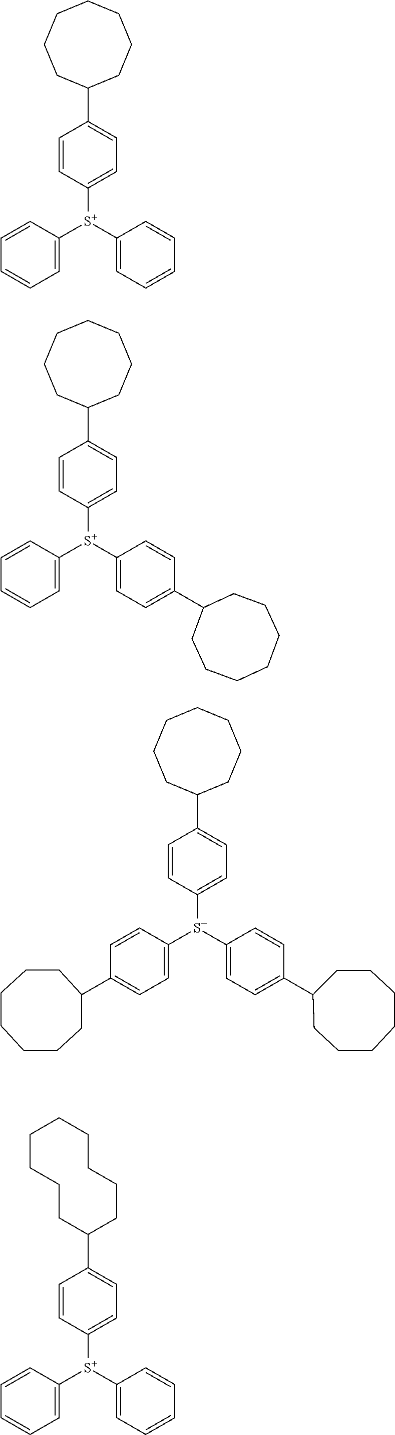 [SMILES: c1ccc([S+](c2ccccc2)c2ccc(C3CCCCCCC3)cc2)cc1 ; c1ccc([S+](c2ccc(C3CCCCCCC3)cc2)c2ccc(C3CCCCCCC3)cc2)cc1 ; c1cc([S+](c2ccc(C3CCCCCCC3)cc2)c2ccc(C3CCCCCCC3)cc2)ccc1C1CCCCCCC1 ; c1ccc([S+](c2ccccc2)c2ccc(C3CCCCCCCCC3)cc2)cc1]

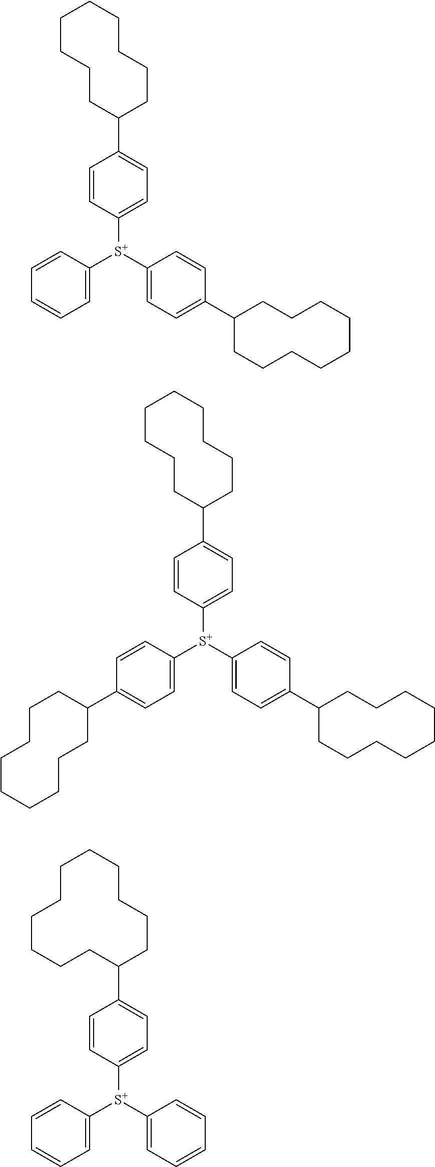 [SMILES: c1ccc([S+](c2ccc(C3CCCCCCCCC3)cc2)c2ccc(C3CCCCCCCCC3)cc2)cc1 ; c1cc([S+](c2ccc(C3CCCCCCCCC3)cc2)c2ccc(C3CCCCCCCCC3)cc2)ccc1C1CCCCCCCCC1 ; c1ccc([S+](c2ccccc2)c2ccc(C3CCCCCCCCCCC3)cc2)cc1]

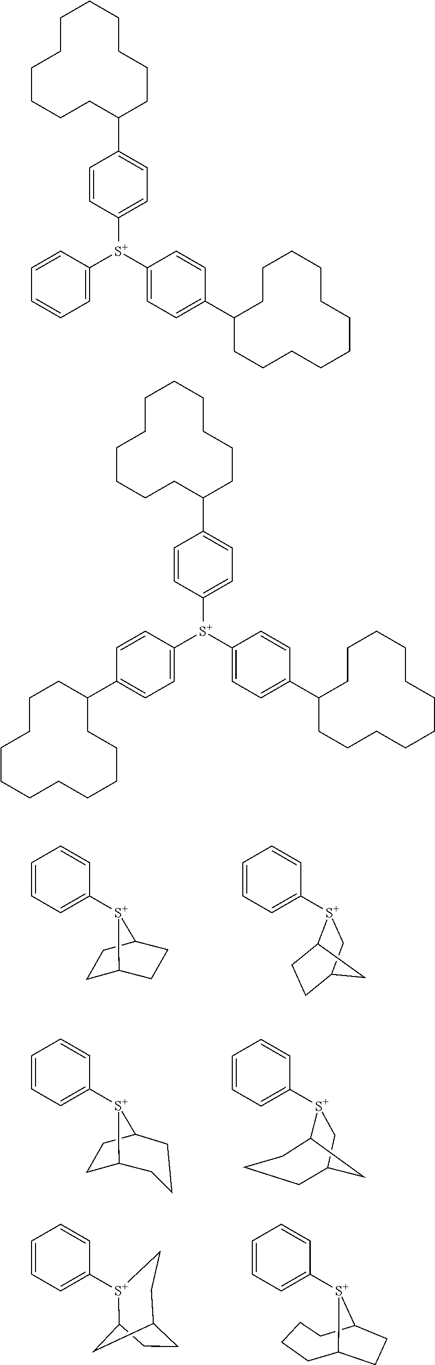

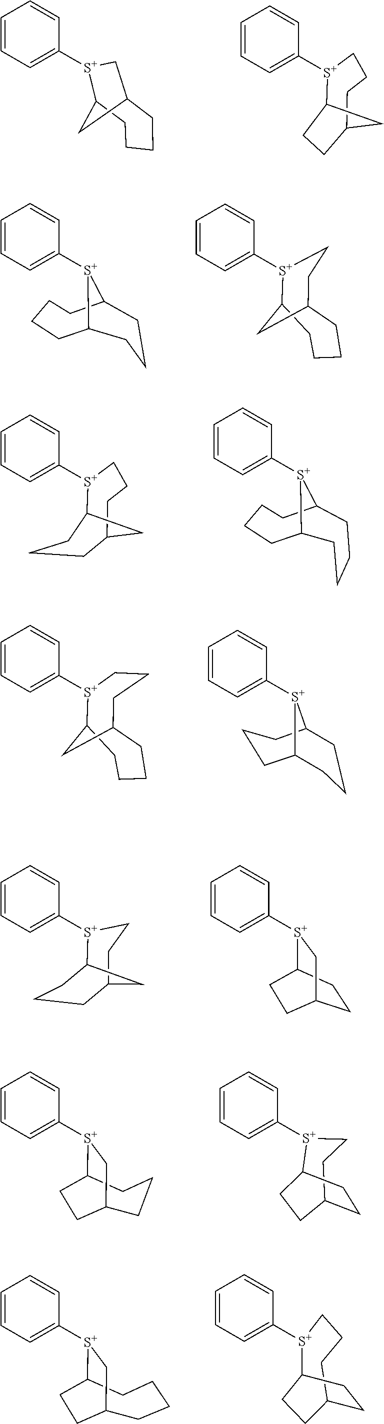

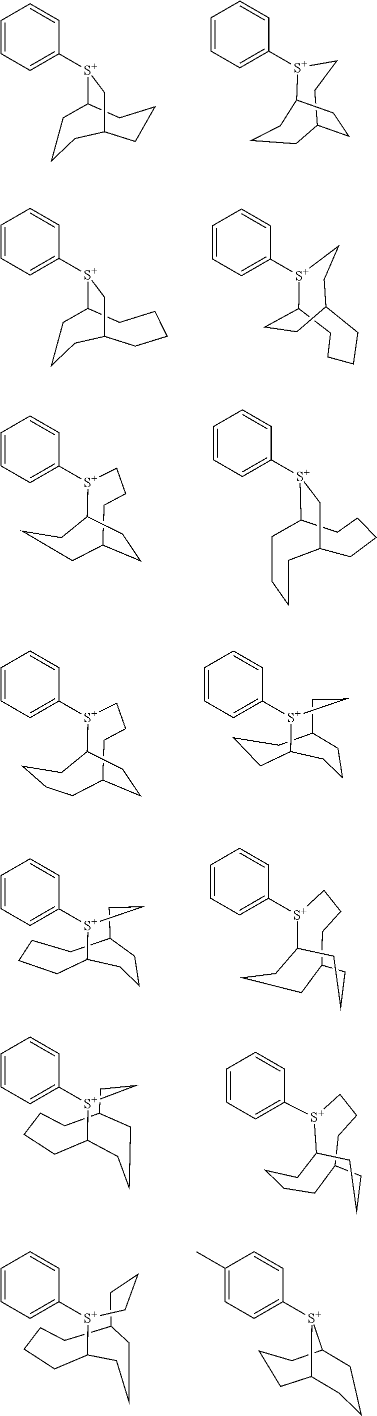

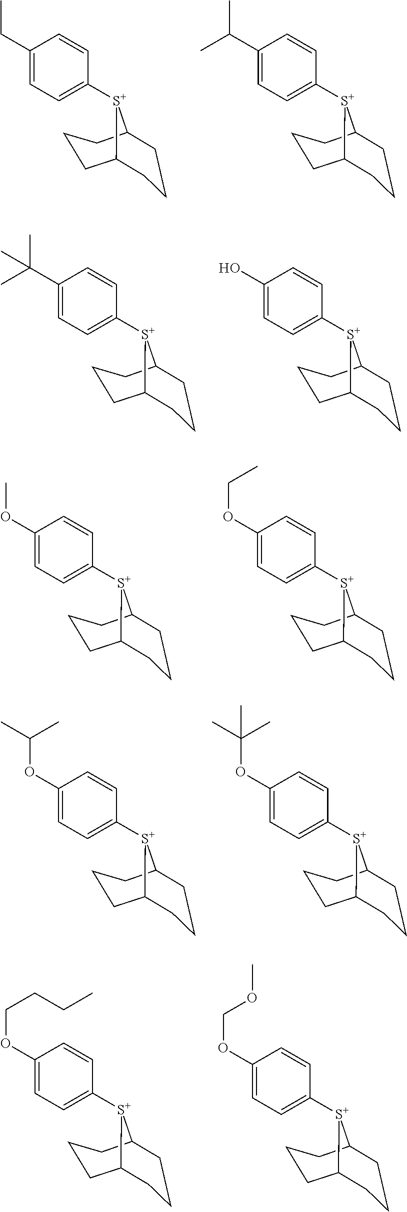

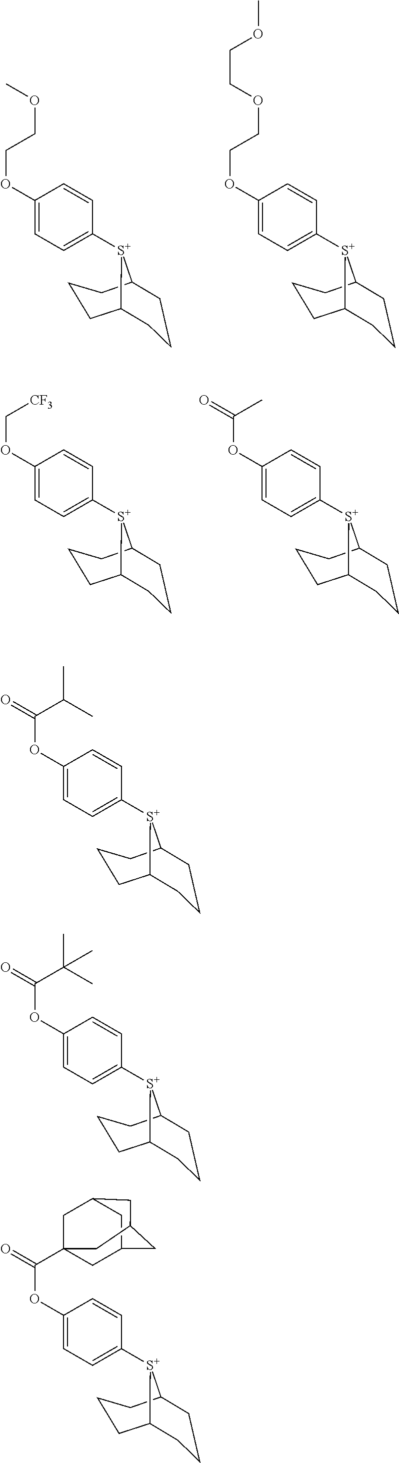

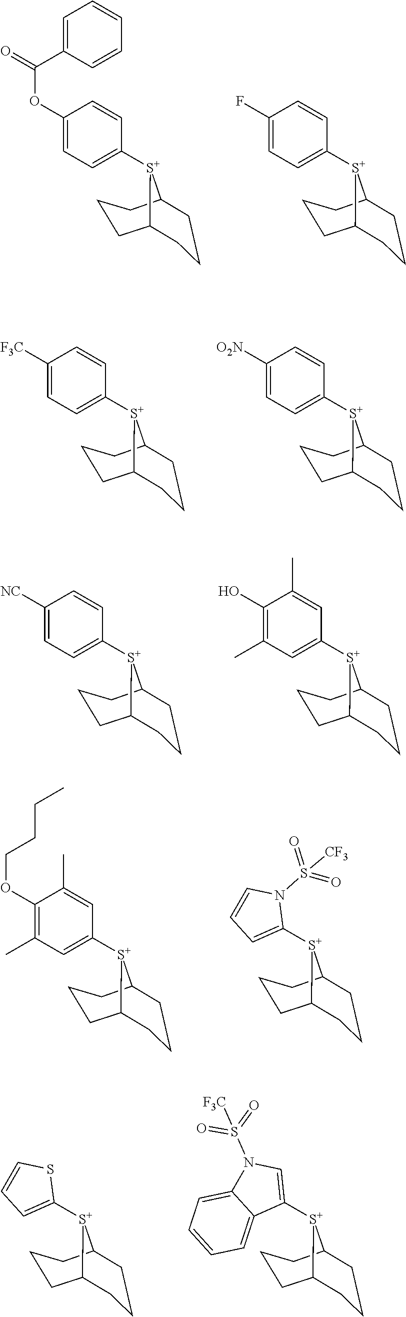

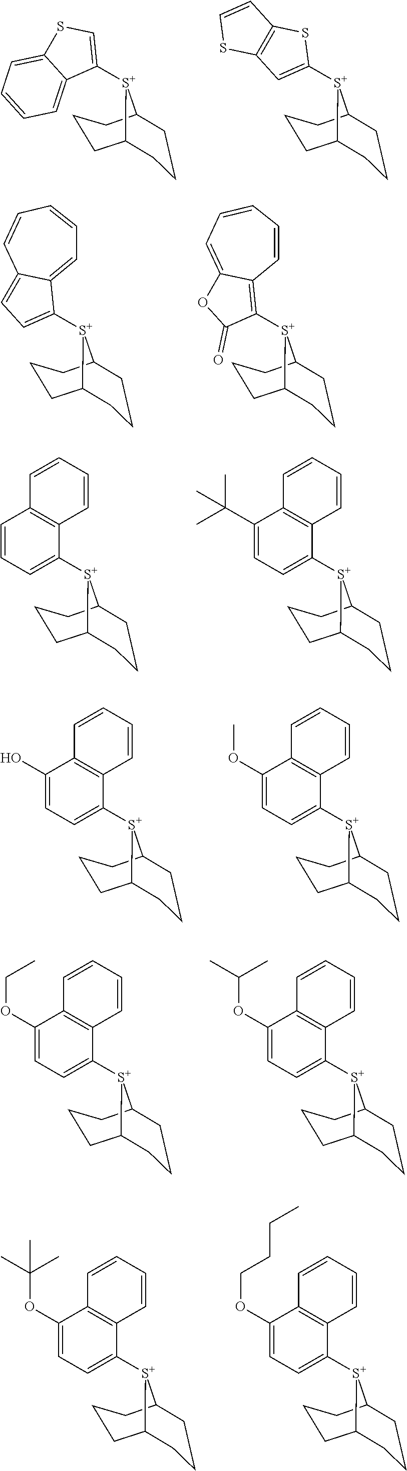

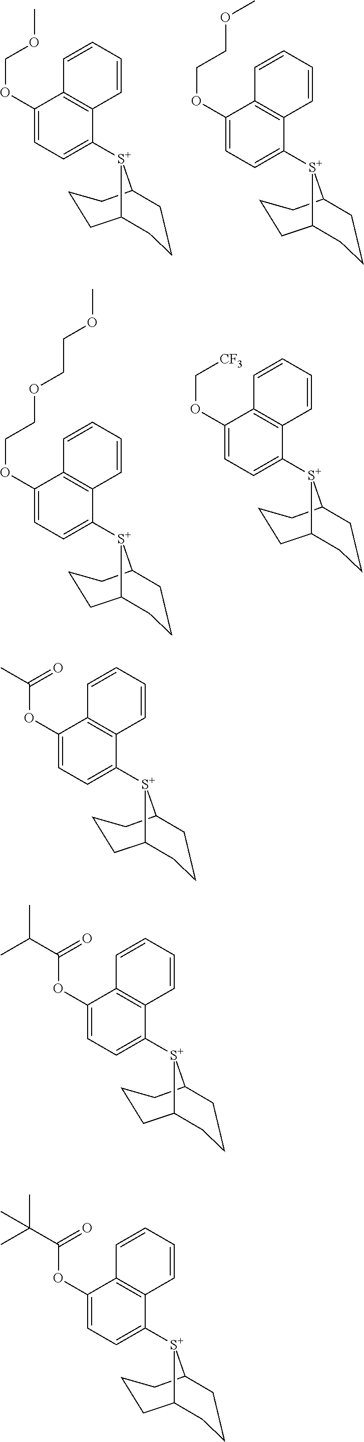

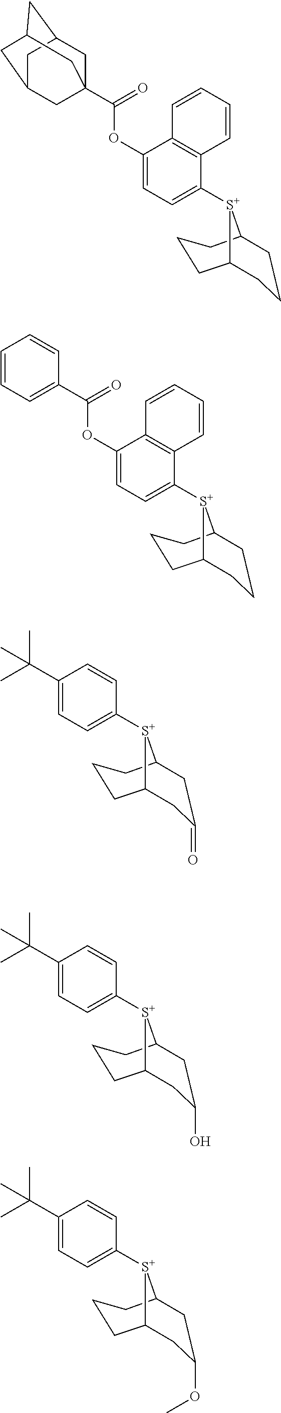 [SMILES: O=C(Oc1ccc([S+]2C3CCCC2CCC3)c2ccccc12)C12CC3CC(CC(C3)C1)C2 ; O=C(Oc1ccc([S+]2C3CCCC2CCC3)c2ccccc12)c1ccccc1 ; CC(C)(C)c1ccc([S+]2C3CCCC2CC(=O)C3)cc1 ; CC(C)(C)c1ccc([S+]2C3CCCC2CC(O)C3)cc1 ; COC1CC2CCCC(C1)[S+]2c1ccc(C(C)(C)C)cc1]

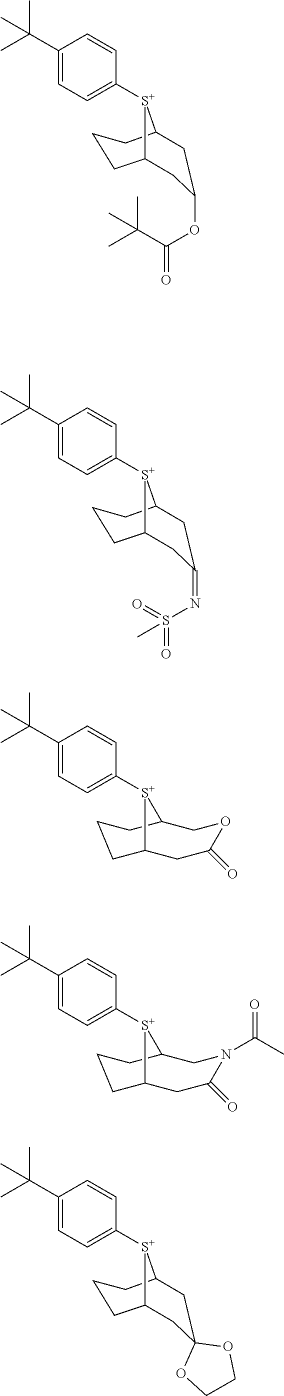

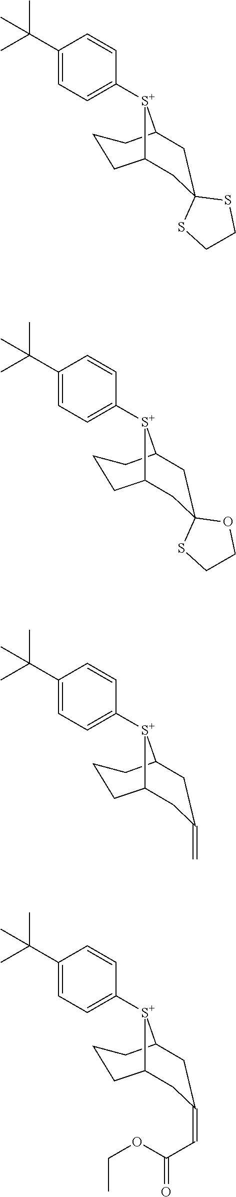

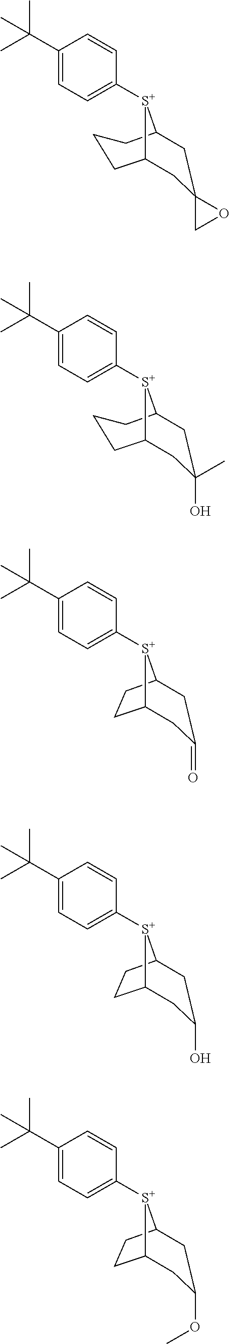

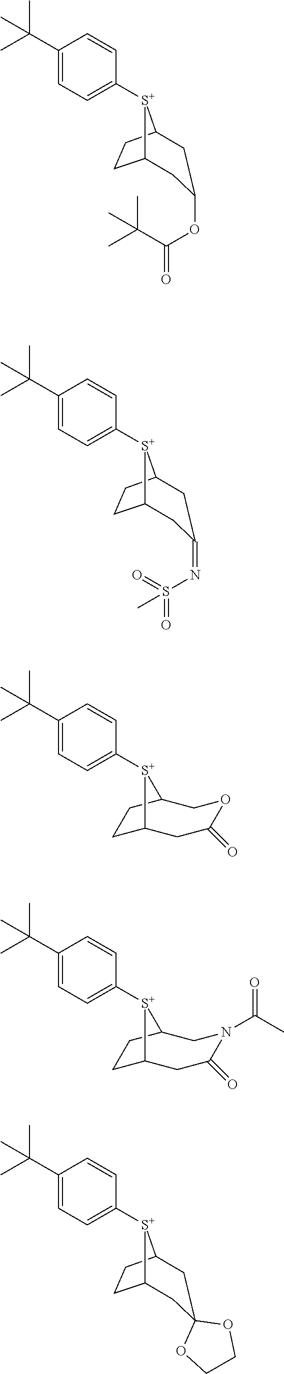

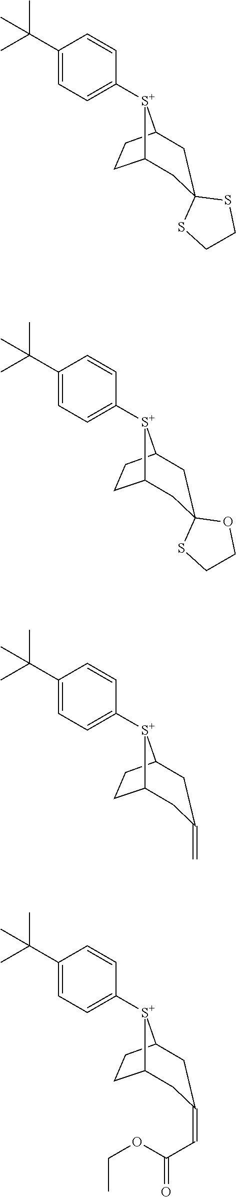

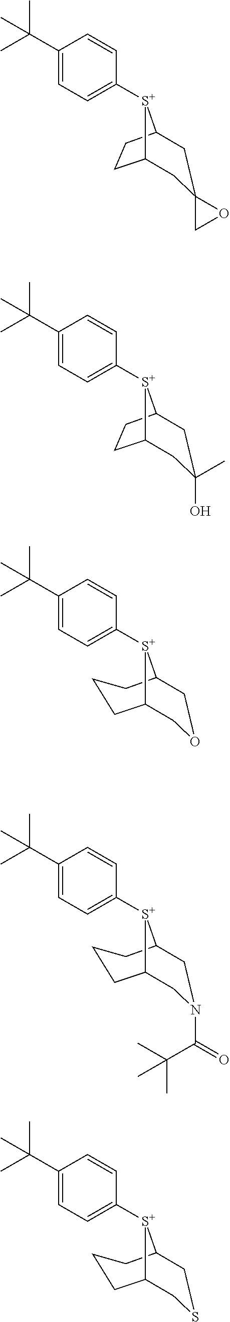

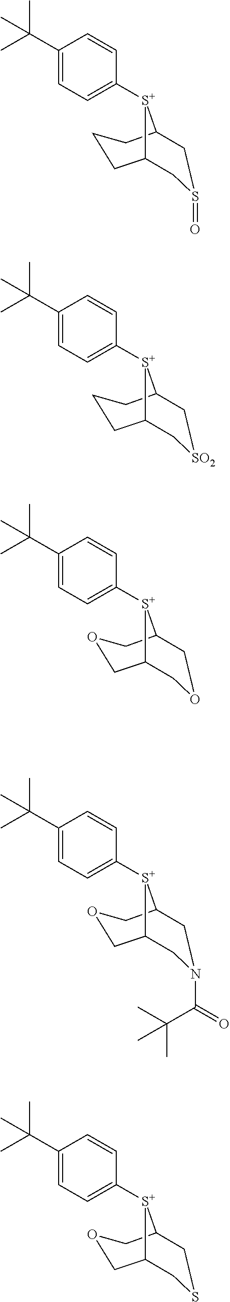 [SMILES: CC(C)(C)c1ccc([S+]2C3CCCC2CS(=O)C3)cc1 ; CC(C)(C)c1ccc([S+]2C3CCCC2CS(=O)(=O)C3)cc1 ; CC(C)(C)c1ccc([S+]2C3COCC2COC3)cc1 ; CC(C)(C)C(=O)N1CC2COCC(C1)[S+]2c1ccc(C(C)(C)C)cc1 ; CC(C)(C)c1ccc([S+]2C3COCC2CSC3)cc1]

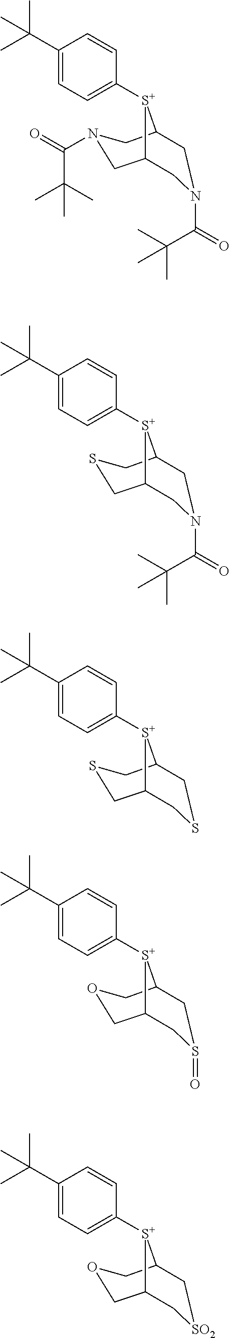 [SMILES: CC(C)(C)C(=O)N1CC2CN(C(=O)C(C)(C)C)CC(C1)[S+]2c1ccc(C(C)(C)C)cc1 ; CC(C)(C)C(=O)N1CC2CSCC(C1)[S+]2c1ccc(C(C)(C)C)cc1 ; CC(C)(C)c1ccc([S+]2C3CSCC2CSC3)cc1 ; CC(C)(C)c1ccc([S+]2C3COCC2CS(=O)C3)cc1 ; CC(C)(C)c1ccc([S+]2C3COCC2CS(=O)(=O)C3)cc1]

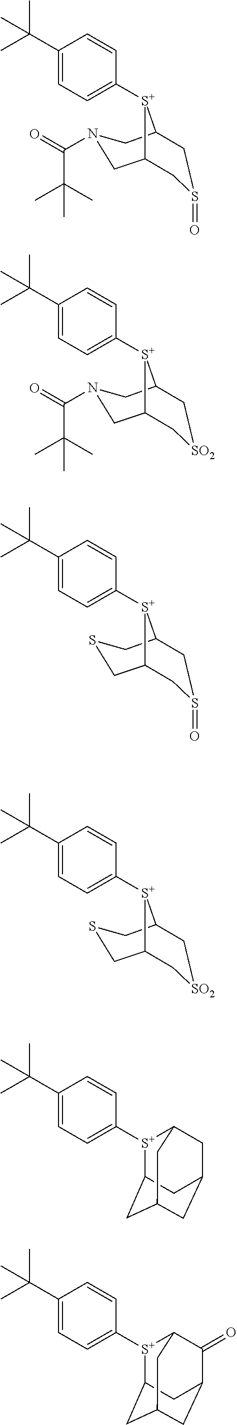 [SMILES: CC(C)(C)C(=O)N1CC2CS(=O)CC(C1)[S+]2c1ccc(C(C)(C)C)cc1 ; CC(C)(C)C(=O)N1CC2CS(=O)(=O)CC(C1)[S+]2c1ccc(C(C)(C)C)cc1 ; CC(C)(C)c1ccc([S+]2C3CSCC2CS(=O)C3)cc1 ; CC(C)(C)c1ccc([S+]2C3CSCC2CS(=O)(=O)C3)cc1 ; CC(C)(C)c1ccc([S+]2C3CC4CC(C3)CC2C4)cc1 ; CC(C)(C)c1ccc([S+]2C3CC4CC(C3)C(=O)C2C4)cc1]

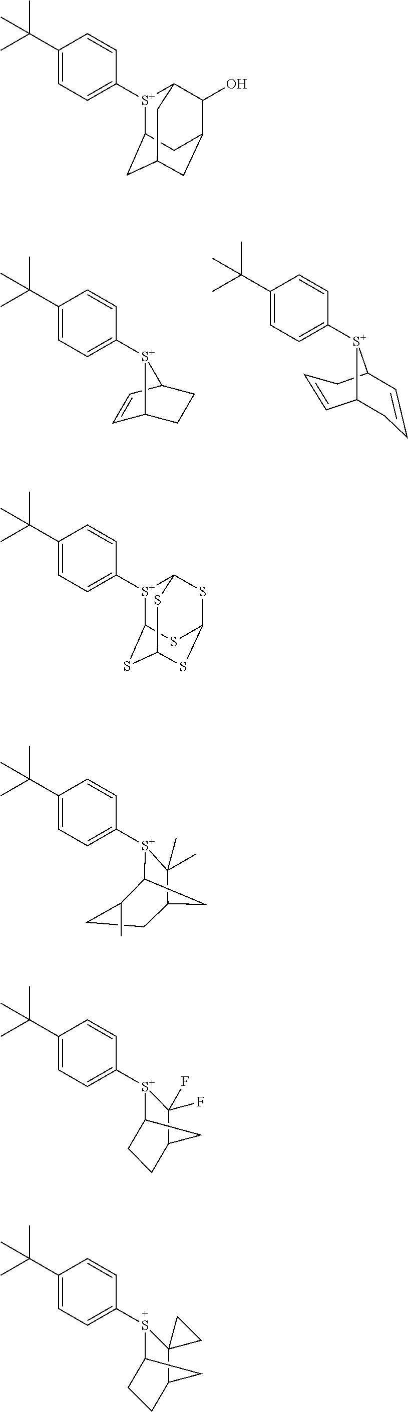

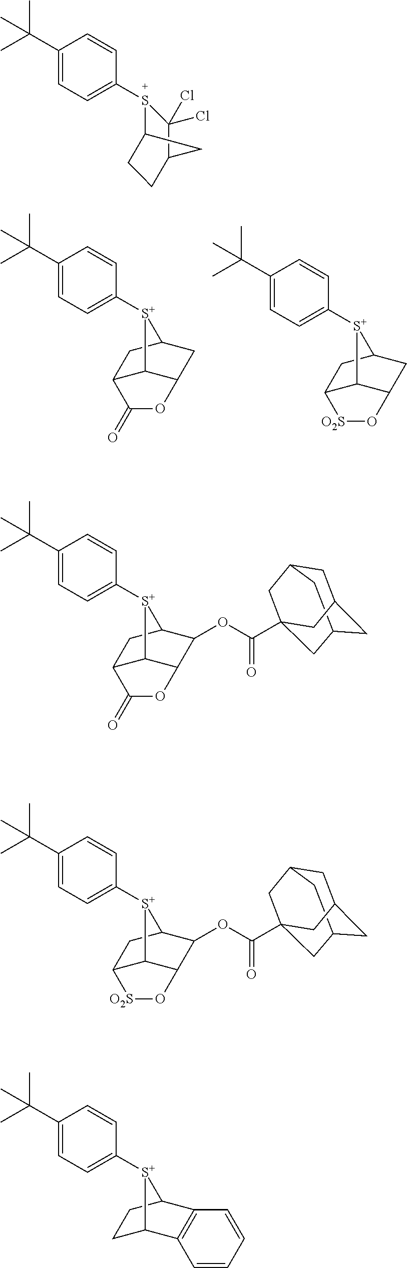

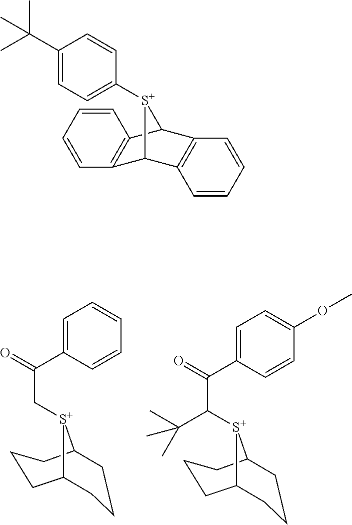 [SMILES: CC(C)(C)c1ccc([S+]2C3c4ccccc4C2c2ccccc23)cc1 ; O=C(C[S+]1C2CCCC1CCC2)c1ccccc1 ; COc1ccc(C(=O)C([S+]2C3CCCC2CCC3)C(C)(C)C)cc1]

Examples of the iodonium cation having formula (c6) are given below, but not limited thereto.

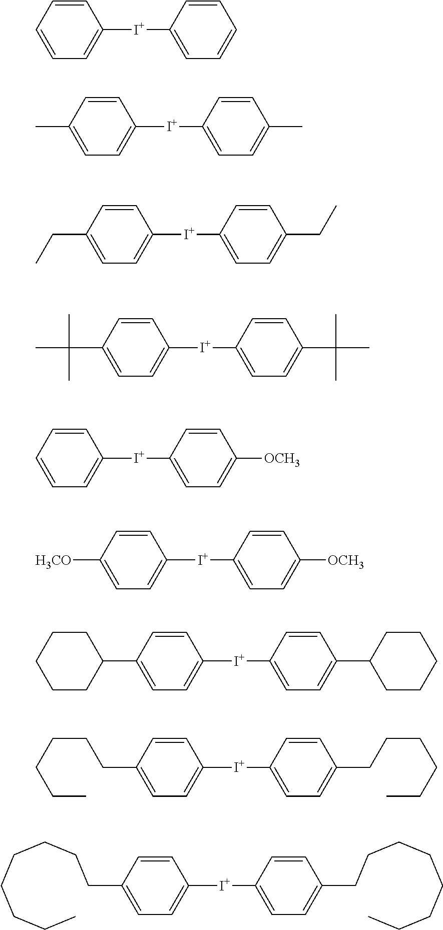

Examples of the repeat units (c1) to (c4) include arbitrary combinations of anions with cations, both as exemplified above.

The base polymer may further comprise repeat units (d) of a structure having a hydroxy group protected with an acid labile group. The repeat unit (d) is not particularly limited as long as the unit includes one or more structures having a hydroxy group protected with a protective group such that the protective group is decomposed to generate the hydroxy group under the action of acid. Repeat units having the formula (d1) are preferred.

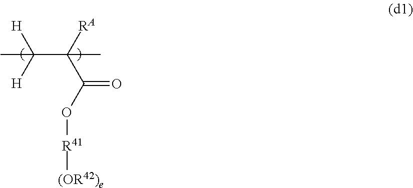

In formula (d1), R^{A} is as defined above, and e is an integer of 1 to 4. R⁴¹ is a C₁-C₃₀ (e+1)-valent hydrocarbon group which may contain a heteroatom. R⁴² is an acid labile group.

In formula (d1), the acid labile group R⁴² is deprotected under the action of acid so that a hydroxy group is generated. The structure of R⁴² is not particularly limited, an acetal structure, ketal structure, alkoxycarbonyl group and alkoxymethyl group having the following formula (d2) are preferred, with the alkoxymethyl group having formula (d2) being more preferred.

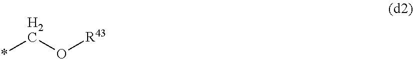

Herein R⁴³ is a C₁-C₁₅ hydrocarbyl group.

Illustrative examples of the acid labile group R⁴², the alkoxymethyl group having formula (d2), and the repeat units (d) are as exemplified for the repeat units (d) in JP-A 2020-111564 (US 20200223796).

In addition to the foregoing units, the base polymer may further comprise repeat units derived from other monomers, for example, substituted acrylic acid esters such as methyl methacrylate, methyl crotonate, dimethyl maleate and dimethyl itaconate, unsaturated carboxylic acids such as maleic acid, fumaric acid, and itaconic acid, cyclic olefins such as norbornene, norbornene derivatives, and tetracyclo[6.2.1.1^{3,6}.0^{2,7}]dodecene derivatives, and unsaturated acid anhydrides such as itaconic anhydride.

The base polymer preferably has a weight average molecular weight (Mw) of 1,000 to 500,000, and more preferably 3,000 to 100,000, as measured versus polystyrene standards by gel permeation chromatography (GPG) using tetrahydrofuran (THF) solvent. The above range of Mw ensures satisfactory etch resistance and eliminates the risk of resolution being reduced due to difficulty to gain a dissolution rate difference before and after exposure.

If a polymer has a wide molecular weight distribution or dispersity (Mw/Mn), which indicates the presence of lower and higher molecular weight polymer fractions, there is a possibility that foreign matter is left on the pattern or the pattern profile is degraded. The influence of Mw/Mn becomes stronger as the pattern rule becomes finer. Therefore, the base polymer should preferably have a narrow dispersity (Mw/Mn) of 1.0 to 2.0 in order to provide a resist composition suitable for micropatterning to a small feature size.

The base polymer may be synthesized, for example, by dissolving a monomer or monomers corresponding to the above-mentioned repeat units in an organic solvent, adding a radical polymerization initiator, and heating for polymerization.

One exemplary method of synthesizing the polymer is by dissolving one or more unsaturated bond-bearing monomers in an organic solvent, adding a radical initiator, and heating for polymerization. Examples of the organic solvent which can be used for polymerization include toluene, benzene, THF, diethyl ether, dioxane, cyclohexane, cyclopentane, methyl ethyl ketone (MEK), propylene glycol monomethyl ether acetate (PGMEA), and γ-butyrolactone (GBL). Examples of the polymerization initiator used herein include 2,2′-azobisisobutyronitrile (AIBN), 2,2′-azobis(2,4-dimethylvaleronitrile), dimethyl 2.2-azobis(2-methylpropionate), 1,1′-azobis(1-acetoxy-1-phenylethane), benzoyl peroxide, and lauroyl peroxide. The initiator is preferably added in an amount of 0.01 to 25 mol % based on the total of monomers to be polymerized. The reaction temperature is preferably 50 to 150° C., more preferably 60 to 100° C. The reaction time is preferably 2 to 24 hours, more preferably 2 to 12 hours in view of production efficiency.

The polymerization initiator may be fed to the reactor either by adding the initiator to the monomer solution and feeding the solution to the reactor, or by dissolving the initiator in a solvent to form an initiator solution and feeding the initiator solution and the monomer solution independently to the reactor. Because of a possibility that in the standby duration, the initiator generates a radical which triggers polymerization reaction to form a ultra-high-molecular-weight polymer, it is preferred from the standpoint of quality control to prepare the monomer solution and the initiator solution separately and add them dropwise. The acid labile group that has been incorporated in the monomer may be kept as such, or polymerization may be followed by protection or partial protection. During the polymer synthesis, any known chain transfer agent such as dodecyl mercaptan or 2-mercaptoethanol may be added for molecular weight control purpose. The amount of chain transfer agent added is preferably 0.01 to 20 mol % based on the total of monomers.

When hydroxystyrene or hydroxyvinylnaphthalene is copolymerized, one method is by dissolving hydroxystyrene or hydroxyvinylnaphthalene and other monomers in an organic solvent, adding a radical polymerization initiator thereto, and heating the solution for polymerization. In an alternative method, acetoxystyrene or acetoxyvinylnaphthalene is used instead, and after polymerization, the acetoxy group is deprotected by alkaline hydrolysis, for thereby converting the polymer product to polyhydroxystyrene or polyhydroxyvinylnaphthalene. For alkaline hydrolysis, a base such as aqueous ammonia or triethylamine may be used. Preferably the reaction temperature is −20° C. to 100° C., more preferably 0° C. to 60° C., and the reaction time is 0.2 to 100 hours, more preferably 0.5 to 20 hours.

The amounts of monomers in the monomer solution may be determined appropriate so as to provide the preferred fractions of repeat units.

It is now described how to use the polymer obtained by the above preparation method. The reaction solution resulting from polymerization reaction may be used as the final product. Alternatively, the polymer may be recovered in powder form through a purifying step such as re-precipitation step of adding the polymerization solution to a poor solvent and letting the polymer precipitate as powder, after which the polymer powder is used as the final product. It is preferred from the standpoints of operation efficiency and consistent quality to handle a polymer solution which is obtained by dissolving the powder polymer resulting from the purifying step in a solvent, as the final product. The solvents which can be used herein are described in JP-A 2008-111103, paragraphs [0144]-[0145](U.S. Pat. No. 7,537,880). Exemplary solvents include ketones such as cyclohexanone and methyl-2-n-pentyl ketone, alcohols such as 3-methoxybutanol, 3-methyl-3-methoxybutanol, 1-methoxy-2-propanol, and 1-ethoxy-2-propanol; keto-alcohols such as diacetone alcohol (DAA), ethers such as propylene glycol monomethyl ether (PGME), ethylene glycol monomethyl ether, propylene glycol monoethyl ether, ethylene glycol monoethyl ether, propylene glycol dimethyl ether, and diethylene glycol dimethyl ether; esters such as propylene glycol monomethyl ether acetate (PGMEA), propylene glycol monoethyl ether acetate, ethyl lactate, ethyl pyruvate, butyl acetate, methyl 3-methoxypropionate, ethyl 3-ethoxypropionate, tert-butyl acetate, tert-butyl propionate, and propylene glycol mono-tert-butyl ether acetate; lactones such as γ-butyrolactone (GBL); and high-boiling alcohols such as diethylene glycol, propylene glycol, glycerol, 1.4-butanediol, and 1.3-butanediol, which may be used alone or in admixture.

The polymer solution preferably has a polymer concentration of 0.01 to 30% by weight, more preferably 0.1 to 20% by weight.

Prior to use, the reaction solution or polymer solution is preferably filtered through a filter. Filtration is effective for consistent quality because foreign particles and gel which can cause defects are removed.

Suitable materials of which the filter is made include fluorocarbon, cellulose, nylon, polyester, and hydrocarbon base materials. Preferred for the filtration of a resist composition are filters made of fluorocarbons commonly known as Teflon®, hydrocarbons such as polyethylene and polypropylene, and nylon. While the pore size of the filter may be selected appropriate to comply with the desired cleanness, the filter preferably has a pore size of up to 100 nm, more preferably up to 20 nm. A single filter may be used or a plurality of filters may be used in combination. Although the filtering method may be single pass of the solution, preferably the filtering step is repeated by flowing the solution in a circulating manner. In the polymer preparation process, the filtering step may be carried out any times, in any order and in any stage. The reaction solution as polymerized or the polymer solution may be filtered, preferably both are filtered.

The proportion (mol %) of various repeat units in the base polymer is in the following range, but not limited thereto:
(1) preferably 1 to 60 mol %, more preferably 5 to 50 mol %, even more preferably 10 to 50 mol % of repeat units of at least one type selected from repeat units (a1) and (a2);
(II) preferably 40 to 99 mol %, more preferably 50 to 95 mol %, even more preferably 50 to 90 mol % of repeat units of at least one type selected from repeat units (b1) and (b2);
(III) preferably 0 to 30 mol %, more preferably 0 to 20 mol %, even more preferably 0 to 15 mol % of repeat units of at least one type selected from repeat units (c1) to (c4); and
(IV) preferably 0 to 80 mol %, more preferably 0 to 70 mol %, even more preferably 0 to 50 mol % of repeat units of at least one type derived from other monomers.

The base polymer (B) may be used alone or as a blend of two or more polymers which differ in compositional ratio. Mw and/or Mw/Mn. Component (B) may also be a blend of the base polymer defined above and a hydrogenated product of ring-opening metathesis polymer (ROMP). For the ROMP, reference is made to JP-A 2003-066612.

(C) Organic Solvent

The resist composition may comprise (C) an organic solvent. The organic solvent used herein is not particularly limited as long as the foregoing and other components are soluble therein. Suitable solvents include ketones such as cyclopentanone, cyclohexanone, and methyl 2-n-pentyl ketone; alcohols such as 3-methoxybutanol, 3-methyl-3-methoxybutanol, 1-methoxy-2-propanol, and 1-ethoxy-2-propanol; keto-alcohols such as diacetone alcohol (DAA); ethers such as propylene glycol monomethyl ether (PGME), ethylene glycol monomethyl ether, propylene glycol monoethyl ether, ethylene glycol monoethyl ether, propylene glycol dimethyl ether, and diethylene glycol dimethyl ether; esters such as propylene glycol monomethyl ether acetate (PGMEA), propylene glycol monoethyl ether acetate, ethyl lactate, ethyl pyruvate, butyl acetate, methyl 3-methoxypropionate, ethyl 3-ethoxypropionate, tert-butyl acetate, tert-butyl propionate, and propylene glycol mono-tert-butyl ether acetate; and lactones such as γ-butyrolactone (GBL), and mixtures thereof.

Of the foregoing organic solvents, it is recommended to use 1-ethoxy-2-propanol, PGMEA, cyclohexanone, GBL, DAA and mixtures thereof because the base polymer (B) is most soluble therein.

The organic solvent (C) is preferably added in an amount of 200 to 5,000 parts by weight, and more preferably 400 to 3,500 parts by weight per 80 parts by weight of the base polymer (B). The organic solvent may be used alone or in admixture.

(D) Photoacid Generator

The resist composition may comprise (D) a photoacid generator. The PAG is not particularly limited as long as it is capable of generating an acid upon exposure to KrF excimer laser radiation, ArF excimer laser radiation, EB, or EUV, collectively referred to as high-energy radiation. The preferred PAG is a salt having the formula (2-1) or (2-2).

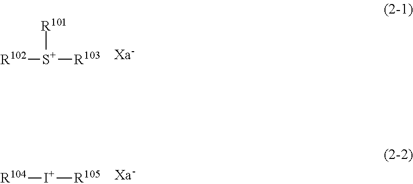

In formulae (2-1) and (2-2), R¹⁰¹ to R¹⁰⁵ are each independently a C₁-C₂₀ hydrocarbyl group which may contain a heteroatom. R¹⁰¹ and R¹⁰² may bond together to form a ring with the sulfur atom to which they are attached. Examples of the hydrocarbyl group are as exemplified above for R³⁶ to R⁴⁰ in formulae (c5) and (c6).

Examples of the cation in the sulfonium salt having formula (2-1) are as exemplified above for the sulfonium cation having formula (c5). Examples of the cation in the iodonium salt having formula (2-2) are as exemplified above for the iodonium cation having formula (c6).

In formulae (2-1) and (2-2), Xa⁻ is an anion selected from the formulae (2A) to (2D).

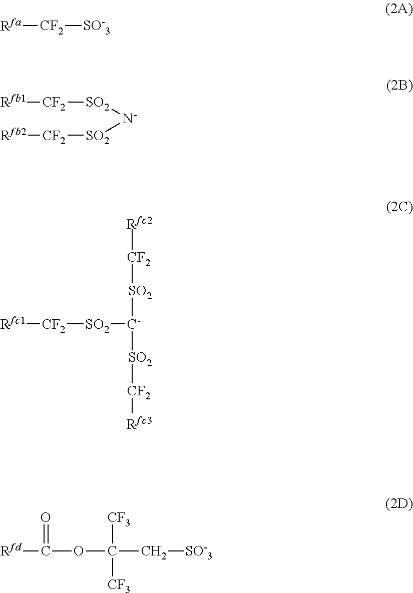

In formula (2A), R^{fa} is fluorine or a C₁-C₄₀ hydrocarbyl group which may contain a heteroatom. The hydrocarbyl group may be saturated or unsaturated and straight, branched or cyclic. Examples thereof are as will be exemplified later for R¹¹¹ in formula (2A′).

Of the anions having formula (2A), anions having the formula (2A′) are preferred.

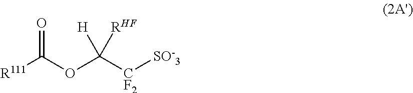

In formula (2A′), R^{HF} is hydrogen or trifluoromethyl, preferably trifluoromethyl.

R¹¹¹ is a C₁-C₃₈ hydrocarbyl group which may contain a heteroatom. Of the hydrocarbyl groups, those of 6 to 30 carbon atoms are preferred because a high resolution is available in fine pattern formation. The hydrocarbyl group R¹¹¹ may be saturated or unsaturated and straight, branched or cyclic. Examples thereof include C₁-C₃₈ alkyl groups such as methyl, ethyl, n-propyl, isopropyl, n-butyl, isobutyl, sec-butyl, tert-butyl, pentyl, neopentyl, hexyl, heptyl, octyl, 2-ethylhexyl, nonyl, decyl, undecyl, dodecyl, tridecyl, pentadecyl, heptadecyl, and icosyl; C₃-C₃₈ cyclic saturated hydrocarbyl groups such as cyclopentyl, cyclohexyl, 1-adamantyl, 2-adamantyl, 1-adamantylmethyl, norbornyl, norbornylmethyl, tricyclodecyl, tetracyclododecyl, tetracyclododecylmethyl, and dicyclohexylmethyl; C₂-C₃₈ unsaturated aliphatic hydrocarbyl groups such as allyl and 3-cyclohexenyl, C₆-C₃₈ aryl groups such as phenyl, l-naphthyl and 2-naphthyl; and C₇-C₃₈ aralkyl groups such as benzyl and diphenylmethyl.

In the foregoing groups, some or all of the hydrogen atoms may be substituted by a moiety containing a heteroatom such as oxygen, sulfur, nitrogen or halogen, or some constituent —CH₂— may be replaced by a moiety containing a heteroatom such as oxygen, sulfur or nitrogen, so that the group may contain a hydroxy, fluorine, chlorine, bromine, iodine, cyan, carbonyl, ether bond, ester bond, sulfonic ester bond, carbonate bond, lactose ring, sultone ring, carboxylic anhydride (—C(═O)—O—C(═O)—) or haloalkyl moiety. Examples of the heteroatom-containing hydrocarbyl group include tetrahydrofuryl, methoxymethyl, ethoxymethyl, methylthiomethyl, acetamidomethyl, trifluoroethyl, (2-methoxyethoxy)methyl, acetoxymethyl, 2-carboxy-1-cyclohexyl, 2-oxopropyl, 4-oxo-1-adamantyl, 5-hydroxy-l-adamantyl, 5-tert-butylcarbonyloxy-l-adamantyl, 4-oxatricyclo[4.2.1.0^{3,7}]nonan-5-on-2-yl, and 3-oxocyclohexyl.

With respect to the synthesis of the sulfonium salt having an anion of formula (2A′), reference may be made to JP-A 2007-145797, JP-A 2008-106045, JP-A 2009-007327, and JP-A 2009-258695. Also useful are the sulfonium salts described in JP-A 2010-215608, JP-A 2012-041320, JP-A 2012-106986, and JP-A 2012-153644.

Examples of the anion having formula (2A) are as exemplified above for the anions having formulae (c1-1) and (c1-2).

In formula (2B), R^{fb1} and R^{fb2} are each independently fluorine or a C₁-C₄₀ hydrocarbyl group which may contain a heteroatom. The hydrocarbyl group may be saturated or unsaturated and straight, branched or cyclic. Examples thereof are as exemplified above for R¹¹¹ in formula (2A′). Preferably R^{fb1} and R^{fb2} are fluorine or C₁-C₄ straight fluorinated alkyl groups. Also, R^{fb1} and R^{fb2} may bond together to form a ring with the linkage: —CF₂—SO₂—N⁻—SO₂—CF₂— to which they are attached. It is preferred that a combination of R^{fb1} and R^{fb2} be a fluorinated ethylene or fluorinated propylene group.

In formula (2C), R^{fc1}, R^{fc2} and R^{fc3} are each independently fluorine or a C₁-C₄₀ hydrocarbyl group which may contain a heteroatom. The hydrocarbyl group may be saturated or unsaturated and straight, branched or cyclic. Examples thereof are as exemplified for R¹¹¹. Preferably R^{fc1}, R^{fc2} and R^{fc3} are fluorine or C₁-C₄ straight fluorinated alkyl groups. Also, R^{fc1} and R^{fc2} may bond together to form a ring with the linkage: —CF₂—SO₂—C⁻—SO₂—CF₂— to which they are attached. It is preferred that a combination of R^{fc1} and R^{fc2} be a fluorinated ethylene or fluorinated propylene group.

In formula (2D), R^{fd} is a C₁-C₄₀ hydrocarbyl group which may contain a heteroatom. The hydrocarbyl group may be saturated or unsaturated and straight, branched or cyclic. Examples thereof are as exemplified above for R¹¹¹.

With respect to the synthesis of the sulfonium salt having an anion of formula (2D), reference may be made to JP A 2010-215608 and JP-A 2014-133723.

Examples of the anion having formula (2D) are as exemplified for the anion having formula (1D) in JP-A 2018-197853.

Notably, the compound having the anion of formula (2D) does not have fluorine at the α-position relative to the sulfo group, but two trifluoromethyl groups at the β-position. For this reason, it has a sufficient acidity to sever the acid labile groups in the base polymer. Thus the compound is an effective PAG.

Also, a compound having the formula (3) is preferred as the PAG (D).

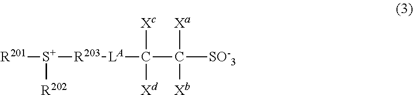

In formula (3). R²⁰¹ and R²⁰² are each independently a C₁-C₃₀ hydrocarbyl group which may contain a heteroatom. R²⁰³ is a C₁-C₃₀ hydrocarbylene group which may contain a heteroatom. Any two of R²⁰¹, R²⁰² and R²⁰³ may bond together to form a ring with the sulfur atom to which they are attached.

The hydrocarbyl groups R²⁰¹ and R²⁰² may be saturated or unsaturated and straight, branched or cyclic. Examples thereof include C₁-C₃₀ alkyl groups such as methyl, ethyl, n-propyl, isopropyl, n-butyl, sec-butyl, tert-butyl, n-pentyl, tert-pentyl, n-hexyl, n-octyl, 2-ethylhexyl, n-nonyl, and n-decyl; C₃-C₃₀ cyclic saturated hydrocarbyl groups such as cyclopentyl, cyclohexyl, cyclopentylmethyl, cyclopentylethyl, cyclopentylbutyl, cyclohexylmethyl, cyclohexylethyl, cyclohexylbutyl, norbornyl, oxanorbornyl, tricyclo[5.2.1.0^{2,6}]decanyl, and adamantyl; and C₆-C₃₀ aryl groups such as phenyl, methylphenyl, ethylphenyl, n-propylphenyl, isopropylphenyl, n-butylphenyl, isobutylphenyl, sec-butylphenyl, tert-butylphenyl, naphthyl, methylnaphthyl, ethylnaphthyl, n-propylnaphthyl, isopropylnaphthyl, n-butylnaphthyl, isobutylnaphthyl, sec-butylnaphthyl, tert-butylnaphthyl, and anthracenyl, and combinations thereof. In these hydrocarbyl groups, some or all of the hydrogen atoms may be substituted by a moiety containing a heteroatom such as oxygen, sulfur, nitrogen or halogen, and some constituent —CH₂— may be replaced by a moiety containing a heteroatom such as oxygen, sulfur or nitrogen, so that the group may contain a hydroxy, fluorine, chlorine, bromine, iodine, cyano, carbonyl, ether bond, ester bond, sulfonic ester bond, carbonate bond, lactone ring, sultone ring, carboxylic anhydride (—C(═O)—O—C(═O)—) or haloalkyl moiety.

The hydrocarbylene group R²⁰³ may be saturated or unsaturated and straight, branched or cyclic. Examples thereof include C₁-C₃₀ alkanediyl groups such as methanediyl, ethane-1,1-diyl, ethane-1,2-diyl, propane-1,3-diyl, butane-1,4-diyl, pentane-1,5-diyl, hexane-1,6-diyl, heptane-1,7-diyl, octane-1,8-diyl, nonane-1,9-diyl, decane-1,10-diyl, undecane-1,11-diyl, dodecane-1,12-diyl, tridecane-1,13-diyl, tetradecane-1,14-diyl, pentadecane-1,15-diyl, hexadecane-1,16-diyl, and heptadecane-1,17-diyl; C₃-C₃₀ cyclic saturated hydrocarbylene groups such as cyclopentanediyl, cyclohexanediyl, norbornanediyl and adamantanediyl; and C₆-C₃₀ arylene groups such as phenylene, methylphenylene, ethylphenylene, n-propylphenylene, isopropylphenylene, n-butylphenylene, isobutylphenylene, sec-butylphenylene, tert-butylphenylene, naphthylene, methylnaphthylene, ethylnaphthylene, n-propylnaphthylene, isopropylnaphthylene, n-butylnaphthylene, isobutylnaphthylene, sec-butylnaphthylene, and tert-butylnaphthylene, and combinations thereof. In these hydrocarbylene groups, some or all of the hydrogen atoms may be substituted by a moiety containing a heteroatom such as oxygen, sulfur, nitrogen or halogen, or some constituent —CH₂— may be replaced by a moiety containing a heteroatom such as oxygen, sulfa or nitrogen, so that the group may contain a hydroxy, fluorine, chlorine, bromine, iodine, cyan, carbonyl, ether bond, ester bond, sulfonic ester bond, carbonate bond, lactone ring, sultone ring, carboxylic anhydride (—C(═O)—O—C(═O)—) or haloalkyl moiety. Of the heteroatoms, oxygen is preferred.

In formula (3), L^{A} is a single bond, ether bond or a C₁-C₂₀ hydrocarbylene group which may contain a heteroatom. The hydrocarbylene group may be saturated or unsaturated and straight, branched or cyclic. Examples thereof are as exemplified above for the hydrocarbylene group R²⁰³.

In formula (3), X^{a}, X^{b}, X^{c} and X^{d} are each independently hydrogen, fluorine or trifluoromethyl, with the proviso that at least one of X^{a}, X^{b}, X^{c} and X^{d} is fluorine or trifluoromethyl.

Of the PAGs having formula (3), those having formula (3′) are preferred.

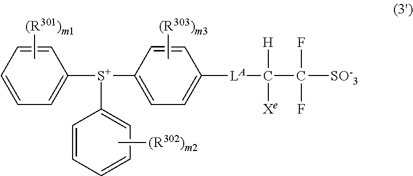

In formula (3′), L^{A} is as defined above. X^{e} is hydrogen or trifluoromethyl, preferably trifluoromethyl. R³⁰¹, R³⁰² and R³⁰³ are each independently hydrogen or a C₁-C₂₀ hydrocarbyl group which may contain a heteroatom. The hydrocarbyl group may be saturated or unsaturated and straight, branched or cyclic. Examples thereof are as exemplified above for R¹¹¹ in formula (2A′). The subscripts m¹ and m² are each independently an integer of 0 to 5, and m³ is an integer of 0 to 4.

Examples of the PAG having formula (3) include those exemplified for the PAG having formula (2) in JP-A 2017-026980.

Of the foregoing PAGs, those having an anion of formula (2A′) or (2D) are especially preferred because of reduced acid diffusion and high solubility in solvents. Also those having formula (3′) are especially preferred because of extremely reduced acid diffusion.

When used, the PAG (D) is preferably added in an amount of 0.1 to 40 parts, and more preferably 0.5 to 20 parts by weight per 80 parts by weight of the base polymer (B). As long as the amount of the PAG is in the range, good resolution is achievable and the risk-of foreign particles being formed after development or during stripping of resist film is avoided. The PAG may be used alone or in admixture.

(E) Other Quencher

The resist composition may further comprise (E) a quencher other than the amuse compound having formula (1). Onium salts having the formulae (4-1) and (4-2) are useful as the other quencher (E).

R⁴⁰¹—SO₃⁻Mq⁺ (4-1)

R⁴⁰²—CO₂⁻Mq⁺ (4-2)

In formula (4-1), R⁴⁰¹ is hydrogen or a C₁-C₄₀ hydrocarbyl group which may contain a heteroatom, exclusive of the hydrocarbyl group in which the hydrogen atom bonded to the carbon atom at α-position of the sulfo group is substituted by fluorine or fluoroalkyl.

The hydrocarbyl group R⁴⁰¹ may be saturated or unsaturated and straight, branched or cyclic. Examples thereof include C₁-C₄₀ alkyl groups such as methyl, ethyl, n-propyl, isopropyl, n-butyl, isobutyl, sec-butyl, tert-butyl, n-pentyl, tert-pentyl, n-hexyl, n-octyl, 2-ethylhexyl, n-nonyl, and n-decyl, C₃-C₄₀ cyclic saturated hydrocarbyl groups such as cyclopentyl, cyclohexyl, cyclopentylmethyl, cyclopentylethyl, cyclopentylbutyl, cyclohexylmethyl, cyclohexylethyl, cyclohexylbutyl, norbornyl, oxanorbornyl, tricyclo[5.2.1.0^{2,6}]decanyl, and adamantyl; C₆-C₄₀ aryl groups such as phenyl, naphthyl and anthracenyl, and combinations thereof. In these hydrocarbyl groups, some or all hydrogen atoms may be substituted by a moiety containing a heteroatom such as oxygen, sulfur, nitrogen or halogen, or some constituent —CH₂— may be replaced by a moiety containing a heteroatom such as oxygen, sulfur or nitrogen, so that the group may contain a hydroxy, fluorine, chlorine, bromine, iodine, cyan, carbonyl, ether bond, ester bond, sulfonic ester bond, carbonate bond, lactone ring, sultone ring, carboxylic anhydride (—C(═O)—O—C(═O)—) or haloalkyl moiety.

In formula (4-2), R⁴⁰² is hydrogen or a C₁-C₄₀ hydrocarbyl group which may contain a heteroatom. Examples of the hydrocarbyl group R⁴⁰² include those exemplified above for R⁴⁰¹ and fluoroalkyl groups such as trifluoromethyl and trifluoroethyl, and fluorinated aryl groups such as pentafluorophenyl and 4-trifluoromethylphenyl.

Examples of the anion in the onium salt having formula (4-1) are shown below, but not limited thereto.

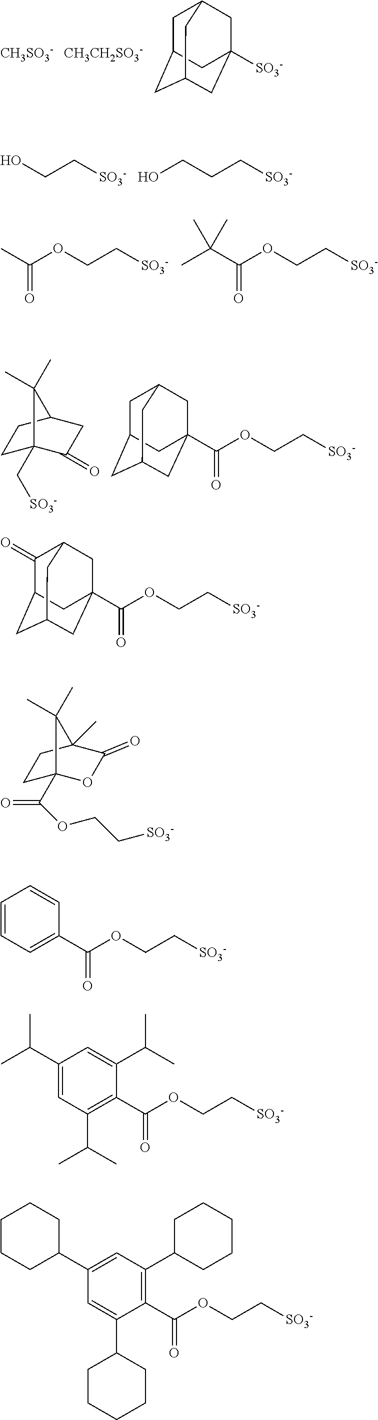

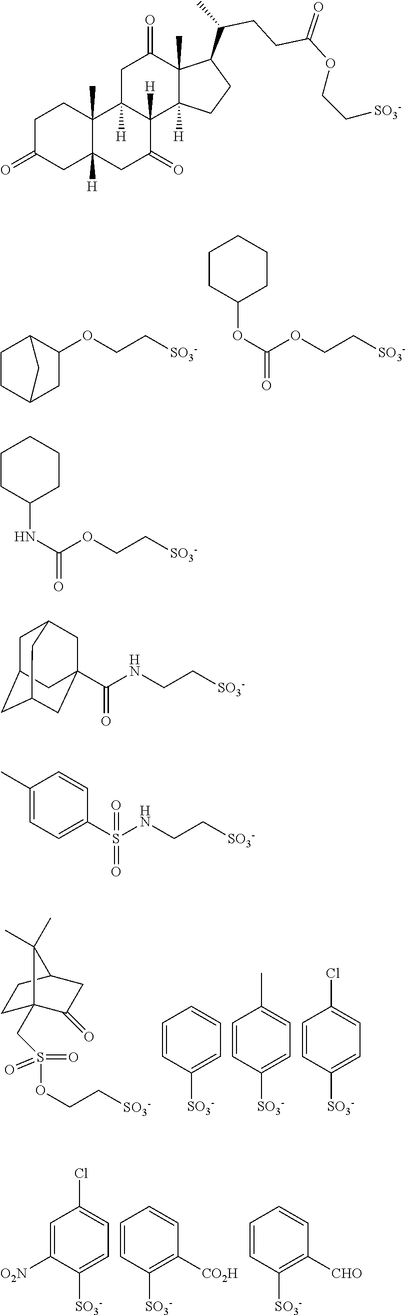

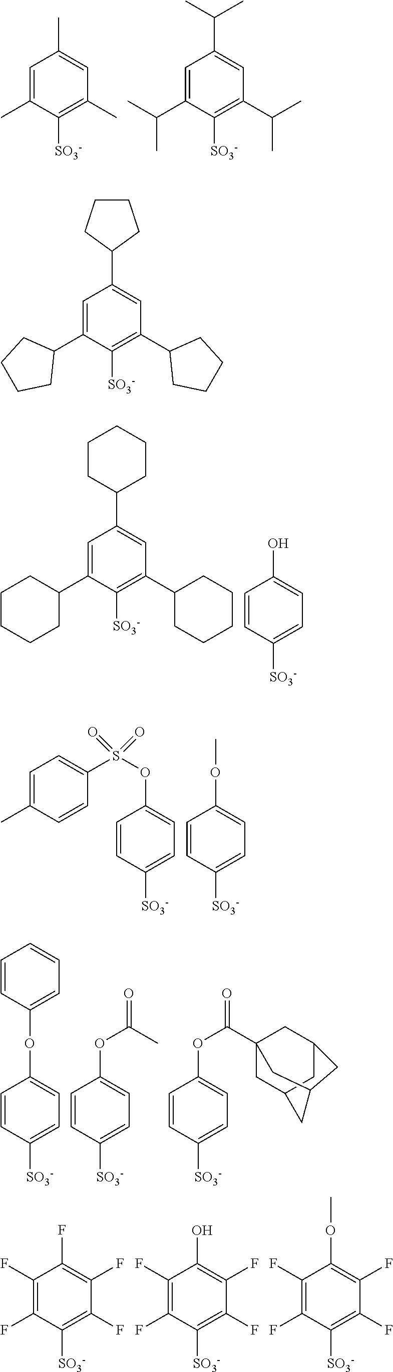

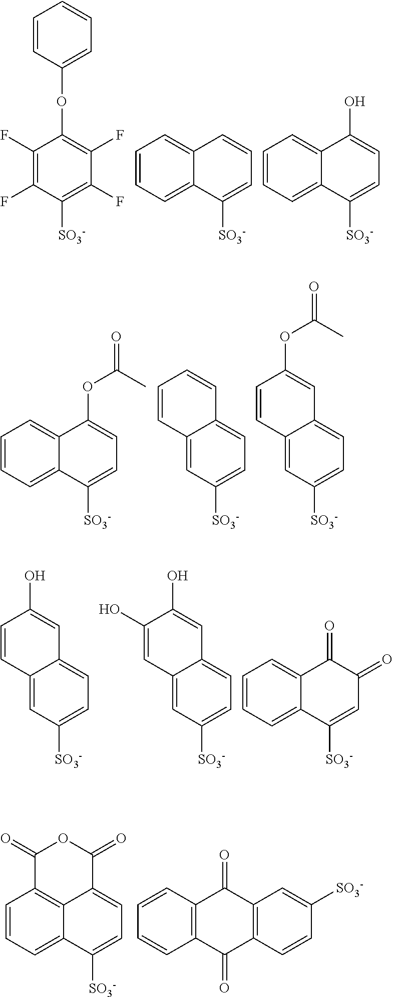

Examples of the anion in the onium salt having formula (4-2) are shown below, but not limited thereto.

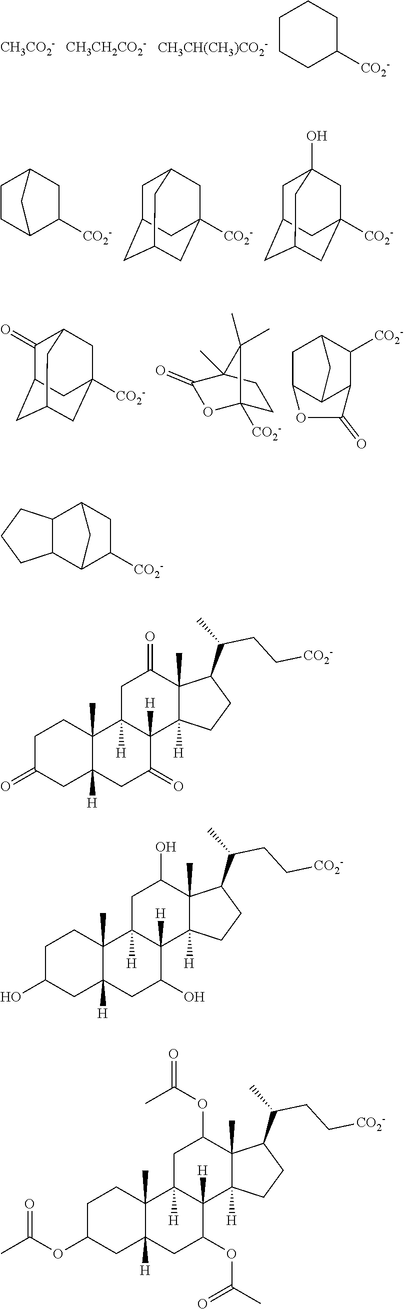

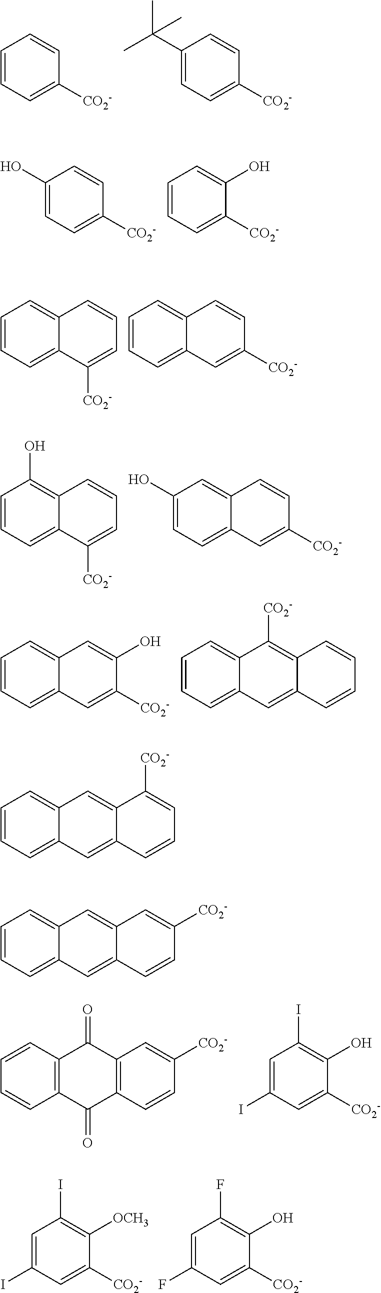

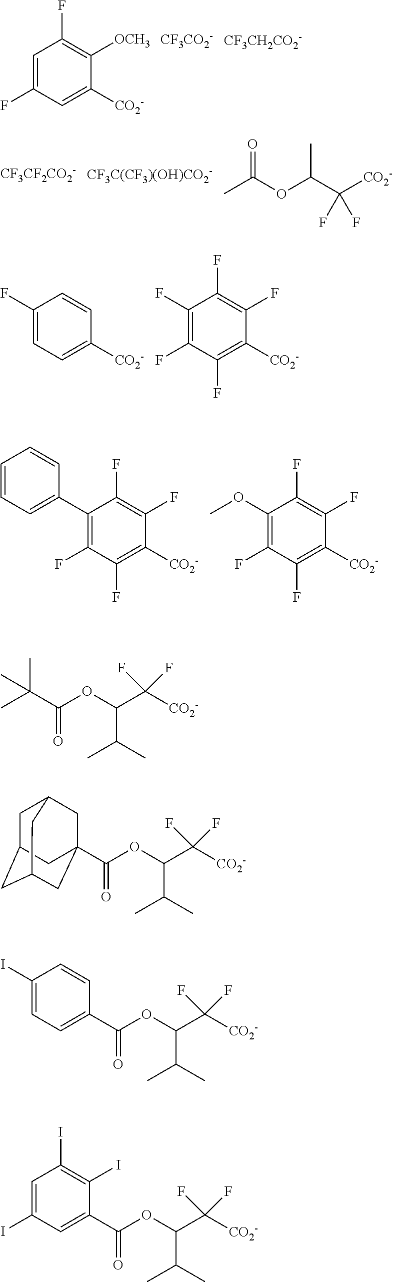

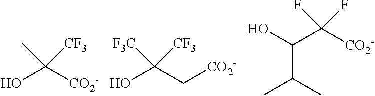

In formulae (4-1) and (4-2), Mq⁺ is an onium cation, which is preferably selected from cations having the formulae (4A), (4B) and (4C).

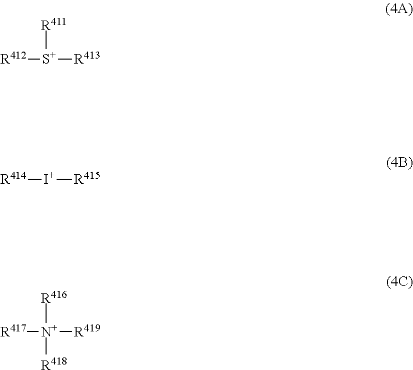

In formulae (4A) to (4C), R⁴¹¹ to R⁴¹⁹ are each independently a C₁-C₄₀ hydrocarbyl group which may contain a heteroatom. A pair of R⁴¹¹ and R⁴¹² may bond together to form a ring with the sulfur atom to which they are attached. A pair of R⁴¹⁶ and R⁴¹⁷ may bond together to form a ring with the nitrogen atom to which they are attached. Examples of the hydrocarbyl group are as exemplified above for R⁴⁰¹ in formula (4-1).

Examples of the onium cation represented by Mq⁺ are shown below, but not limited thereto.

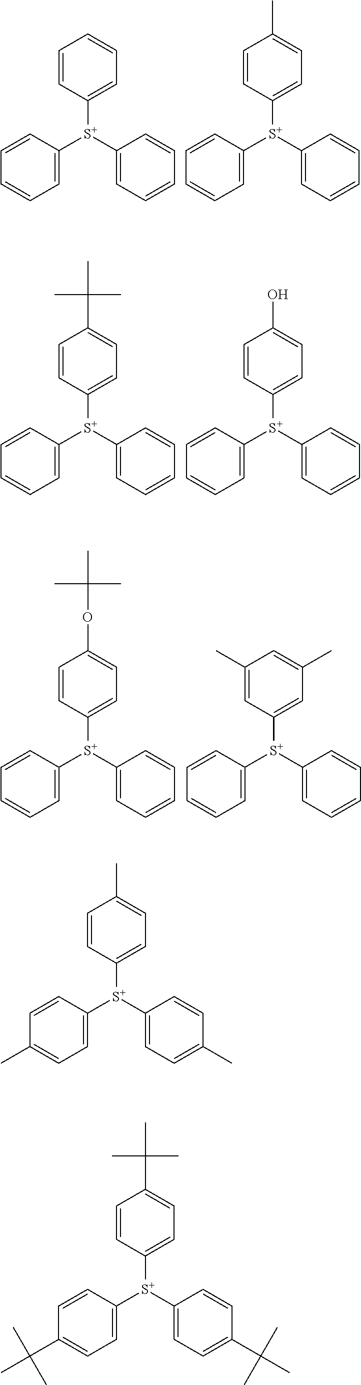

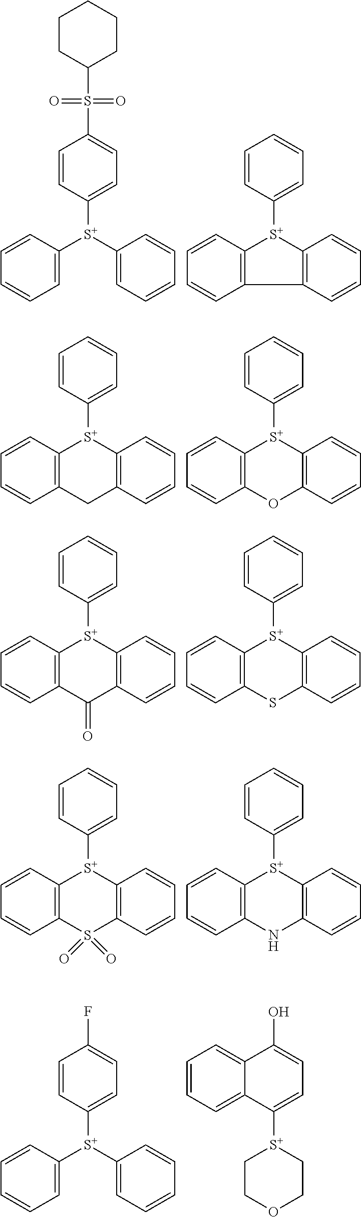

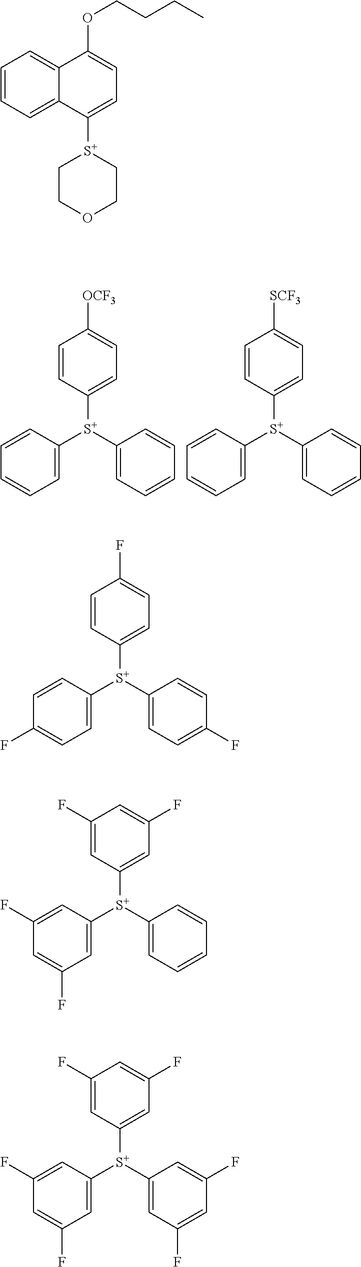

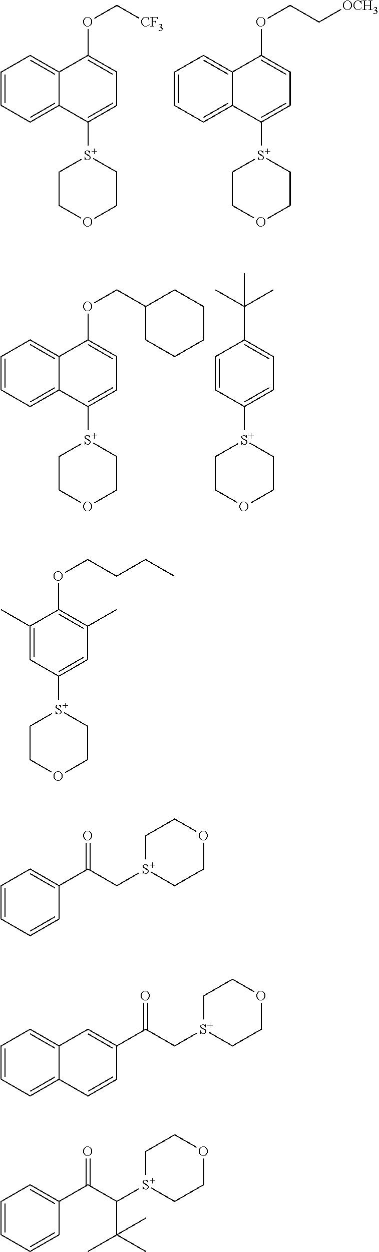

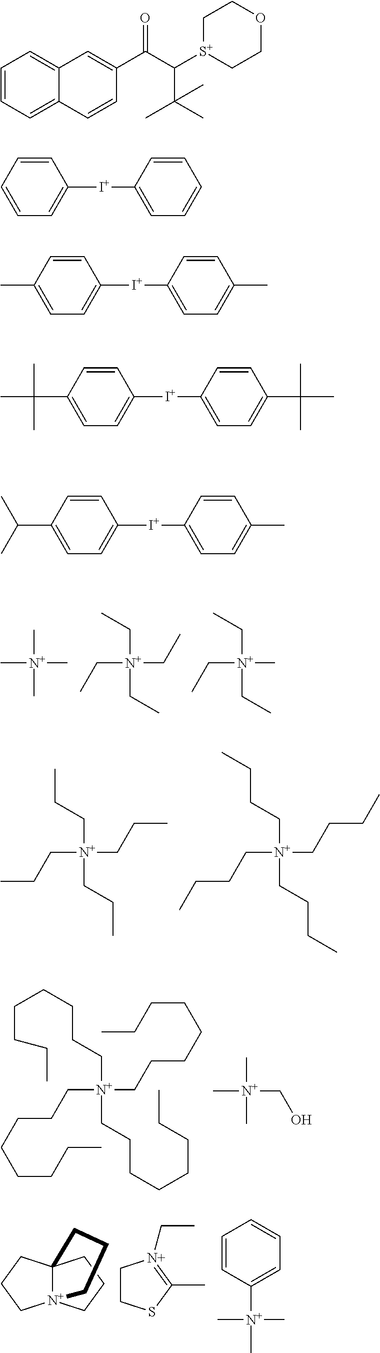

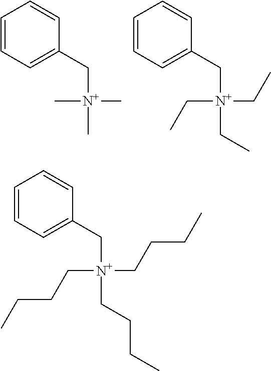 [SMILES: C[N+](C)(C)Cc1ccccc1 ; CC[N+](CC)(CC)Cc1ccccc1 ; CCCC[N+](CCCC)(CCCC)Cc1ccccc1]

Examples of the onium salt having formula (4-1) or (4-2) include arbitrary combinations of anions with cations, both as exemplified above. These onium salts may be readily prepared by ion exchange reaction using any well-known organic chemistry technique. For the ion exchange reaction, reference may be made to JP-A 2007-145797, for example.

The onium salt having formula (4-1) or (4-2) functions as a quencher in the chemically amplified resist composition because the counter anion of the onium salt is a conjugated base of a weak acid. As used herein, the weak acid indicates an acidity insufficient to deprotect an acid labile group from an acid labile group-containing unit in the base polymer. The onium salt having formula (4-1) or (4-2) functions as a quencher when used in combination with an onium salt type PAG having a conjugated base of a strong acid (typically a sulfonic acid which is fluorinated at α-position) as the counter anion. In a system using a mixture of an onium salt capable of generating a strong acid (e.g., α-position fluorinated sulfonic acid) and an onium salt capable of generating a weak acid (e.g., non-fluorinated sulfonic acid or carboxylic acid), if the strong acid generated from the PAG upon exposure to high-energy radiation collides with the unreacted onium salt having a weak acid anion, then a salt exchange occurs whereby the weak acid is released and an onium salt having a strong acid anion is formed. In this course, the strong acid is exchanged into the weak acid having a low catalysis, incurring apparent deactivation of the acid for enabling to control acid diffusion.

If a PAG capable of generating a strong acid is an onium salt, an exchange from the strong acid generated upon exposure to high-energy radiation to a weak acid as above can take place, but it rarely happens that the weak acid generated upon exposure to high-energy radiation collides with the unreacted opium salt capable of generating a strong acid to induce a salt exchange. This is because of a likelihood of an onion cation forming an ion pair with a stronger acid anion.

When the onium salt having formula (4-1) or (4-2) is used as the other quencher (E), the amount of the onium salt used is preferably 0.1 to 10 parts by weight, more preferably 0.1 to 5 parts by weight per 80 parts by weight of the base polymer (B). As long as the amount of component (E) is in the range, a satisfactory resolution is available without a substantial lowering of sensitivity. The opium salt having formula (4-1) or (4-2) may be used alone or in admixture.

Also, nitrogen-containing compounds other than component (A) may be used as the other quencher (E). Suitable nitrogen-containing compounds include primary, secondary and tertiary amine compounds, specifically amine compounds having a hydroxy group, ether bond, ester bond, lactone ring, cyano group or sulfonic ester bond, as described in JP-A 2008-111103, paragraphs [0146]-[0164] (U.S. Pat. No. 7,537,880), and primary or secondary amine compounds protected with a carbamate group, as described in JP 3790649.

A sulfonic acid sulfonium salt having a nitrogen-containing substituent may also be used as the nitrogen-containing compound. This compound functions as a quencher in the unexposed region, but as a so-called photo-degradable base in the exposed region because it loses the quencher function in the exposed region due to neutralization thereof with the acid generated by itself. Using a photo-degradable base, the contrast between exposed and unexposed regions can be further enhanced. With respect to the photo-degradable base, reference may be made to JP-A 2009-109595 and JP-A 2012-046501, for example.

When the nitrogen-containing compound is used as the other quencher (E), the amount of the nitrogen-containing compound used is preferably 0.001 to 12 parts by weight, more preferably 0.01 to 8 parts by weight per 80 parts by weight of the base polymer (B). The nitrogen-containing compound may be used alone or in admixture.

(F) Surfactant

The resist composition may further include (F) a surfactant which is insoluble or substantially insoluble in water and soluble in alkaline developer, and/or a surfactant which is insoluble or substantially insoluble in water and alkaline developer. For the surfactant, reference should be made to those compounds described in JP-A 2010-215608 and JP-A 2011-016746.

While many examples of the surfactant which is insoluble or substantially insoluble in water and alkaline developer are described in the patent documents cited herein, preferred examples are surfactants FC-4430 (3M), Olfine® E1004 (Nissin Chemical Co., Ltd.), Surflon® S-381, KH-20 and KH-30 (AGC Seimi Chemical Co., Ltd.). Partially fluorinated oxetane ring-opened polymers having the formula (surf-1) are also useful.

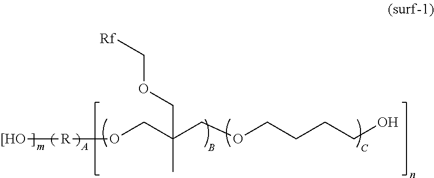

It is provided herein that R, Rf, A, B, C, m, and n are applied to only formula (surf-1), independent of their descriptions other than for the surfactant. R is a di- to tetra-valent C₂-C₅ aliphatic group. Exemplary divalent aliphatic groups include ethylene, 1,4-butylene, 1,2-propylene, 2,2-dimethyl-1,3-propylene and 1,5-pentylene. Exemplary tri- and tetra valent groups are shown below.

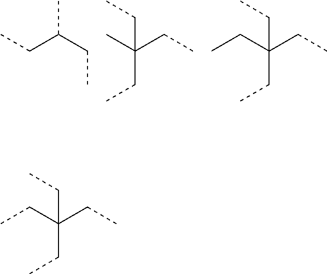

Herein the broken line denotes a valence bond. These formulae are partial structures derived from glycerol, trimethylol ethane, trimethylol propane, and pentaerythritol, respectively. Of these, 1,4-butylene and 2,2-dimethyl-1,3-propylene are preferably used.

Rf is trifluoromethyl or pentafluoroethyl, and preferably trifluoromethyl. The letter in is an integer of 0 to 3, n is an integer of 1 to 4, and the sum of m and n, which represents the valence of R, is an integer of 2 to 4. “A” is equal to 1, B is an integer of 2 to 25, and C is an integer of 0 to 10. Preferably, B is an integer of 4 to 20, and C is 0 or 1. Note that the formula (surf-1) does not prescribe the arrangement of respective constituent units while they may be arranged either block-wise or randomly. For the preparation of surfactants in the form of partially fluorinated oxetane ring-opened polymers, reference should be made to U.S. Pat. No. 5,650,483, for example.

The surfactant which is insoluble or substantially insoluble in water and soluble in alkaline developer is useful when ArF immersion lithography is applied to the resist composition in the absence of a resist protective film. In this embodiment, the surfactant has a propensity to segregate on the resist surface for achieving a function of minimizing water penetration or leaching. The surfactant is also effective for preventing water-soluble components from being leached out of the resist film for minimizing any damage to the exposure tool. The surfactant becomes solubilized during alkaline development following exposure and PEB, and thus forms few or no foreign particles which become defects. The preferred surfactant is a polymeric surfactant which is insoluble or substantially insoluble in water, but soluble in alkaline developer, also referred to as “hydrophobic resin” in this sense, and especially which is water repellent and enhances water sliding.

Suitable polymeric surfactants include those containing repeat units of at least one type selected from the formulae (5A) to (5E).

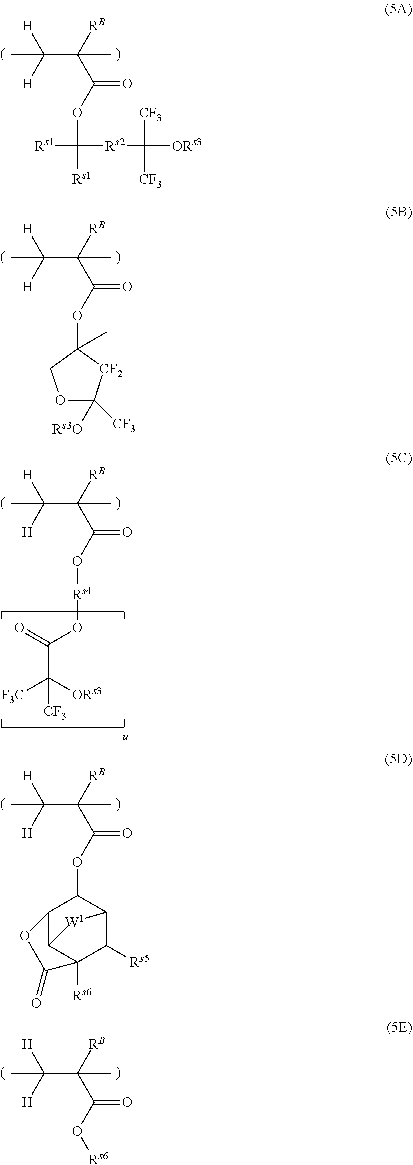

Herein, R^{B} is hydrogen, fluorine, methyl or trifluoromethyl. W¹ is —CH₂—, —CH₂CH₂— or —O—, or two separate —H. R^{s1} is each independently hydrogen or a C₁-C₁₀ hydrocarbyl group. R^{s2} is a single bond or a C₁-C₅ straight or branched hydrocarbylene group. R^{s3} is each independently hydrogen, a C₁-C₁₅ hydrocarbyl or fluorinated hydrocarbyl group, or an acid labile group. When R^{s3} is a hydrocarbyl or fluorinated hydrocarbyl group, an ether bond or carbonyl moiety may intervene in a carbon-carbon bond. R^{s4} is a C₁-C₂₀ (u+1)-valent hydrocarbon or fluorinated hydrocarbon group, and u is an integer of 1 to 3. R^{s5} is each independently hydrogen or a group: —C(═O)—O—R^{sa} wherein R^{sa} is a C₁-C₂₀ fluorinated hydrocarbyl group. R^{s6} is a C₁-C¹⁵ hydrocarbyl or fluorinated hydrocarbyl group in which an ether bond or carbonyl moiety may intervene in a carbon-carbon bond.

The hydrocarbyl group represented by R^{s1} may be straight, branched or cyclic. Examples thereof include methyl, ethyl, n-propyl, isopropyl, cyclopropyl, n-butyl, isobutyl, sec-butyl, tert-butyl, cyclobutyl, n-pentyl, cyclopentyl, n-hexyl, cyclohexyl, n-heptyl, n-octyl; n-nonyl, n-decyl, adamantyl, and norbornyl. Inter alia, C₁-C₆ hydrocarbyl groups are preferred.

The hydrocarbylene group represented by R^{s2} may be straight, branched or cyclic. Examples thereof include methylene, ethylene, propylene, butylene and pentylene.

The hydrocarbyl group represented by R^{s3} or R^{s6} may be straight, branched or cyclic. Examples thereof include alkyl, alkenyl and alkynyl groups, with the alkyl groups being preferred. Suitable alkyl groups include those exemplified for the hydrocarbyl group represented by R^{s1} as well as n-undecyl, n-dodecyl, tridecyl, tetradecyl, and pentadecyl. Examples of the fluorinated hydrocarbyl group represented by R^{s3} or R^{s6} include the foregoing hydrocarbyl groups in which some or all carbon-bonded hydrogen atoms are substituted by fluorine atoms. In these groups, an ether bond or carbonyl moiety may intervene in a carbon-carbon bond as mentioned above.

Examples of the acid labile group represented by R^{s3} include groups of the above formulae (L1) to (L4), C₄-C₂₀, preferably C₄-C₁₅ tertiary hydrocarbyl groups, trialkylsilyl groups in which each alkyl moiety has 1 to 6 carbon atoms, and C₄-C₂₀ oxoalkyl groups.

The (u+1)-valent hydrocarbon or fluorinated hydrocarbon group represented by R^{s4} may be straight, branched or cyclic and examples thereof include the foregoing hydrocarbyl or fluorinated hydrocarbyl groups from which “u” number of hydrogen atoms are eliminated.

The fluorinated hydrocarbyl group represented by R^{sa} may be straight, branched or cyclic. Examples thereof include the foregoing hydrocarbyl groups in which some or all hydrogen atoms are substituted by fluorine atoms. Illustrative examples include trifluoromethyl, 2,2,2-trifluoroethyl, 3,3,3-trifluoro-1-propyl, 3,3,3-trifluoro-2-propyl, 2,2,3,3-tetrafluoropropyl, 1,1,1,3,3,3-hexafluoroisopropyl, 2,2,3,3,4,4,4-heptafluorobutyl, 2,2,3,3,4,4,5,5-octafluoropentyl, 2,2,3,3,4,4,5,5,6,6,7,7-dodecafluoroheptyl, 2-(perfluorobutyl)ethyl, 2-(perfluorohexyl)ethyl, 2-(perfluorooctyl)ethyl, and 2-(perfluorodecyl)ethyl.

Examples of the repeat units having formulae (5A) to (5E) are shown below, but not limited thereto. Herein R^{B} is as defined above.

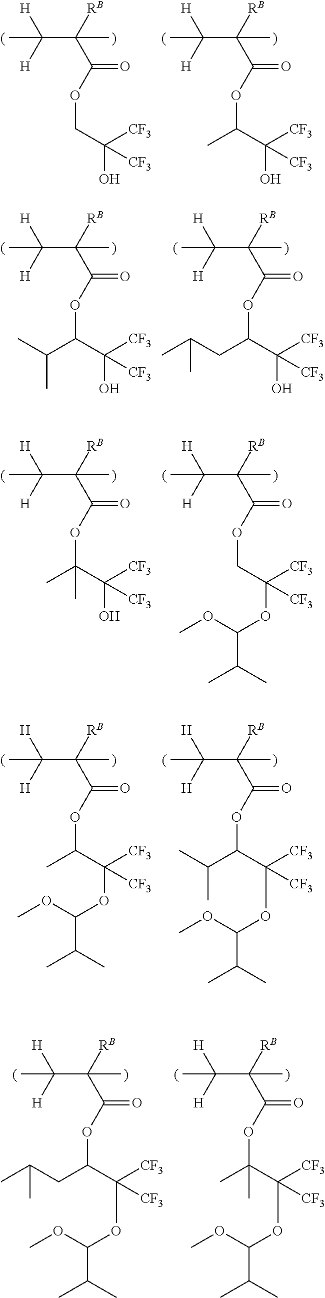

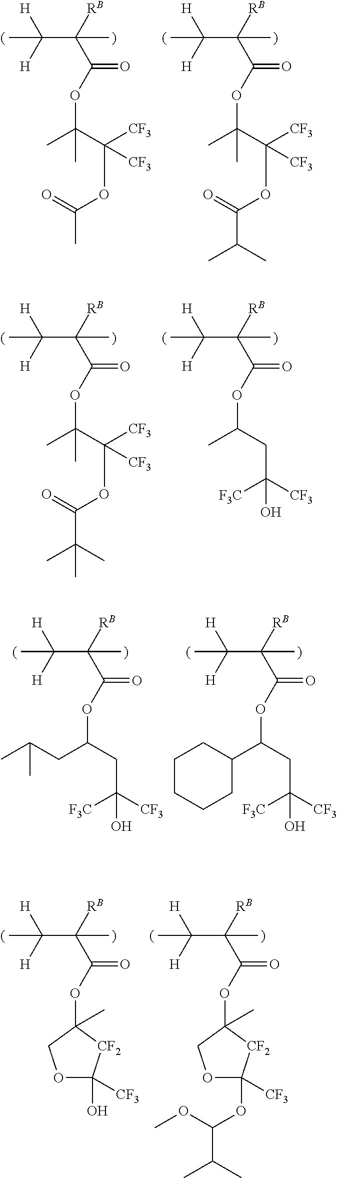

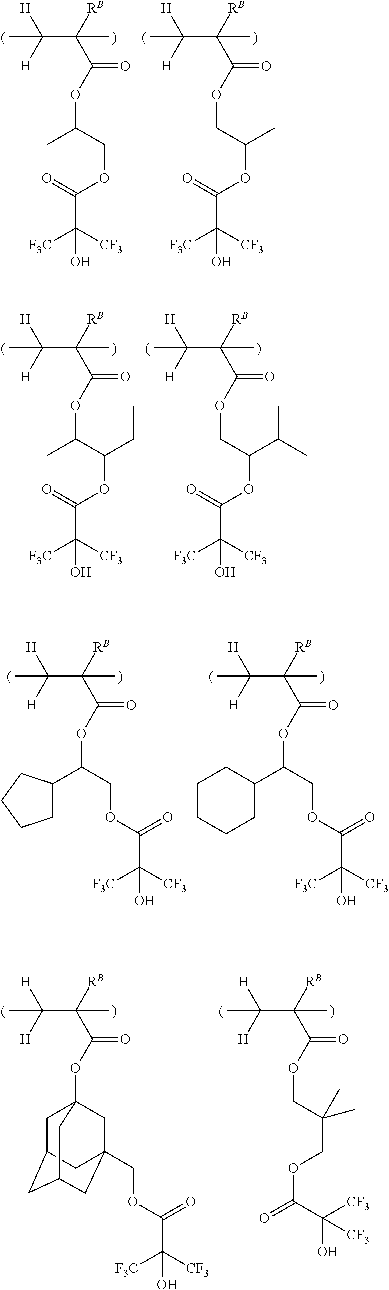

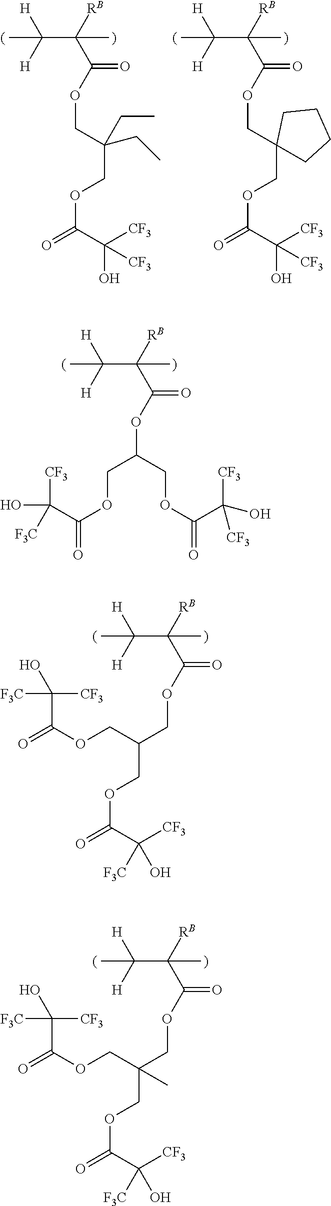

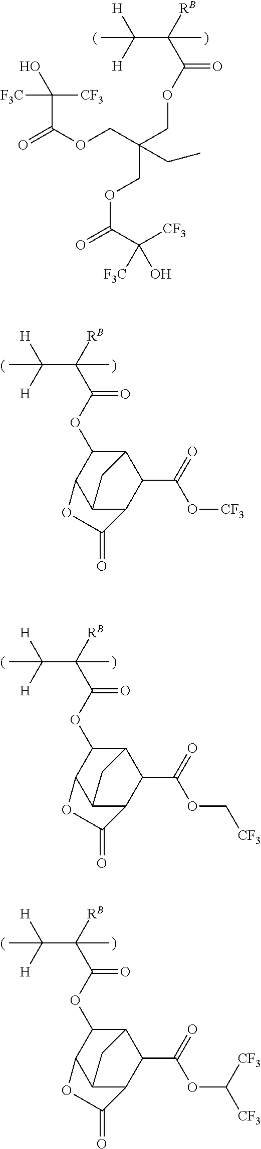

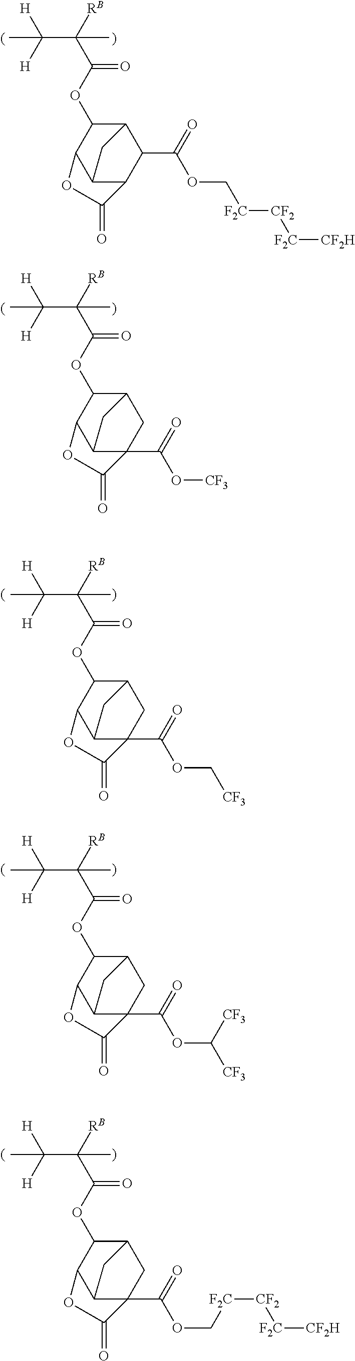

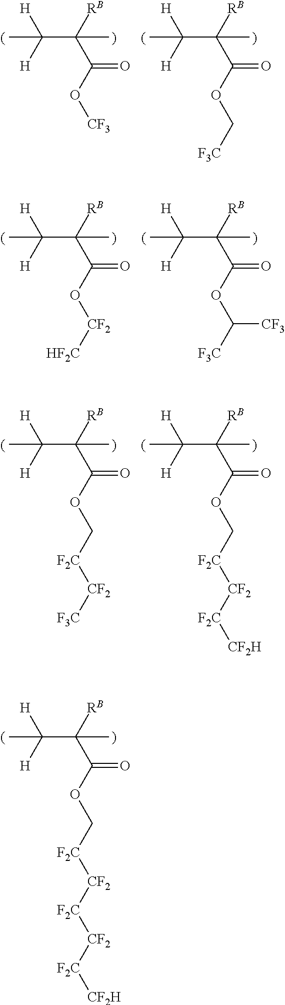

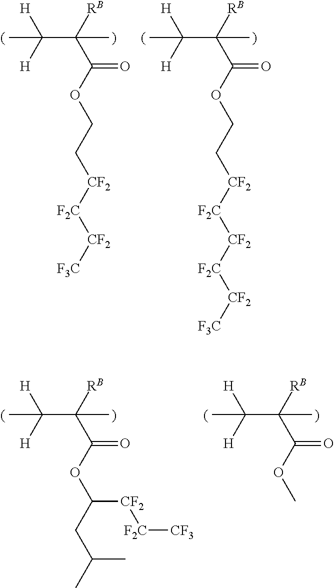

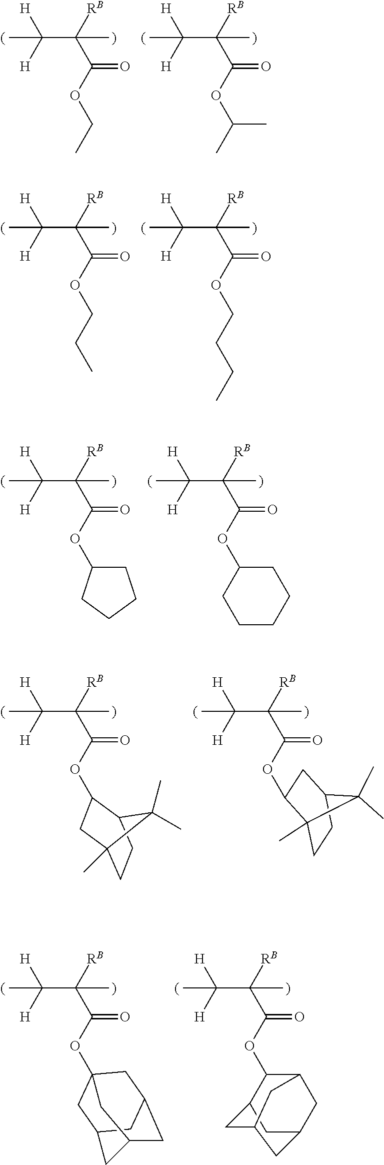

The polymeric surfactant may further contain repeat units other than the repeat units having formulae (5A) to (5E). Typical other repeat units are those derived from methacrylic acid and α-trifluoromethylacrylic acid derivatives. In the polymeric surfactant, the content of the repeat units having formulae (5A) to (5E) is preferably at least 20 mol %, more preferably at least 60 mol %, most preferably 100 mol % of the overall repeat units.

The polymeric surfactant preferably has a Mw of 1,000 to 500,000, more preferably 3,000 to 100,000 and a Mw/Mn of 1.0 to 2.0, more preferably 1.0 to 1.6.

The polymeric surfactant may be synthesized by any desired method, for example, by dissolving an unsaturated bond-containing monomer or monomers providing repeat units having formula (5A) to (5E) and optionally other repeat units in an organic solvent, adding a radical initiator, and heating for polymerization. Suitable organic solvents used herein include toluene, benzene, THF, diethyl ether, and dioxane. Examples of the polymerization initiator used herein include AIBN, 2,2′-azobis(2,4-dimethylvaleronitrile), dimethyl 2,2-azobis(2-methylpropionate), benzoyl peroxide, and lauroyl peroxide. Preferably the reaction temperature is 50 to 100° C. and the reaction time is 4 to 24 hours. The acid labile group that has been incorporated in the monomer may be kept as such, or the polymer may be protected or partially protected therewith at the end of polymerization.

During the synthesis of polymeric surfactant, any known chain transfer agent such as dodecyl mercaptan or 2-mercaptoethanol may be added for molecular weight control purpose. The amount of chain transfer agent added is preferably 0.01 to 10 mol % based on the total moles of monomers to be polymerized.

When the resist composition contains a surfactant (F), the amount thereof is preferably 0.1 to 50 parts by weight, and more preferably 0.5 to 10 parts by weight per 80 parts by weight of the base polymer (B). At least 0.1 part of the surfactant is effective in improving the receding contact angle with water of the resist film at its surface. Up to 50 parts of the surfactant is effective in forming a resist film having a low rate of dissolution in a developer and capable of maintaining the height of a fine pattern formed therein.

Other Components

The resist composition may further comprise another component, for example, a compound which is decomposed with an acid to generate another acid (i.e., acid amplifier compound), an organic acid derivative, a fluorinated alcohol, and a compound having a Mw of up to 3,000 which changes its solubility in developer under the action of an acid (i.e., dissolution inhibitor). Specifically, the acid amplifier compound is described in JP-A 2009 269953 and JP-A 2010-215608 and preferably used in an amount of 0 to 5 parts, more preferably 0 to 3 parts by weight per 80 parts by weight of the base polymer (B). An extra amount of the acid amplifier compound can make the acid diffusion control difficult and cause degradations to resolution and pattern profile. With respect to the remaining additives, reference should be made to JP-A 2009-269953 and JP-A 2010-215608.

Process

A further embodiment of the invention is a process of forming a pattern from the resist composition defined above by lithography. The preferred process includes the steps of applying the resist composition to form a resist film on a substrate, exposing the resist film to high-energy radiation, and developing the exposed resist film in a developer. Any desired steps may be added to the process if necessary.

The substrate used herein may be a substrate for integrated circuitry fabrication, e.g., Si, SiO₂, SiN, SiON, TiN, WSi, BPSG, SOG, organic antireflective film, etc. or a substrate for mask circuitry fabrication, e.g., Cr, CrO, CrON, MoSi₂, SiO₂, etc.

The resist composition is applied onto a substrate by a suitable coating technique such as spin coating. The coating is prebaked on a hot plate preferably at a temperature of 60 to 150° C. for 1 to 10 minutes, more preferably at 80 to 140° C. for 1 to 5 minutes. The resulting resist film preferably has a thickness of 0.05 to 2 μm.

Then the resist film is exposed patternwise to high-energy radiation, typically KrF or ArF excimer laser. EUV or EB. On use of KrF excimer laser. ArF excimer laser or EUV of wavelength 13.5 nm, the resist film is exposed through a mask having a desired pattern, preferably in a dose of 1 to 200 mJ/cm², more preferably 10 to 100 mJ/cm². On use of EB, a pattern may be written directly or through a mask having the desired pattern, preferably in a dose of 1 to 300 μC/cm², more preferably 10 to 200 μC/cm².

The exposure may be performed by conventional lithography whereas the immersion lithography of holding a liquid having a refractive index of at least 1.0 between the resist film and the projection lens may be employed if desired. The liquid is typically water, and in this case, a protective film which is insoluble in water may be formed on the resist film.

While the water-insoluble protective film serves to prevent any components from being leached out of the resist film and to improve water sliding on the film surface, it is generally divided into two types. The first type is an organic solvent-strippable protective film which must be stripped, prior to alkaline development, with an organic solvent in which the resist film is not dissolvable. The second type is an alkali-soluble protective film which is soluble in an alkaline developer so that it can be removed simultaneously with the removal of solubilized regions of the resist film. The protective film of the second type is preferably of a material comprising a polymer having a 1,1,1,3,3,3-hexafluoro-2-propanol residue (which is insoluble in water and soluble in an alkaline developer) as a base in an alcohol solvent of at least 4 carbon atoms, an ether solvent of 8 to 12 carbon atoms or a mixture thereof. Alternatively, the aforementioned surfactant which is insoluble in water and soluble in an alkaline developer may be dissolved in an alcohol solvent of at least 4 carbon atoms, an ether solvent of 8 to 12 carbon atoms or a mixture thereof to form a material from which the protective film of the second type is formed.

After the exposure, the resist film may be baked (PFB), for example, on a hotplate at 60 to 150° C. for 1 to 5 minutes, preferably at 80 to 140° C. for 1 to 3 minutes.

The resist film is then developed with a developer in the form of an aqueous base solution, for example, 0.1 to 5 wt %, preferably 2 to 3 wt % aqueous solution of tetramethylammonium hydroxide (TMAH) for 0.1 to 3 minutes, preferably 0.5 to 2 minutes by conventional techniques such as dip, puddle and spray techniques. In the development step, the exposed region of resist film is dissolved away, and a desired resist pattern is formed on the substrate.

Any desired step may be added to the pattern forming process. For example, after the resist film is formed, a step of rinsing with pure water may be introduced to extract the acid generator or the like from the film surface or wash away particles. After exposure, a step of rinsing may be introduced to remove any water remaining on the film after exposure.

Also, a double patterning process may be used for pattern formation. The double patterning process includes a trench process of processing an underlay to a 1:3 trench pattern by a first step of exposure and etching, shifting the position, and forming a 1:3 trench pattern by a second step of exposure, for forming a 1:1 pattern; and a line process of processing a first underlay to a 1:3 isolated left pattern by a first step of exposure and etching, shifting the position, processing a second underlay formed below the first underlay by a second step of exposure through the 1:3 isolated left pattern, for forming a half-pitch 1:1 pattern.

In the pattern forming process, negative tone development may also be used. That is, an organic solvent may be used instead of the aqueous alkaline solution as the developer for developing and dissolving away the unexposed region of the resist film.

The organic solvent used as the developer is preferably selected from 2-octanone, 2-nonanone, 2-heptanone, 3-heptanone, 4-heptanone, 2-hexanone, 3-hexanone, diisobutyl ketone, methylcyclohexanone, acetophenone, methylacetophenone, propyl acetate, butyl acetate, isobutyl acetate, pentyl acetate, isopentyl acetate, butenyl acetate, propyl formate, butyl formate, isobutyl formate, pentyl formate, isopentyl formate, methyl valerate, methyl pentenoate, methyl crotonate, ethyl crotonate, methyl propionate, ethyl propionate, ethyl 3-ethoxypropionate, methyl lactate, ethyl lactate, propyl lactate, butyl lactate, isobutyl lactate, pentyl lactate, isopentyl lactate, methyl 2-hydroxyisobutyrate, ethyl 2-hydroxyisobutyrate, methyl benzoate, ethyl benzoate, phenyl acetate, benzyl acetate, methyl phenylacetate, ethyl phenylacetate, benzyl formate, phenylethyl formate, methyl 3-phenylpropionate, benzyl propionate, and 2-phenylethyl acetate. These organic solvents may be used alone or in admixture of two or more.

### EXAMPLES

Synthesis Examples, Examples and Comparative Examples are given below by way of illustration and not by way of limitation. The abbreviation “pbw” is parts by weight. THE stands for tetrahydrofuran, and PGMEA for propylene glycol monomethyl ether acetate. Analysis is made by IR and ¹H-NMR spectroscopy using analytic instruments as shown below.

IR: NICOLET 6700 by Thermo Fisher Scientific Inc.

¹H-NMR: ECA-500 by JEOL Ltd.

### [1] Synthesis of Amine Compounds

### Example 1-1 Synthesis of Amine Compound AQ-1

### (1) Synthesis of Intermediate In-1

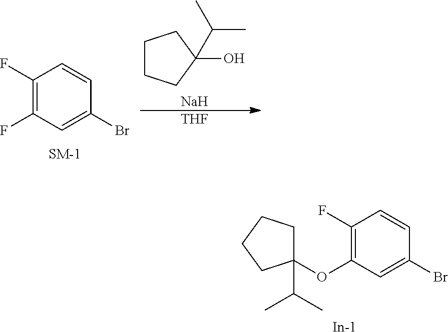

In nitrogen atmosphere, 10.9 g of sodium hydride (purity 55 wt %) was suspended in 60 g of THF. A mixture of 35.3 g of 1-isopropylcyclopentanol and 30 g of THE was added dropwise to the suspension. At the end of addition, the reaction solution was heated under reflux for 4 hours, yielding a metal alkoxide. Thereafter, 48.3 g of reactant SM-1 was added dropwise to the reaction solution, which was heated under reflux and aged for 18 hours. The reaction solution was cooled in an ice bath, whereupon 100 g of water was added to quench the reaction. The desired compound was extracted twice with a solvent mixture of 100 g of toluene and 100 g of hexane, followed by ordinary aqueous workup. After the solvent was distilled off, the desired product was purified by distillation, obtaining 51.2 g of Intermediate In-1 as colorless oily matter (yield 68%, remaining impurities).

### (2) Synthesis of Intermediate In-2

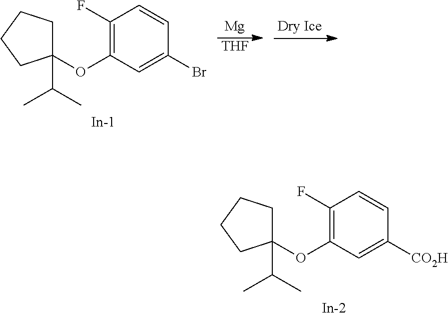

In nitrogen atmosphere, a Grignard reagent was prepared from 4.1 g of metallic magnesium, 51.2 g of Intermediate In-1, and 200 g of THF. The Grignard reagent was added dropwise to a suspension of 200 g of dry ice in 500 g of THF. At the end of addition, the reaction solution was aged until dry ice sublimated. Thereafter, while the reaction solution was kept below 10° C., 150 g of 5 wt % hydrochloric acid was added dropwise to quench the reaction. This was followed by extraction with 600 g of ethyl acetate, ordinary aqueous workup, and solvent distillation. On recrystallization from hexane, there was obtained Intermediate In-2 as white crystals (amount 26.8 g, yield 58%).

### (3) Synthesis of Amine Compound AQ-1

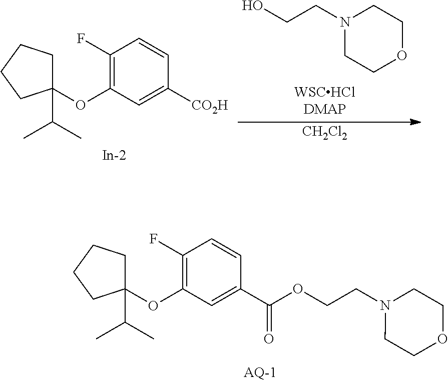

In nitrogen atmosphere, 15.1 g of 1-ethyl-3-(3-dimethylaminopropyl)carbodiimide hydrochloride (WSC—HCl) was added to a solution of 15.0 g of Intermediate In-2, 8.9 g of morpholine ethanol, 0.7 g of dimethylaminopyridine, and 50 g of methylene chloride, which was stirred for 12 hours. The reaction solution was cooled in an ice bath, whereupon 100 g of 1 wt % hydrochloric acid aqueous solution was added to quench the reaction. This was followed by ordinary aqueous workup and solvent distillation. There was obtained 17.9 g of Amine Compound AQ-1 as colorless oily matter (yield 84%).

### Example 1-2 Synthesis of Amine Compound AQ-2

### (1) Synthesis of Intermediate In-3

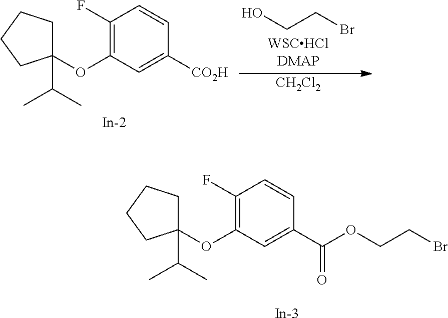

In nitrogen atmosphere, 15.1 g of WSC—HCl was added to a solution of 15.0 g of Intermediate In-2, 8.5 g of bromoethanol, 0.7 g of dimethylaminopyridine, and 50 g of methylene chloride, which was stirred for 12 hours. The reaction solution was cooled in an ice bath, whereupon 100 g of 1 wt % hydrochloric acid aqueous solution was added to quench the reaction. This was followed by ordinary aqueous workup and solvent distillation. There was obtained 18.2 g of Intermediate In-3 as colorless oily matter (yield 87%).

### (2) Synthesis of Amine Compound AQ-2

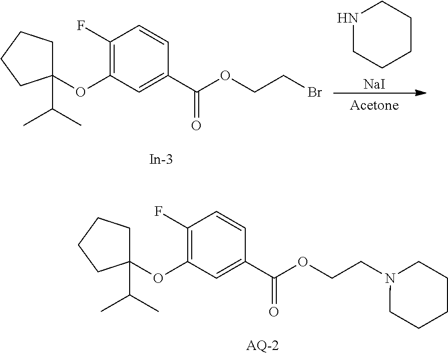

A reactor in nitrogen atmosphere was charged with 18.2 g of Intermediate In-3, 0.7 g of sodium iodide, and 70 g of acetone. At room temperature, 4.7 g of piperidine was added dropwise to the reactor. At the end of addition, the reaction solution was heated under reflux and aged for 24 hours. After the disappearance of Intermediate In-3 was confirmed by TLC, the reaction solution was cooled down to room temperature. Then 35 g of saturated sodium bicarbonate aqueous solution was added to quench the reaction. On an evaporator, the acetone was evaporated off. Then 105 g of methylene chloride was added to the residue for extraction, from which the organic layer was taken out. The organic layer was washed 4 times with 35 g of water and once with 35 g of saturated brine. The organic layer was taken out and concentrated obtaining Amine Compound AQ-2 as oily matter (amount 16.2 g, yield 89%).

### Examples 1-3 to 1-7 Synthesis of Amine Compounds AQ-3 to AQ-7

Amine Compounds AQ-3 to AQ-7 were synthesized by various organic synthesis methods. The structure of Amine Compounds AQ-3 to AQ-7 is shown below.

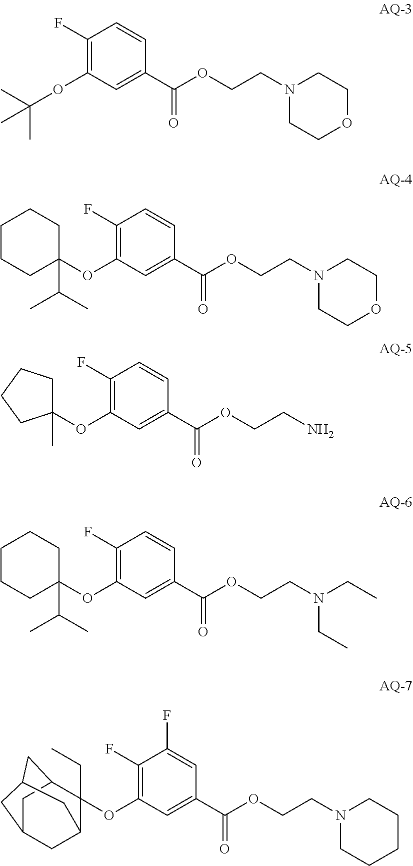 [SMILES: CC(C)(C)Oc1cc(C(=O)OCCN2CCOCC2)ccc1F ; CC(C)C1(Oc2cc(C(=O)OCCN3CCOCC3)ccc2F)CCCCC1 ; CC1(Oc2cc(C(=O)OCCN)ccc2F)CCCC1 ; CCN(CC)CCOC(=O)c1ccc(F)c(OC2(C(C)C)CCCCC2)c1 ; CCC1(Oc2cc(C(=O)OCCN3CCCCC3)cc(F)c2F)C2CC3CC(C2)CC1C3]

### [2] Synthesis of Base Polymers

Base polymers used in resist compositions were synthesized by the following procedure. The polymers were analyzed for Mw and Mw/Mn by GPC versus polystyrene standards using THE solvent.

### Synthesis Example 1 Synthesis of Polymer P-1

A flask in nitrogen atmosphere was charged with 5.0 g of 3-hydroxy-1-adamantyl methacrylate, 14.4 g of α-methacryloxy-γ-butyrolactone, 20.8 g of 1-isopropylcyclopentyl methacrylate, 0.49 g of dimethyl 2,2′-azobis(2-methylpropionate) (V-601 by Fuji Film Wako Prue Chemical Industries, Ltd.), and 56 g of PGMEA to form a monomer/initiator solution. Another flask in nitrogen atmosphere was charged with 19 g of PGMEA, which was heated at 80° C. with stirring. With stirring, the monomer/initiator solution was added dropwise to the flask over 4 hours. After the completion of dropwise addition, the polymerization solution was continuously stirred for 2 hours while maintaining the temperature of 80° C. The polymerization solution was cooled to room temperature, whereupon it was added dropwise to 640 g of methanol with vigorous stirring. The precipitate was collected by filtration, washed twice with 240 g of methanol, and vacuum dried at 50° C. for 20 hours, obtaining Polymer P-1 in white powder form (amount 35.3 g, yield 88%). On GPC analysis, Polymer P-1 had a Mw of 8,500 and a Mw/Mn of 1.56.

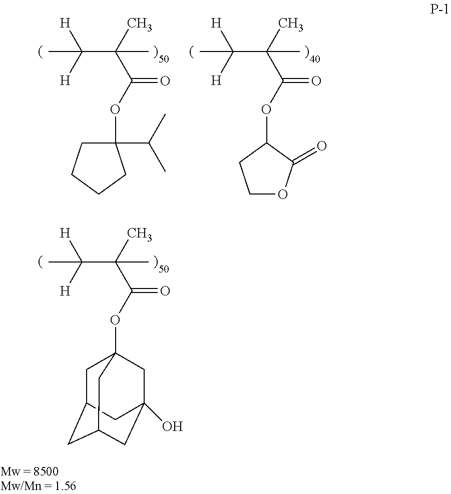

### Synthesis Examples 2 to 7 Synthesis of Polymers P-2 to P-7

Polymers P-2 to P-7 were synthesized by the same procedure as in Synthesis Example 1 aside from changing the type and amount of monomers. Table 1 tabulates the type and molar ratio (mol %) of repeat units in Polymers P-1 to P-7.

TABLE 1

| | | Ratio | | Ratio | | Ratio | | Ratio | | Ratio | | |
|---|---|---|---|---|---|---|---|---|---|---|---|---|
| Polymer | Unit 1 | (mol %) | Unit 2 | (mol %) | Unit 3 | (mol %) | Unit 4 | (mol %) | Unit 5 | (mol %) | Mw | Mw/Mn |
| P-1 | a1-1 | 50 | b1-1 | 40 | b1-4 | 10 | — | — | — | — | 8,500 | 1.56 |
| P-2 | a1-2 | 40 | a1-1 | 10 | b1-1 | 20 | b1-2 | 20 | b1-4 | 10 | 8,100 | 1.74 |
| P-3 | a1-2 | 35 | a1-1 | 15 | b1-1 | 40 | b1-4 | 10 | — | — | 8,300 | 1.70 |
| P-4 | a1-2 | 10 | a1-3 | 40 | b1-1 | 10 | b1-3 | 25 | b1-4 | 15 | 9,400 | 1.67 |
| P-5 | a1-4 | 55 | b2-2 | 30 | c2-1 | 15 | — | — | — | — | 10,600 | 2.01 |
| P-6 | a1-2 | 10 | a2-1 | 30 | b1-2 | 30 | b2-1 | 20 | c2-2 | 10 | 11,200 | 2.08 |
| P-7 | a1-4 | 50 | b2-2 | 50 | — | — | — | — | — | — | 8,500 | 1.68 |

The repeat units in Table 1 are shown below.

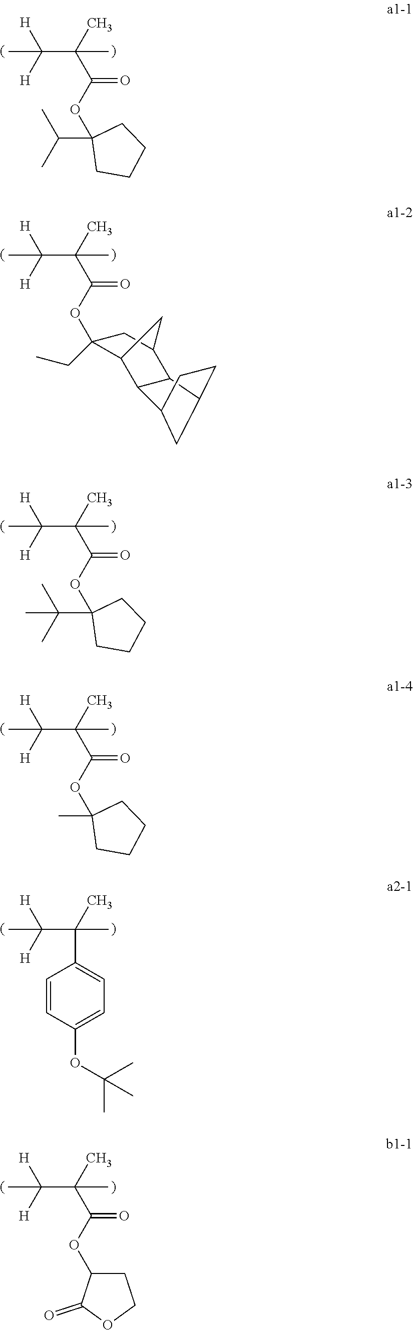

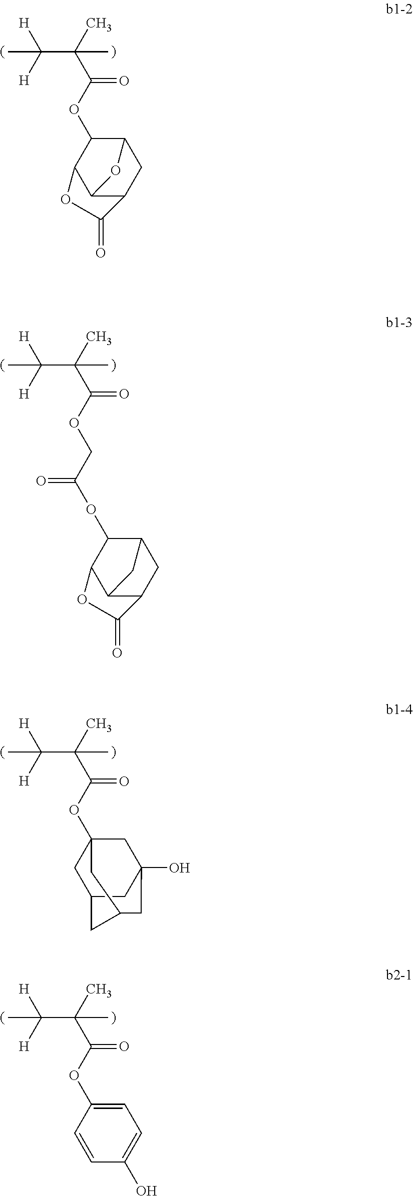

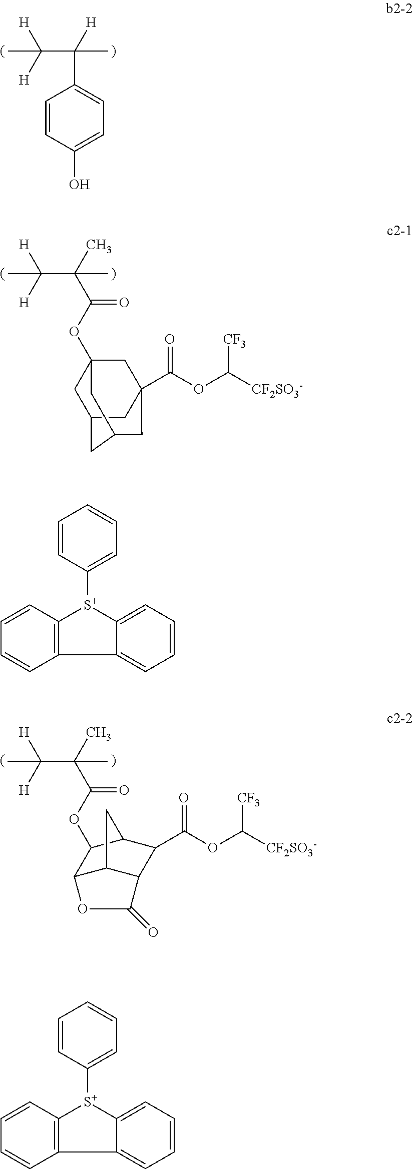

### [3] Preparation of Chemically Amplified Resist Compositions

### Examples 2-1 to 2-22 and Comparative Examples 1-1 to 1-14

Chemically amplified resist compositions (R-1 to R-22, CR-1 to CR-14) in solution form were prepared by dissolving an amine compound (AQ-1 to AQ-7), comparative amine quencher (AQ-A to AQ-F), base polymer (Polymers P-1 to P-7), photoacid generator (PAG-1 to PAG-3), quencher (Q-1. Q-2), and alkali-soluble surfactant (SF-1) in a solvent containing 0.01 wt % of surfactant A in accordance with the formulation shown in Tables 2 and 3, and filtering through a Teflon® filter with a pore size of 0.2 μm.

TABLE 2

| | | | Amine | Base | Photoacid | | | | |
|---|---|---|---|---|---|---|---|---|---|
| | | Resist | compound | polymer | generator | Quencher | Surfactant | Solvent 1 | Solvent 2 |
| | | composition | (pbw) | (pbw) | (pbw) | (pbw) | (pbw) | (pbw) | (pbw) |
| Example | 2-1 | R-1 | AQ-1 | P-1 | PAG-1 | Q-1 | SF-1 | PGMEA | GBL |
| | | | (3.0) | (80) | (12.0) | (5.0) | (3.0) | (1400) | (400) |
| | 2-2 | R-2 | AQ-2 | P-1 | PAG-1 | Q-1 | SF-1 | PGMEA | GBL |
| | | | (3.0) | (80) | (12.0) | (5.0) | (3.0) | (1400) | (400) |
| | 2-3 | R-3 | AQ-3 | P-1 | PAG-1 | Q-1 | SF-1 | PGMEA | GBL |
| | | | (3.0) | (80) | (12.0) | (5.0) | (3.0) | (1400) | (400) |
| | 2-4 | R-4 | AQ-4 | P-1 | PAG-1 | Q-1 | SF-1 | PGMEA | GBL |
| | | | (3.0) | (80) | (12.0) | (5.0) | (3.0) | (1400) | (400) |
| | 2-5 | R-5 | AQ-5 | P-1 | PAG-1 | Q-1 | SF-1 | PGMEA | GBL |
| | | | (3.0) | (80) | (12.0) | (5.0) | (3.0) | (1400) | (400) |
| | 2-6 | R-6 | AQ-6 | P-1 | PAG-1 | Q-1 | SF-1 | PGMEA | GBL |
| | | | (3.0) | (80) | (12.0) | (5.0) | (3.0) | (1400) | (400) |
| | 2-7 | R-7 | AQ-7 | P-1 | PAG-1 | Q-1 | SF-1 | PGMEA | GBL |
| | | | (3.0) | (80) | (12.0) | (5.0) | (3.0) | (1400) | (400) |
| | 2-8 | R-8 | AQ-1 | P-2 | PAG-2 | Q-1 | SF-1 | PGMEA | GBL |
| | | | (5.0) | (80) | (10.0) | (2.0) | (3.0) | (1400) | (400) |
| | 2-9 | R-9 | AQ-1 | P-3 | PAG-1 | Q-1 | SF-1 | PGMEA | GBL |
| | | | (5.0) | (80) | (12.0) | (2.0) | (3.0) | (1400) | (400) |
| | 2-10 | R-10 | AQ-2 | P-3 | PAG-1 | Q-1 | SF-1 | PGMEA | GBL |
| | | | (5.0) | (80) | (12.0) | (2.0) | (3.0) | (1400) | (400) |
| | 2-11 | R-11 | AQ-4 | P-4 | PAG-2 | Q-1 | SF-1 | PGMEA | GBL |
| | | | (3.0) | (80) | (10.0) | (2.0) | (3.0) | (1400) | (400) |
| | 2-12 | R-12 | AQ-6 | P-4 | PAG-2 | Q-1 | SF-1 | PGMEA | GBL |
| | | | (3.0) | (80) | (10.0) | (2.0) | (3.0) | (1400) | (400) |
| | 2-13 | R-13 | AQ-7 | P-4 | PAG-2 | Q-1 | SF-1 | PGMEA | GBL |
| | | | (3.0) | (80) | (10.0) | (2.0) | (3.0) | (1400) | (400) |
| | 2-14 | R-14 | AQ-1 | P-5 | — | Q-2 | — | PGMEA | DAA |
| | | | (5.0) | (80) | | (7.0) | | (2200) | (900) |
| | 2-15 | R-15 | AQ-2 | P-5 | — | Q-2 | — | PGMEA | DAA |
| | | | (5.0) | (80) | | (7.0) | | (2200) | (900) |
| | 2-16 | R-16 | AQ-1 | P-5 | PAG-3 | Q-2 | — | PGMEA | DAA |
| | | | (5.0) | (80) | (8.0) | (7.0) | | (2200) | (900) |
| | 2-17 | R-17 | AQ-7 | P-5 | — | Q-2 | — | PGMEA | DAA |
| | | | (5.0) | (80) | | (7.0) | | (2200) | (900) |
| | 2-18 | R-18 | AQ-3 | P-6 | — | Q-2 | — | PGMEA | DAA |
| | | | (5.0) | (80) | | (7.0) | | (2200) | (900) |
| | 2-19 | R-19 | AQ-2 | P-6 | — | Q-2 | — | PGMEA | DAA |
| | | | (5.0) | (80) | | (7.0) | | (2200) | (900) |
| | 2-20 | R-20 | AQ-1 | P-7 | PAG-3 | Q-2 | — | PGMEA | DAA |
| | | | (5.0) | (80) | (20.0) | (7.0) | | (2200) | (900) |
| | 2-21 | R-21 | AQ-2 | P-7 | PAG-3 | — | — | PGMEA | DAA |
| | | | (5.0) | (80) | (20.0) | | | (2200) | (900) |
| | 2-22 | R-22 | AQ-7 | P-7 | PAG-3 | — | — | PGMEA | DAA |
| | | | (5.0) | (80) | (20.0) | | | (2200) | (900) |

TABLE 3

| | | | Amine | Base | Photoacid | | | | |
|---|---|---|---|---|---|---|---|---|---|
| | | Resist | compound | polymer | generator | Quencher | Surfactant | Solvent 1 | Solvent 2 |
| | | composition | (pbw) | (pbw) | (pbw) | (pbw) | (pbw) | (pbw) | (pbw) |
| Comparative | 1-1 | CR-1 | AQ-A | P-1 | PAG-1 | Q-1 | SF-1 | PGMEA | GBL |
| Example | | | (3.0) | (80) | (12.0) | (5.0) | (3.0) | (1400) | (400) |
| | 1-2 | CR-2 | AQ-B | P-1 | PAG-1 | Q-1 | SF-1 | PGMEA | GBL |
| | | | (3.0) | (80) | (12.0) | (5.0) | (3.0) | (1400) | (400) |
| | 1-3 | CR-3 | AQ-C | P-1 | PAG-1 | Q-1 | SF-1 | PGMEA | GBL |
| | | | (3.0) | (80) | (12.0) | (5.0) | (3.0) | (1400) | (400) |
| | 1-4 | CR-4 | AQ-D | P-1 | PAG-1 | Q-1 | SF-1 | PGMEA | GBL |
| | | | (3.0) | (80) | (12.0) | (5.0) | (3.0) | (1400) | (400) |
| | 1-5 | CR-5 | AQ-E | P-1 | PAG-1 | Q-1 | SF-1 | PGMEA | GBL |
| | | | (3.0) | (80) | (12.0) | (5.0) | (3.0) | (1400) | (400) |
| | 1-6 | CR-6 | AQ-F | P-1 | PAG-1 | Q-1 | SF-1 | PGMEA | GBL |
| | | | (3.0) | (80) | (12.0) | (5.0) | (3.0) | (1400) | (400) |
| | 1-7 | CR-7 | AQ-A | P-3 | PAG-1 | Q-1 | SF-1 | PGMEA | GBL |
| | | | (3.0) | (80) | (12.0) | (2.0) | (3.0) | (1400) | (400) |
| | 1-8 | CR-8 | AQ-C | P-4 | PAG-2 | Q-1 | SF-1 | PGMEA | GBL |
| | | | (3.0) | (80) | (10.0) | (2.0) | (3.0) | (1400) | (400) |
| | 1-9 | CR-9 | AQ-A | P-5 | — | Q-2 | — | PGMEA | DAA |
| | | | (5.0) | (80) | | (7.0) | | (2200) | (900) |
| | 1-10 | CR-10 | AQ-C | P-5 | — | Q-2 | — | PGMEA | DAA |
| | | | (5.0) | (80) | | (7.0) | | (1400) | (900) |
| | 1-11 | CR-11 | AQ-E | P-5 | — | Q-2 | — | PGMEA | DAA |
| | | | (5.0) | (80) | | (7.0) | | (1400) | (900) |
| | 1-12 | CR-12 | AQ-B | P-6 | — | Q-2 | — | PGMEA | DAA |
| | | | (5.0) | (80) | | (7.0) | | (1400) | (900) |
| | 1-13 | CR-13 | AQ-D | P-6 | — | Q-2 | — | PGMEA | DAA |
| | | | (5.0) | (80) | | (7.0) | | (1400) | (900) |
| | 1-14 | CR-14 | AQ-A | P-7 | PAG-3 | Q-2 | — | PGMEA | DAA |
| | | | (5.0) | (80) | (20.0) | (7.0) | | (1400) | (900) |

The solvents, alkali-soluble surfactant SF-1, photoacid generators PAG-1 to PAG-3, quenchers Q-1 and Q-2, and comparative amine quenchers AQ-A to AQ-F in Tables 2 and 3 are identified below.

Solvent:
PGMEA (propylene glycol monomethyl ether acetate)
GBL (γ-butyrolactone)
DAA (diacetone alcohol)

### Alkali-Soluble Surfactant SF-1: poly(2,2,3,3,4,4,4-heptafluoro-1-isobutyl-1-butyl methacrylate/9-(2,2,2-trifluoro-1-trifluoroethyloxycarbonyl)-4-oxatricyclo[4.2.1.0^{3,7}]nonan-5-on-2-yl methacrylate)

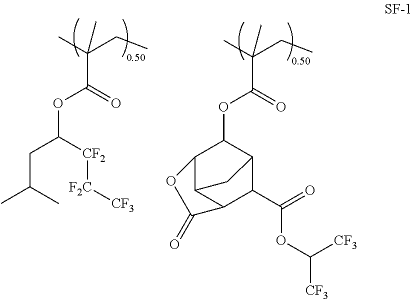 [0329] Mw=7,700 Mw/Mn=1.82
Photoacid generator: PAG-1 to PAG-3

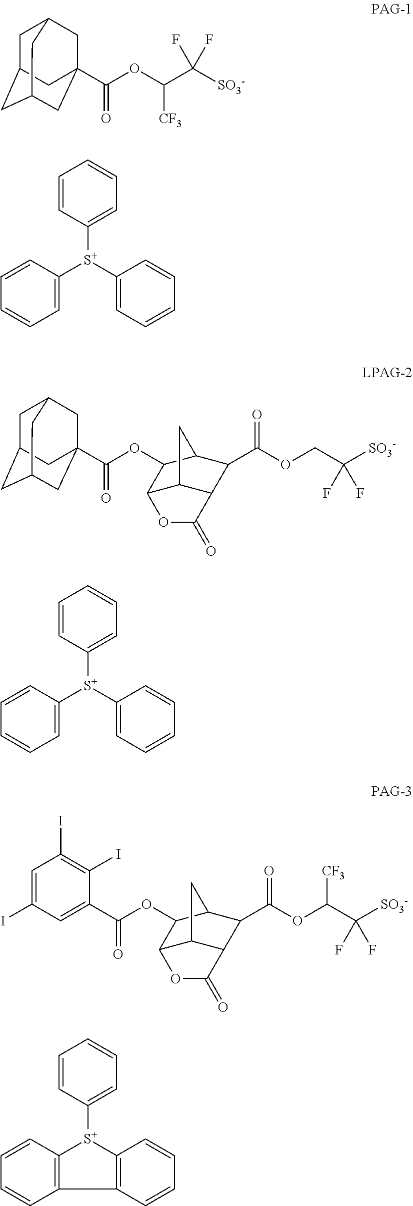

Quencher: Q-1 and Q-2

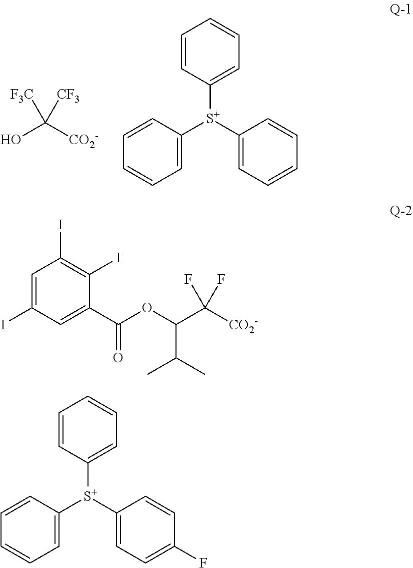

Comparative amine quenchers: AQ-A to AQ-F

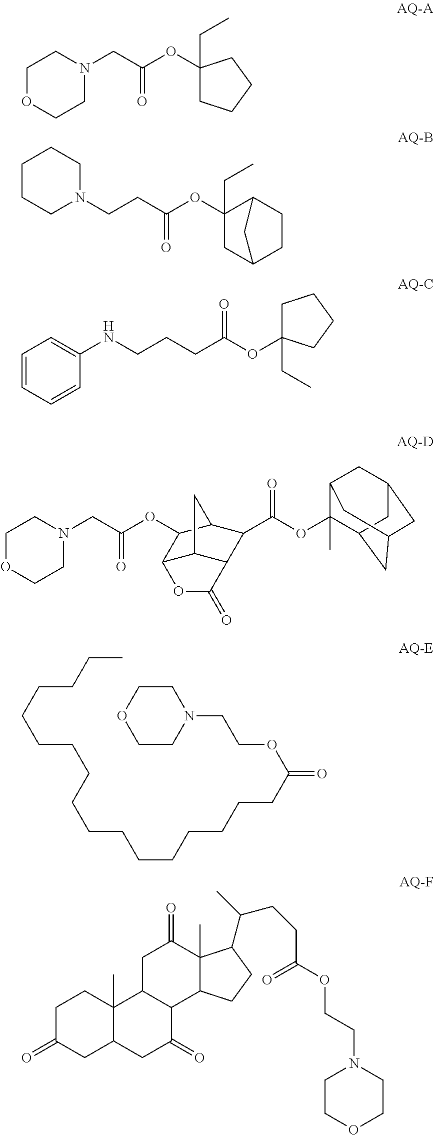

### Surfactant A: 3-methyl-3-(2,2,2-trifluoroethoxymethyl)oxetane/tetrahydrofuran/2,2-dimethyl-1,3-propane diol copolymer (Omnova Solutions, Inc.)

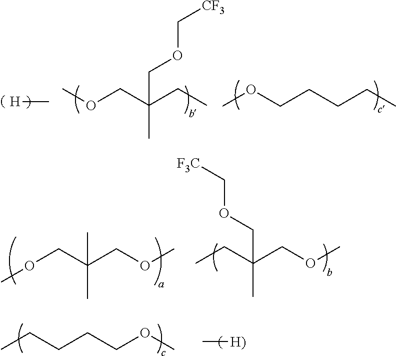 [0334] a:(b+b′):(c+c′)=1:4-7:0.01-1 (molar ratio)
Mw=1,500

### [4] Evaluation of Resist Composition: ArF Lithography Patterning Test 1

### Examples 3-1 to 3-7 and Comparative Examples 2-1 to 2-6

On a silicon substrate, an antireflective coating solution (ARC29A, Nissan Chemical Corp.) was coated and baked at 200° C. for 60 seconds to form an ARC of 100 urn thick. Each of the resist compositions (R-1 to R-7, CR-1 to CR-6) was spin coated on the ARC and prebaked on a hotplate at 100° C. for 60 seconds to form a resist film of 90 urn thick on the ARC. The wafer was exposed on an ArF excimer laser immersion lithography scanner (NSR-S610C by Nikon Corp., NA 1.30, dipole illumination) through a Cr mask having a line-and-space (LS) pattern with a line width of 40 nm and a pitch of 80 nm (on-wafer size), while varying the exposure dose and focus at a dose pitch of 1 mJ/cm² and a focus pitch of 0.025 μm. The immersion liquid used herein was water. After exposure, the resist film was baked (PEB) at the temperature shown in Table 4 for 60 seconds. The resist film was puddle developed in a 2.38 wt % tetramethylammonium hydroxide (TMAH) aqueous solution for 30 seconds, rinsed with deionized water and spin dried, forming a positive pattern. The LS pattern after development was observed under CD-SEM (CG4000 by Hitachi High-Technologies Corp.), whereupon sensitivity, EL, MEF, and LWR were evaluated by the following methods. The results are shown in Table 4.

Evaluation of Sensitivity

The optimum exposure dose Eop (mJ/cm²) which provided a LS pattern having a line width of 40 nm and a pitch of 80 nm was determined as an index of sensitivity. A smaller dose value indicates a higher sensitivity.

Evaluation of Exposure Latitude (EL)

The exposure dose which provided a LS pattern with a space width of 40 nm±10% (i.e., 36 nm to 44 nm) was determined. EL (%) is calculated from the exposure doses according to the following equation:

EL (%)=(|E1−E2|/Eop)×100
wherein E1 is an optimum exposure dose which provides a LS pattern with a line width of 36 nm and a pitch of 80 nm, E2 is an optimum exposure dose which provides a LS pattern with a line width of 44 nm and a pitch of 80 nm, and Eop is an optimum exposure dose which provides a LS pattern with a line width of 40 nm and a pitch of 80 nm. A larger value indicates better performance.

Evaluation of Mask Error Factor (MEF)

A LS pattern was formed by exposure in the optimum dose Eop through the mask with the pitch fixed and the line width varied. MEF was calculated from the mask line width and a variation of the pattern line width according to the following equation:

MEF=(pattern line width)/(mask line width)−b
wherein b is a constant. A value closer to unity (1) indicates better performance.

Evaluation of Line Width Roughness (LWR)

A LS pattern was formed by exposure in the optimum dose Eop. The line width was measured at longitudinally spaced apart 10 points, from which a 3-fold value (3σ) of the standard deviation (σ) was determined and reported as LWR. A smaller value of 3a indicates a pattern having a lower roughness and more uniform line width.

TABLE 4

| | | Resist | PEB temp. | Eop | EL | | LWR |
|---|---|---|---|---|---|---|---|
| | | composition | (° C.) | (mJ/cm²) | (%) | MEF | (nm) |
| Example | 3-1 | R-1 | 95 | 45 | 15 | 1.9 | 1.9 |
| | 3-2 | R-2 | 95 | 45 | 14 | 1.8 | 2.2 |
| | 3-3 | R-3 | 90 | 46 | 18 | 2.0 | 2.1 |
| | 3-4 | R-4 | 90 | 46 | 15 | 2.0 | 2.2 |
| | 3-5 | R-5 | 100 | 49 | 16 | 2.1 | 2.3 |
| | 3-6 | R-6 | 95 | 47 | 15 | 1.9 | 2.1 |
| | 3-7 | R-7 | 90 | 48 | 19 | 2.3 | 2.2 |
| Comparative | 2-1 | CR-1 | 95 | 47 | 10 | 2.8 | 2.8 |
| Example | 2-2 | CR-2 | 90 | 51 | 10 | 2.7 | 2.9 |
| | 2-3 | CR-3 | 95 | 49 | 11 | 2.7 | 3.2 |
| | 2-4 | CR-4 | 95 | 58 | 10 | 2.5 | 2.7 |
| | 2-5 | CR-5 | 90 | 54 | 9 | 3.3 | 3.0 |
| | 2-6 | CR-6 | 100 | 56 | 10 | 3.2 | 3.1 |

As is evident from Table 4, the chemically amplified resist compositions containing amine compounds within the scope of the invention exhibit a satisfactory sensitivity, improved values of EL, MEF and LWR. The resist compositions are useful as the ArF immersion lithography material.

### [5] Evaluation of Resist Composition: ArF Lithography Patterning Test 2

### Examples 4-1 to 4-6 and Comparative Examples 3-1 to 3-2

On a substrate, a spin-on carbon film ODL-180 (Shin-Etsu Chemical Co., Ltd.) having a carbon content of 80 wt % was deposited to a thickness of 180 nm and a silicon-containing spin-on hard mask SHB-A941 having a silicon content of 43 wt % was deposited thereon to a thickness of 35 nm. On this substrate for trilayer process, each of the resist compositions (R-8 to R-13, CR-7, CR-8) was spin coated, then baked on a hot plate at 100° C. for 60 seconds to form a resist film of 100 nm thick.

Using an ArF excimer laser immersion lithography scanner NSR-S610C (Nikon Corp., NA 1.30, σ0.90:0.72, cross-pole opening 35 deg., cross-pole illumination, azimuthally polarized illumination), exposure was performed through a 6% halftone phase shift mask bearing a contact hole (CH) pattern with a hole size of 45 nm and a pitch of 110 nm (on-wafer size) while varying the dose and focus (dose pitch: 1 mJ/cm², focus pitch: 0.025 μm). The immersion liquid used herein was water. After the exposure, the wafer was baked (PEB) at the temperature shown in Table 5 for 60 seconds. Thereafter, the resist film was puddle developed in n-butyl acetate for 30 seconds, rinsed with 4-methyl-2-pentanol, and spin dried, obtaining a negative pattern. The CH pattern after development was observed under CD-SEM CG4000 (Hitachi High Technologies Corp.) whereupon sensitivity, MEF, CDU, and DOF were evaluated by the following methods. The results are shown in Table 5.

Evaluation of Sensitivity

The optimum dose Eop (mJ/cm²) which provided a CH pattern with a hole size of 45 nm and a pitch of 110 nm was determined as an index of sensitivity. A smaller dose value indicates a higher sensitivity.

Evaluation of MEF

A CH pattern was formed by exposure at the optimum dose Eop by ArF lithography patterning test 2 with the pitch fixed and the mask size varied. MEF was calculated from the mask size and a variation of the CH pattern size according to the following equation:

MEF=(CH pattern size)/(mask size)−b
wherein b is a constant. A value closer to unity (1) indicates better performance.

Evaluation of Critical Dimension Uniformity (CDU)

For the CH pattern formed by exposure at the optimum dose Eop, the hole size was measured at 10 areas subject to an identical dose of shot (9 contact holes per area), from which a 3-fold value (3σ) of the standard deviation (σ) was determined and reported as CDU. A smaller value of 3σ indicates a CH pattern having improved CDU.

Evaluation of Depth of Focus (DOF)

As an index of DOF, a range of focus which provided a CH pattern with a size of 45 nm±10% (i.e., 40.5 to 49.5 nm) was determined. A greater value indicates a wider DOF.

TABLE 5

| | | Resist | PEB temp. | Eop | | CDU | DOF |
|---|---|---|---|---|---|---|---|
| | | composition | (° C.) | (mJ/cm²) | MEF | (nm) | (nm) |
| Example | 4-1 | R-8 | 85 | 40 | 2.3 | 2 | 120 |
| | 4-2 | R-9 | 80 | 42 | 2.4 | 2.1 | 120 |
| | 4-3 | R-10 | 85 | 41 | 2.5 | 1.9 | 120 |
| | 4-4 | R-11 | 85 | 42 | 2.2 | 1.8 | 120 |
| | 4-5 | R-12 | 80 | 41 | 2.4 | 1.9 | 120 |
| | 4-6 | R-13 | 80 | 40 | 2.2 | 1.9 | 120 |
| Comparative | 3-1 | CR-7 | 80 | 55 | 3 | 2.3 | 80 |
| Example | 3-2 | CR-8 | 85 | 54 | 3.1 | 2.5 | 80 |

As is evident from Table 5, the chemically amplified resist compositions containing amine compounds within the scope of the invention exhibit a satisfactory sensitivity and unproved values of MEF. CDU and DOF. The resist compositions are useful in the ArF immersion lithography process.

### [6] Evaluation of Resist Composition: EUV Lithography Test

### Examples 5-1 to 5-9 and Comparative Examples 4-1 to 4-6

Each of the chemically amplified resist compositions (R-14 to R-22, CR-9 to CR-14) was spin coated on a silicon substrate having a 20-nm coating of silicon-containing spin-on hard mask SHB-A940 (Shin-Etsu Chemical Co., Ltd., silicon content 43 wt %) and prebaked on a hotplate at 100° C. for 60 seconds to form a resist film of 50 nm thick. Using an EUV scanner NXE3300 (ASML, NA 0.33, a 0.9:0.6, dipole illumination), the resist film was exposed to EUV through a mask bearing a LS pattern having a size of 18 mm and a pitch of 36 nw (on-wafer size) while varying the dose and focus (dose pitch: 1 mJ/cm², focus pitch: 0.020 μm). The resist flint was baked (PEB) on a hotplate at the temperature shown in Table 6 for 60 seconds and puddle developed in a 2.38 wt % TMAH aqueous solution for 30 seconds, rinsed with a rinse fluid containing surfactant, and spin dried to form a positive pattern.

The LS pattern as developed was observed under CD-SEM (CG6300, Hitachi High-Technologies Corp.) whereupon sensitivity, EL, LWR, and DOF were evaluated by the following methods. The results are shown in Table 6.

Evaluation of Sensitivity

The optimum dose Eop (mJ/cm²) which provided a LS pattern with a line width of 18 nm and a pitch of 36 nm was determined as an index of sensitivity.

Evaluation of EL

The exposure dose which provided a LS pattern with a space width of 18 nm±10% (i.e., 16.2 to 19.8 nm) was determined. EL (%) is calculated from the exposure doses according to the following equation:

EL (%)=(|E1−E2|/Eop)×100
wherein E1 is an optimum exposure dose which provides a LS pattern with a line width of 16.2 nm and a pitch of 36 nm, E2 is an optimum exposure dose which provides a LS pattern with a line width of 19.8 nm and a pitch of 36 nm, and Eop is an optimum exposure dose which provides a LS pattern with a line width of 18 nm and a pitch of 36 nm. A larger value indicates better performance.

Evaluation of LWR

For the LS pattern formed by exposure at the optimum dose Eop, the line width was measured at 10 longitudinally spaced apart points, from which a 3-fold value (3σ) of the standard deviation (σ) was determined and reported as LWR. A smaller value of 3σ indicates a pattern having small roughness and uniform line width.

Evaluation of DOF

As an index of DOF, a range of focus which provided a LS pattern with a size of 18 nm±10% (i.e., 16.2 to 19.8 nm) was determined. A greater value indicates a wider DOF.

TABLE 6

| | | Resist | PEB temp. | Eop | EL | LWR | DOF |
|---|---|---|---|---|---|---|---|
| | | composition | (° C.) | (mJ/cm²) | (%) | (nm) | (nm) |
| Example | 5-1 | R-14 | 95 | 42 | 18 | 2.9 | 100 |
| | 5-2 | R-15 | 95 | 45 | 18 | 3.0 | 100 |
| | 5-3 | R-16 | 95 | 44 | 17 | 2.8 | 100 |
| | 5-4 | R-17 | 95 | 41 | 16 | 2.8 | 100 |
| | 5-5 | R-18 | 95 | 43 | 16 | 2.7 | 100 |
| | 5-6 | R-19 | 85 | 45 | 15 | 3.3 | 120 |
| | 5-7 | R-20 | 85 | 51 | 18 | 3.2 | 120 |
| | 5-8 | R-21 | 90 | 48 | 16 | 3.4 | 120 |
| | 5-9 | R-22 | 90 | 52 | 17 | 3.5 | 110 |
| Comparative | 4-1 | CR-9 | 95 | 48 | 8 | 3.9 | 80 |
| Example | 4-2 | CR-10 | 95 | 48 | 13 | 3.8 | 80 |
| | 4-3 | CR-11 | 95 | 52 | 10 | 3.8 | 80 |
| | 4-4 | CR-12 | 95 | 50 | 12 | 3.7 | 80 |
| | 4-5 | CR-13 | 95 | 48 | 9 | 3.6 | 80 |
| | 4-6 | CR-14 | 85 | 53 | 10 | 3.9 | 80 |

It is demonstrated in Table 6 that chemically amplified resist compositions comprising amine compounds within the scope of the invention exhibit a high sensitivity and improved values of EL, LWR and DOF. The resist compositions are useful in the EUV lithography process.

Japanese Patent Application No. 2022-178631 is incorporated herein by reference. Although some preferred embodiments have been described, many modifications and variations may be made thereto in light of the above teachings. It is therefore to be understood that the invention may be practiced otherwise than as specifically described without departing from the scope of the appended claims.

1. An amine compound having the formula (1): 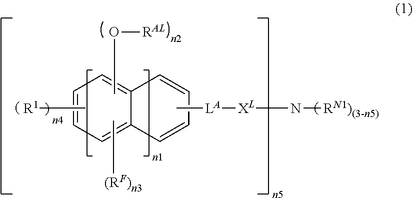
wherein n1 is an integer of 0 or 1, n2 is an integer of 1 to 3, n3 is an integer of 1 to 4, n4 is an integer of 0 to 4, with the proviso that n2+n3+n4≤5 in case of n1=0, and n2+n3+n4≤7 in case of n1=1, n5 is an integer of 1 to 3,
R^{AL} forms an acid labile group with the neighboring oxygen atom,
R^{F} is fluorine, a C₁-C₆ fluorinated saturated hydrocarbyl group, C₁-C₆ fluorinated saturated hydrocarbyloxy group, or C₁-C₆ fluorinated saturated hydrocarbylthio group, a plurality of R^{F} may be the same or different in case of n3≥2,
R^{F} and —O—R^{AL} are attached to adjacent carbon atoms,
R¹ is a C₁-C₂₀ hydrocarbyl group which may contain a heteroatom,
L^{A} is a single bond, ether bond, ester bond, sulfonic ester bond, carbonate bond or carbamate bond,
X^{L} is a single bond or a C₁-C₄₀ hydrocarbylene group which may contain a heteroatom,
R^{N1} is hydrogen or a C₁-C₂₀ hydrocarbyl group in which some or all of the hydrogen atoms may be substituted by halogen and some constituent —CH₂— may be replaced by —O— or —C(═O)—, and in case of n5=1, two R^{N1} may bond together to form a ring with the nitrogen atom to which they are attached, the ring optionally containing —O— or —S—, excluding that two R^{N1} are hydrogen at the same time.

2. The amine compound of claim 1 wherein R^{AL} is a group having the formula (AL-1) or (AL-2): 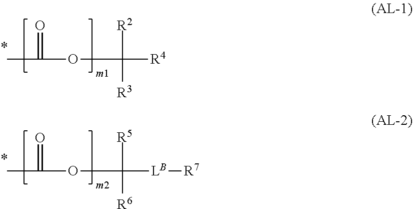
wherein R², R³ and R⁴ are each independently a C₁-C₁₂ hydrocarbyl group in which some constituent —CH₂— may be replaced by —O— or —S—, when the hydrocarbyl group contains an aromatic ring, some or all of the hydrogen atoms on the aromatic ring may be substituted by halogen, cyano, nitro, optionally halogenated C₁-C₄ alkyl moiety, or optionally halogenated C₁-C₄ alkoxy moiety, R² and R³ may bond together to form a ring with the carbon atom to which they are attached, some constituent —CH₂— in the ring may be replaced by —O— or —S—,
R⁵ and R⁶ are each independently hydrogen or a C₁-C₁₀ hydrocarbyl group, R⁷ is a C₁-C₂₀ hydrocarbyl group in which some constituent —CH₂— may be replaced by —O— or —S—, R⁶ and R⁷ may bond together to form a C₃-C₂₀ heterocyclic group with the carbon atom and L^{B} to which they are attached, some constituent —CH₂— in the heterocyclic group may be replaced by —O— or —S—,
L^{B} is —O— or —S—,
m1 is 0 or 1, m2 is 0 or 1,
* designates a point of attachment to the neighboring —O—.

3. The amine compound of claim 1, having the formula (1A): 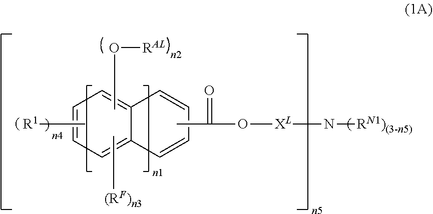
wherein R^{AL}, R^{F}, R¹, R^{N1}, X^{L} and n1 to n5 are as defined above.

4. The amine compound of claim 3, having the formula (1B): 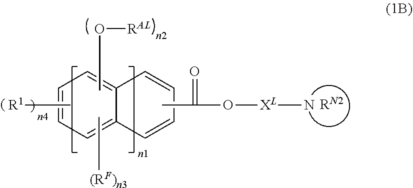
wherein R^{AL}, R^{F}, R¹, X^{L} and n1 to n4 are as defined above,
the ring R^{N2} forms a C₃-C₂₀ alicyclic hydrocarbon group with the nitrogen atom in the formula, some constituent —CH₂— in the ring R^{N2} may be replaced by —O— or —S—.

5. A chemically amplified resist composition comprising a quencher in the form of the amine compound of claim 1.

6. The resist composition of claim 5, further comprising a base polymer comprising repeat units having the formula (a1) or (a2): 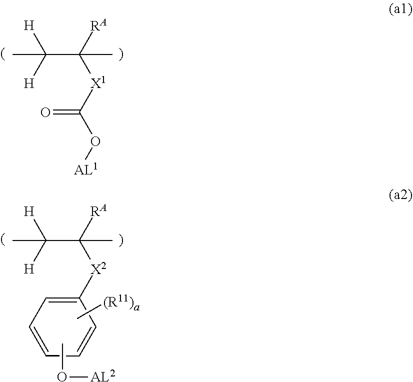
wherein R^{A} is each independently hydrogen, fluorine, methyl or trifluoromethyl,
X¹ is a single bond, phenylene, naphthylene, or *—C(═O)—O—X¹¹—, the phenylene or naphthylene group may be substituted with an optionally fluorinated C₁-C₁₀ alkoxy moiety or halogen, X¹¹ is a C₁-C₁₀ saturated hydrocarbylene group which may contain a hydroxy moiety, ether bond, ester bond or lactone ring, or phenylene group or naphthylene group,
X² is a single bond or *—C(═O)—O—,
the asterisk (*) designates a point of attachment to the carbon atom in the backbone,
R¹¹ is a C₁-C₂₀ hydrocarbyl group which may contain a heteroatom,
AL¹ and AL² are each independently an acid labile group, and
a is an integer of 0 to 4.

7. The resist composition of claim 6 wherein the base polymer further comprises repeat units having the formula (b1) or (b2): 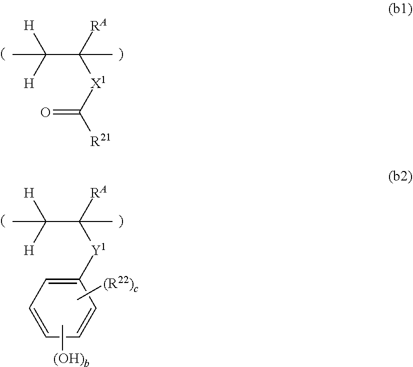
wherein R^{A} is each independently hydrogen, fluorine, methyl or trifluoromethyl,
Y¹ is a single bond or *—C(═O)—O—, the asterisk (*) designates a point of attachment to the carbon atom in the backbone,
R²¹ is hydrogen or a C₁-C₂₀ group containing at least one structure selected from a hydroxy moiety other than phenolic hydroxy, cyano moiety, carbonyl moiety, carboxy moiety, ether bond, ester bond, sulfonic ester bond, carbonate bond, lactone ring, sultone ring, and carboxylic anhydride (—C(═O)—O—C(═O)—),
R²² is a C₁-C₂₀ hydrocarbyl group which may contain a heteroatom,
b is an integer of 1 to 4, c is an integer of 0 to 4, and 1≤b+c≤5.

8. The resist composition of claim 6 wherein the base polymer further comprises repeat units of at least one selected from repeat units having the formulae (c1) to (c4): 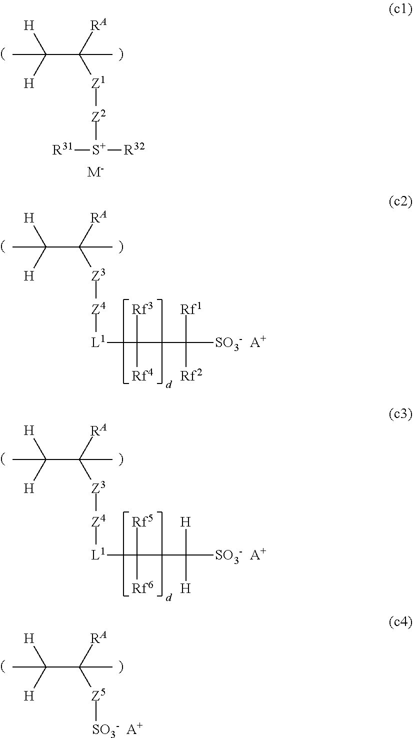
wherein R^{A} is each independently hydrogen, fluorine, methyl or trifluoromethyl,
Z¹ is a single bond or phenylene group,
Z² is *—C(═O)—O—Z²¹—, *—C(═O)—NH—Z²¹— or *—O—Z²¹—, Z²¹ is a C₁-C₆ aliphatic hydrocarbylene group, phenylene group or a divalent group obtained by combining the foregoing, which may contain a carbonyl moiety, ester bond, ether bond or hydroxy moiety,
Z³ is a single bond, phenylene group, naphthylene group or *—C(═O)—O—Z³¹—, Z³¹ is a C₁-C₁₀ aliphatic hydrocarbylene group which may contain a hydroxy moiety, ether bond, ester bond or lactone ring, phenylene group or naphthylene group,
Z⁴ is a single bond or **—Z⁴¹—C(═O)—O—, Z⁴¹ is a C₁-C₂₀ hydrocarbylene group which may contain a heteroatom,
Z⁵ is a single bond, methylene group, ethylene group, phenylene group, fluorinated phenylene group, trifluoromethyl-substituted phenylene group, *—C(═O)—O—Z⁵¹—, *—C(═O)—N(H)—Z⁵¹—, or *—O—Z⁵¹—, Z⁵¹ is a C₁-C₆ aliphatic hydrocarbylene group, phenylene group, fluorinated phenylene group, trifluoromethyl-substituted phenylene group, which may contain a carbonyl moiety, ester bond, ether bond or hydroxy moiety,
* designates a point of attachment to the carbon atom in the backbone, ** designates a point of attachment to Z³,
R³¹ and R³² are each independently a C₁-C₂₀ hydrocarbyl group which may contain a heteroatom, R³¹ and R³² may bond together to form a ring with the sulfur atom to which they are attached,
L¹ is a single bond, ether bond, ester bond, carbonyl group, sulfonic ester bond,
carbonate bond or carbamate bond,
Rf¹ and Rf² are each independently fluorine or a C₁-C₆ fluorinated saturated hydrocarbyl group,
Rf³ and Rf⁴ are each independently hydrogen, fluorine or a C₁-C₆ fluorinated saturated hydrocarbyl group,
Rf⁵ and Rf⁶ are each independently hydrogen, fluorine or a C₁-C₆ fluorinated saturated hydrocarbyl group, excluding that all Rf⁵ and Rf⁶ are hydrogen at the same time,
M⁻ is a non-nucleophilic counter ion,
A⁺ is an onium cation, and
d is an integer of 0 to 3.

9. The resist composition of claim 5, further comprising an organic solvent.

10. The resist composition of claim 5, further comprising a photoacid generator.

11. The resist composition of claim 5, further comprising a quencher other than the amine compound having formula (1).

12. The resist composition of claim 5, further comprising a surfactant.

13. A pattern forming process comprising the steps of applying the chemically amplified resist composition of claim 5 onto a substrate to form a resist film thereon, exposing the resist film to high-energy radiation, and developing the exposed resist film in a developer.

14. The process of claim 13 wherein the high-energy radiation is KrF excimer laser radiation, ArF excimer laser radiation, EB or EUV of wavelength 3 to 15 nm.
